(19) United States
(12) Patent Application Publication
(10) Pub. No.: US 2008/0050719 A1
(43) Pub. Date: Feb. 28, 2008

(54) Gene expression profiles in liver disease

(76) Inventors: Sang Seok Koh, Daejeon (KR); Qing Liu, Gaithersburg, MD (US); Hyun-Ho Chung, Gaithersburg, MD (US); Weu Zeng, Gaithersburg, MD (US); Bogman Lee, Daejeon (KR); Subrahmanyam Yeramilli, Gaithersburg, MD (US); Si Young Song, Daejeons (KR)

Correspondence Address: MORGAN LEWIS & BOCKIUS LLP, 1111 PENNSYLVANIA AVENUE NW, WASHINGTON, DC, 20004, US

(21) Appl. No.: 10/499,695

(22) PCT Filed: Dec. 20, 2002
(86) PCT No.: PCT/US02/40718

§ 371 Date: Feb. 24, 2005

Related U.S. Application Data
(60) Provisional application No. 60/343,185, filed on Dec. 31, 2001.
(60) Provisional application No. 60/341,815, filed on Dec. 21, 2001.

(51) Int. Cl. C12Q 1/68; G01N 33/574; A61K 38/17; A61K 48/00; G06F 19/00; C12M 1/34

(52) U.S. Cl. 435/6; 435/7.23; 435/287.2; 702/20; 514/12; 514/44; 977/924

(57) ABSTRACT

The present invention results from the examination of tissue from hepatic carcinomas to identify genes differentially expressed between cancerous liver tissue and diseased but non-cancerous liver tissue. The invention includes diagnostic, screening, drug design and therapeutic methods using these genes, as well as solid supports comprising oligonucleotide arrays that are complementary to or hybridize to the differentially expressed genes.

### RELATED APPLICATIONS

This application claims the benefit of U.S. Provisional Applications 60/341,815 and 60/343,185, both of which are herein incorporated by reference in their entirety.

### FIELD OF THE INVENTION

The invention relates generally to the changes in gene expression in liver tissue from patients with hepatic carcinomas. The invention specifically relates to a set of human genes that are differentially expressed in cancerous liver tissue compared to diseased, but non-cancerous liver tissue.

### BACKGROUND OF THE INVENTION

### Liver Disease

Generally, liver disease is classified as a disorder that causes the liver to malfunction or cease functioning all together. Cirrhosis, for example, is a group of chronic liver diseases in which liver cells are damaged and then replaced with scar tissue, thereby decreasing the amount of normal liver tissue. While it is most often caused by alcohol abuse, patients with hepatitis infections and other biliary diseases can also develop cirrhosis. Chronic hepatitis-B infection, hepatitis-C infection, and cirrhosis have all been shown to have strong associations with primary liver cancer, although the mechanisms involved are still not fully understood (Wu et al. (2001), Oncogene, 20: 3674-3682). About 10-20% chronic hepatitis-B infections result in primary liver cancer. Other factors such as alcohol consumption, poor nutrition and aflatoxins (carcinogens produced by molds, which are found in spoiled foods such as peanuts, corn, grains and seeds) are also linked to the development of primary liver cancer and cirrhosis.

In primary liver cancer, liver cells become abnormal, grow out of control and form malignant tumors. This disease is also called hepatocellular carcinoma (HCC) or malignant hepatoma. Cancer that spreads to the liver from another part of the body as a result of metastasis is not the same disease. HCC is difficult to detect at an early stage because the symptoms are not specific. They include loss of appetite and weight, fever, fatigue and weakness. As the cancer progresses, pain may develop in the upper abdomen, extending to the back and right shoulder. Swelling or a palpable mass may also be present in the upper abdomen, along with jaundice and darkened urine. When the cancer metastasizes, it typically targets the lungs and brain.

Diagnosis of HCC may be made by blood tests, in particular, tests for tumor markers such as alpha-fetoprotein. About 50-70% of HCC patients show elevated levels of alpha-fetoprotein. Additional diagnostic methods include non-radioactive imaging (abdominal or chest x-rays, angiograms, CT scans and MRIs), liver scans using radioactive materials and liver biopsies. Treatment of HCC is often not successful, because detection is often too late, but methods include surgical removal of the cancer, chemotherapy and radiation, alone or in combination. Although HCC is not very common in the United States, it is very prevalent in parts of Asia and Africa, largely due to the higher incidence of infection with hepatitis viruses (http://cis.nci.nih.gov/; http://cancer.med.upenn.edu/disease/liver/intro_liver.html).

The number of new cases of acute and chronic viral hepatitis has been estimated at approximately 200,000 per year in the United States. The viruses that commonly cause hepatitis are hepatitis A, hepatitis B (which is also oncogenic), hepatitis D (or delta hepatitis, a “defective” RNA virus that is infective only in the presence of hepatitis B virus), hepatitis C, hepatitis E (or epidemic non-A non-B hepatitis), hepatitis F and G (epidemic non-A non-B non-C variants which may be mutants of hepatitis B, but which do not express the B antigens), cytomegalovirus, Epstein-Barr virus and herpes simplex virus. The last three are prominent in patients receiving immunosuppressive treatments following liver, kidney or bone marrow transplants. Hepatitis viruses B, C and D are typically associated with chronic viral hepatitis.

Among hepatitis viruses, hepatitis B is associated with the greatest mortality. This virus is a double-shelled DNA virus with an endogenous DNA polymerase and a single, circular molecule of DNA 3200 base pairs in length. The virus replicates via an RNA intermediate requiring reverse transcriptase. In patients infected with hepatitis B, three types of particles can be detected in the serum, 20 nm spheres, tubules 20 nm in diameter and 100 nm in length, and complex, 42 nm Dane particles. Similar to the human hepatitis B virus are hepatitis B viruses found in ducks, herons, squirrels and woodchucks.

Diagnosis of hepatitis B is usually made by finding the surface antigen (HBsAg) in serum. The presence of HBsAg for six months or more signifies a carrier state or chronic infection. Anti-HBs (HBsAb) accounts for recovery and immunity.

Although the core antigen (HBcAg) is not detectable in the blood, the antibody can be detected. In cases of acute hepatitis, an IgM antibody to the core antigen (HBcAg) is found. But, if this antibody persists, it is an indication of chronic viral hepatitis. Another indication of chronic infection is a high level of IgG HBcAb, without HBsAb, but with HBsAg.

HBeAg correlates with active viral synthesis and infectivity. Appearance of the antibody (HBeAb) is a sign of reduced infectivity and that the patient will recover.

Only about 2-8% of adults infected with hepatitis B develop chronic infection, but about 90% of infected neonates become carriers. Chronic hepatitis B infection is associated with cirrhosis, as well as with liver carcinomas, as about 25% of these patients eventually develop cirrhosis. Although there is no universally effective treatment for either chronic or acute hepatitis B infection, current treatment methods involve administration of interferon alpha-2a, at dosages of, e.g., 10 million units 3 times a week for 16 weeks, or 5 million units daily for 4 months.

Hepatitis C virus is considered to be the major cause of post-transfusion hepatitis. Another important source of infection is intravenous drug use. This virus is classified in the togavirus family of lipid envelope viruses, producing particles of 30-60 nm. It is a single-stranded RNA virus with a genome of about 10.5 kb. About 50% of patients infected with hepatitis C develop chronic infections, and about 20% of patients chronically infected develop cirrhosis. As mentioned above, it has been noted that many hepatitis C patients go on to develop liver cancer, but the percentage has not yet been established. Efforts at treating hepatitis C have been frustrated by the results that current antivirals, including alpha interferon, have not been very effective (http://www.arens.com/brian/viral.htm).

Cirrhosis of the liver, typically caused by toxins, inflammation or metabolic disorders, is characterized by widespread nodules combined with fibrosis. Damaged or dead liver cells are replaced by fibrous scar tissue, which to leads to fibrosis. Liver cells regenerate in an abnormal pattern, producing nodules surrounded by fibrous tissue. The fibrosis and nodule formation cause distortion and blockage of the liver's structural components, causing impaired blood flow and biochemical function.

In the circulatory system, blood from the intestines and spleen flows to the liver via the portal vein, before returning to the heart via the hepatic vein. Blood also flows directly to the liver from the hepatic artery. In the esophagus, stomach, small intestine and rectum, the body's systemic circulation is connected to the liver's portal circulation, and, under normal conditions, there is no backflow from the portal circulation into the systemic circulation. In cirrhosis, however, the fibrous scar tissue decreases blood flow to and through the liver. Blood then backs up in the portal vein and portal circulation, causing complications in other organs, such as enlargement of the spleen with sequestered blood cells, reduced platelet count and abnormal bleeding. Backflow of blood into the systemic circulation can cause varicose veins in the esophagus, stomach and rectum, which can rupture and bleed profusely. Hypertension in the portal circulation can also produce fluid accumulation in the abdomen (ascites) and surrounding tissue (peripheral edema), while decreased bilirubin secretion can lead to elevated levels of bilirubin in the blood and jaundice. Abnormal biochemical changes due to cirrhosis include decreased levels of albumin (which aggravates the ascites and edema), decreased levels of clotting factors, gynecomastia in men (impaired estrogen metabolism), and decreased metabolism of sugars, triglycerides and cholesterol. In advanced stages of cirrhosis, abnormalities in the brain can occur, because toxic substances normally removed by the liver flow to the brain. Changes include decreased mental function (concentration and cognitive abilities), stupor, coma, brain swelling and death.

In patients displaying some of the above symptoms, diagnosis of cirrhosis is usually easy, but cirrhosis may be difficult to detect in its early stages. Subtle changes occurring in the early stages include red palms, red spots on the upper body that blanch, hypertrophy of the parotid glands, fibrosis of the tendons in the palms and gynecomastia. X-rays and radioactive tracer tests may be effective, but diagnosis must often be by liver biopsy.

The structural damage to the liver is irreversible, although the underlying causes may be treated to stop progression of the disease (alcoholism in particular). The sequelae of the disease may also be treated (such varicose veins, ascites and edema). In alcoholics who have stopped drinking for extended periods of time, liver transplants have been successful (http://cpmcnet.columbia.edu/dept/gi/cirrhosis.html).

### Molecular Changes in Liver Disease

Little is known about the molecular changes in liver cells associated with the development and progression of liver disease. Accordingly, there exists a need for the investigation of the changes in global gene expression levels as well as the need for the identification of new molecular markers associated with the development and progression of liver disease. Furthermore, if intervention is expected to be successful in halting or slowing down liver disease, means of accurately assessing the early manifestations of liver disease need to be established. One way to accurately assess the early manifestations of liver disease is to identify markers which are uniquely associated with disease progression (see for example Kim et al. (2001) Oncogene 20: 4568-4575). Likewise, the development of therapeutics to prevent or stop the progression of liver disease relies on the identification of genes responsible for the cancerous transformation of liver cells and the growth of cancerous liver cells.

To date, researchers have been able to identify a few genetic alterations believed to underlie tumor development. These genetic alterations include amplification of oncogenes and mutations that result in the loss of tumor suppressor genes. Tumor suppressor genes are genes that, in their wild-type alleles, express proteins that suppress abnormal cellular proliferation. When the gene coding for a tumor suppressor protein is mutated or deleted, the resulting mutant protein or the complete lack of tumor suppressor protein expression may fail to correctly regulate cellular proliferation, and abnormal proliferation may take place, particularly if there is already existing damage to the cellular regulatory mechanism. A number of well-studied human tumors and tumor cell lines have missing or non-functional tumor suppressor genes. Examples of tumor suppressor genes include, but are not limited to, the retinoblastoma susceptibility gene or RB gene, the p53 gene, the deletion in colon carcinoma (DCC) gene and the neurofibromatosis type 1 (NF-1) tumor suppressor gene (Weinberg, R. A. Science, 1991, 254:1138-1146). Loss of function or inactivation of tumor suppressor genes may play a central role in the initiation and/or progression of a significant number of human cancers.

Classification of heterogeneous populations of tumor types is a daunting task; yet, initial studies utilizing gene expression patterns to identify subtypes of cancer produced rather intriguing results (see Perou et al., Proc Natl Acad Sci USA 96:9212-9217, 1999; Golub et al., Science 286:531-537, 1999; Alizadeh et al., Nature 403:503-511, 2000; Alon et al. Proc Natl Acad Sci USA 96:6745-6750, 1999; and Bittner et al., Nature 406:536-540, 2000). Molecular classification of B-cell lymphoma by gene expression profiling elucidated clinically distinct diffuse large-B-cell lymphoma subgroups (see Alizadeh supra). Stratification of patients based on their distinctive gene expression profiles may allow researchers to precisely group similar patient populations for evaluating chemotherapeutic agents. The more homogenous population of patients decreases the variability of patient-to-patient responses leading to the development of agents capable of eradicating specific subtypes of cancers previously unknown using standard classification techniques.

The utilization of gene expression profiles to classify tumors, to identify drug targets, to identify diagnostic markers and/or to gain further insights into the consequences of chemotherapeutic treatments could facilitate the design of more efficacious patient-specific stratagems for treating a variety of cancers. In breast cancer, studies utilizing limited numbers of genes (8,102 genes) have classified tumors into subtypes based on gene expression profiles, and this study indicated a diversity of molecular phenotypes associated with breast tumors (Perou et al., Nature 406:747-752, 2000). The advent of cDNA and oligonucleotide arrays has enabled researchers to map tissue-specific expression levels for thousands of genes (Alon et al., Proc Natl Acad Sci USA 96:6745-6750, 1999; Iyer et al., Science 283:83-87, 1999; Khan et al., Cancer Res 58:5009-5013, 1998; Lee et al., Science 285:1390-1393, 1999; Wang et al., Gene 229:101-108, 1999; Whitney et al., Ann Neurol 46:425-428, 1999). The study by Martin et al. (Cancer Res 60:2232-2238, 2000) used a custom microarray composed of 124 genes discovered by differential display associated with either normal breast epithelial cells or from the MDA-MB-435 malignant breast tumor cell line. Using the custom microarray, researchers examined the relationship between expression patterns discovered by clustering a number of genes with clinical stages of breast cancer indicating that gene expression patterns were capable of grouping breast tumors into distinct categories (Martin et al., supra).

Although these studies have demonstrated that expression profiling may be used to produce improvements in diagnosis of human diseases such as cancer, as well as in the development of improved therapeutic strategies, further studies are needed. Accordingly, there remains a need in the art for materials and methods that permit a more accurate diagnosis of hepatic carcinomas, as well as of other chronic liver diseases. In addition, there remains a need in the art for methods to treat and methods to identify agents that can effectively treat liver disease. The present invention meets these and other needs.

### SUMMARY OF THE INVENTION

The present invention is based on the discovery of the genes and their expression profiles associated with various types and stages of liver disease, in particular hepatocellular carcinoma (HCC), chronic hepatitis (CH) and liver cirrhosis (LC).

The invention includes methods of diagnosing liver disease in a patient comprising the step of detecting the level of expression in a tissue sample of one or more genes from Table 1; wherein differential expression of the genes in Table 1 is indicative of liver disease. The invention also includes methods of detecting the progression of liver disease. For instance, methods of the invention include detecting the progression of liver disease in a patient comprising the step of detecting the level of expression in a tissue sample of one or more genes from Table 1; wherein differential expression of the genes in Table 1 is indicative of liver disease progression. In some preferred embodiments, PCA analysis based on all or a portion of the group of genes identified in Table 1 may be used to differentiate between the different stages of liver disease, such as in the metastasis of liver carcinomas. In some preferred embodiments, one or more genes may be selected from Table 1.

In some aspects, the present invention provides a method of monitoring the treatment of a patient with liver disease, comprising administering a pharmaceutical composition to the patient, preparing a gene expression profile from a cell or tissue sample from the patient and comparing the patient gene expression profile to a gene expression from a cell population comprising normal liver cells, or to a gene expression profile from a cell population comprising disease state liver cells, or to both. In some preferred embodiments, the gene profile will include the expression level of one or more genes in Table 1.

Another aspect of the present invention includes a method of treating a patient with liver disease, comprising administering to the patient a pharmaceutical composition, wherein the composition alters the expression of at least one gene in Table 1, preparing a gene expression profile from a cell or tissue sample from the patient comprising diseased cells and comparing the patient expression profile to a gene expression profile from an untreated cell population comprising disease state liver cells.

In another aspect, the present invention provides a method of detecting the progression of carcinogenesis in a patient, comprising detecting the level of expression in a tissue sample of one or more genes from Table 1; wherein differential expression of the genes in Table 1 is indicative of hepatic carcinogenesis.

The invention further includes methods of screening for an agent capable of modulating the onset or progression of liver disease, comprising the steps of exposing a cell to the agent; and detecting the expression level of one or more genes from Table 1. In some embodiments, the liver disease may be a hepatocellular carcinoma. In some preferred embodiments, one or more genes may be selected from a group consisting of those listed in Table 1. In some preferred methods, it may be desirable to detect all or nearly all of the genes in the tables.

The invention further includes compositions comprising at least two oligonucleotides, wherein each of the oligonucleotides comprises a sequence that specifically hybridizes to a gene in Table 1, as well as solid supports comprising at least two probes, wherein each of the probes comprises a sequence that specifically hybridizes to a gene in Table 1. In some preferred embodiments, one or more genes may be selected from a group consisting of those listed in Table 1.

The invention further includes computer systems comprising a database containing information identifying the expression level in liver tissue of a set of genes comprising at least two genes in Table 1 and a user interface to view the information. In some preferred embodiments, one or more genes may be selected from a group consisting of those listed in Table 1. The database may further include sequence information for the genes, information identifying the expression level for the set of genes in non-cancerous liver tissue and in cancerous liver tissue and may contain links to external databases such as GenBank.

Lastly, the invention includes methods of using the databases, such as methods of using the disclosed computer systems to present information identifying the expression level in a tissue or cell of at least one gene in Table 1, comprising the step of comparing the expression level of at least one gene in Table 1 in the tissue or cell to the level of expression of the gene in the database. In some preferred embodiments, one or more genes may be selected from a group consisting of those listed in Table 1.

### DETAILED DESCRIPTION OF THE PREFERRED EMBODIMENTS

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall, Cell 64:313-326, 1991; Weinberg, Science, 254:1138-1146, 1991). Thus, changes in the expression levels of particular genes (e.g., oncogenes or tumor suppressors) serve as signposts for the presence and progression of various diseases.

Monitoring changes in gene expression may also provide certain advantages during drug screening and development. Often drugs are pre-screened for the ability to interact with a major target without regard to other effects the drugs have on cells. Often such other effects cause toxicity in the whole animal, which prevent the development and use of the potential drug.

Using pairs of samples from subjects, applicants have examined samples from diseased but non-cancerous liver tissue and from cancerous liver tissue to identify global changes in gene expression between tumor biopsies and surrounding non-cancerous tissue. Diseased but non-cancerous liver tissue was either inflamed tissue from chronic viral hepatitis patients or fibrotic tissue from liver cirrhosis patients. Non-cancerous tissue was removed from a point in the liver adjacent to a tumor biopsy site. These global changes in gene expression, also referred to as expression profiles, provide useful markers for diagnostic uses as well as markers that can be used to monitor disease states, disease progression, drug toxicity, drug efficacy and drug metabolism.

The gene expression profiles described herein were derived from diseased liver biopsy samples from Korean patients 34-65 years old. These patients had been diagnosed with chronic hepatitis or cirrhosis and, in each case, had subsequently developed liver cancer. The disease state associated with each sample is indicated in Table 2.

The present invention provides compositions and methods to detect the level of expression of genes that may be differentially expressed dependent upon the state of the cell, i.e., non-cancerous versus cancerous. These expression profiles of genes provide molecular tools for evaluating toxicity, drug efficacy, drug metabolism, development, and disease monitoring. Changes in the expression profile from a baseline profile can be used as an indication of such effects. Those skilled in the art can use any of a variety of known techniques to evaluate the expression of one or more of the genes and/or gene fragments identified in the instant application in order to observe changes in the expression profile in a tissue or sample of interest.

### Definitions

In the description that follows, numerous terms and phrases known to those skilled in the art are used. In the interest of clarity and consistency of interpretation, the definitions of certain terms and phrases are provided.

As used herein, the phrase “detecting the level of expression” includes methods that quantify expression levels as well as methods that determine whether a gene of interest is expressed at all. Thus, an assay which provides a yes or no result without necessarily providing quantification of an amount of expression is an assay that requires “detecting the level of expression” as that phrase is used herein.

As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity or more preferably about 90% or 95% or more nucleotide sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 or more different nucleic acid hybridizations.

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases that are not complementary to the corresponding bases of the target sequence.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

As used herein a “probe” is defined as a nucleic acid, preferably an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Homology or identity may be determined by BLAST (Basic Local Alignment Search Tool) analysis using the algorithm employed by the programs blastp, blastn, blastx, tblastn and tblastx (Karlin et al., Proc Natl Acad Sci USA 87:2264-2268, 1990 and Altschul, J Mol Evol 36:290-300, 1993, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genet 6:119-129, 1994) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc Natl Acad Sci USA 89:10915-10919, 1992, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

### Uses of Differentially Expressed Genes

The present invention identifies those genes differentially expressed between cancerous and non-cancerous liver tissue. One of skill in the art can select one or more of the genes identified as being differentially expressed in Table 1 and use the information and methods provided herein to interrogate or test a particular sample. For a particular interrogation of two conditions or sources, it may be desirable to select those genes which display a great deal of difference in the expression pattern between the two conditions or sources. In other instances, it may be appropriate to select genes whose expression changes only slightly between the two conditions. At least a 1.5-fold difference may be desirable, but a three-fold, five-fold or ten-fold difference may be preferred in some instances. The data are subjected to statistical evaluation to ensure that the observed differences and-the disease association are statistically significant. Interrogations of the genes or proteins can be performed to yield different information.

### Diagnostic Uses for the Liver Cancer Markers

As described herein, the genes and gene expression information provided in Table 1 may be used as diagnostic markers for the prediction or identification of a disease state of liver tissue. For instance, a liver tissue sample or other sample from a patient may be assayed by any of the methods known to those skilled in the art, and the expression levels from one or more genes from Table 1 may be compared to the expression levels found in non-cancerous liver tissue, cancerous liver tissue or both. Expression profiles generated from the tissue or other samples that substantially resemble an expression profile from non-cancerous or cancerous liver tissue may be used, for instance, to aid in disease diagnosis. Comparison of the expression data, as well as available sequence or other information, may be done by a researcher or diagnostician or may be done with the aid of a computer and databases as described herein.

### Use of the Liver Cancer Markers for Monitoring Disease Progression

Molecular expression markers for liver disease can be used to confirm the type and progression of disease made on the basis of morphological criteria. For example, non-cancerous liver tissue could be distinguished from cancerous tissue based on the level and type of genes expressed in a tissue sample. In some situations, identifications of cell type or source is ambiguous based on classical criteria. In these situations, the molecular expression markers of the present invention are useful for identifying the region of the liver from which a sample came, as well as whether or not normal levels of gene expression have been altered (signs of metabolic disturbances).

In addition, progression of hepatic carcinoma to new areas of the liver can be monitored by following the expression patterns of the involved genes using the molecular expression markers of the present invention. Monitoring of the efficacy of certain drug regimens can also be accomplished by following the expression patterns of the molecular expression markers.

As described above, the genes and gene expression information provided in Table 1 may also be used as markers for the direct monitoring of disease progression, for instance, the development of liver cancer. A liver tissue sample or other sample from a patient may be assayed by any of the methods known to those of skill in the art, and the expression levels in the sample from a gene or genes from Table 1 may be compared to the expression levels found in non-cancerous liver tissue, tissue from a hepatic carcinoma or both. Comparison of the expression data, as well as available sequence or other information may be done by a researcher or diagnostician or may be done with the aid of a computer and databases as described herein.

### Use of the Liver Cancer Markers for Drug Screening

According to the present invention, potential drugs can be screened to determine if application of the drug alters the expression of one or more of the genes identified herein. This may be useful, for example, in determining whether a particular drug is effective in treating a particular patient with liver disease. In the case where a gene's expression is affected by the potential drug such that its level of expression returns to normal, the drug is indicated in the treatment of liver cancer. Similarly, a drug which causes expression of a gene which is not normally expressed by healthy liver cells may be contra-indicated in the treatment of liver cancer.

According to the present invention, the genes identified in Table 1 may also be used as markers to evaluate the effects of a candidate drug or agent on a cell, particularly a cell undergoing malignant transformation, for instance, a liver cancer cell or tissue sample. A candidate drug or agent can be screened for the ability to stimulate the transcription or expression of a given marker or markers (drug targets) or to down-regulate or inhibit the transcription or expression of a marker or markers. According to the present invention, one can also compare the specificity of a drug's effects by looking at the number of markers affected by the drug and comparing them to the number of markers affected by a different drug. A more specific drug will affect fewer transcriptional targets. Similar sets of markers identified for two drugs indicates a similarity of effects.

Assays to monitor the expression of a marker or markers as defined in Table 1 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agents action. Agents can be selected or designed by utilizing the peptide sequences that make up these sites. For example, a rationally selected peptide agent can be a peptide whose amino acid sequence is identical to or a derivative of any functional consensus site.

The agents of the present invention can be, as examples, peptides, small chemical molecules, vitamin derivatives, as well as carbohydrates, lipids, oligonucleotides and covalent and non-covalent combinations thereof. Dominant negative proteins, DNA encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see Grant, in Molecular Biology and Biotechnology, Meyers (ed.), VCH Publishers, 1995). A skilled artisan can readily recognize that there is no limit as to the structural nature of the agents of the present invention.

### Use of the Liver Cancer Markers as Therapeutic Agents

Agents that up- or down-regulate or modulate the expression of the nucleic acid molecules of Table 1, or at least one activity of a protein encoded by the nucleic acid molecules of Table 1, such as agonists or antagonists, may be used to modulate biological and pathologic processes associated with the function and activity of the proteins encoded by these nucleic acid molecules. The agents can be the nucleic acid molecules of Table 1 themselves, the encoded proteins, or portions of these molecules, such as all or part of the open reading frames of these nucleic acid molecules.

Anti-sense oligonucleotide molecules derived from the nucleic acid sequences of Table 1 may also be used to down-regulate the expression of one or more of the genes in Table 1 that are expressed at elevated levels in liver cancer, the use of antisense gene therapy being an example. Down-regulation of expression of one or more of the genes of Table 1 is accomplished by administering an effective amount of antisense oligonucleotides. These antisense molecules can be fashioned from the DNA sequences of these genes or sequences containing various mutations, deletions, insertions or spliced variants. Isolated RNA or DNA sequences derived from these genes may also be used therapeutically in gene therapy. These agents may be used to induce gene expression in liver cancers associated with an absence of or considerably decreased expression of one or more of the proteins encoded by genes in Table 1.

As used herein, a subject can be any mammal, so long as the mammal is in need of modulation of a pathological or biological process mediated by a gene of the invention. The term “mammal” is defined as an individual belonging to the class Mammalia. The invention is particularly useful in the treatment of human subjects.

Pathological processes refer to a category of biological processes which produce a deleterious effect. For example, expression of a gene of the invention may be associated with hyperplasia in the liver, in particular malignant hyperplasia. As used herein, an agent is said to modulate a pathological process when the agent reduces the degree or severity of the process. For instance, liver cancer may be prevented or disease progression modulated by the administration of agents which up- or down-regulate or modulate in some way the expression or at least one activity of a gene of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of a protein of the invention. While individual needs vary, determination of optimal ranges of effective amounts of each component is within the skill of the art. Typical dosages comprise 0.1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g. ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice. The compounds of this invention can be utilized in vivo, ordinarily in mammals, such as humans, rats, mice, dogs, cats, sheep, horses, cattle and pigs, or in vitro.

### Assay Formats

The genes identified as being differentially expressed in liver disease may be used in a variety of nucleic acid detection assays to detect or quantify the expression level of a gene or multiple genes in a given sample. For example, traditional Northern blotting, nuclease protection, RT-PCR and differential display methods may be used for detecting gene expression levels. In methods where small numbers of genes are assayed, such as 5-50 genes, high-throughput PCR may be used.

The protein products of the genes identified herein can also be assayed to determine the amount of expression. Methods for assaying for a protein include Western blot, immunoprecipitation and radioimmunoassay. In some methods, it is preferable to assay the mRNA as an indication of expression. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays of the invention are most efficiently designed with array or chip hybridization-based methods for detecting the expression of a large number of genes.

Any hybridization assay format may be used, including solution-based and solid support-based assay formats. A preferred solid support is a high density array also known as a DNA chip or a gene chip. One variation of the DNA chip contains hundreds of thousands of discrete microscopic channels that pass completely through it. Probe molecules are attached to the inner surface of these channels, and molecules from the samples to be tested flow throughout the channels, coming into close proximity with the probes for hybridization. In one assay format, gene chips containing probes to at least two genes from Table 1 may be used to directly monitor or detect changes in gene expression in the treated or exposed cell as described herein.

The genes of the present invention may be assayed in any convenient sample form. For example, samples may be assayed in the form mRNA or reverse transcribed mRNA. Samples may be cloned or not, and the samples or individual genes may be amplified or not. The cloning itself does not appear to bias the representation of genes within a population. However, it may be preferable to use polyA+ RNA as a source, as it can be used with less processing steps. In some embodiments, it may be preferable to assay the protein or peptide expressed by the gene.

The sequences of the expression marker genes of Table 1 are available in the public databases. Table 1 provides the Accession number, Sequence Number ID and name for each of the sequences. The sequences of the genes in GenBank are herein expressly incorporated by reference in their entirety (see www.ncbi.nim.nih.gov).

Additional assay formats may be used to monitor the ability of the agent to modulate the expression of a gene identified in Table 1. For instance, as described above, mRNA expression may be monitored directly by hybridization of probes to the nucleic acids of the invention. Cell lines are exposed to an agent to be tested under appropriate conditions and time and total RNA or mRNA is isolated by standard procedures such those disclosed in Sambrook et al., Molecular Cloning—A Laboratory Manual, Third ed., Cold Spring Harbor Laboratory Press, Cold Spring Harbor, N.Y., 2001. In some embodiments, it may be desirable to amplify one or more of the RNA molecules isolated prior to application of the RNA to the gene chip. Using techniques well known in the art, the RNA may be reverse transcribed and amplified in the form of DNA or may be reverse transcribed into DNA and the DNA used as a template for transcription to generate recombinant RNA. Any method that results in the production of a sufficient quantity of nucleic acid to be hybridized effectively to the gene chip may be used.

In another format, cell lines that contain reporter gene fusions between the open reading frame and/or the 3′ or 5′ regulatory regions of a gene in Table 1 and any assayable fusion partner may be prepared. Numerous assayable fusion partners are known and readily available including the firefly luciferase gene and the gene encoding chloramphenicol acetyltransferase (Alam et al., Anal Biochem 188:245-254, 1990). Cell lines containing the reporter gene fusions are then exposed to the agent to be tested under appropriate conditions and time. Differential expression of the reporter gene between samples exposed to the agent and control samples identifies agents which modulate the expression of the nucleic acid.

In another assay format, cells or cell lines are first identified which express one or more of the gene products of the invention physiologically. Cells and/or cell lines so identified would preferably comprise the necessary cellular machinery to ensure that the transcriptional and/or translational apparatus of the cells would faithfully mimic the response of normal or cancerous liver tissue to an exogenous agent. Such machinery would likely include appropriate surface transduction mechanisms and/or cytosolic factors. Such cell lines may be, but are not required to be, derived from liver tissue. The cells and/or cell lines may then be contacted with an agent and the expression of one or more of the genes of interest may then be assayed. The genes may be assayed at the mRNA level and/or at the protein level.

In some embodiments, such cells or cell lines may be transduced or transfected with an expression vehicle (e.g., a plasmid or viral vector) containing an expression construct comprising an operable 5′-promoter containing end of a gene of interest identified in Table 1 fused to one or more nucleic acid sequences encoding one or more antigenic fragments. The construct may comprise all or a portion of the coding sequence of the gene of interest which may be positioned 5′- or 3′-to a sequence encoding an antigenic fragment. The coding sequence of the gene of interest may be translated or un-translated after transcription of the gene fusion. At least one antigenic fragment may be translated. The antigenic fragments are selected so that the fragments are under the transcriptional control of the promoter of the gene of interest and are expressed in a fashion substantially similar to the expression pattern of the gene of interest. The antigenic fragments may be expressed as polypeptides whose molecular weight can be distinguished from the naturally occurring polypeptides.

In some embodiments, gene products of the invention may further comprise an immunologically distinct tag. Such a process is well known in the art (see Sambrook et al., supra). Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

Another embodiment of the present invention provides methods for identifying agents that modulate the levels, concentration or at least one activity of a protein(s) encoded by the genes in Table 1. Such methods or assays may utilize any means of monitoring or detecting the desired activity.

In one format, the relative amounts of a protein of the invention produced in a cell population that has been exposed to the agent to be tested may be compared to the amount produced in an unexposed control cell population. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

### Probe Design

Probes based on the sequences of the genes described herein may be prepared by any commonly available method. Oligonucleotide probes for assaying the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40, or 50 nucleotides will be desirable.

One of skill in the art will appreciate that an enormous number of array designs are suitable for the practice of this invention. The high density array will typically include a number of probes that specifically hybridize to the sequences of interest. See WO 99/32660 for methods of producing probes for a given gene or genes. In addition, in a preferred embodiment, the array will include one or more control probes.

High density array chips of the invention include “test probes.” Test probes may be oligonucleotides that range from about 5 to about 500 or about 5 to about 50 nucleotides, more preferably from about 10 to about 40 nucleotides and most preferably from about 15 to about 40 nucleotides in length. In other particularly preferred embodiments, the probes are about 20 or 25 nucleotides in length. In another preferred embodiment, test probes are double or single strand DNA sequences. DNA sequences may be isolated or cloned from natural sources or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to the β-actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe may have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (I(PM)-I(MM)) provides a good measure of the concentration of the hybridized material.

### Nucleic Acid Samples

As is apparent to one of ordinary skill in the art, nucleic acid samples used in the methods and assays of the invention may be prepared by any available method or process. Methods of isolating total mRNA are also well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen (ed.) Elsevier Press, New York, 1993. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it may be desirable to inhibit or destroy RNase present in homogenates before homogenates can be used.

Biological samples may be of any biological tissue or fluid or cells from any organism as well as cells raised in vitro, such as cell lines and tissue culture cells. Frequently the sample will be a “clinical sample” which is a sample derived from a patient. Typical clinical samples include, but are not limited to, liver tissue biopsy, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

### Solid Supports

Solid supports containing oligonucleotide probes for differentially expressed genes can be any solid or semisolid support material known to those skilled in the art. Suitable examples include, but are not limited to, membranes, filters, tissue culture dishes, polyvinyl chloride dishes, beads, test strips, silicon or glass based chips and the like. Suitable glass wafers and hybridization methods are widely available, for example, those disclosed by Beattie (WO 95/11755). Any solid surface to which oligonucleotides can be bound, either directly or indirectly, either covalently or non-covalently, can be used. In some embodiments, it may be desirable to attach some oligonucleotides covalently and others non-covalently to the same solid support.

A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be on the order of a square centimeter.

Oligonucleotide probe arrays for expression monitoring can be made and used according to any techniques known in the art (see for example, Lockhart et al., Nat Biotechnol 14:1675-1680, 1996; McGall et al., Proc Nat Acad Sci USA 93: 13555-13460, 1996). Such probe arrays may contain at least two or more oligonucleotides that are complementary to or hybridize to two or more of the genes described herein. Such arrays may also contain oligonucleotides that are complementary or hybridize to at least 3, 4, 5, 6, 7, 8, 9, 10, 20, 30, 50, 70 or more the genes described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al., (1992) U.S. Pat. No. 5,143,854; Fodor et al., (1998) U.S. Pat. No. 5,800,992; Chee et al., (1998) U.S. Pat. No. 5,837,832).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al. WO 93/09668. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

### Hybridization

Nucleic acid hybridization simply involves contacting a probe and target nucleic acid under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low, stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

### Signal Detection

The hybridized nucleic acids are typically detected by detecting one or more labels attached to the sample nucleic acids. The labels may be incorporated by any of a number of means well known to those of skill in the art (see Lockhart et al., (1999) WO 99/32660).

### Databases

The present invention includes relational databases containing sequence information, for instance for one or more of the genes of Table 1, as well as gene expression information in various liver tissue samples. Databases may also contain information associated with a given sequence or tissue sample such as descriptive information about the gene associated with the sequence information, descriptive information concerning the clinical status of the tissue sample, or information concerning the patient from which the sample was derived. The database may be designed to include different parts, for instance a sequence database and a gene expression database. The databases of the invention may be stored on any available computer-readable medium. Methods for the configuration and construction of such databases are widely available, for instance, see Akerblom et al., (U.S. Pat. No. 5,953,727), which is specifically incorporated herein by reference in its entirety.

The databases of the invention may be linked to an outside or external database. In a preferred embodiment, as described in Table 1, the external database is GenBank and the associated databases maintained by the National Center for Biotechnology Information or NCBI (http://www.ncbi.nlm.nih.gov/Entrez/). Other external databases that may be used in the invention include those provided by Chemical Abstracts Service (http://stnweb.cas.org/) and Incyte Genomics (http://www.incyte.com/sequence/index.shtml).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northern blots (E-Northerns) to allow the user to determine the cell type or tissue in which a given gene is expressed and to allow determination of the abundance or expression level of a given gene in a particular tissue or cell. The E-northern analysis can be used as a tool to discover tissue specific candidate therapeutic targets that are not over-expressed in tissues such as the liver, kidney, or heart. These tissue types often lead to detrimental side effects once drugs are developed and a first-pass screen to eliminate these targets early in the target discovery and validation process would be beneficial.

The databases of the invention may also be used to present information identifying the expression level in a tissue or cell of a set of genes comprising at least one gene in Table 1, comprising the step of comparing the expression level of at least one gene in Table 1 in the tissue to the level of expression of the gene in the database. Such methods may be used to predict the physiological state of a given tissue by comparing the level of expression of a gene or genes in Table 1 from a sample to the expression levels found in normal liver tissue, tissue from liver carcinomas or both. Such methods may also be used in the drug or agent screening assays as described herein.

Without further description, it is believed that one of ordinary skill in the art can, using the preceding description and the following illustrative examples, make and utilize the compounds of the present invention and practice the claimed methods. The preceding working examples therefore, are illustrative only and should not be construed as limiting in any way the scope of the invention.

### EXAMPLES

### Example 1

### Preparation of Liver Disease Profiles

### Tissue Sample Acquisition and Preparation

The patient tissue samples were derived from ten Korean patients, aged 34 to 65, and classified into two groups of five patients each. Each group contained samples from four men and one woman. One group of consisted of patients who had been diagnosed with chronic viral hepatitis B and who later developed hepatic carcinomas. The second group of patients had been diagnosed with cirrhosis of the liver. These people also later developed hepatic carcinomas. For each patient, tissue was obtained from two areas of the liver to produce a set of biopsy samples. In the first patient group (cancer/hepatitis), samples were removed from liver tumors and from the non-cancerous surrounding area composed of inflamed tissue (inflammation due to hepatitis). In the second group (cancer/cirrhosis), liver tissue was removed from tumors and from the non-cancerous surrounding area composed of fibrotic tissue (areas of fibrosis due to cirrhosis).

Histological analysis of each of the tissue samples was performed and samples were segregated into either non-cancerous or cancerous categories.

With minor modifications, the sample preparation protocol followed the Affymetrix GeneChip Expression Analysis Manual. Frozen tissue was first ground to powder using the Spex Certiprep 6800 Freezer Mill. Total RNA was then extracted using Trizol (Life Technologies). The total RNA yield for each sample (average tissue weight of 300 mg) was 200-500 μg. Next, mRNA was isolated using the Oligotex mRNA Midi kit (Qiagen). Since the mRNA was eluted in a final volume of 400 μl, an ethanol precipitation step was required to bring the concentration to 1 μg/μl. Using 1-5 μg of mRNA, double stranded cDNA was created using the SuperScript Choice system (Gibco-BRL). First strand cDNA synthesis was primed with a T7-(dT₂₄) oligonucleotide. The cDNA was then phenol-chloroform extracted and ethanol precipitated to a final concentration of 1 μg/μl.

From 2 μg of cDNA, cRNA was synthesized according to standard procedures. To biotin label the cRNA, nucleotides Bio-11-CTP and Bio-16-UTP (Enzo Diagnostics) were added to the reaction. After a 37° C. incubation for six hours, the labeled cRNA was cleaned up according to the Rneasy Mini kit protocol (Qiagen). The cRNA was then fragmented (5× fragmentation buffer: 200 mM Tris-Acetate (pH 8.1), 500 mM KOAc, 150 mM MgOAc) for thirty-five minutes at 94° C.

55 μg of fragmented cRNA was hybridized on the human and the Human Genome U95 set of arrays for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Following hybridization and scanning, the microarray images were analyzed for quality control, looking for major chip defects or abnormalities in hybridization signal. After all chips passed QC, the data was analyzed using Affymetrix GeneChip software (v3.0), and Experimental Data Mining Tool (EDMT) software (v1.0).

### Gene Expression Analysis

All samples were prepared as described and hybridized onto the Affymetrix Human Genome U95 array. Each chip contains 16-20 oligonucleotide probe pairs per gene or cDNA clone. These probe pairs include perfectly matched sets and mismatched sets, both of which are necessary for the calculation of the average difference. The average difference is a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. Using the average difference value that has been calculated, an absolute call for each gene is made.

The absolute call of present, absent or marginal is used to generate a Gene Signature, a tool used to identify those genes that are commonly present or commonly absent in a given sample set, according to the absolute call.

The Gene Signature Curve is a graphic view of the number of genes consistently present in a given set of samples as the sample size increases, taking into account the genes commonly expressed among a particular set of samples, and discounting those genes whose expression is variable among those samples. The curve is also indicative of the number of samples necessary to generate an accurate Gene Signature. As the sample number increases, the number of genes common to the sample set decreases. The curve is generated using the positive Gene Signatures of the samples in question, determined by adding one sample at a time to the Gene Signature, beginning with the sample with the smallest number of present genes and adding samples in ascending order. The curve displays the sample size required for the most consistency and the least amount of expression variability from sample to sample. The point where this curve begins to level off represents the minimum number of samples required for the Gene Signature. Graphed on the x-axis is the number of samples in the set, and on the y-axis is the number of genes in the positive Gene Signature. As a general rule, the acceptable percent of variability in the number of positive genes between two sample sets should be less than 5%.

For the purposes of this study, the following statistical methods were used for the data analysis. A gene set consists of genes that have a certain percentage of present calls in at least one group of samples. These genes are analyzed, and others are excluded. For example, a gene having 40% present calls (2 out of 5 samples) in at least in one sample group, cancerous cells from either hepatitis or cirrhosis patients, or non-cancerous cells from either type of patient, is included in the analysis if 40% is above the lower limit for percent present calls. Also, the genes are divided into two groups depending on their expression values across samples. For the genes in the high expression group, the average difference value is transformed to log scale before the analysis. For the genes in the low expression group, the original values are used in the analysis. An Analysis of Variance (ANOVA) method is used for data analysis (Steel et al., Principles and Procedures of Statistics: A Biometrical Approach, Third Ed., McGraw-Hill, 1997). Prior to the final analysis, a leave-one-out approach is used for outlier detection. One sample is left out of the ANOVA analysis to see whether omitting a specific sample from the analysis has any significant effect on the final result. If so, that particular sample is excluded from the final analysis. After outlier detection, the final analysis produces a list of genes that are differentially expressed with a p-value ≦0.001 as determined by the contrast from the ANOVA.

Differentially expressed genes were discovered by comparing biopsy samples from cancerous and non-cancerous regions of the same liver in patients with chronic viral hepatitis (CH) or liver cirrhosis (LC) who went on to develop primary liver cancer (hepatocellular carcinoma or HCC). Genes which showed no difference in expression level between a the cancerous and non-cancerous samples were not included in Table 1. Group 1 of Table 1 (23 genes) lists the genes that were found to be differentially expressed when the level in liver tumor cells was compared to the level in non-cancerous cells from inflamed areas or from fibrotic areas. Group 2 (12 genes) lists the genes whose expression level differed in liver tumor cells compared to cells from areas of inflammation, and group 3 contains those genes whose expression level differed in liver tumor cells compared to cells from fibrotic regions of the liver (74 genes).

### Fold Change Analysis

The data was first filtered to exclude all genes that showed no expression in any of the samples. The ratio (cancerous/non-cancerous, HCC/CH or HCC/LC) was calculated by comparing the mean expression value for each gene in the cancerous sample set against the mean expression value of that gene in the non-cancerous sample set. Genes were included in the analysis if they had a fold change ≧1.5 in either direction, and a p-value <0.0007 as determined by an Analysis of Variance Test (ANOVA). According to the criteria of the test, differences having p-values below 0.0007 were determined to be statistically significant. Out of the ˜60,000 genes surveyed by the Human Genome U95 set, 109 genes were present in the overall fold change analysis. In Table 1, numbers representing a comparison, or fold change, between the level of expression of a gene in two disease state liver biopsy samples can be positive or negative. Positive values indicate a higher expression level in the cancerous sample compared to the non-cancerous sample (up-regulation), while negative values indicate a lower expression level in the cancerous sample compared to the non-cancerous sample (down-regulation).

### Expression Profiles of Genes Differentially Expressed in Liver Disease

Using the above described methods, genes that were predominantly over-expressed in liver cancer, or predominantly under-expressed in liver cancer, were identified. Genes with consistent differential expression patterns provide potential targets for broad range diagnostics and therapeutics.

Table 1 lists the genes determined to be differentially expressed in cancerous liver tissue compared to non-cancerous liver tissue, with the fold change value for each gene. More specifically, the level of expression of the genes of Table 1 in liver cancer cells was compared to the level of expression in tissue from inflamed and/or fibrotic areas of the liver. The set of genes in each group, along with their relative expression levels, creates a profile for the diseases examined, chronic hepatitis with hepatic carcinoma and cirrhosis with hepatic carcinoma.

These genes or subsets of these genes confirm an overall liver disease gene expression profile. The genes in Table 1 may be used alone, or in combination with the methods, compositions, databases and computer systems of the invention.

### Example 2

### Diagnostic Subset of Liver Disease Associated GeneCluster Analysis

Table 1 lists the members of diagnostic subsets of genes selected by p-value in groups 2 and 3 (12 and 74 genes, respectively). In addition to their diagnostic, monitoring, drug screening and therapeutic uses, these groups of genes can be used to differentiate between liver tumor samples from subjects with chronic hepatitis and liver tumor samples from subjects with cirrhosis. Assays measuring the expression level of these genes are capable of distinguishing between carcinomas arising in chronic hepatitis patients versus carcinomas arising in cirrhosis patients.

The gene subsets of Table 1 can, therefore, be used to identify the presence of a malignant tumor in liver tissue from chronic hepatitis or cirrhosis patients, to monitor the progression of the tumor (e.g., during cancer treatment or combined disease treatments), to evaluate the effects of therapeutic agents for treating the tumor or to distinguish the origin or predisposing condition of the tumor.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

TABLE 1
Genes Differentially Expressed in Liver Cancer

| Fragment | Seq. | Accession | UniGene | | | HCC/ | p-Values for | |
|---|---|---|---|---|---|---|---|---|
| Name | ID | Number | ID | Description | HCC/CH | LC | HCC/CH | HCC/LC |

Group 1: HCC/CH and HCC/LC

| | | | | | | | | |
|---|---|---|---|---|---|---|---|---|
| 33428_s_at | 1 | AF034957 | Hs.194019 | attractin | 4.10 | 2.84 | 0.0002690 | 0.0002720 |
| 36785_at | 2 | Z23090 | Hs.76067 | heat shock 27 kD protein 1 | 2.71 | 3.49 | 0.0001100 | 0.0000150 |
| 74893_g_at | 3 | AA928646 | Hs.75864 | endoplasmic reticulum glycoprotein | 2.47 | 2.25 | 0.000748 | 0.000034 |
| 51788_at | 4 | AW023096 | Hs.3887 | proteasome (prosome, macropain) 26S subunit, | 2.03 | 1.74 | 0.000464 | 0.000647 |
| | | | | non-ATPase, 1 | | | | |
| 44143_at | 5 | AA399076 | Hs.46743 | McKusick-Kaufman syndrome | 1.65 | 1.52 | 0.000645 | 0.00038 |
| 832_at | 6 | U39317 | Hs.108332 | ubiquitin-conjugating enzyme E2D 2 (homologous to | 1.50 | 1.69 | 0.000222 | 0.000274 |
| | | | | yeast UBC4/5) | | | | |
| 57042_at | 7 | W74749 | Hs.285818 | similar to Caenorhabditis elegans protein C42C1.9 | −1.80 | −2.71 | 0.000394 | 0.000056 |
| 55107_at | 8 | AI916306 | Hs.87125 | EH-domain containing 3 | −1.91 | −3.32 | 0.000099 | 0 |
| 33766_at | 9 | X77777 | Hs.198726 | vasoactive intestinal peptide receptor 1 | −1.96 | −2.43 | 0.000039 | 0.00001 |
| 37206_at | 10 | X63359 | Hs.294039 | UDP glycosyltransferase 2 family, polypeptide B10 | −2.03 | −4.69 | 0.000016 | 4.00E−06 |
| 37059_at | 11 | Z48475 | Hs.89771 | glucokinase (hexokinase 4) regulatory protein | −2.41 | −10.31 | 0.000679 | 0.000872 |
| 533_g_at | 12 | U17418 | Hs.1019 | parathyroid hormone receptor 1 | −2.77 | −3.31 | 0.000064 | 0.00003 |
| 35803_at | 13 | S82240 | Hs.6838 | ras homolog gene family, member E | −3.62 | −8.20 | 1.00E−06 | 0 |
| 33862_at | 14 | AF017786 | Hs.173717 | phosphatidic acid phosphatase type 2B | −4.27 | −3.55 | 0.000161 | 0.000757 |
| 44982_s_at | 15 | AI985046 | Hs.24395 | small inducible cytokine subfamily B (Cys-X-Cys), | −5.65 | −12.23 | 0 | 0 |
| | | | | member 14 (BRAK) | | | | |
| 32666_at | 16 | U19495 | Hs.237356 | stromal cell-derived factor 1 | −6.47 | −6.54 | 0.000041 | 0.000012 |
| 35118_at | 17 | M12625 | Hs.325507 | lecithin-cholesterol acyltransferase | −6.48 | −23.56 | 0.000032 | 0.000165 |
| 55063_at | 18 | AL042399 | Hs.75668 | glutamate decarboxylase 1 (brain, 67 kD) | −7.52 | −23.54 | 0.000099 | 7.00E−06 |
| 34602_at | 19 | D63160 | Hs.54517 | ficolin (collagen/fibrinogen domain-containing lectin) | −7.54 | −10.65 | 0.000032 | 0.00002 |
| | | | | 2 (hucolin) | | | | |
| 34708_at | 20 | D88587 | Hs.333383 | ficolin (collagen/fibrinogen domain-containing) 3 | −9.43 | −21.71 | 0.000271 | 0.000059 |
| | | | | (Hakata antigen) | | | | |
| 39120_at | 21 | AA224832 | Hs.94360 | metallothionein 1 L | −9.97 | −35.38 | 0.000089 | 3.00E−06 |
| 45943_at | 22 | AI052592 | Hs.35718 | cytochrome P450, subfamily VIIIB | −11.93 | −26.66 | 0.000178 | 0.000914 |
| | | | | (sterol 12-alpha-hydroxylase), polypeptide 1 | | | | |
| 56641_at | 23 | AI937227 | Hs.8821 | liver-expressed antimicrobial peptide | −19.90 | −66.71 | 0.000708 | 0.00097 |

Group 2: HCC/CH

| | | | | | | | | |
|---|---|---|---|---|---|---|---|---|
| 45313_at | 24 | AA167715 | Hs.296244 | fatty acid synthase | 4.22 | 2.44 | 0.000396 | — |
| 85972_at | 25 | AI424433 | Hs.306000 | solute carrier family 4 (anion exchanger), member 1, | 1.92 | 2.24 | 0.000318 | — |
| | | | | adapter protein | | | | |
| 1840_g_at | | HG1112-HT11 | Hs.10842 | RAN, member RAS oncogene family | 1.96 | 1.88 | 0.000705 | — |
| 33667_at | 26 | X52851 | Hs.182937 | peptidylprolyl isomerase A (cyclophilin A) | 1.74 | 1.35 | 0.000879 | — |
| 53474_at | 27 | AF072812 | Hs.7765 | chromosome 16 open reading frame 5 | −1.91 | −2.04 | 0.000253 | — |
| 34367_at | 28 | AF006043 | Hs.3343 | phosphoglycerate dehydrogenase | −4.06 | −2.72 | 0.000976 | — |
| 38862_at | 29 | Y11215 | Hs.19126 | src kinase-associated phosphoprotein of 55 kDa | −3.73 | −3.09 | 0.000593 | — |
| 40325_at | 30 | AB014460 | Hs.66196 | nth (E. coli endonuclease III)-like 1 | −2.52 | −3.18 | 0.000312 | — |
| 32727_at | 31 | AF037062 | Hs.172914 | retinol dehydrogenase 5 (11-cisand 9-cis) | −4.50 | −3.42 | 0.000216 | — |
| 37319_at | 32 | M35878 | Hs.77326 | insulin-like growth factor binding protein 3 | −5.33 | −3.71 | 0.000339 | — |
| 35063_at | 33 | D50030 | Hs.104 | HGF activator | −3.99 | −8.29 | 0.000159 | — |
| 1391_s_at | 34 | L04751 | Hs.1645 | cytochrome P450, subfamily IVA, polypeptide 11 | −1.76 | −14.90 | 0.000824 | — |
| 32966_at | 35 | L27050 | Hs.2388 | apolipoprotein F | −6.33 | −16.30 | 0.000026 | — |

Group 3: HCC/LC

| | | | | | | | | |
|---|---|---|---|---|---|---|---|---|
| 37482_at | 36 | U37100 | Hs.116724 | aldo-keto reductase family 1, member B11 | 11.79 | 27.19 | — | 0.000874 |
| | | | | (aldose reductase-like) | | | | |
| 33404_at | 37 | U02390 | Hs.296341 | adenylyl cyclase-associated protein 2 | 2.60 | 7.77 | — | 0.0002 |
| 63545_at | 38 | AW006831 | Hs.337478 | RAB, member of RAS oncogene family-like 2B | 3.71 | 7.20 | — | 0.000793 |
| 34390_at | 39 | U90441 | Hs.3622 | procollagen-proline, 2-oxoglutarate 4-dioxygenase | 3.58 | 6.27 | — | 0.000211 |
| | | | | (proline 4-hydroxylase), alpha polypeptide II | | | | |
| 33873_at | 40 | D43642 | Hs.2430 | transcription factor-like 1 | 2.29 | 5.88 | — | 0.000472 |
| 893_at | 41 | M91670 | Hs.174070 | ubiquitin carrier protein | 2.51 | 5.85 | — | 0.000206 |
| 59749_at | 42 | AI478190 | Hs.324178 | solute carrier family 25 (mitochondrial carrier; | 1.21 | 5.18 | — | 0.000638 |
| | | | | adenine nucleotide translocator), member 6 | | | | |
| 39749_at | 43 | U51007 | Hs.148495 | proteasome (prosome, macropain) | 2.01 | 4.91 | — | 0.000288 |
| | | | | 26S subunit, non-ATPase, 4 | | | | |
| 44695_at | 44 | AI953020 | Hs.324618 | HSPC142 protein | 2.71 | 4.66 | — | 0.000153 |
| 43836_s_at | 45 | AI971969 | Hs.282997 | glucosidase, beta; acid (includes glucosylceramidase) | 1.91 | 4.21 | — | 9.00E−06 |
| 39801_at | 46 | AF046889 | Hs.153357 | procollagen-lysine, 2-oxoglutarate 5-dioxygenase 3 | 3.32 | 4.11 | — | 0.000257 |
| 44219_at | 47 | AI937030 | Hs.287883 | X11L-binding protein 51 | 1.44 | 3.92 | — | 0.00032 |
| 37256_at | 48 | AI829890 | Hs.78524 | TcD37 homolog | 1.79 | 3.76 | — | 0.00064 |
| 37399_at | 49 | D17793 | Hs.78183 | aldo-keto reductase family 1, member C3 (3-alpha | 2.53 | 3.76 | — | 4.00E−06 |
| | | | | hydroxysteroid dehydrogenase, type II) | | | | |
| 35820_at | 50 | X62078 | Hs.289082 | GM2 ganglioside activator protein | 2.10 | 3.69 | — | 0.00034 |
| 1100_at | 51 | L76191 | Hs.182018 | interleukin-1 receptor-associated kinase 1 | 3.76 | 3.60 | — | 0.000816 |
| 146_at | 52 | U81802 | Hs.154846 | phosphatidylinositol 4-kinase, catalytic, beta | 1.90 | 3.42 | — | 0.000767 |
| | | | | polypeptide | | | | |
| 77990_at | 53 | AW004018 | Hs.268281 | CGI-201 protein | 1.76 | 3.39 | — | 0.000842 |
| 32799_at | 54 | AF023268 | Hs.200600 | secretory carrier membrane protein 3 | 1.81 | 3.25 | — | 0.00009 |
| 32260_at | 55 | X86809 | Hs.194673 | phosphoprotein enriched in astrocytes 15 | 2.14 | 3.17 | — | 0.000599 |
| 497_at | 56 | U32680 | Hs.194660 | ceroid-lipofuscinosis, neuronal 3, juvenile (Batten, | 1.97 | 3.14 | — | 0.00013 |
| | | | | Spielmeyer-Vogt disease) | | | | |
| 33154_at | 57 | D26600 | Hs.89545 | proteasome (prosome, macropain) subunit, beta type, 4 | 1.62 | 3.08 | — | 0.000019 |
| 39062_at | 58 | AL008726 | Hs.118126 | protective protein for beta-galactosidase | 2.18 | 3.05 | — | 0.00004 |
| | | | | (galactosialidosis) | | | | |
| 56378_at | 59 | W22366 | Hs.337078 | NICE-5 protein | 1.45 | 3.02 | — | 0.000292 |
| 45155_at | 60 | AI433892 | Hs.38738 | claudin 15 | 2.14 | 2.95 | — | 0.000669 |
| 44082_at | 61 | AA029831 | Hs.238928 | HT002 protein; hypertension-related | 1.63 | 2.94 | — | 0.00064 |
| | | | | calcium-regulated gene | | | | |
| 57136_at | 62 | AI279571 | Hs.23528 | HSPC038 protein | 1.54 | 2.93 | — | 0.000293 |
| 64501_at | 63 | AI982714 | Hs.93832 | putative membrane protein | 1.97 | 2.70 | — | 0.000498 |
| 34835_at | 64 | D87442 | Hs.4788 | nicastrin | 1.62 | 2.69 | — | 0.000332 |
| 41322_s_at | 65 | AI816034 | Hs.23990 | nucleolar protein family A, member 2 | 1.89 | 2.61 | — | 0.000756 |
| | | | | (H/ACA small nucleolar RNPs) | | | | |
| 44821_at | 66 | AI634570 | Hs.301005 | purine-rich element binding protein B | 1.96 | 2.58 | — | 0.000034 |
| 48913_at | 67 | AI023344 | Hs.12865 | p47 | 1.51 | 2.56 | — | 0.000807 |
| 35685_at | 68 | Z14000 | Hs.35384 | ring finger protein 1 | 1.43 | 2.52 | — | 0.000061 |
| 64886_at | 69 | AA632300 | Hs.65648 | RNA binding motif protein 8A | 1.58 | 2.40 | — | 0.000325 |
| 74577_s_at | 70 | AI798743 | Hs.183994 | RAD9 (S. pombe) homolog | 1.77 | 2.38 | — | 0.000159 |
| 45712_at | 71 | H98166 | Hs.279868 | SUMO-1 activating enzyme subunit 1 | 1.52 | 2.30 | — | 0.000214 |
| 90637_at | 72 | AA039699 | Hs.7101 | anaphase-promoting complex subunit 5 | 1.40 | 2.28 | — | 0.000854 |
| 1659_s_at | 73 | D78132 | Hs.279903 | Ras homolog enriched in brain 2 | 1.98 | 2.20 | — | 0.00049 |
| 38719_at | 74 | U03985 | Hs.108802 | N-ethylmaleimide-sensitive factor | 2.03 | 2.14 | — | 0.000546 |
| 37669_s_at | 75 | U16799 | Hs.78629 | ATPase, Na+/K+ transporting, beta 1 polypeptide | 1.58 | 2.12 | — | 0.000082 |
| 45255_at | 76 | AI354351 | Hs.237924 | CGI-69 protein | 1.99 | 2.11 | — | 0.000599 |
| 1309_at | 77 | D26598 | Hs.82793 | proteasome (prosome, macropain) subunit, beta type, 3 | 1.91 | 1.83 | — | 0.000106 |
| 33659_at | 78 | X95404 | Hs.180370 | cofilin 1 (non-muscle) | 1.64 | 1.63 | — | 0.000997 |
| 35752_s_at | 79 | M15036 | Hs.64016 | protein S (alpha) | −1.42 | −2.03 | — | 0.000865 |
| 64369_s_at | 80 | AA219354 | Hs.282804 | ceruloplasmin (ferroxidase) | −1.79 | −2.31 | — | 0.000802 |
| 260_at | 81 | M16447 | Hs.75438 | quinoid dihydropteridine reductase | −1.76 | −2.65 | — | 0.000586 |
| 40082_at | 82 | D10040 | Hs.154890 | fatty-acid-Coenzyme A ligase, long-chain 2 | −1.26 | −2.98 | — | 0.000059 |
| 46746_s_at | 83 | W42636 | Hs.5326 | porcupine | −1.34 | −3.05 | — | 0.000074 |
| 90033_at | 84 | T66157 | Hs.154437 | phosphodiesterase 2A, cGMP-stimulated | −2.22 | −3.41 | — | 0.000467 |
| 36097_at | 85 | M62831 | Hs.737 | immediate early protein | −1.64 | −4.02 | — | 0.000036 |
| 74184_at | 86 | T98839 | Hs.30299 | IGF-II mRNA-binding protein 2 | −1.48 | −4.78 | — | 0.000181 |
| 37022_at | 87 | U41344 | Hs.76494 | proline arginine-rich end leucine-rich repeat protein | −2.63 | −4.89 | — | 0.000047 |
| 58322_at | 88 | AI765890 | Hs.16341 | MAWD binding protein | −1.45 | −5.43 | — | 0.000166 |
| 65867_at | 89 | AL043089 | Hs.3807 | FXYD domain-containing ion transport regulator 6 | −1.70 | −5.44 | — | 0.000143 |
| 38634_at | 90 | M11433 | Hs.101850 | retinol-binding protein 1, cellular | −6.44 | −5.51 | — | 0.00016 |
| 38772_at | 91 | Y11307 | Hs.8867 | cysteine-rich angiogenic inducer, 61 | −4.09 | −5.72 | — | 0.000487 |
| 46694_at | 92 | AI078144 | Hs.9315 | HNOEL-iso protein | −4.66 | −5.88 | — | 0.000875 |
| 48502_at | 93 | AA122235 | Hs.113052 | RNA cyclase homolog | −3.30 | −6.27 | — | 0.000765 |
| 64390_at | 94 | AI342377 | Hs.44281 | CDK4-binding protein p34SEI1 | −2.10 | −6.59 | — | 0.000087 |
| 1212_at | 95 | U86529 | Hs.26403 | glutathione transferase zeta 1 | −3.51 | −7.30 | — | 0.000367 |
| | | | | (maleylacetoacetate isomerase) | | | | |
| 42363_r_at | 96 | AI680350 | Hs.296176 | STAT induced STAT inhibitor 3 | −1.71 | −7.37 | — | 0.000016 |
| 91311_at | 97 | AA576961 | Hs.82101 | pleckstrin homology-like domain, family A, member 1 | −2.77 | −7.52 | — | 0.000083 |
| 37972_at | 98 | U75744 | Hs.88646 | deoxyribonuclease I-like 3 | −3.21 | −7.76 | — | 0.000691 |
| 1379_at | 99 | M59371 | Hs.171596 | epithelial receptor protein-tyrosine kinase | −1.67 | −7.95 | — | 0 |
| 35925_at | 100 | AF040639 | Hs.284236 | aldo-keto reductase family 7, member A3 | −2.65 | −9.29 | — | 0.000105 |
| | | | | (aflatoxin aldehyde reductase) | | | | |
| 34638_r_at | 101 | M12963 | Hs.73843 | alcohol dehydrogenase 1 (class I), alpha polypeptide | −1.85 | −9.89 | — | 0.00081 |
| 35556_at | 102 | K02402 | Hs.1330 | coagulation factor IX (plasma thromboplastic | −1.84 | −10.42 | — | 0.00065 |
| | | | | component, Christmas disease, hemophilia B) | | | | |
| 41376_i_at | 103 | J05428 | Hs.10319 | UDP glycosyltransferase 2 family, polypeptide B7 | −3.64 | −10.49 | — | 0.000071 |
| 61370_at | 104 | AI819354 | Hs.301528 | L-kynurenine/alpha-aminoadipate aminotransferase | −3.46 | −10.90 | — | 0.000621 |
| 33564_at | 105 | L32140 | Hs.531 | afamin | −1.72 | −11.83 | — | 0.000065 |
| 31622_f_at | 106 | M10943 | Hs.203936 | metallothionein 1F (functional) | −3.56 | −11.84 | — | 0.000364 |
| 35730_at | 107 | X03350 | Hs.4 | alcohol dehydrogenase 2 (class I), beta polypeptide | −4.24 | −13.10 | — | 0.000706 |
| 37394_at | 108 | J03507 | Hs.78065 | complement component 7 | −3.50 | −15.87 | — | 0.000247 |
| 31623_f_at | 109 | K01383 | Hs.173451 | metallothionein 1A (functional) | −5.12 | −32.78 | — | 0.000409 |

| | | | | | | | |
|---|---|---|---|---|---|---|---|
| Fragment | | | HCC(CH)/ | | Mean(HCC | | Mean(HCC |
| Name | Seq. ID | CH/LC | HCC(LC) | Mean(CH) | from CH) | Mean(LC) | from LC) |

Group 1: HCC/CH and HCC/LC

| | | | | | | | |
|---|---|---|---|---|---|---|---|
| 33428_s_at | 1 | −1.35 | 1.06 | 36.99 | 151.48 | 50.05 | 142.36 |
| 36785_at | 2 | 1.15 | −1.12 | 1300.25 | 3526.56 | 1133.51 | 3956.77 |
| 74893_g_at | 3 | −1.19 | −1.09 | 1381.07 | 3404.95 | 1647.94 | 3710.79 |
| 51788_at | 4 | −1.36 | −1.17 | 725.34 | 1470.84 | 987.9 | 1714.14 |
| 44143_at | 5 | −1.38 | −1.27 | 193.1 | 319.1 | 265.76 | 404.33 |
| 832_at | 6 | 1.06 | −1.06 | 151.86 | 227.89 | 143.41 | 242.01 |
| 57042_at | 7 | −1.46 | 1.03 | 875.09 | 485.4 | 1278.17 | 472.25 |
| 55107_at | 8 | −1.31 | 1.33 | 387.69 | 203.24 | 506.63 | 152.43 |
| 33766_at | 9 | −1.24 | 1.00 | 39.13 | 20 | 48.58 | 20 |
| 37206_at | 10 | −2.07 | 1.12 | 386.63 | 190.39 | 799.12 | 170.46 |
| 37059_at | 11 | −1.25 | 3.43 | 258.26 | 107.34 | 322.94 | 31.33 |
| 533_g_at | 12 | 1.54 | 1.84 | 192.89 | 69.68 | 125.26 | 37.81 |
| 35803_at | 13 | −2.03 | 1.11 | 211.11 | 58.27 | 429.09 | 52.34 |
| 33862_at | 14 | 1.00 | −1.20 | 1039.82 | 243.42 | 1035.68 | 291.65 |
| 44982_s_at | 15 | −1.60 | 1.35 | 152.67 | 27.03 | 244.6 | 20 |
| 32666_at | 16 | −1.15 | −1.14 | 206.74 | 31.94 | 238.32 | 36.44 |
| 35118_at | 17 | −1.55 | 2.34 | 469.05 | 72.38 | 728.59 | 30.93 |
| 55063_at | 18 | −2.08 | 1.50 | 494.25 | 65.7 | 1028.8 | 43.7 |
| 34602_at | 19 | −1.33 | 1.06 | 260.1 | 34.51 | 345.97 | 32.48 |
| 34708_at | 20 | −1.76 | 1.31 | 761.37 | 80.76 | 1337.08 | 61.6 |
| 39120_at | 21 | −2.27 | 1.56 | 2259.52 | 226.61 | 5124.11 | 144.85 |
| 45943_at | 22 | −2.46 | −1.10 | 1674.55 | 140.37 | 4117.95 | 154.45 |
| 56641_at | 23 | −3.95 | −1.18 | 4054.26 | 203.69 | 16004.11 | 239.9 |

Group 2: HCC/CH

| | | | | | | | |
|---|---|---|---|---|---|---|---|
| 45313_at | 24 | −1.53 | 1.13 | 158.25 | 667.72 | 241.75 | 590.69 |
| 85972_at | 25 | −1.56 | −1.82 | 24.11 | 46.33 | 37.62 | 84.41 |
| 1840_g_at | | −1.10 | −1.06 | 353.18 | 691.4 | 388.94 | 730.83 |
| 33667_at | 26 | −1.11 | 1.15 | 2848.2 | 4949.09 | 3175.61 | 4297.99 |
| 53474_at | 27 | −1.62 | −1.52 | 246.2 | 128.65 | 398.43 | 195.14 |
| 34367_at | 28 | −1.04 | −1.55 | 605.74 | 149.24 | 630.02 | 231.53 |
| 38862_at | 29 | −1.14 | −1.38 | 93.76 | 25.12 | 106.98 | 34.66 |
| 40325_at | 30 | 1.14 | 1.43 | 211 | 83.67 | 185.74 | 58.46 |
| 32727_at | 31 | 1.17 | −1.12 | 89.92 | 20 | 77.01 | 22.5 |
| 37319_at | 32 | −1.02 | −1.46 | 2159.87 | 404.88 | 2196.02 | 591.49 |
| 35063_at | 33 | −1.25 | 1.66 | 423.71 | 106.19 | 529.69 | 63.87 |
| 1391_s_at | 34 | −1.48 | 5.71 | 1096.07 | 622.87 | 1625.85 | 109.13 |
| 32966_at | 35 | −1.42 | 1.82 | 371.31 | 58.66 | 525.9 | 32.26 |

Group 3: HCC/LC

| | | | | | | | |
|---|---|---|---|---|---|---|---|
| 37482_at | 36 | −2.68 | −6.18 | 30.34 | 357.74 | 81.37 | 2212.61 |
| 33404_at | 37 | 1.27 | −2.36 | 25.3 | 65.78 | 20 | 155.35 |
| 63545_at | 38 | 1.15 | −1.68 | 52.84 | 196.23 | 45.81 | 329.84 |
| 34390_at | 39 | −1.12 | −1.95 | 22.56 | 80.76 | 25.18 | 157.82 |
| 33873_at | 40 | 1.39 | −1.84 | 83.99 | 192.42 | 60.26 | 354.05 |
| 893_at | 41 | 1.49 | −1.56 | 58.78 | 147.44 | 39.34 | 230.06 |
| 59749_at | 42 | 2.80 | −1.53 | 100.08 | 120.75 | 35.71 | 184.96 |
| 39749_at | 43 | 1.04 | −2.34 | 108.86 | 219.04 | 104.32 | 511.93 |
| 44695_at | 44 | 1.71 | −1.01 | 34.2 | 92.53 | 20 | 93.12 |
| 43836_s_at | 45 | 1.15 | −1.92 | 484.14 | 924.31 | 422.21 | 1775.62 |
| 39801_at | 46 | 1.15 | −1.08 | 157.64 | 524.07 | 137.41 | 564.88 |
| 44219_at | 47 | 1.32 | −2.06 | 173.41 | 250.32 | 131.61 | 515.98 |
| 37256_at | 48 | 1.32 | −1.59 | 46.64 | 83.47 | 35.25 | 132.39 |
| 37399_at | 49 | −1.23 | −1.83 | 642.62 | 1628.75 | 791.81 | 2974.46 |
| 35820_at | 50 | 1.78 | 1.01 | 91.39 | 192.04 | 51.31 | 189.52 |
| 1100_at | 51 | −1.07 | −1.03 | 41.77 | 157.11 | 44.84 | 161.26 |
| 146_at | 52 | 1.11 | −1.61 | 45.26 | 86.08 | 40.62 | 138.85 |
| 77990_at | 53 | 1.31 | −1.47 | 92.53 | 163.29 | 70.82 | 240.01 |
| 32799_at | 54 | 1.13 | −1.59 | 201.78 | 365.98 | 179.36 | 582.69 |
| 32260_at | 55 | 1.17 | −1.26 | 215.21 | 460.32 | 183.36 | 581.74 |
| 497_at | 56 | 1.04 | −1.53 | 163.55 | 323.01 | 157.23 | 493.58 |
| 33154_at | 57 | −1.01 | −1.92 | 476.51 | 771.16 | 481.66 | 1481.48 |
| 39062_at | 58 | −1.01 | −1.42 | 323.59 | 705.43 | 328.02 | 1000.16 |
| 56378_at | 59 | 1.10 | −1.89 | 586.6 | 847.94 | 530.86 | 1603.61 |
| 45155_at | 60 | 1.39 | 1.01 | 509.58 | 1088.23 | 367.1 | 1081.19 |
| 44082_at | 61 | 1.05 | −1.71 | 145.39 | 237.67 | 138.47 | 407.05 |
| 57136_at | 62 | −1.02 | −1.94 | 856.14 | 1320.28 | 873.09 | 2557.79 |
| 64501_at | 63 | −1.16 | −1.59 | 939.04 | 1846.8 | 1086.52 | 2938.37 |
| 34835_at | 64 | 1.02 | −1.63 | 289.56 | 469.17 | 284.43 | 766.35 |
| 41322_s_at | 65 | 1.03 | −1.34 | 84.42 | 159.51 | 81.92 | 213.73 |
| 44821_at | 66 | −1.08 | −1.43 | 389.21 | 762.25 | 421.21 | 1088.69 |
| 48913_at | 67 | 1.06 | −1.60 | 380.42 | 575.38 | 358.46 | 918.11 |
| 35685_at | 68 | 1.64 | −1.07 | 289.35 | 413.86 | 176.34 | 443.67 |
| 64886_at | 69 | −1.48 | −2.25 | 442.13 | 697.6 | 654 | 1568.34 |
| 74577_s_at | 70 | −1.07 | −1.44 | 1553.52 | 2748.37 | 1669.16 | 3966.04 |
| 45712_at | 71 | −1.01 | −1.53 | 336.68 | 512.07 | 340.41 | 782.95 |
| 90637_at | 72 | 1.41 | −1.15 | 216.23 | 302.5 | 153.02 | 348.45 |
| 1659_s_at | 73 | 1.15 | 1.04 | 265.54 | 526.93 | 230.24 | 506.23 |
| 38719_at | 74 | −1.11 | −1.17 | 56.48 | 114.7 | 62.54 | 134.02 |
| 37669_s_at | 75 | −1.03 | −1.38 | 1005.93 | 1588.92 | 1034.68 | 2197.29 |
| 45255_at | 76 | 1.15 | 1.09 | 1106.18 | 2205.3 | 960.49 | 2029 |
| 1309_at | 77 | −1.20 | −1.15 | 416.77 | 795.57 | 500.68 | 914.19 |
| 33659_at | 78 | −1.00 | 1.01 | 1604.43 | 2635.01 | 1605.92 | 2620.97 |
| 35752_s_at | 79 | −1.06 | 1.34 | 266.49 | 187.67 | 283.7 | 139.98 |
| 64369_s_at | 80 | −1.23 | 1.05 | 1096.17 | 613.12 | 1345.19 | 582.06 |
| 260_at | 81 | −1.50 | 1.00 | 330.36 | 187.35 | 494.66 | 186.66 |
| 40082_at | 82 | −2.20 | 1.07 | 456.38 | 360.83 | 1003.4 | 336.73 |
| 46746_s_at | 83 | −1.96 | 1.16 | 889.16 | 664.38 | 1739.7 | 571.32 |
| 90033_at | 84 | −1.09 | 1.41 | 596.98 | 268.41 | 648.03 | 190.08 |
| 36097_at | 85 | −1.17 | 2.09 | 860.22 | 522.96 | 1004.81 | 249.96 |
| 74184_at | 86 | −2.48 | 1.30 | 159.53 | 107.88 | 395.36 | 82.7 |
| 37022_at | 87 | 1.25 | 2.33 | 315.21 | 119.91 | 251.61 | 51.46 |
| 58322_at | 88 | −2.17 | 1.72 | 2508.42 | 1725.07 | 5453.68 | 1004.8 |
| 65867_at | 89 | 1.21 | 3.87 | 2602.05 | 1527.55 | 2143.83 | 394.41 |
| 38634_at | 90 | 1.50 | 1.28 | 682.93 | 106.06 | 455.03 | 82.61 |
| 38772_at | 91 | 1.12 | 1.56 | 193.88 | 47.36 | 173.31 | 30.31 |
| 46694_at | 92 | 1.99 | 2.52 | 322.34 | 69.24 | 161.78 | 27.53 |
| 48502_at | 93 | −1.64 | 1.16 | 935.83 | 283.76 | 1535.68 | 245.08 |
| 64390_at | 94 | −1.68 | 1.88 | 269.34 | 128.48 | 451.21 | 68.52 |
| 1212_at | 95 | 1.06 | 2.20 | 374.12 | 106.47 | 352.4 | 48.29 |
| 42363_r_at | 96 | −3.60 | 1.19 | 851 | 496.58 | 3065.8 | 416.23 |
| 91311_at | 97 | −2.98 | −1.10 | 334.15 | 120.6 | 994.75 | 132.35 |
| 37972_at | 98 | −1.34 | 1.80 | 450.49 | 140.24 | 604.69 | 77.9 |
| 1379_at | 99 | −3.28 | 1.45 | 48.46 | 29 | 158.9 | 20 |
| 35925_at | 100 | −1.96 | 1.79 | 163.57 | 61.72 | 320.94 | 34.54 |
| 34638_r_at | 101 | −2.60 | 2.06 | 351.38 | 189.82 | 912.75 | 92.25 |
| 35556_at | 102 | −2.34 | 2.42 | 621.13 | 337.88 | 1452.17 | 139.35 |
| 41376_i_at | 103 | −2.77 | 1.04 | 1104.52 | 303.33 | 3061.38 | 291.7 |
| 61370_at | 104 | −1.76 | 1.79 | 161.16 | 46.57 | 283.6 | 26.03 |
| 33564_at | 105 | −2.99 | 2.29 | 237.07 | 137.46 | 708.95 | 59.9 |
| 31622_f_at | 106 | −1.89 | 1.76 | 3587.58 | 1009.05 | 6782.49 | 572.84 |
| 35730_at | 107 | −1.82 | 1.69 | 539.69 | 127.19 | 982.94 | 75.05 |
| 37394_at | 108 | −1.44 | 3.15 | 506.19 | 144.63 | 728.66 | 45.9 |
| 31623_f_at | 109 | −1.93 | 3.31 | 3516.77 | 686.61 | 6798.83 | 207.38 |

TABLE 2
Patient Information

| | Donor | Donor | Donor Age | Date of | Organ/ | | Normal or | |
|---|---|---|---|---|---|---|---|---|
| Sample ID | Gender | Race | at Excision | Collection | Fluid | Tissue Site | Diseased | Specimen Diagnosis |
| YUMC-034-01 | Male | Korean | 42 | Apr. 2, 2001 | Liver | left lobe | Diseased | Liver cirrhosis (HBV) |
| YUMC-034-02 | Male | Korean | 42 | Apr. 2, 2001 | Liver | left lobe | Malignant | Hepatoma |
| YUMC-035-01 | Male | Korean | 34 | Jan. 29, 2001 | Liver | right lobe | Diseased | Chronic hepatitis B |
| YUMC-035-02 | Male | Korean | 34 | Jan. 29, 2001 | Liver | right lobe | Malignant | Hepatoma |
| YUMC-036-01 | Female | Korean | 43 | Feb. 16, 2001 | Liver | left lobe | Diseased | Liver cirrhosis (HBV) |
| YUMC-036-02 | Female | Korean | 43 | Feb. 16, 2001 | Liver | left lobe | Malignant | Hepatoma |
| YUMC-037-01 | Female | Korean | 65 | Feb. 14, 2001 | Liver | right lobe | Diseased | Chronic hepatitis B |
| YUMC-037-02 | Female | Korean | 65 | Feb. 14, 2001 | Liver | right lobe | Malignant | Hepatoma |
| YUMC-038-01 | Male | Korean | 37 | Feb. 21, 2001 | Liver | right lobe | Diseased | Liver cirrhosis (HBV) |
| YUMC-038-02 | Male | Korean | 37 | Feb. 21, 2001 | Liver | right lobe | Malignant | Hepatoma |
| YUMC-039-01 | Male | Korean | 62 | Apr. 5, 2001 | Liver | right lobe | Diseased | Liver cirrhosis (HBV) |
| YUMC-039-02 | Male | Korean | 62 | Apr. 5, 2001 | Liver | right lobe | Malignant | Hepatoma |
| YUMC-040-01 | Male | Korean | 40 | Mar. 30, 2001 | Liver | right lobe | Diseased | Liver cirrhosis (HBV) |
| YUMC-040-02 | Male | Korean | 40 | Mar. 30, 2001 | Liver | right lobe | Malignant | Hepatoma |
| YUMC-042-01 | Male | Korean | 61 | Dec. 18, 2000 | Liver | left lobe | Diseased | Chronic hepatitis B |
| YUMC-042-02 | Male | Korean | 61 | Dec. 18, 2000 | Liver | left lobe | Malignant | Hepatoma |
| YUMC-043-01 | Male | Korean | 63 | Mar. 27, 2001 | Liver | left lobe | Diseased | Chronic hepatitis B |
| YUMC-043-02 | Male | Korean | 63 | Mar. 27, 2001 | Liver | left lobe | Malignant | Hepatoma |
| YUMC-059-01 | Male | Korean | 62 | Mar. 26, 2001 | Liver | right lobe | Diseased | chronic hepatitis B |
| YUMC-059-02 | Male | Korean | 62 | Mar. 26, 2001 | Liver | right lobe | Malignant | hepatocellular carcinoma |

1. A method of diagnosing liver cancer in a patient, comprising:
(a) detecting the level of expression in a tissue sample of one or more genes from Table 1; wherein differential expression of the genes in Table 1 is indicative of liver cancer.

2. A method of detecting the progression of liver cancer in a patient, comprising:
(a) detecting the level of expression in a tissue sample of one or more genes from Table 1; wherein differential expression of the genes in Table 1 is indicative of liver cancer progression.

3. A method of monitoring the treatment of a patient with liver cancer, comprising:
(a) administering a pharmaceutical composition to the patient;
(b) preparing a gene expression profile of one or more of the genes in Table 1 from a cell or tissue sample from the patient; and
(c) comparing the patient gene expression profile to a gene expression profile from a cell population selected from the group consisting of non-cancerous liver cells and cancerous liver cells.

4. A method of treating a patient with liver cancer, comprising:
(a) administering to the patient a pharmaceutical composition;
(b) preparing a gene expression profile of one or more of the genes in Table 1 from a cell or tissue sample from the patient; and
(c) comparing the patient expression profile to a gene expression profile selected from the group consisting of non-cancerous liver cells and cancerous liver cells.

5. A method of typing liver disease in a patient, comprising:
(a) detecting the level of expression in a tissue sample of one or more genes from Table 1; wherein differential expression of the genes in Table 1 is indicative of a type of liver disease selected from a group consisting of chronic hepatitis with hepatic carcinoma and cirrhosis with hepatic carcinoma.

6. A method of detecting the presence or progression of liver cancer in a patient with chronic hepatitis, comprising:
(a) detecting the level of expression in a tissue sample of one or more genes from Table 1; wherein differential expression of the genes in Table 1 is indicative of chronic hepatitis with liver cancer.

7. A method of detecting the presence or progression of liver cancer in a patient with cirrhosis, comprising:
(a) detecting the level of expression in a tissue sample of one or more genes from Table 1; wherein differential expression of the genes in Table 1 is indicative of cirrhosis with liver cancer.

8. A method of diagnosing liver cancer according to claim 1, wherein the liver cancer is accompanied by chronic hepatitis or cirrhosis.

9. A method of differentiating liver cancer related to chronic hepatitis from liver cancer related to cirrhosis in a patient, comprising:
(a) detecting the level of expression in a tissue sample of one or more genes from Table 1; wherein differential expression of the genes in Table 1 is indicative of either liver cancer related to chronic hepatitis or liver cancer related to cirrhosis.

10. A method of screening for an agent capable of modulating the onset or progression of liver cancer, comprising:
(a) preparing a first gene expression profile of a cell population comprising cancerous liver cells, wherein the expression profile comprises the expression level of one or more genes from Table 1;
(b) exposing the cell population to the agent;
(c) preparing second gene expression profile of the agent-exposed cell population; and
(d) comparing the first and second gene expression profiles.

11. The method of claim 10, wherein the liver cancer is chronic hepatitis with liver cancer.

12. The method of claim 10, wherein the liver disease is cirrhosis with liver cancer.

13. A composition comprising at least two oligonucleotides, wherein each of the oligonucleotides comprises a sequence that specifically hybridizes to a gene in Table 1.

14. A composition according to claim 13, wherein the composition comprises at least 3 oligonucleotides.

15. A composition according to claim 13, wherein the composition comprises at least 5 oligonucleotides.

16. A composition according to claim 13, wherein the composition comprises at least 7 oligonucleotides.

17. A composition according to claim 13, wherein the composition comprises at least 10 oligonucleotides.

18. A composition according to any one of claims 13, wherein the oligonucleotides are attached to a solid support.

19. A composition according to claim 18, wherein the solid support is selected from a group consisting of a membrane, a glass support, a filter, a tissue culture dish, a polymeric material, a bead and a silica support.

20. A solid support comprising at least two oligonucleotides, wherein each of the oligonucleotides comprises a sequence that specifically hybridizes to a gene in Table 1.

21. A solid support according to claim 20, wherein the oligonucleotides are covalently attached to the solid support.

22. A solid support according to claim 20, wherein the oligonucleotides are non-covalently attached to the solid support.

23. A solid support according to claim 20, wherein the support comprises at least about 10 different oligonucleotides in discrete locations per square centimeter.

24. A solid support according to claim 20, wherein the support comprises at least about 100 different oligonucleotides in discrete locations per square centimeter.

25. A solid support according to claim 20, wherein the support comprises at least about 1000 different oligonucleotides in discrete locations per square centimeter.

26. A solid support according to claim 20, wherein the support comprises at least about 10,000 different oligonucleotides in discrete locations per square centimeter.

27. A computer system comprising:
(a) a database containing information identifying the expression level in liver tissue of a set of genes comprising at least one gene in Table 1; and
(b) a user interface to view the information.

28. A computer system of claim 27, wherein the database further comprises sequence information for the genes.

29. A computer system of claim 27, wherein the database further comprises information identifying the expression level for the genes in normal liver tissue.

30. A computer system of claim 27, wherein the database further comprises information identifying the expression level for the genes in tissue from a hepatic carcinoma.

31. A computer system of claim 30, wherein the hepatic carcinoma is from a patient with chronic hepatitis.

32. A computer system of claim 30, wherein the hepatic carcinoma is from a patient with cirrhosis.

33. A computer system of claim 27, further comprising records including descriptive information from an external database, which information correlates said genes to records in the external database.

34. A computer system of claim 33, wherein the external database is GenBank.

35. A method of using a computer system of a claim 27 to present information identifying the expression level in a tissue or cell of at least one gene in Table 1, comprising:
(a) comparing the expression level of at least one gene in Table 1 in the tissue or cell to the level of expression of the gene in the database.

36. A method of claim 35, wherein the expression level of at least two genes are compared.

37. A method of claim 35, wherein the expression level of at least five genes are compared.

38. A method of claim 35, wherein the expression level of at least ten genes are compared.

39. A method of claim 35, further comprising displaying the level of expression of at least one gene in the tissue or cell sample compared to the expression level in liver disease.

40. A method of claim 39, wherein the liver disease is hepatic carcinoma, chronic hepatitis or cirrhosis.

41. A therapeutic agent for slowing or halting the progression of liver cancer, wherein the agent is selected from the group consisting of the genes in Table 1, functional fragments of the genes in Table 1, proteins encoded by the genes in Table 1 and functional fragments of said proteins.

42. A method of treating a patient with liver cancer, comprising:
(a) administering to a patient with liver cancer a pharmaceutical composition comprising all or a portion of at least one gene in Table 1, or a protein encoded therein.


SEQUENCE LISTING (109 sequences)

SEQ ID NO: 1 - DNA, 3597 bp, Homo sapiens
  Genbank Accession No. AF034957
atggtggccgcagcggcggcaactgaggcaaggctgaggaggaggacggcggcgacggcagcgctcgcgggcaggagcggcgggccgcactgtgtcaacggcggtcgctgcaaccctggcaccggccagtgcgtctgccccgccggctgggtgggcgagcaatgccagcactgcgggggccgcttcagactaactggatcttctgggtttgtgacagatggacctggaaattataaatacaaaacgaagtgcacgtggctcattgaaggacagccaaatagaataatgagacttcgtttcaatcattttgctacagagtgtagttgggaccatttatatgtttatgatggggactcaatttatgcaccgctagttgctgcatttagtggcctcattgttcctgagagagatggcaatgagactgtccctgaggttgttgccacatcaggttatgccttgctgcatttttttagtgatgctgcttataatttgactggatttaatattacttacagttttgatatgtgtccaaataactgctcaggccgaggagagtgtaagatcagtaatagcagcgatactgttgaatgtgaatgttctgaaaactggaaaggtgaagcatgtgacattcctcactgtacagacaactgtggttttcctcatcgaggcatctgcaattcaagtgatgtcagaggatgctcctgcttctcagactggcagggtcctggatgttcagttcctgtaccagctaaccagtcattttggactcgagaggaatattctaacttaaagctccccagagcatctcataaagctgtggtcaatggaaacattatgtgggttgttggaggatatatgttcaaccactcagattataacatggttctagcgtatgaccttgcttctagggagtggcttccactaaaccgttctgtgaacaatgtggttgttagatatggtcattctttggcattatacaaggataaaatttacatgtatggaggaaaaattgattcaactgggaatgtgaccaatgagttgagagtttttcacattcataatgagtcatgggtgttgttgacccctaaggcaaaggagcagtatgcagtggttgggcactctgcacacattgttacactgaagaatggccgagtggtcatgctggtcatctttggtcactgccctctctatggatatataagcaatgtgcaggaatatgatttggataagaacacatggagtatattacacacccagggtgcccttgtgcaagggggttacggccatagcagtgtttacgaccataggaccagggccctatacgttcatggtggctacaaggctttcagtgccaataagtaccggcttgcagatgatctctaccgatatgatgtggatacccagatgtggaccattcttaaggacagccgatttttccgttacttgcacacagctgtgatagtgagtggaaccatgctggtgtttgggggaaacacacacaatgacacatctatgagccatggcgccaaatgcttctcttcagatttcatggcctatgacattgcctgtgaccgctggtcagtgcttcccagacctgattccaccatgatgtcaacagatttggccattccagcagtcttacacaacagcaccatgtatgtgttcggtggtttcaatagtctcctcctcagcgacatcctggtattcacctcggaacagtgtgatgcgcatcggagtgaagccgcttgtttagcagcaggacctggtattcggtgtgtgtggaacacagggtcgtctcagtgtatctcgtgggcgctggcaactgatgaacaagaagaaaagttaaaatcagaatgtttttccaaaagaactcttgaccatgacagatgtgaccagcacacagattgttacagctgcacagccaacaccaatgactgccactggtgcaatgaccattgtgtccccaggaaccacagctgctcagaaggccagatctccatttttaggtatgagaattgccccaaggataaccctatgtactactgtaacaagaagaccagctgcaggagctgtgccctggaccagaactgccagtgggagccccggaatcaggagtgcattgccctgcccgaaaatatctgtggcattggctggcatttggttggaaactcatgtttgaaaattactactgccaaggagaattatgacaatgctaaattgttctgtaggaaccacaatgcccttttggcttctcttacaacccagaagaaggtagaatttgtccttaagcagctgcgaataatgcagtcatctcagagcatgtccaagctcaccttaaccccatgggtcggccttcggaagatcaatgtgtcctactggtgctgggaagatatgtccccatttacaaatagtttactacagtggatgccgtctgagcccagtgatgctggattctgtggaattttatcagaacccagtactcggggactgaaggctgcaacctgcatcaacccactcaatggtagtgtctgtgaaaggcctgcaaaccacagtgctaagcagtgccggacaccatgtgccttgaggacagcatgtggagattgcaccagcggcagctctgagtgcatgtggtgcagcaacatgaagcagtgtgtggactccaatgcctatgtggcctccttcccttttggccagtgtatggaatggtatacgatgagcacctgcccccctgaaaattgttcaggctactgtacctgtagtcattgcttggagcaaccaggctgtggctggtgtactgatcccagcaatactggcaaagggaaatgcatagagggttcctataaaggaccagtgaagatgccttcgcaagcccctacaggaaatttctatccacagcccctgctcaattccagcatgtgtctagaggacagcagatacaactggtctttcattcactgtccagcttgccaatgcaacggccacagtaaatgcatcaatcagagcatctgtgagaagtgtgagaacctgaccacaggcaagcactgcgagacctgcatatctggcttctacggtgatcccaccaatggagggaaatgtcagccatgcaagtgcaatgggcacgcgtctctgtgcaacaccaacacgggcaagtgcttctgcaccaccaagggcgtcaagggggacgagtgccagctatgtgaggtagaaaatcgataccaaggaaaccctctcagaggaacatgttattatactcttcttattgactatcagttcacctttagtctatcccaggaagatgatcgctattacacagctatcaattttgtggctactcctgacgaacaaaacagggatttggacatgttcatcaatgcctccaagaatttcaacctcaacatcacctgggctgccagtttctcagctggaacccaggctggagaagagatgcctgttgtttcaaaaaccaacattaaggagtacaaagatagtttctctaatgagaagtttgattttcgcaaccacccaaatatcactttctttgtttatgtcagtaatttcacctggcccatcaaaattcaggtgcaaactgaacaatga

SEQ ID NO: 2 - DNA, 1231 bp, Homo sapiens
  Genbank Accession No. Z23090
gagggaactggtaatagaatttattcaaatgtgccttaatataggcgctggggcttgcccatggtgctcttgatatataaggcccggacattctgccagtttttcttgagcaatgacaccaagaagttgacagccaggtgaatgttatacacaagctcatcgtctgtcatcttcacgtgaccaacagctacagccagacataacaccttcttcatttggaacttgattgtggacttcacctcatccactttggccaccatgttttcgttgtgtgtgagcagggaagggaactttcctgccttatttaaacctgggccgaggattcgtggaatctgcttgatcagagactctgaggccaaaacgcatcatacttcttggccagcttcttgaccagttttttattcttgttgagttttttcagcgcctcgatgtccatgtgggggatatccacggccttagcctcgtcaagacccacaccagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcccggatgcccacccctgctgccgccactggctgtgcctcccccgccacctgtgtgttcttttgatacatttatcttctgtttttctcaaataaagttcaaagcaaccacctgt

SEQ ID NO: 3 - DNA, 488 bp, Homo sapiens
  Genbank Accession No. AA928646
tgggtatttcactttatttagcaaacggtcacactcggccccaccacaccgcgcctgcagcctcacttgagcatctgtctcaggaagcagctcagggtgaacaggaaggcctgggtttcccagttcaggccttatccgtcgccacacgtgccacacaccacaaaacaaaatcacttccccacgtcctcaggagcccaccccagtgtggtccgggggacgctccagtgttcaacagctgcctgcaggggccacagccagtctgtagacacaggccctggcacgaggcggtcccgggtgtaccacaaggcgatgcgtnctccatggaggttcttcttccctgtgccgtggactttgaagtggacgtgcatttcccagtctttgaggaagcacggctggtggttccagatagagccctctttgctgcgcccgtcaggggtcagacgtacgtactggctcgtgagcatagtgctgccctggaagttccagag

SEQ ID NO: 4 - DNA, 521 bp, Homo sapiens
  Genbank Accession No. AW023096
gcacgagcccagcccgagttatgcctgcccagcttaaggtcctaaccatgccggagacctgtngataccagcctttnaaaccactctctattggaggcatcatcattctgaaggataccagtgaanacattgaggagctggtggaacctgtggcagcacatggcccaaaaatcgaggaggaggaacaagagccagaacccccagaaccatttgagtatattgatgattaanggccagaggatctcacttgcttatctgaagaagattgtccaggctcatattgggaatgcttatgaggaaattcatgccgagacctgctattcaatgcatgtatcgttgcctctgcactgacctgaagaaccctgtcttccaagtcttttggttgaaagagaagatatantgactgttngagttgtggctcttttacaagaaacnttngnntttcaaataaatattaaagatctttccngatggacnnggaaaaanattaaacacttattttcctcgagggggggggcccc

SEQ ID NO: 5 - DNA, 619 bp, Homo sapiens
  Genbank Accession No. AA399076
caactagggggaaaacactttaggttgagtgctgagagaaaggctctctgagtaggtgacatctgaactaagagctgaagcagaagaataagccatcctctgtggaccaaaggggacaagattctctacttttctgctacgcactaggaatatagcagagaagacaagtggctgctaccttctgttatcgtgggtggcagtgactagtttttctagatcttgactccctgccgtaactaaagatggagtacactgatgctgaagtactatgagccttcggaacttgtggagagactacaaagttttggttgttatggtccctttagttgggctcatacatttggggtggtacagaatcaaaagcagccctgttttccaaatacctaaaaacgacgacattcctgagcaagatagtctgggactttcaaatcttcagaagagccaaatccaggggaagtagcaggcttgcaatcttcaggtaaagaagcagctttgaatctgagcttcatatcgaaagaagagatgaaaaataccagttggattagaaagaactggcttcttgtagctgggatatctttcataggtgtccatcttggaacatactttttgcagaggtctg

SEQ ID NO: 6 - DNA, 509 bp, Homo sapiens
  Genbank Accession No. U39317
gcacgggtcaccgcatcacaccatggctctgaagagaatccacaaggaattgaatgatctggcacgggaccctccagcacagtgttcagcaggtcctgttggagatgatatgttccattggcaagctacaataatggggccaaatgacagtccctatcagggtggagtatttttcttgacaattcatttcccaacagattaccccttcaaaccacctaaggttgcatttacaacaagaatttatcatccaaatattaacagtaatggcagcatttgtcttgatattctacgatcacagtggtctccagcactaactatttcaaaagtactcttgtccatctgttctctgttgtgtgatcccaatccagatgatcctttagtgcctgagattgctcggatctacaaaacagatagagaaaagtacaacagaatagctcgggaatggactcagaagtatgcgatgtaattaaacaaattattggataacctctacaaataaagatagggga

SEQ ID NO: 7 - DNA, 591 bp, Homo sapiens
  Genbank Accession No. W74749
gatagtaaaataattttatttagtttcttattttaaaaaaggtagcatttcaacatataaatttagactcaagattacagtgtatatttgccaaaaggaacaccccgaaagacggccagcctcagagctgagagggcacagggaggcacactcctcacacatggagtttaagcagaagtgctttaagacttcacagcggcatttccctgttcctcagccccgcctccagggccatcactttggggcaacagcttttgctcatgtaactataaaacatctctaggaatgaaagcacagaggtcaatgatccagattttccacaacaatcatctgcagcaacaattcgacaggtaaagattttatttttataattcaaaagttcttttaaggagaactaaagaacacaaaattcatttataaagagatttatagaattaaatggagccaaaatggaaaaattattttccccatattaccaatggcctctatanggataaggnccaggggggaagacccatttccaatctgggctttcccttccccaataagtgttttgggcacaacccacaactggtcatgagctgncaggcc

SEQ ID NO: 8 - DNA, 532 bp, Homo sapiens
  Genbank Accession No. AI916306
ttaccaatttaataaatgtactgtattaacatgaagactaatgacaaatgcactgcagtagtaagcacgtcatagatgcatagaatattctctatatagtctgaatatggatataaaataagttatactcattttgttttccatcacagtaggagcatagcatacaaagtgattggttcagtggccatgaagcaagccaggggagagaccacagaagagaatgtagggcattgagtacagtggggatttgccaaggacactgcagagtccctggggaccctctggggaacaaggccccaaacctctcaagttagccttctgagaatagtatgaggtcccacttgacaagagacagtggctgcatctgagctgggatgattcaggcaactgggtgggaggcagagcagggacaggcacgaacagcatccagcaggctagcccactctcctgctggtgctgttggatgtctatgatgctgggaatatggggcgcagagccagaaggcacggtttgcttctgtgacgtgacagat

SEQ ID NO: 9 - DNA, 2834 bp, Homo sapiens
  Genbank Accession No. X77777
tcggagcctgcggagggtggtggtggtggtggtggtggccctcgcccgcctcactcatgcctcctcctcctctgctctcgctcaggcgcctcggtggcggttggtcggcggttacgcggctggtggtcgcggcggccggggctcgctctcggggaggccggggcggatctcgcggcgcaggcggcggcggccgaggtggggtcgcgcggcggaggcggctcgagcttcgtgctgcgcgctcgctcttgggctcctcgctgcaggaggagtgtgactatgtgcagatgatcgaggtgcagcacaagcagtgcctggaggaggcccagctggagaatgagacaataggctgcagcaagatgtgggacaacctcacctgctggccagccacccctcggggccaggtagttgtcttggcctgtcccctcatcttcaagctcttctcctccattcaaggccgcaatgtaagccgcagctgcaccgacgaaggctggacgcacctggagcctggcccgtaccccattgcctgtggtttggatgacaaggcagcgagtttggatgagcagcagaccatgttctacggttctgtgaagaccggctacaccattggctacggcctgtccctcgccacccttctggtcgccacagctatcctgagcctgttcaggaagctccactgcacgcggaactacatccacatgcacctcttcatatccttcatcctgagggctgccgctgtcttcatcaaagacttggccctcttcgacagcggggagtcggaccagtgctccgagggctcggtgggctgtaaggcagccatggtctttttccaatattgtgtcatggctaacttcttctggctgctggtggagggcctctacctgtacaccctgcttgccgtctccttcttctctgagcggaagtacttctgggggtacatactcatcggctggggggtacccagcacattcaccatggtgtggaccatcgccaggatccattttgaggattatggtctgctcaggtgctgggacaccatcaactcctcactgtggtggatcataaagggccccatcctcacctccatcttggtaaacttcatcctgtttatttgcatcatccgaatcctgcttcagaaactgcggcccccagatatcaggaagagtgacagcagtccatactcaaggctagccaggtccacactcctgctgatccccctgtttggagtacactacatcatgttcgccttctttccggacaattttaagcctgaagtgaagatggtctttgagctcgtcgtggggtctttccagggttttgtggtggctatcctctactgcttcctcaatggtgaggtgcaggcggagctgaggcggaagtggcggcgctggcacctgcagggcgtcctgggctggaaccccaaataccggcacccgtcgggaggcagcaacggcgccacgtgcagcacgcaggtttccatgctgacccgcgtcagcccaggtgcccgccgctcctccagcttccaagccgaagtctccctggtctgaccaccaggatcccagcccaagcggcccctcccgccccttcccactcgcagcagacgccggggacagaggcctgcccgggcgcgccagccccggccctgggctcggaggctgcccccggccccctggtctctggtccggacactcctagagaacgcagccctagagcctgcctggagcgtttctagcaagtgagagagatgggagctcctctcctggaggatgcaggtggaactcagtcattagactcctcctccaaaggccccctacgccaatcaagggcaaaaagtctacatactttcatcctgactctgccccctgctggctcttctgcccaattggaggaaagcaaccggtggatcctcaaacaacactggtgtgacctgagggcagaaaggttctgcccgggaaggtcaccagcaccaacaccacggtagtgcctgaaatttcaccattgctgtcaagttcctttgggttaagcattaccactcaggcatttgactgaagatgcagctcactaccctattctctctttacgcttagttatcagctttttaaagtgggttattctggagtttttgtttggagagcacacctatcttagtggttccccaccgaagtggactggcccctgggtcagtctggtgggaggacggtgcaacccaaggactgagggactctgaagcctctgggaaatgagaaggcagccaccagcgaatgctaggtctcggactaagcctacctgctctccaagtctcagtggcttcatctgtcaagtgggactctgtcacaccagccattcttatctctctgtgctgtggaagcaacaggaatcaagagactgccctccttgtccacccacctatgtgccaactgttgtaactaggctcagagatgtgcacccatgggctctgacagaaagcagatcctcaccctgctacacatacaggatttgaactcagatctgtctgataggaatgtgaaagcacggactcttactgctaacttttgtgtatcgtaaccagccagatcctcttggttatttgtttaccacttgtattattaatgccattatccctgaattccccttgccaccccaccctccctggagtgtggctgaggaggcctccatctcatgtatcatctggataggagcctgctggtcacagcctcctctgtctgcccttcaccccagtggccactcagcttcctacccacacctctgccagaagatcccctcaggactgcaacaggcttgtgcaacaataaatgttggcttggaaaaaaaaaaaa

SEQ ID NO: 10 - DNA, 2799 bp, Homo sapiens
  Genbank Accession No. X63359
ctgcacaaggatggctctgaaatggactacagttctgctgatacaactcagtttttactttagctctgggagttgtggaaaggtgctggtatgggccgcagaatacagcctttggatgaatatgaagacaatcctgaaagaacttgttcagagaggtcatgaggtgactgtactggcatcttcagcttccattctttttgatcccaacgactcatccactcttaaacttgaagtttatcctacatctttaactaaaactgaatttgagaatatcatcatgcaattggttaagagattgtcagaaattcaaaaagatacattttggttacctttttcacaagaacaagaaatcctgtgggcaattaatgacataattagaaacttctgtaaagatgtagtttcaaataagaaacttatgaaaaaactacaagagtcaagatttgacatcgtttttgcagatgcttatttaccctgtggtgagctgctggctgagctatttaacataccctttgtgtacagtcacagcttcagtcctggctactcatttgaaaggcacagtggaggatttattttccctccttcctacgtacctgttgttatgtcaaaattaagtgatcaaatgactttcatggagagggtaaaaaatatgctctatgtgctttattttgacttttggttccaaatatttaatatgaagaagtgggatcagttttacagtgaagttttaggaagacccactacattatctgagacaatgaggaaagctgacatatggcttatgcgaaactcctggaattttaaatttcctcatccattcttaccaaatgttgattttgttggaggactccactgcaaacctgccaaacccctacctaaggaaatggaggagtttgtacagagctctggagaaaatggtgttgtggtgttttctctggggtcaatggtcagtaacatgacagaagaaagggccaacgtaattgcaacagcccttgccaagatcccacaaaaggttctttggagatttgatgggaataaaccagatgccttaggtctcaatactcgactgtacaagtggataccccagaatgaccttctaggtcatccaaaaaccagagcttttataactcatggtggagccaatggcatctatgaggcaatctaccatgggatccctatggtgggcattccattgttttttgatcaacctgataatattgctcacatgaaggccaagggagcagctgttagagtggacttcaacacaatgtcgagtacagacctgctgaatgcactgaagacagtaattaatgatccttcatataaagagaatattatgaaattatcaagaattcaacatgatcaaccagtgaagcccctggatcgagcagtcttctggattgaatttgtcatgcgccacaaaggagccaaacatcttcgagttgcagcccacaacctcacctggttccagtaccactctttggatgtgattgggttcctgctggcttgtgtggcaaccgtgctatttatcatcacaaagtgttgtctgttttgtttctggaagtttgctagaaaaggaaagaagggaaaaagggattagttatatctgagatttgaagctggggaattccgtttattgaagattcaggttaacctgaatcaagttaacccagtctcaaatgctcacttatcctatctccttggcacaaatctctcctctcctggattgccaaagaaaattcaaattattcttcaattagtcaggatgatttgactatcagcagttcatagtacccatcttcataactaagccacctagggatccggcagaaaaaaaagggatgcaggggagtcatcatacagggggtggatcatttaccaggatccacacttcctacaaagcggttgtaatattaaataacaaaactgttttttattccaatcttcacataaaacaggaataattgtatactttcttactaatgtgttccatggagtttttcctccaagaagtggcttaggggaaaatgagccccagtaatgctctgtggcatccaatccttctaccccgaccctttgactttctgccccagcccctcttagttctcctagaattaggactaaggttaagtgccctcttgggatatgacttccttcccttcctcttgatacaaaaagagcctattaccaaccctcatacacacaagagttcccttcctagttgcagactcttctgctccagctggactcccctagctctggactcccactagatcacacaggggtccctgcatgtcagtaaactttggatgaccttgggagaccaaaaaatggaatatcattttttgatctaaacaaaatagtttcctgatttaacactggccaggaaggtgggctgcaccctcagtctctctctcccatcatggttttcacatgatatcaaaggactctcataacagtctgattcttatgagttgggcatcctgtgtttccctttagggcctgcttccttcaaatagaggagatgggtgctatgaaacctattcactctggacttgggatggctcttctccatcttcccaagtctgagctggagcctccatgcccaactctgctctgctcttctatttcctgacagcagctaaggcatgttcctgttctgcccccaaattgaccttactaacagtgagaacttggaggagtcttcgggtcttgggaaatccaagttttcccggaaacgttttgttgtaaacagtgtccacactctttgctccaataaagctcggttccttaagggaattc

SEQ ID NO: 11 - DNA, 2194 bp, Homo sapiens
  Genbank Accession No. Z48475
gtgaccagaggggtttgtgtggctgaagaggcaggaggaacagtgtatccacagcgtgggaccatgccaggcacaaaacggtttcaacatgtcattgagaccccggagcctggcaagtgggagttgtctgggtacgaggcagctgtgccaatcacggagaagtcaaacccactgacccaggatctagacaaagcagatgctgagaacattgttcgactgctagggcaatgtgatgctgagatcttccaggaggaggggcaagccctgtccacataccagagactctacagcgaatccattctgaccaccatggtacaggtggctgggaaagttcaggaagtgctgaaggagccagatggggggctggttgtgctgagtggagggggcacctctggccggatggcattcctcatgtcggtgtcctttaatcagctgatgaaaggtctgggacagaaacctctttacacctacctcattgcaggtggtgacaggtctgtggtggcctctagggaggggacagaagatagtgccttgcacgggattgaggaactgaagaaggtggctgccgggaagaagagagtgattgtcattggcatttctgtgggactctctgctccctttgtggcaggccagatggactgctgcatgaacaacacagctgtcttcttgccagtcctggttggcttcaatccagtgagcatggccagaaatgaccccattgaagactggagttcaacattccgacaagtagcagagcggatgcagaaaatgcaggagaaacagaaagcttttgtgctcaatcctgccatcgggcccgagggtctcagcggctcctcccggatgaaaggtggaagtgccaccaagattctgctggaaaccctgttattagcagcccataagactgtggaccagggcattgcagcatctcaaagatgcctcctggaaatcttgcggacatttgagcgagctcatcaggtgacctacagccaaagccccaagattgccaccctgatgaagagtgtcagcaccagtctggagaagaaaggccacgtgtacctggttggctggcagaccctgggtatcattgccatcatggatggagtagagtgcatccacacctttggtgctgatttccgagatgtccgtggctttctcattggtgatcacagtgacatgtttaaccagaaggctgagctcaccaaccagggtccccagttcaccttctcccaggaggacttcccgacttccatccttccctctctcacggaaatcgatactgtggtcttcattttcaccctggatgacaacctcacggaggtgcagactatagtggagcaggtgaaagagaagaccaaccacatccaggccctggcacacagcaccgtgggtcagaccttgccgatccctctgaagaagctctttccctccatcatcagcatcacatggccactgcttttctttgaatatgaagggaacttcatccagaagttccagcgtgagctaagcaccaaatgggtgctgaatacagtgagtacaggtgctcatgtgcttcttggtaagatcctacaaaaccacatgttggaccttcggattagcaactccaagctcttctggcgggcgctggccatgctgcagcggttctctggacagtccaaggctcgatgcatcgagagcctcctccgagcgatccactttccccagccactgtcagatgatattcgggctgctcccatctcctgccgtgtccaggttgcacatgagaaggaacaggtgatacccatcgccttgctgagcctcctattccggtgctcgatcactgaggctcaggcacacctggctgcagctccttctgtctgtgaggctgtcaggagtgctcttgctgggccaggtcagaagcgcactgcggaccccctcgagatcctagagcctgacgttcagtgaacccatgtttctgggtgggtgaaaggggcccaaccctgcccacttcagcccagcccgcccaaggggacttgtgccagcagaacatgtgggaggaagaagccccgtttccagggcatccgcagcccagggtagggagaaatattctctccactttgggggagagttcttgctctcgacctagtggtttctactctcaccgacttattctgatttcagaaataaaatgaaatgtcttattttggaaaaaaaaaaa

SEQ ID NO: 12 - DNA, 2171 bp, Homo sapiens
  Genbank Accession No. U17418
tcaacacgtgaattcccacagaagctgctcagggactatccatggcctccccgtggccaacttgagtctgctctgcagctttaggcccgacttggaaggcccatgggctgcagatgaggaaactgaggtccagacagccgaagagtggtagtgtccaggacacacaactgggccggcggcggcggctgccccgagggacgcggccctaggcggtggcgatggggaccgcccggatcgcacccggcctggcgctcctgctctgctgccccgtgctcagctccgcgtacgcgctggtggatgcagatgacgtcatgactaaagaggaacagatcttcctgctgcaccgtgctcaggcccagtgcgaaaaacggctcaaggaggtcctgcagaggccagccagcataatggaatcagacaagggatggacatctgcgtccacatcagggaagcccaggaaagataaggcatctgggaagctctaccctgagtctgaggaggacaaggaggcacccactggcagcaggtaccgagggcgcccctgtctgccggaatgggaccacatcctgtgctggccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttaggcggctgcactgcacgcgcaactacatccacatgcacctgttcctgtccttcatgctgcgcgccgtgagcatcttcgtcaaggacgctgtgctctactctggcgccacgcttgatgaggctgagcgcctcaccgaggaggagctgcgcgccatcgcccaggcgcccccgccgcctgccaccgccgctgccggctacgcgggctgcagggtggctgtgaccttcttcctttacttcctggccaccaactactactggattctggtggaggggctgtacctgcacagcctcatcttcatggccttcttctcagagaagaagtacctgtggggcttcacagtcttcggctggggtctgcccgctgtcttcgtggctgtgtgggtcagtgtcagagctaccctggccaacaccgggtgctgggacttgagctccgggaacaaaaagtggatcatccaggtgcccatcctggcctccattgtgctcaacttcatcctcttcatcaatatcgtccgggtgctcgccaccaagctgcgggagaccaacgccggccggtgtgacacacggcagcagtaccggaagctgctcaaatccacgctggtgctcatgcccctctttggcgtccactacattgtcttcatggccacaccatacaccgaggtctcagggacgctctggcaagtccagatgcactatgagatgctcttcaactccttccagggattttttgtcgcaatcatatactgtttctgcaacggcgaggtacaagctgagatcaagaaatcttggagccgctggacactggcactggacttcaagcgaaaggcacgcagcgggagcagcagctatagctacggccccatggtgtcccacacaagtgtgaccaatgtcggcccccgtgtgggactcggcctgcccctcagcccccgcctactgcccactgccaccaccaacggccaccctcagctgcctggccatgccaagccagggaccccagccctggagaccctcgagaccacaccacctgccatggctgctcccaaggacgatgggttcctcaacggctcctgctcaggcctggacgaggaggcctctgggcctgagcggccacctgccctgctacaggaagagtgggagacagtcatgtgaccaggcgctgggggctggacctgctgacatagtggatggacagatggaccaaaagatgggtggttgaatgatttcccactcagggcctggggccaagaggaaaaacagggaaaaaaagaaaaaaaaaaagaaaagaaaaaaaggaaaaaaaaaaaaaaaaaaaaaaaaaaa

SEQ ID NO: 13 - DNA, 833 bp, Homo sapiens
  Genbank Accession No. S82240
cagaaattatccagcaaatctatcatggatcctaatcagaacgtgaaatgcaagatagttgtggtgggagacagtcagtgtggaaaaactgcgctgctccatgtcttcgccaaggactgcttccccgagaattacgttcctacagtgtttgagaattacacggccagttttgaaatcgacacacaaagaatagagttgagcctgtgggacacttcgggttctccttactatgacaatgtccgccccctctcttaccctgattcggatgctgtgctgatttgctttgacatcagtagaccagagaccctggacagtgtcctcaaaaagtggaaaggtgaaatccaggaattttgtccaaataccaaaatgctcttggtcggctgcaagtctgatctgcggacagatgttagtacattagtagagctctccaatcacaggcagacgccagtgtcctatgaccagggggcaaatatggccaaacagattggagcagctacttatatcgaatgctcagctttacagtcggaaaatagcgtcagagacatttttcacgttgccaccttggcatgtgtaaataagacaaataaaaacgttaagcggaacaaatcacagagagccacaaagcggatttcacacatgcctagcagaccagaactctcggcagttgctacggacttacgaaaggacaaagcgaagagctgcactgtgatgtgaatctttcattatctttaatgaagacaaaggaatctagtgtaaaaaacaacagcaaacaaaaaggtgagtctaaatgaagtgcacagccaaagtcatgtataccagaggcttaggaggcg

SEQ ID NO: 14 - DNA, 1445 bp, Homo sapiens
  Genbank Accession No. AF017786
gagttggggctggcgctccggagttgctgggctcagcgcagctcccattcattaaggaaccagctgcggaggaaggtggccgagcgcccgcgctgcccactcgctcgctcgcgcactcagacgcgcgccacaacagcgcgccccaagctgcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgcctgggtgtgtggctgctgttgcgggacgtcttcgcggggcgggaggctcgcgccgcagccagcgccatgcaaaactacaagtacgacaaagcgatcgtcccggagagcaagaacggcggcagcccggcgctcaacaacaacccgaggaggagcggcagcaagcgggtgctgctcatctgcctcgacctcttctgcctcttcatggcgggcctccccttcctcatcatcgagacaagcaccatcaagccttaccaccgagggttttactgcaatgatgagagcatcaagtacccactgaaaactggtgagacaataaatgacgctgtgctctgtgccgtggggatcgtcattgccatcctcgcgatcatcacgggggaattctaccggatctattacctgaagaagtcgcggtcgacgattcagaacccctacgtggcagcactctataagcaagtgggctgcttcctctttggctgtgccatcagccagtctttcacagacattgccaaagtgtccatagggcgcctgcgtcctcacttcttgagtgtctgcaaccctgatttcagccagatcaactgctctgaaggctacattcagaactacagatgcagaggtgatgacagcaaagtccaggaagccaggaagtccttcttctctggccatgcctccttctccatgtacactatgctgtatttggtgctatacctgcaggcccgcttcacttggcgaggagcccggctgctccggcccctcctgcagttcaccttgatcatgatggccttctacacgggactgtctcgcgtatcagaccacaagcaccatcccagtgatgttctggcaggatttgctcaaggagccctggtggcctgctgcatagttttcttcgtgtctgacctcttcaagactaagacgacgctctccctgcctgcccctgctatccggaaggaaatcctttcacctgtggacattattgacaggaacaatcaccacaacatgatgtaggtgccacccacctcctgagctgtttttgtaaaatgactgctgacagcaagttcttgctgctctccaatctcatcagacagtagaatgtagggaaaaacttttgcccgactgatttttaaaaaaaaaaaaaaaaaa

SEQ ID NO: 15 - DNA, 388 bp, Homo sapiens
  Genbank Accession No. AI985046
tttttttacatttcaaatatattttattactttccatcttagaaagaatatgaaacctgcatgcaatgctaatggtttctgacatgtacatagcatataacacagcagtacaatgcggcatatactggggggcagtgtgtggagggggcgttcttaagggtatatgtacagaggaaagggcgcatggtcatcttagctttcgaaagaggactgcactgtttaacattgaagaattacatggggaatcacaaatatattgctttagtactgcatgttctgttgtggtgagggaaagaaacatgctttgaaggttttcccttgtcaacagaatgtgtgtctgtagctgtgtattgcgcatgtattcatatatttttaagttttctcctaa

SEQ ID NO: 16 - DNA, 2244 bp, Homo sapiens
  Genbank Accession No. U19495
gcacgggacaggccgggccacacccaccggggcgagctcggagggcggcgctctgggcggagggcccggcggctcggcccagggcgcgttacctcgtcgccggggccggagagggcgggcggaggcacggggcctggaggcgccaggcggaggatgcgggcgacacggtggcggcggcgaccgcgcgaccgggcgggcgggcgggcaggggcgagcggagggagggagcggactgcggcaggatctgtcgaggaaaaatcttgcggccggcgattccccgccttttaagcgcagcctgcactccccccaccccacgcaggggcgggccttccccaacgcgggcgcccactggccgccgcgcgccgctcccctccagctcgcctgcgcctctcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaatttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcattaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggta

SEQ ID NO: 17 - DNA, 1744 bp, Homo sapiens
  Genbank Accession No. M12625
tgaggcctgactttttcaataaaacattgtgtagttctgggcctcctgctgccccggctctgtttcccctggcgccaagagaagaaggcggaactgaacccaggcccagagccggctccctgaggctgtgcccctttccggcaatctctggccacaacccccactggccaggccgtccctcccactggccctagggcccctcccactcccacaccagataaggacagcccagtgccgctttctctggcagtaggcaccagggctggaatggggccgcccggctccccatggcagtgggtgacgctgctgctggggctgctgctccctcctgccgcccccttctggctcctcaatgtgctcttccccccgcacaccacgcccaaggctgagctcagtaaccacacacggcccgtcatcctcgtgcccggctgcctggggaatcagctagaagccaagctggacaaaccagatgtggtgaactggatgtgctaccgcaagacagaggacttcttcaccatctggctggatctcaacatgttcctaccccttggggtagactgctggatcgataacaccagggttgtctacaaccggagctctgggctcgtgtccaacgcccctggtgtccagatccgcgtccctggctttggcaagacctactctgtggagtacctggacagcagcaagctggcagggtacctgcacacactggtgcagaacctggtcaacaatggctacgtgcgggacgagactgtgcgcgccgccccctatgactggcggctggagcccggccagcaggaggagtactaccgcaagctcgcagggctggtggaggagatgcacgctgcctatgggaagcctgtcttcctcattggccacagcctcggctgtctacacttgctctatttcctgctgcgccagccccaggcctggaaggaccgctttattgatggcttcatctctcttggggctccctggggtggctccatcaagcccatgctggtcttggcctcaggtgacaaccagggcatccccatcatgtccagcatcaagctgaaagaggagcagcgcataaccaccacctccccctggatgtttccctctcgcatggcgtggcctgaggaccacgtgttcatttccacacccagcttcaactacacaggccgtgacttccaacgcttctttgcagacctgcactttgaggaaggctggtacatgtggctgcagtcacgtgacctcctggcaggactcccagcacctggtgtggaagtatactgtctttacggcgtgggcctgcccacgccccgcacctacatctacgaccacggcttcccctacacggaccctgtgggtgtgctctatgaggatggtgatgacacggtggcgacccgcagcaccgagctctgtggcctgtggcagggccgccagccacagcctgtgcacctgctgcccctgcacgggatacagcatctcaacatggtcttcagcaacctgaccctggagcacatcaatgccatcctgctgggtgcctaccgccagggtccccctgcatccccgactgccagcccagagcccccgcctcctgaataaagaccttcctttgctaccgtaagccctgatggctatgtttcaggttgaagggaggcactagagtcccacactaggtttcactcctcaccagccacaggctcagtgctgtgtgcagtgaggcaagatgggctctgctgaggcctgggactgagct

SEQ ID NO: 18 - DNA, 686 bp, Homo sapiens
  Genbank Accession No. AL042399
gtgcctgtcatcttcgttgctgcccacatgatgtcatctggctgctcttgggcagggctgggagccactttggggttctgggaagggcaggaggggctctgtggctctaaagcacctccctttaaccccctttgtccatccacagctctcggccactttaaccattcccatctggcagtggttcttgacccggtttggcaagaagacagctgtatatgttgggatctcagtgagtggggttgaagagcagagcctgggttgagttgggatgtctggtgggaacctcccagctgattcatcttcctgcacccccttccctagtcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggcttnccataagttctcatcctgctgggcctc

SEQ ID NO: 19 - DNA, 364 bp, Homo sapiens
  Genbank Accession No. D63160
cctgcacaggagattccctgacgttccacaacaaccagtccttctccaccaaagaccaggacaatgatcttaacaccggaaattgtgctgtgatgtttcagggagcttggtggtacaaaaactgccatgtgtcaaacctgaatggtcgctacctcagggggactcatggcagctttgcaaatggcatcaactggaagtcggggaaaggatacaattatagctacaaggtgtcagagatgaaggtgcgacctgcctagcccaggccggcctcagggtcaggacgcctccacacatagttggttggggggtagggtttgggagcttggccctacggtttgtaaaagaaacacatgtcgtgattcta

SEQ ID NO: 20 - DNA, 1564 bp, Homo sapiens
  Genbank Accession No. D88587
cgggcaggcccagccttgtagtctgcaaagaccatgcaagccttggttgataaaccgggattctactggaagtttgcactttccccacatcagccaagatgggtaaactgccttaacacaaaagtcacatgccccacaaaatatttccttctatctcaaaacagtattagcttctttaatctaagaaggtaatgagaggtttttaaaactgagttaagtcagcagttcagcaagtgcttacgaaagacagtcaaaatataggcaagttactttaaatgggtattatttgtccttactttcccaatgtaggcaagtgcctgtgatataacaatttagggaataatgggagaataaaaagcatattagtttcaatcagatttcctggcatatatcacagaccctaaggaattacaaagacaaaaaagcaagctctttggtttctgcatggttctttgcagactacaaggctgcccctgcaagcacttggatgaaaagaccgggattctactggaagtttgcactttccccacatcagccaagatggatctactgtggatcctgccctccctgtggcttctcctgcttggggggcctgcctgcctgaagacccaggaacaccccagctgcccaggacccagggaactggaagccagcaaagttgtcctcctgcccagttgtcccggagctccaggaagtcctggggagaagggagccccaggtcctcaagggccacctggaccaccaggcaagatgggccccaagggtgagccaggagatccagtgaacctgctccggtgccaggaaggccccagaaactgccgggagctgttgagccagggcgccaccttgagcggctggtaccatctgtgcctacctgagggcagggccctcccagtcttttgtgacatggacaccgaggggggcggctggctggtgtttcagaggcgccaggatggttctgtggatttcttccgctcttggtcctcctacagagcaggttttgggaaccaagagagtgaattctggctgggaaatgagaatttgcaccagcttactctccagggtaactgggagctgcgggtagagctggaagactttaatggtaaccgtactttcgcccactatgcgaccttccgcctcctcggtgaggtagaccactaccagctggcactgggcaagttctcagagggcactgcaggggattccctgagcctccacagtgggaggccctttaccacctatgacgctgaccacgattcaagcaacagcaactgtgcagtgattgtccacggtgcctggtggtatgcatcctgttaccgatcaaatctcaatggtcgctatgcagtgtctgatgctgccgcccacaaatatggcattgactgggcctcaggccgtggtgtgggccacccctaccgcagggttcggatgatgcttcgatagggcactctggcagccagtgcccttatctctcctgtacagcttccggatcgtcagccaccttgcctttgccaaccacctctgcttgcctgtccacatttaaaaataaaatcattttagccctttc

SEQ ID NO: 21 - DNA, 447 bp, Homo sapiens
  Genbank Accession No. AA224832
agcaggccancatactactactaanttcgcggccngtcngactctgtcccgctgcgtgttctcctctcagatcggggactcctgcttctccttgccantaaatggaccccaactgctcctgctcgcctgttggctcctgtgcctgtgccggctcctgcaaatgcaaagagtgcaaatgcacctcctgcaagaagagctgctgctcctgctgccctgtgngtgctgccaagtgtgcccagggctgcatctgcaaagggacgtcagacaaagtgcagctgctgtgcctgattgccaggacagctgtgctnctcagatgtaaataagagcaacctatataaacctggatttttttttttttttttttttgtacaaccctgacccgtttggcaaatctttttttctatgaaatatgtgaatgggcaataaattcatctagactaaaaaaaa

SEQ ID NO: 22 - DNA, 567 bp, Homo sapiens
  Genbank Accession No. AI052592
caggtttctaacatttaatctccacttagggaattggaggtcagaaatgaataaaataccatgccccagtaggctccagtctagggaagtgggcagcaggcagaaaccaagacacagaaagcaacgacacataacagatatgctatgatacaaatggtgctgcaggagctgcacagttcggaggccatttctggccagaggatgggatgcgtgaaatccaggaaggcttccaggaaggagtagctggcactgggatgggcaggaacagcaatgtctctactgatggggtnttttttnccaaaattttttttttggagaaagctggcaaagttgagaagtttggagactaaccgtgacaaaatttaaacatcaggtatgtgaaggcaagggggaccaactgtgacaaaatgtaaacgtcaagtgtgtgaagtcaaaggggaaccaggaaagtaaaagagtgacaatcagcagtgcttttggcaaaaaggcctctggcagcagtgtagaagagaaacgcagagatcacaggtataagaatgacagtaggagaggggaggagggtacata

SEQ ID NO: 23 - DNA, 430 bp, Homo sapiens
  Genbank Accession No. AI937227
ttttttggaaatcaaaagaaccagccattttattccaagaggggtgttttggggcagcaggaataaataaggaagggaggggacgggggcagggcaggtaggttctacgtcttgcagcacatcccacactttgatcgatgacagcagccgcagcagaaaatgcagatggggaagtgggtgtctcgcctccttcgcctctggaacatgggcatccagctggccctggctccagctctgtcctggggttgcagctctgcaagttgtcccgtctgttgtgggaaaacagagccactggtcaggctggcgaggaggaggaggagcaggaggcaagcggcccagatctgggagctcagtgccatcgtgccgtctgtctggctgtcccactgctgggtcttgagcttgctctggtgtctgggaccgagtgacagtc

SEQ ID NO: 24 - DNA, 630 bp, Homo sapiens
  Genbank Accession No. AA167715
tttttttttttttttttttttttttttttttttttttaaatttcaagacaactttatccagacaggcgcctcncaaatagaacacagggaagttaggcagcagttactaaaatacagtcncgccaaatgatttacaacagaacacaacaggagcaggggatctgtgggtggggctgggctgggccnctatctcacagggcctgagtcaagccagcccgccctgcaaggcagggctgacctgcaagcggagatctcacttcctcttaccccaaattcatacctccattttccccgcccccatctctccccagggtcctcaagtgggaaagggagaggtagcatccctcggatccaggcccactccactccgtctccggcaccagtgggcaggctgagtctgggcctcaaggggcctgggcttagggtatctatggcagtaggaaaatgacatggacaggctcttcaggggtaggctaaagtcctctggccagcagtacccagagaaaatgggcagcagcaggtaaaccagccaggaggtggagtcctctgaanccacagcagancccacctctggccagccctgnccaaattgggggtaagacatgaaatctggtaagaaatggtgcctca

SEQ ID NO: 25 - DNA, 489 bp, Homo sapiens
  Genbank Accession No. AI424433
cctcttcctcctcctcttctacttcaggctcatctttcattctcattagagttggagggagttctggacgcttggggggtaacttccctactgttccagtttttaatttgaatttgcttcctcctttcatggcaccaaatagaggcaatgtaagctttttagctttgttttctgcacctgtagtctgagtttcagtcttttttagttctggaatctctgctggctttacaatttttattaaccctttaagtctctgttgttctttcctcagttcaaaagttctcaggtgaagtttcttccgggacacaccatctaatgtactgcctgatttcatttctgacatgaacgcatctaaagaatcctgagatggagactctgatagaacttggcttgaggctttcaatctctcagaaatttcagaaagttccctctcagcatcatttaattttgcaaccaatgattcaaaggtctctggcttctcatcaatct

SEQ ID NO: 26 - DNA, 6711 bp, Homo sapiens
  Genbank Accession No. X52851
gaattcccttgtaaggttttcttaacaaaacaccagtcacataagtgcattttattttatatttttgtttatttatttgagacggagtctcttgtctctcaggctggagtgcagtggcgccatctctgctcgctgcaacctccacctcctgggttccagcgattctcctgcctcagcctcccgagggggtagctgggactacaggtgcgcaccaccatgcccagctaattttgtatttttcgtagagatggggtttcaccatgttgtccaggctggtcttgaactcctgacctcaggtgatcctcccgcctcggcctcccaaagtgctggaattacaggcgtgatccaccgcacccggcctattttttgagagagggtcacactctgtcgtcccggctggaatgcagtgatgcgatcaccgcccactacagcctcgacctccgggctcaagcaatcctccccgcccagcctcctgagtagcgagcgcctcgacgcccagctaatttttatttttatttatttttttgtagagacggcgtctctctaagatgcccaggctggtggccggtgtcgaactcctaagatgaagcgatcctccccggccttggcctccgcgcctcctaaagcgccaggtatgagccaccgcgcctggcctacaagtgcattttaattaaagtattattaatgtctttgcctgaagaaattcgcttttaaattgtgacttatctttcacccaaaaatcaaagcacaattcagccccgaggcgggggcggtaggagctgggcggggcgggggcagggaaagaccaggagcagagattcaaaaagagtaagagggcaaaatgtgcataatgcatcttcacaggtaagagcctggccaggctcctgttttaatggcttcctcctgaagaagattcaagcagagtgtaagatattttcggaaagtagagcattttgaaagcatttcataatctctcaaaaccggagactgctcctgtcccacctcgttagagaaaacagcgatgctcaaaggcaacctccttcctgacattgcctggtaggacgcgacgtggtgtttgcccgcgcggaatgcggacgcaaggctgctcctaggtctcggggacgcgccatccccatttccgctcgcggaggcgtagggtccgggcgcgggaccccagtcgaccttgactggcggcgcgaccttgaggcctgcgttcgcctcagttgccccctctgtgcaatggggagacgcgcctcatcgcttgacaacggccgaagagccgccgcgcttccgtctcccgcgtgcgcgcgccatgctgcccacccccgttccgcactgaccctcccccgtgccccgcgtcccgtactgccgccccgccccgagtcccatgccgcagccaccgcgacggagcccgcaggcgggaacctgcctccgcgcgttagcgcgcacgcgcgcctcatgtgtcgtccccatcagcgccggcttccgtctataggccagatgcactgtcactctggcgaagtcgcagacccgattggccgggacggaggcgcgagaccgggttgcgggcggggccgaacgtggtataaaacgggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgaggtcgggcgggcggcggcgtgcgggaatggggcccagaaagtgggccggggtcggggtgggtggtagcgccccaaaggcccgggcgcggggcgaccctgcttgaggggcgagcgcgggcgggctgcggcgccatttcctgacgaggggccattttgggaggtccgcgagtcgcgggaggaggccgggacgcggcggacaaaggcaggcggggcggctgcgaggccgttgggggagggggcccgcgtccgcccgcccgcctcatgtggccgcgccctgtcctgtccgacgcacgtgctcggcggccgcgctcaggtccgcgccttgagagtcgttgtccgccctagcttggcctgggcgccgcagaccggagccagaagcacgctcgcgggggcttgcgaccgccttcctgggaagctgtcccctggcaggcatgggtgctttacatcctgagctgggaagctgtttgcttgagggtttttctcaaggatcgaggcgcggtgtgagcccgtccatgctcggtcctgtagatcccgggaggccatgttataaaaggagacttgctgggatgtgacgggttgccacttgaaatatcttccatttggataaagtaggaatatttatacatgtgccccaaacgtccctccgtgtcccccacccccaagcggaaatgtgaaaatgggccttgcctttgctggtgcccaaggaccgccttccactgcagtgacggcgctggcgggggaggcgctcttgagcccctcccgattgtccctctgcctagcaagcaagttgcgactggccacaaggcaggcctcttccgaccaaggtggattaccagtgattacctaattagttttgagagcgttaaatgagttcttaaagatcagttgtaattatagcatagtatctaaacttggcgcgtgtcttcaaagttaaatattgagtacgattccgttccagttaacatggatagaccttagggagtagcgaaataggatgttagtggttttattcctttaaatcacatctcaaaaggccaccaatggctagtttggatcttattccgaaaatagattgatcctcatgcagtcttcgtgaggacagagcgatttccttgttgcctaccctgtccatagtgcctggcacataggcactgaaacactgcatgttaatccacaccccaccccacctatgagtgtagtcaaagctggtaagtgacaagggctttcgtggaaacttggcctgacctaatgttgggcatcaggttacccaaagagcttcagggaaatgagaaaggacttgcaggtcttgatgagaatggaggggtaactgccaatgagggctttggctttagcgaaagtctgaaagggaagccataggaacttaaacgtaccgactataaagctctgagaaaagctgatgttttagaaagaccatacattctaggtacaaatacctaaaaactaaaaaataagtacgttggccaggcgggcggatcacgaagtcaggagattgagaccatcctgggcccctggtgaaaccccacctctattaaaaatacaaaaattagctgggcgtggtggcgcttgcctgtaatctcagctactctagaggctgaggcaggagatcgcttgaaccccggaggcggaggctgcagtgagccgagatcgtgccactgcactccagcctggtgacagcgagactcttgtctcaaaaaaaaaaaagtacattgctataagagaagtgcacacggatactagtagttaattcagtcacatctgtgaaatagcttataaaatgctacttttaaacaagctgtttttatgaaagggcttgtaaatgtttatggtatttaagctacctctctagccataacgtattatacattcaagaaaggttcaaaaccagatatactagaaaccaatctttattttttaccccactactaggtaagggcctggataccaagaagtgactgctcatctaatccataaagctatgttaacagattggaggtagtagcattttcattacaagtgactaaaagaacagctgtttacccctgatcgtgcagcagtgcttgctgttccttagaattttgccttgtaagttctagctcaagttggggggtggtgatagacatttaagaagccatatatcttttcagaagtaggtgtgatgtactaaaagtttgagacactttctagaagtctcactatttaagttatgactagtattggatttttggcatgtctttgggtttcatgtttcttaacccaactgcctgcagggccttatggctgtcaggagcagttcttgggaattaaagtaattactgaagaagtattctagtgagaaaatgaatttatgactcagaagcccctaaagacatgggtactaagcaacaaaataagcagatgttaattaactgtaattttctcttacagctgtttgcagacaaggtcccaaagacagcaggttggtccattttctaagtttaacaaagatgttccaattgtgacagtttgtgtgtgtgtgtgtatatatatatttttatgtatgtatatatgtgtttaatttttttttaaacagaaaattttcgtgctctgagcactggagagaaaggatttggttataagggttcctgctttcacagaattattccagggtttatgtgtcaggtacgaaatttactgaattttattttatttgggttgctcccttcatttgggattgagccagaatatttcaggatacacatatctgaactgttactctaccatttcggttctatttaacccttctattcagtttgaacttgggtttaaagtttgaaccttgcagatttggcacacttcatggttatgttgtcagaagtgacatttttcctatatgttgacagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggtaagggtacaacatggcacactaaccacctgactaaatgaaaagttgccctggggggaacggaacaaacactacttttcttcaacctttgcttccacagactttttcatccctaagatactagaagaagagcatacataaatgacaaatatagccaatgtgatacagaatgtcagatactatgatagaaacttggcccttagctgggtggttgaattaggtgctacttttttgagatggagttttgctctgttgccaggttggagtgcagtggcacaatctgggctcactgcaacctctgcctcctgggttcaagcgattctcctgccttggcctcctgagtagctgagaatacagatgtgtgccagcatgcctggctaattttttgtatttttgtggagacggggtttcatcatgttggccaagctggtcttgaactcgtgacttaaggtgaaccacctgccttggccccccaaagtgctgggatttcaggcatgagccactgcgcccaaccaattaagtgcttttttttttttttttcttttctcagactggatctcgctcttatctcccaggttggagtgcagtggtgccatctcagctcactgcaacctcctcccgggttcaagcaattcttctgcctcagcctctcaagtagctggaactacaggcatgcaccaccactcccagctaaattgtgtattattagtagagcgggatttaccatgttgtccaggctggtctcgaactcctgggctcaagtgatctgcctgccttgacccccccgaagtgctgggattacaggcatgagccactgtgcccacccaattaagtgctgcttttatgttactattaataacatgcggttggttgggttttttgtttctttggggtttttgttttgttttgtttgtttttgggggaggggggcgcaattcattctatatgtgtaactcttttttgagatggagtttcgctctgtcgcccaggctggagtgcagtggcgcgatctcggctcactgcaagctccgcctcccaggttcacgccattctcctgcctcagcctcccgagtagctgggactataggcacatgccaccatgcccggctaattttttgtatttttagtagagacagggtttcaccgtgttagccaggatggtctcgatctcctgacctcgtgatccgcccgccttggcctcccaaagtgctgggattacaggcgtgagccaccgcacccggcctatatgtgtaactctttaatggtaattggagaatcatgtttaatgacatttagtacaaaaggcttcagttaaaaaaaaaaaaaaaaagctacctttctcgtcttggttcatgacacatggaggctgcttgtttgtggttgccagtcataatgattgttcttccttttcaaggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagtgttgatgtaggctttattttaagcagtaatgggttacttctgaaacatcacttgtttgcttaattctacacagtacttagattttttttactttccagtcccaggaagtgtcaatgtttgttgagtggaatatt

SEQ ID NO: 27 - DNA, 542 bp, Homo sapiens
  Genbank Accession No. AF072812
ggttgtgcgcgcaaagccgcgcggggctgagggtcttgttctgccggcgcctgtccggacccgggcccctctccgctttcggccggcctcgggcaccgggcttctggagctcccggagccctgcctgttccctgtccatccaggccagcagctgaaggagcctcacctgcctcccttctctgagtagcacggatttgaggagaagcagcgaagatgtccagcgagcctccccctccttatcctgggggccccacagccccacttctggaagagaaaagtggagccccgcccaccccaggccgttcctccccagctgtgatgcagccccctccaggcatgccactgccccctgcggacattggccccccaccctatgagccgccgggtcacccaatgccccagcctggcttcatcccaccacacatgagtgcagatggcacctacatgcctccgggtttctaccctcctccaggcccccacccacccatgggctactaccccccagggccctacacgccagggccctaccctggccctggggg

SEQ ID NO: 28 - DNA, 2478 bp, Homo sapiens
  Genbank Accession No. AF006043
cacctttccgcgggccgcggggatggcggcgcagggcgtagggcctgggccggggtcggcggcgcccccggggctggaggcggcccggcagaagctggcgctgcggcggaagaaggtgctgagcaccgaaggagatggagctgtacgagctggcgcaggcggcgggcggcgctatcgaccccgacgtgttcaagatcctggtggacctgctgaagctgaacgtggcccccctcgccgtcttccagatgctcaagtccatgtgtgccgggcagaggctagcgagcgagccccaggaccctgcggccgtgtctctgcccacgtcgagcgtgcccgagacccgagggagaaacaaaggcagcgctgccctcgggggagcattggccctggcggaacgcagcagccgcgaaggatccagccagaggatgccacgccagcccagcgctaccaggctgcccaaggggggcgggcctgggaagagccctacacggggcagcacctaggatggggcagagacttgttgcatctttgtccccagcaaaggctacatgttacctccttcaattgataataaacctttctgagatgcaaactcgagaatactgcccagttactctagcgcgccaggccgaaccgcagcttcttggcttaggtacttctactcacagcggccgattccgaggccaactccagcaatggcttttgcaaatctgcggaaagtgctcatcagtgacagcctggacccttgctgccggaagatcttgcaagagggagggctgcaggtggtggaaaagcagaaccttagcaaagaggagctgatagcggagctgcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaacacccccaatgggaacagcctcagtgccgcagaactcacttgtggaatgatcatgtgcctggccaggcagattccccaggcgacggcttcgatgaaggacggcaaatgggagcggaagaagttcatgggaacagagctgaatggaaagaccctgggaattcttggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggcccttaccagtgccttctctccacacaccaagccttggattggtctggcagaagctctggggacactgatgcgagcctgggctgggtcccccaaagggaccatccaggtgataacacagggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtcaccacctcccacagccctgctgcaccaggggagcaaggcttcggggaatgcctcctggccgtggccctggcaggcgccccttaccaggctgtgggcttggtccaaggcactacacctgtactgcaggggctcaatggagctgtcttcaggccagaagtgcctctccgcagggacctgcccctgctcctattccggactcagacctctgaccctgcaatgctgcctaccatgattggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaaccttggagctcactggtccctgcctctggggcttttctgaagaaacccacccactgtgatcaatagggagagaaaatccacattcttgggctgaacgcgggcctctgacactgcttacactgcactctgaccctgtagtacagcaataaccgtctaataaagagcctacccccaaaaaaaaaaa

SEQ ID NO: 29 - DNA, 1524 bp, Homo sapiens
  Genbank Accession No. Y11215
gtcgccttccagcccgtccgcctcccgaccagggcccgcgccccgtcccgcctctctcccgcccagccaaatgcaggccgccgccctccctgaggagatccgttggctcctggaagatgctgaagagtttctggcagaaggtttgcggaatgagaacctcagcgctgttgcaagggatcacagagaccatattctacggggctttcagcaaatcaaagccaggtactattgggattttcagccccaagggggagacattggacaggacagctctgatgataatcacagcgggactcttggcctgtccctcacatccgatgcaccctttttgtcagattatcaggatgagggaatggaagacatcgtaaaaggagctcaagaacttgataacgtaatcaagcaaggatacttggagaagaaaagcaaagatcatagtttctttggatcggagtggcagaagcgatggtgtgttgtcagcagaggtctcttctactactatgctaatgagaagagcaagcagcccaaagggaccttcctcattaagggctacagtgtacggatggccccccacctgcgaagagattccaagaaagaatcctgctttgaactgacctcccaggataggcgcacgtatgagtttacagctactagtccagcagaagccagagactgggtggatcaaataagtttcttgttaaaggatctgagctccttaaccattccatatgaagaggatgaggaggaagaagaaaaagaagagacatatgatgatattgatggttttgactccccaagttgtggttcccagtgcagacccactatcttgcctgggagtgtggggataaaagagcctacagaggagaaagaagaagaagatatttatgaagtcttgccagatgaagagcatgatctagaagaggatgagagtggcactcgacgaaaaggagtagactatgccagttactaccagggcctatgggattgccatggtgaccagccagatgaactgtccttccaacggggtgacctcatccgtattctgagcaaggagtataacatgtatggctggtgggtgggagaactgaacagcctcgttgggattgttccaaaggagtatctcaccactgcctttgaagtggaagaaagatgaaacccaggaaatatattcttccctctctcctcctttatgaggaaactgatcatcaaaagttcccactccctacttctgcacccaccaacgcctgactcctctctttgctgaagagacccaagtctcttgacacctcagagtgactgtaagctaccagtaagacaagtgggaagaggcacgttcatcaaacctgttactaaaccagcctagtcatagctcatccccatgtgtaaatgtgtccacacaaccacatctgccttttccacaagcttttcacaaagaaggtgagagagaaggaaaccttgggaggaggacattactggttgttctggctggtttgaaaagcacaaataaacttgggatgtggttccttg

SEQ ID NO: 30 - DNA, 19798 bp, Homo sapiens
  Genbank Accession No. AB014460
gatctcaattcttttcttactgaaccatccttgcgatgtactcgtcgaggtaacagctattgaatttgaccaagttcaccagcaccagaaggaattccgaggacaagccaacatccatccactgcaggacaaagtcagctaggggagaagcggcggcagtttgcgaggacgccgaatctacatctcccactcacctccccctccccccgcaggacagtgtgtccaaacccgtcactgccagaaaccccatcatcagtcccccggcagcaacagacagaccacgcactgggcccgaagaggctcaggggagcccagggcaggccggctacgcacggacactaggatctaccgcccctggggctgcacagagggtctgtgccgaggcggcagcagaagtgtgtatgccgtacactcagggcccaacccgaggctgggagacgccgacatcaccagactcctgggcgttccccacccccagcagagagcggctcggaggagacgtgtgcacacttggctgccggccacgtcacctcagacaggaaacctgacggtaggcacctggccggcacctgtcctgcaggcagcccagccactgaggcccttttctgggcgacagaacagcaaagccagagtcagaccgatctcttcctcctctttctctacttcccagaatcatgacacagagacccaagactaatggaaacctcccaccacaggatggcaggaggaaggtccacaaagtggagaaaggaggaggaagacgggaccagtacgctccggttgagaccaagtcagcagcaccacctcccagtgacagaactcgcccagctcctgccgttgcagaggtgttcggaggccccaagccccagagactcacagtcagcaggtctggggtgaaggccaggactctgcatttttaacacgtgcctcaggggcactccggccacgcaggtgagcccaggtgcatgaagcagggtgggcatacccgctgcaggccatccctgagaacggcagcccctccccagccccaggtccttcccatccaggttacacttgatcgtcaaggccagagaaacctccaacccaaggtggcacccacccagctcttcctccaagtaggtgatgtgtctcccattgtctgtgagggccttgaaaacctccagcctttcgtgaaggtcttcgttggaagggtaatccttgatgaccttaaagaagagggctctgaggacccccaaacgctcgccctaaagacaaaagccacaggatcagcagagcctgccagcgtcgcccacacggctgccaagaagtccctgaagtgcaggggcacccggactccagccctggacccctctccttcccacagaggcgccctgcaagagacaggtctgcagcatcagcattgtacagggcagggggccggatgcaccggccgaccgcagagcggcgtatggggagcaactggtgtgtgtcgggacctggcacagcgggtggcgcagagcaggggatgacacagttcatgcccgacccctgcaatcggtgcagggcagacagcgttgtgatcagagagtgaggacaaggcctgagtgggggaaacccaggagagcctgtgagagtggacggcccttctgagcagctgggacatgaaggagtagaagtggccgaccaggggaggcaggaacaccaagaggcctgggtgttcagggtcagccaagaccagcacagatggaatgcagtgagcaggggagtggccccgaaagggtgcaagggcaaaacaacaccgtagccccacaccagctgccaggtacgatctcagcactctgcgacaactgcctcatatgatccccagaacatcccataagaggaaggtgcagtgaccatctgtttacaggcgaggaggcagtgcctacggtcacacagacctcatgacaccaggagaccccacaggccatccagctccagagcccatcagcccctagaccctgtgcccagcaactcaccgaggcaggagtccaaacacggcccagcagggacaggacagtcagtgggcagcctgacgtcacccatcccagcgtcgccctgggccttacctgcccctgcacgatggccttcagcagagccagcaccgcgtgccgggcctccagcggccgctccggctgcaacagatccgcgaccgccttccagagtgcttccactgcgtgctgtcaccagcagaggggacagcggtgaggatgtgctctccaagaaccctggccccggcaactggagcagtgccgtgagcggaggacagggaggaacaacgggggcttgtgactgagagccccacctccaaagctcgtatctcctgtgtctcctgcagcctgagatgaccacaggcttcgtacagggaacggctcagagcttcctcaaaccccagcaggtccctttaactcccaccaggtgacagactgggggctgagaggacagcagcaagccagggcagcaatggtggcctcacctggaccaggagggagtcccactagagctgccggaatgctggcaggaagtcatgggacagtctcagaaccacaagtgcacactaagccctaggcctgcacctgacccagaatccctgcttgtggctctcctctagtgaacacaccagcatcgggccctcgaccccatgcccctgcccgacctctgctgcctgcctgacctgctgagaaagagcgctgcgtggccttctgaaatctgcctcctctaactaccacccagcctgtgaggggctcatccctgctccctaaagctgccagaatcagtctcactcggcaggggtttgggctggctctcaccaccttcccttctccacattaaccaacagccacggctttcagaccacaggctcattcctggccttactcctctaaggaggggaactaaaatggccgtgttccggccaagtgcggtggctcacacctgtaatctcagcacttcgggtggcagaggggagagggtcacgtcagcccaggagtttcagactagcctgggcaacacagtgagaccctgtcacgacaaaaaatacaaaacttagccaggcgtggtgctgcatgcctgtagtcccagctacttgagaggctaaagtaggaggatcacgtgagcctggcaggttgaggctgtagtgagccatgattgtgacactgcaccccggcctgggtgacagagtgagaccttgtctctaaccaaataataacaaaatggccgtgttcttttagatgtcatctgaccaacatcccaaccactggcactctcacctcctttgtctcattgacaatactcttttttttttttttttttttttttttttttgagacggagtctcgctctgtcgcccaggccggactgcggactgcagtggcgcaatctcggctcactgcaagctccgcttcccgggttcacgccattctcctgcctcagcctccccagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccctcattgacaatactcttaccgttgcaacctgagctgcaaagatccgtggacacagggcccgggaccatcccagcctcggaggcctatttatccactcacaggctgccccgtacccacacaaacaactttcagaacaggatgcaggacgcttgttcttttttttgttttctgagacagtcttgctctgttgcccaggctgcagtgcaatggcacaatctcagctcaccacaacctctgcctcctgagtttaagcgattctcctccctcagcctcccaagtagctgggattacaggtgcccgacaccatgcccagctaatttttgtatctttagtagaaacagggttttgccacattggccaggcgggtctcgaactcctcacctcaggtgatccatccaccttggcctcccaaagtgctgggattacaggcgtgaaccactgcgcccggctaaggacacctgttctttacacctatctttggtagtgtacggccagcacgtttcagatgagctgctgcagaggtagcaatcactctcctcttagcagatctgagcatgagaggtccaggtctcactgctctcccacacacaaatgctgggcaaggcaagggcagagccctcaggtgctctaccagcaacctgcactcccaaaatgaccacttcctgtcctgtccacaagtcatcagggaaagtctggcaatgtttgcccaaaataaatatttctatgtccgtgaggtcgcccaataccaccgaccagtcaccctcccaaaatggcaccaagaccagcgtgcctaggattgttctctgtggaacaaaccctttcttgatttttcttggcctgaacttgtaacaggagagagcgagtgagcatgcgtttctatcagccactacaaactcttccttttttttttttttgagacagagtcttgctccgtcacccagactggagtgcagtggggcgatctcggctcactgcaacctgtgcctcccgggttcaagcgattctcctgcctcagcctcccaagtagctgggattacaggcatgtgccaccatgcccggctcatcatcttttagtacagacggggtttcgccatgttggccagagtggtcttgaactcctgacctcgtgatccacccacctcggcctcccaaagtcctaggattacaggcataagcccccgcacccccgctcggcatagaaatcctttctggtgttgctagaaaagcctaatccctttgctccccatttccacgcttgattggtccctcaaggttgtattcggggcatttccagtagcttccccatcactacgatgaaagctaaattcccctaaaacagtgcaaactggctgggcgcagtggctcacgcctgtaatcccagcactttgggaggctgaggcatatggttcacgaggtcaggagttcaagaccagcctggccaacatggcgaaaccccgtctctactaaaaatacaaaaagccgggtgtggtggcgcgtgcctgtaatcccagctactcaggaggctgaggcaggagaactgcttgaacccaggaggcagaggctgcagtgagccaagattgtgccagcctgggcgacagagcaagactccatctcaaaaaaaaaaaaaaaaaaaagtgcaaaccagatcatcggcagtcagctgtcaaccatgttcctaaaaaaagcaaactcaggctctgaatagtctacgtgcctctctctagtagctcaactggataaacctacctcttcaaatttcttggtttttgcgacttcacaaatctgccctatcatccggatgcgattgttgaggccacattccatgctcagttcctggagagagatgaaagaagaaaaaggggccagtgctcaggccacggtctccttaactgcaagtcacaaaccacctaaagactcccactgcattttccagactagagacttaaaacagatctttctggaatcaagattcacttgacgagccgcggtggctcaggcctgtaatccctgcactttggggggccaaggcaggtggatcaccagaggtcaggagtttgagaccagcctgcccaacatgatgaaaccctgtctctactaaaaatacaaaaaatttgctgggcgtggtggtgggcgtctataatcccagctactcgggagagtgaggcaggagaatcccttgaacccaggaggcggagattgcagtgagccgagatcgcgcccctgcgctccagcctgggtgacaagagcgaaactcggtctcaaaaaaaaaaaaaaaaaaagatttactgaggggctttactgctccatcccgaggagttgagaatgtgcaattttttttcacaactgcaaatgttgtattgagataaaacataacatgaaatacaatttcaaactgcacaatttttttttagacggagttttgctcttcttccccaggatggagtgcagtggcgcgatcacggctcactgaaacctccgcctcccgggttcaagcaattctcgtctcagccccccgagtagctggaattacaggcgcccaccactatgaccggctaatttttggtatttttagtagagacggggtttcgccatgttggccaggctggtctcaaactcctgacctcaggtgatccacccgcctcggcctcccaaagtgctgggatgacaggcgtgagccaccgcacccagccaggatggactatattttattcatcagttgatgaacgttttagttgtttctaagtagaaaactttacaagtgcaaaaattcagcatttactcatgggcaaaacacaatgccaggcatgtgtgacaaagacacaaacacacataaaagataacgacctagcccatgtgcgatggctcatgcctgtaatcccagcactttgggaggccaaagcgggcagatcacctgaggtcagcagttcaagaccagcctggccaagatagtgaaactccatctctactaaaaatacaaaaattaaatgggtgtggtggcacgtgtctgtagtcccagctactcaggaggctgaggcaggagaaccacttgaaactgggaggcagaggtagcagcgagcagagattgtgccactgcactccagcctaggcaacagagcaagactcagtctcaaaaaaaaaataaaataaagaacctacttctcccaggaactcacatcttatcatgtaacacctggacatatccttacctgtgaagatgatgatactcaggcctcatctataagatgggctttctttcatcaacatctgaatagtaactcaagtgtgcctgaaccaggtcaccaagatcctgactttcgaagtctcctgaggaaggacatgtcggcctccaagtactggagacagcattcctgtgtagacagttgccagcaacccgtctcccacagaacctggtgcaagaccaaacccagccttctctgcatttccctctagcctagcaaagacacaggtagctcactcactctcagtatttccgcggtgatgataaactccgtctgtttaccctctgcagacctgggatttggcctcggtgttcccagtcccaacagaatcttaaacttctccttcaagcctgaatctttgcttgttggtttggccatggtggaccaggacgcaccagaaaacccctctgtgcaaacgaaacaggaatggggacatctgagcaacacctctgcccaccttccccactgcggacctccagccgtcaggaccctgggtctctggtgggcataacctttcctcccacactccctgggagaactcaactacagcccagctgaccagctcgcaaacaggcacctaggaaagcccgggagacaccaggcctggtctagagagctctctactcagacgctgccgcccgccccgcccgactcagtgcacgggttaggactgccgccgcgaagtcctgggagccccgacgtttgccgtctctcctctacccatgtgaagcaggagctgtcacggagccgcctgcagcagagacccgttcgcacccgccatgacttaaaactaagggatgctcggagctccggagtcgggttgcagtgggcgcgggtgagggcccgggacccggacactgcggggtccgcctctctaggccgctgccccgtggggaccgccacttgccccgtggggaccgccacttaccccgcgccgcgccaccgcgctccgggccgccccgacgcggagaaaggcgcacccccctggccccgggacgccgccggaagcgcgacccgcacttccggtagcggcattgcgcggagggccgccttgggagcttgctgggagttgtagttctgtgccgccctctggcggaaaggccggcgatccttgcatcacgtgacggggccgcatgggccgccgggatgtgtagtccgcaggagtccggcatgaccgccttgagcgcgaggatgctgacccggagccggagcctgggacccggggctgggccgcgggggtgtagggaggagcccgggcctctccggagaagagaggctgcagcaggtagcgccgcgccgtggctggagcgtgggaggaggcgggataggggctgcaggctggagtcccgggccgcctcctgcggctcccagctgcgcgggcaggggctgcaaacccggcagccgcgaacggttcccacgtgcggccctgcgttcccaccttgcgcctttttcccaaagattgcggtccgcaggggctgggtttcgggcgctccgctccgctgccccctttcgccccaagcaagaaaacgcttcctttcccaccactctgtccgggactctccactccaggctctgtgggtcccatccccgccctcctgcagtgaacttgctgtcctcggagaggctgggtgccaaggacacacgcagccagaagcctgctcccccagctcgggggagggtgcctctgagggaaaacaggaaagggcatggcacttattatatttatcctgcagccgttgaggacctgctgggggccaagcactgcagtgagcagtgagggacacggacagggcctctgcgctagtcccgctgggacttctgcagaattggccaaagttggattcccataggcacccagggaccccaggggtagtccaaactatggcaggaggaactggtgcagctgggagcaggcaggtggtgatgccagccagggtggcaggagctttgatgccaggatagagtttggagtttattatgacagtaaacggaagtcaattaggaggttttgttgtttgttttgagatcgggtcttgctctgtcacccaggctgtagtggagtgttgtgatctcagctcactgcaacctccgccccccaggttcaagcagttctcctgcctcagcctcccaagtagctgagactataggcccctgccaccacacccggctaacttttgtatttttagtagagatggggttttgccatattggccaggctggtcttgaactcctgacctcaagtgatccacccacctcggcctcccaaagtgctgggattacaggcttgatccaccgcgcccggccggccaggattttttaaaagggagtgacacaacctgatgtgggtgttagcagactgtctctggtgcagtatagaagatgtattgggggcaccttggtacaggctgttgctagagtcctcatgagctaacatggaccttgtcagggtcgctggcataaggagggcccacataccccagactgcagagggcagagcaccccaagtgtattagatgatggacagttgtggcatgaacgagtgaggggtgagctagcaggcgggatggcacccctaccctaccttcacctgtggccccaccagagggactgcgtggtgctttttgcagaagcgaggaaaagccacagccccgtgaagcgtccgcggaaagcacagagactgcgtgtggcctatgagggctcggacagtgagaaaggtgagggggctgagcccctcaaggtgccagtctgggagccccaggactggcagcaacagctggtcaacatccgtgccatgaggaacaaaaaggatgcacctgtggaccatctggggactgagcactgctatgactccagtgcccccccaaaggtaggcaggggccccatctgcccaaatgaccccccatcttagcaaggtccttgttttctttctacccggtggcttttggctggcaccctcccctgcagacagcaccttgggaggatggacacccggtgccccagagcatcgcctgcatcactgtagccactcagcaggtcaggacaccagtggtttagggcctcgtgctcgaacttctgcccagtgcaggcgtgacctgggctttgtgtgatatcaggaaggaagagttccttttgcttcacggtcccagggcctgaaacccttagaccaagctgagttctaatccctattctgctaccaggtggcttggatacaggcctctctgagcctcagtttgcttgtctgtaaaatgggtagtatgggccaggcatggtggttcaggcctgtaatcccagcacttcgggaggccaaggcgggtggatcacctgaggtcaggagttcgagaccagcctggccaatgtggcgaaaccacgtctctactaaaaatacaaaaattagccaggtgtggtggcaggcacctgtaaccccagctacttaggagggcgaggcaggaaaatcgcttgaaccccggaggcggaggttgcagtgagccaagatggcaccaccacacttcagcctgggtgaaagagtgatactgcatctcaaaaaaaaaaatttgttttaaacgggtagtgtgatgattcacgaagcaatcataatagtagtggccaatggctgttcagtagtgatgggccagatacgatcgtgttgggtttttttttgagaccaagtcttgctcttgttgcccaggctggagtgcagtggtgaaatcttagctcactgcaaccttcgcctccgaggttcaaattattctcctgcctcggcctcccgagtagctgggattacaggcatgcaccaccgcacccagctaatgtttgtatttttagtagagacggggtttcaccacgttgatcaggctggtctcaaactcctgacttcaggtgatccgcctgccttggcctcccgaagtgctgggatgacgggtgcgagccaccgcgcccggccacatacgattctaagcactgagataattcatttaagcttcacattactccagtgagtcaggtactattattgtcctggtgttgtagatgaccaaacatacccaaaccagacctccccaccccacagggggaggctgatgcctcaagtgtggcccagggcaacaaaccagggtgtgtcgagggcggggggcggagcctggccagggaatcacccaggacccgccgccaccccggtccctgtcactgcacaagcaggtacgcaggtaccaggtgctgctgtcactgatgctctccagccaaaccaaagaccaggtgacggcgggcgccatgcagcgactgcgggcgcggggcctgacggtggacagcatcctgcagacagatgatgccacgctgggcaagctcatctaccccgtcggtttctggagggtgagccctgcctgtggcaacgggggccgggtggcagtggcctgagagagacctcagaaggctgaggggggtggcgacggtgcaggaagtgagggtcaagcatctcaggctcggggggtccccctgaagccccccccaggccgtggggccctgaggcctgacgcccagtcccagcagctccttggccttcctgttcgcatcctcccttccttcctctgcttccccgcctcgccgcctgcccacccttcctcaccgccaaggctgcctgcccacccgcctttccctttgtttactgctagcctctcccggtgtgtcaccccctcctgtccagagcccctcaccctccaggagccctggggaggctgctccccaaccccgccgagcactggatggtggtgggcggatctgttgaaagcagcttggtgggtggggccgcttccccttggcacccgcgtgggccgcagtttctcagatgggttttaaatagccctctccactcaggcacaggatgggaaccacccggcaggcctggcagctggtggggaagccccagctccagccctccttcctgaaggcgcaggtgcagccaagccccactcctgggagcggctggcctgcttggcctggcctgggtgctggcagggaatgacgggcaggggaaccccagccagagcctacacgtgcatcattggcccacacgggtgggaggagggggtcaggcctaccttgggcctccctgctgccctccgcttgttcccgcctcctggggggcgggggctgagcttgcacacgtcctcagcaggtgcaggtagctctggccacctgtgggtctcctgggggctgcatcctcccaggttgggctgtgcccttgcctccaaggctgaacccacccctgtctttcagagcaaggtgaaatacatcaagcagaccagcgccatcctgcagcagcactacggtggggacatcccagcctctgtggccgagctggtggcgctgccgggtgttgggcccaagatggcacacctggctatggctgtggcctggggcactgtgtcaggcattggtgagtagaggagggcggggctggctccagcccactggctgctcttgggattccccacatccttgtgacctgtgactccacttccagcatgctggtcctccacccagcccatagagtcagaaaggtgctcccagggtggagccaggccagccccactcagagacaggtgctggggcaggaggtcacatgggctgagcacctgctccgtgcagggctctgagagaggccccggagctggactccatctgcgttgtattcgggagaaatgtggagcgaggaattccccactcgtggagtggaaaccctgatggttgtgcagggtgctcttggggcctggcttgggcccagggcctctgggaaggcccccaaggtcatccttcacctgagagctgcagagcaaggtggggtgagccaggccaagaggaggaagtgtgttcccagcagaaggaacagcatctgtaaaaccatgatgcgagagagagggccaggagcaggaagttctggaatgagatgagggcagagtggggaggtaaggactgaggagggaggaggaagagtatcccctcagcaggtggggtggggagggaggtgctggggggataaagggggagggtggggagggaggctctgcggggacaggaggggaatggtagggagggaggcgctgggggaatgggggaagagggtggggagggaggcgctgggaggatggggggagggtggagagggaggtgctgcggggagaagaggggagggttgagagggagaagaggggagggtggggatggaggcactggggggagggccctccttgaatccttttcccaggtcctggtgtctcttcctagcaccggaggcttcttggcagttgggtgcagacagacccctgggtttggggtggggctgcctcctcctcggctttgtccagaggatttggggctcctaaggccacacgcagccgggcccagccagtccagaaaccagggcttggccctgccctctgtgtgcccccatcccaggcccctttgggactcctgcagggggcctgtgccggaggcggggggcttgcagcactttctggtgggcaagagggccaccaagggccgggggcctctattctgagccggggccacctggtgaggctgaacagccacagcagccccaaagagccagagccaggccaggtgcgccggggcagggcctcctcccgactgagactcccagcgtcaccacctgccagcctctcagtggacacttgagcttatctgggcggctgtcctcagctgcccgtgtccctggggtgctgcatcgtggttgggcagggctgtgggggaaggtccctgcccgcagccaggcagggaacgggctttgggagcagcgggcacaagagacagccatgtggaaatacctccctctccaggtgtctgtgggcctgaccacctgcaaggagaccccagggctgcctcctaggggttccccgaaagagaaacgctctcagttaaaatgtcagagctggctgggtacaagggctcaagcctgtaatcccagcactttggaaggccgaggtatgtggatcacctgaggtcaggagttcaagatcagcctggccaacgtggtgaaacccgctctctactaaaaatacaaaaattagccgggtgtggccaggcgtggtggctcacgcctgtaatcccagcactttgggaggccgaggcaggcagatcacgaggtcaggagatcaagaccatccaggctaagatggtgaaaccccgtctctactaaaaaacacaaaaaattagccaggcattgtgtcaggcgcctgttgtcccagctactcgggaggctgaggcaggagaatagtgtgaacctgggaggtggggcttgcagtgagctgagatcgcgccagtgtactccagcctgggcgacaaagcgtgactccgtctcaccaaaaaaaaaaaaattagccgggcgtggtggcaggagcctgtaattccagctcctcgggaggctgaggcaggagaatcgcttgaaacccagaggcgaaagttgcactgagtggagatcgcaccattgtctgtatccagcctgggtgacagagcaagactccgtttaaaaaaaaaaaaaaatgccacagccataaattgctggctccggtcccagctctgcacctcctggctgggtgactctgggccaggcccttggcttctctaagctctggccacaaagtgggtgaggcagtcctcgtcattcactgtccaggtgggcaggaggctcacacaacgtgacgtggcctgactcagccttgaagcatggccacttccgtgttacagaggaggaaactgaggctcagagccccaggggtgtgacccctcaggccttgtggtctccccacaccctcggacacctttttctaggcagcagcgaaggagctggtgcaggcctcggctttctctttgggaaacacgggaggaggaggcagggctggggctgcagctggccggggccaccgggtgggagaagggagccccagtgagcagacgcctggggtggccagctgaggggagcaggagccagaacccaggtggacagcagctgcggccggccagggcatgctcccgctgtgtccctgaccgctgcagtggcaggtgctaggtctgcaggcagtgacctcatgccggcctgccaggcagagctgttgccacccagcctggtgcccagagccagtgcttatgccgagaggagcctcaggccacaggagacagacagagggaagcctggatggacccttccggggacagggccacgtttctggggaggctgagggctgagaggtgcttcggagtcgggatcctgccgggcctcggtttcagcatccgtaggacctcatgaggactcactgagctggccaatctctctggcagggtctggcccccagcaagcgtttgtccaagcgagctgtcgctgttattgtggggaatagagctccaagcagagccgagtcatcacccccaggccctgcccaaagcagagaccccaccacccacacatccaactagcaccagtgttgggagtgggcagcccagaacagagccccccaggctggctcaggtgatgggagagaggcctgcaccccaacagagggctcagcccctgccccagcacctcaggggcagccaagatggtcacatgtaggcttgcagccagggctttgcggttccaatgtctaactggagggccacgggggtggtggcagactcgcctctgggcggggaggggcacgggctaggctggtggagtgtgcccctgttcaccccgcttggcctcccagcagcagtggacacgcatgtgcacagaatcgccaacaggctgaggtggaccaagaaggcaaccaagtccccagaggagacccgcgccgccctggaggagtggctgcctaggtatgagtatgtggggggcttcctagggagaagagctcaccccgcccgccctctgtcagcactgacccagcccccacccccacagggagctgtggcacgagatcaatggactcttggtgggcttcggccagcagacctgtctgcctgtgcaccctcgctgccacgcctgcctcaaccaagccctctgcccggccgcccagggtctctgatggccgcatggctctggccgaggtgccgctgtggccaccgtctgtgaagtggctttacgcttcaggaagccacgcctgttgaataaagctttggtgtgtttgcagatgggatctggctgatgtctgtccctgagggggcagagagcctgggaatcgcatacaaggatgtgttcgtacacacccgtgctcgcgtgtctggagcagggctgcctgggaggccggctggggacgtgacaatctttgctatgtactcagggagggggagggggtgtgcatgaacctgggagggaggaagtggggagagccagctgcagccagctgttggaagtccgctggggaagcaagcagcccccgggcagctggctggggaccctggcccctcatgctatccaccgccggccccccagcttcccagcccggacaccgccagctcctgaactttcatgccctcaaagcccagcctctgccagggtcccacccctgtttacagagctacccggccttgaaagggacactggggacccaggcagggcgccacagggtacaaagcttgacctgtgactgctagaggtcgcagggtgaattgggcaaagggtggcccgtgtgggtgttgtgtggggccggatggcttcacagggaaggtgaaggtgggaggctgagtctgccaaataagagagatgggcatgggcccaggatgtgccacacagcagggggtcccatgggggtcagcgggggagatggagcagggctgggagcagatggatgcagggcacgggctcaggaacgagaccgtttccagctgctgagactcggggagggggctccctgaaacagcctcatccctccacccctctcaccccacggggaggaggctcagaaaagacagaggcgtgtgggtcccaggcagggcctgtttatcctgagagcgcgcatgcgctatggcttacgaggcaacagcggtgccggtcgatcggtggtcggttcgggggtgcggggcccgggagggaagtgcaaagtcaacagggtgtccacgggggagcccgccaagcagggctgagccaaaggccaggcgtccttcccgggtgccgctctcacacacctacgttccccaggcctgggagctgccgcgcggaatgaggggcctgtccaaaggccgccctcctcggggacttggggcctccatcctcaggcccctcggaaagcccaggcccccagcagggcacaggcgcacagggtacctgggtggggagcagggtgtggcctagggacccccacaccagggcaggtgggcaggggcagctcctacggccacatcaggcctccctggccttacccaaggtccccacaaaaaggctgggcgccccaacttggccactgggaagtcccagggactcccactcaggacggccgctgctgaggggagggggcggggaggaaatggtttgcagagaaaggtttgttttattgcaattatttagagcgcgtcccaagggggaggggaagggggaggggaaggggggggggtctctggtattaagtggaaacatgtgtggagctggagatctttttggaaaaaaagcaaaaacacaaattcctttcagtcagcaggccccggcagagcagcaaaggccctcgccccgcggctgccgcgcgctcgctcgctctctctctgtctctctctgtgtctctctctctctctctctgtctctgtctctgactctctctcacatctctgggtctctctccccctatcttgctgccacaggcccccagtacctggtgggcaggggcaggatgggggctcaccggggctggggctgatttctgggcaatggcaggaccaccctccagtccactgaggctgggggagctcggggcaggctgaggcccaaggtccgtgcgggaggggtcccagggtcccgagacaggcaggaaggggctcagaagttgctgaagatttcacgcttcctgttccagtccatctgtggcgcgcgcttgttgactcgcatggctcgagccttctccttggcctcggccgccgtggggctcaggtggaggccgctctcctggaagggatcttgcttccaggcactgccatcctggggtgagcacggtgggtggggtgtctggagtgagccctagtcccacaccccagacggtggcccctcctagggagcaaccaacagaggctgcctgctggggctggggctcacgaggacttcctgaacagagaccccactgcccgggctgacatctccacagacgcctaccccaaggcctctagcaaccccacgtgtgagctcaggagtggagaacatccatacctcagtgtccttgtcggaaccaggcaggtcacttcgggacgagcacgcagagccaccattgagctgggaaaccaagggcagattgtggtttcacagacacacacaacacctgcacacgcacacatgtgcacagaggcacacccccccccagtgtgtgctcctccactcggcagggggcctcgggcctctggtggactccagatgcggcggggcaggcaccgggcgcctggcagtgacaggcaggtggcctggccggctcatggtgcagagggggttcaggtgcaggaatggccggcgtgagcaacgcctggggtgaggccaccgccagctccctgaggaggcagagactggagggcaactccttaccaggtggtgcccaggagaggacagaaggcagtggccccgaccccaggggccccacgtggcttgtgtgtggcacgacccaggatgagacccacaggggcattaaagggaggtgacagggctaaaatcgggtgtcactagcacacctcgaggacggatctccagggagaagggggcctctgtcgggcctgggtacccccagaggcaccctgtgtctcatgggcaccccaccctcttacctgggcagggctggttccattggtgacgggtgacggcagaggacctgtggggacagcgtgtcagtggctgctcagccaccacaccgcacccaccctgtgccctgggccgtggcccaccttccacgtgctcctcggtgggtgtgacccgaagccgcttgaagtgttcatctgtctcggggtccacgaccagcagccgggcctcgtcctcccgtgccttgatgctggccaccacctcagcatggcgcagtccctccacattctgcccgttcacctgccgccaccagcatgtatcagccccgccatgggcccagacagcccccaacgccgcgtggccaggtgtggcatcaaatattcatcacagccaccgcccaccgaggcagcaggaggcgcaccagctcaggttcccagcgtctccctcctccctgcaaggacccagggccccagtgtggacaggtgcggggcactcggcaccccgcagccctggtgggccggtacctcaatgagccggtcctgggcgcggaggccagagcgggcggcaggtgagcccgggtccacagagcggatgtactggccgggccgggacttgtcactatgcaggttgaacccatagccctgaggtccctttcgcaggtggcagagccgagggcgcagctccctcaggggcccactgacatcctgcaggcccaacggacagggtcagtctcttgctccagcctcccgcctccagaggccactccccggtggtgccgccactgcccaaactcggtaccgtgccccccacgggcaggcccaggtctcagtggcaaacactccgggcacccaggctgcctgtgacagagctttagctgtgggaacttgggcaaagcctgttttcaaacccagaagcctccccgacgggcccagccgccttcccgggttgctgggagaattc

SEQ ID NO: 31 - DNA, 6330 bp, Homo sapiens
  Genbank Accession No. AF037062
ccaggttttccctcccttcccccactcagctgcaggaactcctttttggggtttggatctggtatttttctattcagctccgagcttggctctcctggggaatcctgggagtgaaaggaaggagctgggtttatttgcatgtactggtagtcatttgcatcacatccaaaaatggccaaaattataacccctgattcttggctgaactgggactgctgcaatggaatattattcccggaaaccacccccaactagctggagctaatctcctccctcctccaaccccccattttggcccaggcctacataaaccaaaaaaagctggaccataaggtgaaaaccctacaggtccaggctgcccaatttgccaagcaaacaggccattggatcgaaatggtgaaaaacttcaaccaggcactcaaggtgggccatactccctacctcaccaccccaatcctgggcccccattggctgcctccagtcaggttacctcaggtttaggttaaggaggaagtagggtggtcccagaaaccccatctatagccccagtgtcagaaaaggttgagaaagaaagaaaagcagttggtgggtccaagttaaagccttttccaggagatgaataaaacttattccccaatggaagccatagtctacccattctgattcctgggtcccaactcctctccccctttccaggaaattggggatgtggagaatgggcttggagcattgagctggaaatgcgcaccattgccaatgcaatggaatatgtttacaaagggcagctgcagtttgccccttcctagcccctgttccctcccccaaccctatccctcctacctcacccgcagggggaaggagggaggctgacaagccttgaataaaaaacaagcctccgtttttttgtggtgtgtttcagagaggtaatagctccagtgtcgggggtgggagtggaaggttcaaaggtggtttccctgagggacaggtaccttttggggagagggtggaaatagcttccttttactatcccaaattttttttcctccatggcccttgtgcaggtgtttgttaggcaagcagagggtgggagttcccatccctcctgagagaaggtcctagtagccctgccccaagcttcctaattcaggaattgtttcctacagaagagaaacaaggcaagtacacctggtccccagctctggctttctgcctctccacgtgctcatggcctctccccaggctaactctaagcagtgtcatgagtctgagccaggtgggagattaattcctgggggcacttcagggctgagaagggggaggaatgacaggtccagtaaccgttaccaacagagcagtgcagctgccatccttgacagctccctcctccttggagaccatgacatagatggtcaggaacccaggctgagaaagacagccaaggggtggggggagcctaggcaaatctggcctctgccaagtcctggcttcagccaggcaagctccagcctccctggctcctcctcctcctcagtcctatccccaccctgtcacacatacacttaatacgcctggcatccaagtccacccactccggactttggccttagcagtagttagtgtgggaggctgggaagactgggagcagtctcttaaacaaaagcaaaagaataagcttcgggcgctgtagtacctgccagctttcgccacaggaggtaagtggatctgggagctgggggaactgagaagactagccagatattacatgtattgccaactcaaaactttcagcttttaacatgcttcctcacacattatcccctttgatcctccacaactctgaggtggacctggtgggtcttagccccacttggtagatgagaaaataggttgagagagacagtgagatgctcagtatcacacagcaaacctcttggccctatacatcattccaaacacaagacccaggttgcatatagaaggttcagtgtccctggtttagaaggagaggtggtgtgaggcaagcaagaagatgcctctgctgcactccagcctgggcgacagagtgagactccatctcaaaaaaaaaaaaaaacgatgcctctgctccatacagcaggtctgtacacaggatctggctcatgtggttttagttaagttagccacaaatacagggtctgcccacatctttgctttgaacagatgagccatggttggccaattatctgccaaccagataatttctcaatatgctcacaccagatgcttccagctagggagggtattaggggaaagggcttgagggccacagtaaactggacaagtttttctgcccagcctaggctgccacctgtaggtcacttgggctccagctatgtggctgcctcttctgctgggtgccttactctgggcagtgctgtggttgctcagggaccggcagagcctgcccgccagcaatgcctttgtcttcatcaccggctgtgactcaggctttgggcgccttctggcactgcagctggaccagagaggcttccgagtcctggccagctgcctgaccccctccggggccgaggacctgcagcgggtggcctcctcccgcctccacaccaccctgttggatatcactgatccccagagcgtccagcaggcagccaagtgggtggagatgcacgttaaggaagcaggtaagtatggtagaccaccaggaatatggtgtggggtgtcctgatccccacagtcaccccaggagtcacctgcaagggctgtggtaagctaaagggacaatttgaggagaagcagttttcagatgctcccaggaagaagagggagctgtgggagtgcctcacctacccccagcatccttttcatctccccacagggctttttggtctggtgaataatgctggtgtggctggtatcatcggacccacaccatggctgacccgggacgatttccagcgggtgctgaatgtgaacacaatgggtcccatcggggtcacccttgccctgctgcctctgctgcagcaagcccggggccgggtgatcaacatcaccagcgtcctgggtcgcctggcagccaatggtgggggctactgtgtctccaaatttggcctggaggccttctctgacagcctgaggtgaggggtacagggctctgggttccaggactaacagcagcccactcaacaaacgtgggccagcagaggtggttaaaatacagcacattggaatagttaaaaagagacagtttagggctaaacttcatgggttcaatgaagtctacccttatgtaagctttgtgaccataagtagattacttctctttacccatttttaacgtgtttgttttttgttttttgagatggagtcttgctctgtcgccaggctggagtgcagtggcgcgatcttggctcaccacaatttccacccccggggttcaagcgattctcctgcctcagcctcccgagtagctgggactacaggcatgcgccaccatgcctggctaatttttgtatttttagtagagacagggtttcactatgttggccaggttggtctcaaactcctgacctcgtgatccgcccacctcagcctcccaaagtgctgggattacaggtgtgagccaccacgcccggccttgcctctcgtctttaaacaataaggttcaaagttccgtgggagcacaaaggagacatgatgaggacaacgggagttagggcctgagtttttttggttttttttttttaagcgttttgctcttgttgcctaggctggagtgcaatggcgagatctcagctcacagcaacccctgcctctcaggttcatgtgattctcctgcctcagcctcccgattagctgggcttacaggcacgtgccaccactcccagctaaatttttaggtagagatggagtttataccatgtggccagggtgggtttgaattcctgacctcacctgatccaccggaccggccttcccaaagtgctgggattacaggcatgagccaccacacacggcccaaggcctgagttcttagcaggagtataaggcgcctaagcttagtctaccttctaaggaagcctgcgtttgtcaccatcactcagcaaataaccggaattgtctcctgtctctcagccttaatttttcaggcagcatcatgggacacatacttttagttttgagacaaggccttgctctcacccagggtggagtgcagtggtgcagtcacggcccactgaacttcaaactcctaggctcaagcagctcaagcgatatccgcctcagcctcctgagtagctgagaccacaggcgcgtgccagcatgcctggctagtatttttttacagatggggtcttgctgtggtgaccagacttgtctccaactcccggcctcaagcgatgcttccgcctgggcctcccaaagtgttgggattataggtgtgagccactgcatactggaacacatactttatacttgaatttttttttatccccttccttcgtgctcctaacctatacttggatttctacatctgtgccagggcagtgggatgtatccccactttccccatcagcttaccctccagcaaatacgagactatacccttcaatatccagcactcagggctcaaccatgtgttttgggagcaagggaatggggttcctctaggtcaggaatcggcaaactcagtactcaagccagatttggccagctgcctacaagctgataatggttttttttatttttaaatggttacattgtaaactgttatataagtacctgataatatcattaattttgtttcttggcctgccatgcttaaaatattaactctctggccctttaagaaaaaaacgtgctgacccctgctctagatcaaagaaaacaaacctcaaaaatactttcctccctctaccccacttgacccttgtcccggggcagtaggcatctccgtcaaaactcttgtccctggtctgtggtaactttctcagctccccaacccatgtccctcaaagtcccctccctatagggcaagaacccagcaacttcgctctgccccgactctaggcgggatgtagctcattttgggatacgagtctccatcgtggagcctggcttcttccgaacccctgtgaccaacctggagagtctggagaaaaccctgcaggcctgctgggcacggctgcctcctgccacacaggcccactatgggggggccttcctcaccaagtgtgagtagccaggcccacacaggggcacatgaagggaaacaagtaccagaaaggccagtcctgcataagcctgctaggaggtgggtggggcacccagggcagggttgagggtgaacaggatgttacaanagtgcccaggccatgtggaacctgcccactccccacactgaggaggggactgagggtgacaagcccagggccccaaaaaacagtacctaanatgggctggagtgaggaagggaaactgattgcaaccacctatggggctgcagacctgaaaatgcaacagcgcatcatgaacctgatctgtgacccggacctaaccaaggtgagccgatgcctggagcatgccctgactgctcgacacccccgaacccgctacagcccaggttgggatgccaagctgctctggctgcctgcctcctacctgccagccagcctggtggatgctgtgctcacctgggtccttcccaagcctgcccaagcagtctactgaatccagccttccagcaagagattgtttttcaaggacaaggactttgatttatttctgcccccaccctggtactgcctggtgcctgccacaaaataagcactaacaaaagtgtattgtttaaaaaataaaaagaaggtgggcagaaatgtgcccagtggaaggctgaccccatttaagtgccaactactccaaaccgacatgctcacggtctctggcctgttcagtccctgcaaaacagctagcacccacagtggggcgccagggaactgcctcacatctacagctgcacgtcggggagtggccatcaaagggcactttaatacatttcccttattttctgaaggggagtaaggttgcaattcagtgtctgtactgggaatggtcttcatatttcttgggggagaagagcaggtgatgagggttctgggccaggctgggtggcttccatggaagaaaaggcaatattcacataaattctcctgctaaggacactgaccacacaggtgtgcaaggcaacttatcatacttcgaaaggagctggatcccttgaggattggccaggaagggaggtgctgggcccttagcggtgcacagaaggccaggaag

SEQ ID NO: 32 - DNA, 10884 bp, Homo sapiens
  Genbank Accession No. M35878
ctgcagacctgggacctcaagaattgcatttgatgccgaacccagctctaatttcagagtcaaggtctctgcgagtatttaaggaacggatgtaaacctgggggattcgttttgtttccttcaattttccaatgaaatcagagatcctgttcttgggtgtcaacgcagatactagaaggaggtgatacaagagaaaggaaacagcaagcgacgattatggcacggtttcctgtaaacaaggttgagtgtagccacagcctgagcactgtgggagaagagctcataagaaaatgacggtgctgggccttcgtcaccccggggccctccattgttcttgtctttggtctctttttatttgtagaggtccaattatttatttatttagtacaagagggaacgaaattgatctttccattctaaaaggagagtatatatgtataaaaggaagctgtatagatatgggggaagaggtggacagggggaaaaggggagaggacgagagagagaaagggagggagagggacaaggagagacactgggcgagagatcgattaggagagacagaaatgatgaatgaagattaacttcacccaaggcttcgtcgctggaggggaatggaggagctcctgatttgctattactactccaaactgcaaagggctccttcaagtcacctatccacctcctaaggcaagcgtccaatttcaacagcgttcaggaaagtctcctcccgcggaggtctcaccgcttcccactccacccccacaaactctttggaaaagtgccttgaaaaatttaatcctcaatccaatcctggaccaccagcgtcctctgttggtcaccgaaggagggggtgcgcagacaaaactgaagaaactcgagtgccagagaaggccgacaggagttacagcgacctcagcgcgcaattgcgccccgaactttactgaaaagtgtttagattgcagagataagctagaatcccaacgcatcgagaatacagtaatacgaagtcgccttcaaaaaatgacaatgaaaattgcctattaaaggactatttggttaattacgtttcagcagtgcccagtttattgtctttattattcttttgtcgtgggtgtaaactccatttgaaaacataatcagggagaatacccaagacaagaagaacagttgtcatttaaaatatttgaaaagccctgccttaaggagcattcgcttgccggtccactcttaattggggacttgcggtgtagcaacacgtgagagtcttcttgcgttgagaagtaagcctggaaaggcgaaggccccggggcatcttcagatgcgtatttgtgggcccctggggatataaacagcccagcgggtgtaaattaaaccccgcagtgccttggctccctgagacccaaatgtaagtcagaaatgtcccaagacttcgcctgccaacggaattaaattttagaaagctccacgaggtacacacgaatgcggagcgctgtatgccagtttccccgacaccggctcgccgcagggagacctcaccccgagagcggaaggggtaagggcggcggggtcaaggagatcgggggtgctgagttggccaggagtgactggggtgaccgggggtgctgaggtggcctggagtgccggggtggccgggcacaccttggttcttgtagacgacaaggtgacgggctccgggcgtgagcacgaggagcaggtgcccgggcgagtctcgagctgcacgcccccgagctcggccccggctgctcagggcgaagcacgggccccgcagccgtgcctgcgccgacccgcccccctcccaacccccactcctgggcgcgcgttccggggcgtgtcctgggccaccccggcttctatatacgggccggcgcgcccgggccgcccagatgcgagcactgcggctgggcgctgaggatcagccgcttcctgcctggattccacagcttcgcgccgtgtactgtcgccccatccctgcgcgcccagcctgccaagcagcgtgccccggttgcaggcgtcatgcagcgggcgcgacccacgctctgggccgctgcgctgactctgctggtgctgctccgcgggccgccggtggcgcgggctggcgcgagctcggggggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggtgagccgcccgccaggtgcgctgcgtgcagcaccgccactggcgccgaagggcctgggggttgctgggtgccgctgcgggagactccgcttttcttctcactggagataatatgtggggaaactgaaggcgctccgggaaaggtgaaggcggtcgccgagggaccctccccagccggccctctacttgctcgattctctaagtgcagagtacttgtaaattgcaaagcgctttcagtgaaaatgggtaaaggtttccggagctgaggggagcggtaccgatgtttagctgttggaaagatcctggacacaggagattctcctcgccccgcacgggtgcacacggactgcaatcccagggatgcttggggatggggggatataggcggatttggaccaaggaaggtgggtaggcacgttgtaggaaatagtacctctcttttaaaatactgactttgcacagccttttggtttgcaaagcaatgtctagtcccggtatgtccaaaaacaagtaaagtggattcgggttttgatatcttctgcggttggaaaacctgaagctgaaaaagaagtaacttcttaaggttacccagcggccacaacagagtgtaggtttgaactccgcgtgccactttcagtaccataccattcttacaactcgggccacccctgcacctgcgccgacctcaaacaaacttccaggtgcgtggtgggtgcgggcaatgtggactaagtcaatttcaatgacacggcaagggaattggaatcagtcctaggctgtctcccttcttaatctgaaatggggggggggaatgagatgttgttaaggggagccccagaagaggaaaaatgcaaacatttggcagagttaccctcttgcttagccactatcagtatcaggcagacagcgactctggtaagggcatcacattgttcccttaaaaaaaggagcgggggttgtttaaatggatttggcagctgttctttcaagcattcttagccagcctcacctagttatatgagaaataaagttcctgccttgcacagctgaaggctgggagaattctccccatcctaattcccccaactccccaacgatcacgttggacagatgtcactgggcaggcccccatctagggctagcaggatgaacagtccctttataatttatgtagctgtagagttccacgcccgggtgaagttattttctggctcggcaaggctggctctgttcacccctgagaaatgctggattcatggaaaggcaagatgcctgaaacatacactggctctggtcagctgttaaagctgctggaggcatttgtctctcggggcaaagttatgtcatttgccaagtgtcgtacattattgtgcattttggggtattcaaaaagtgatcttagaaatactgatacacatcgtcattcttgggctttagcaatcatcatgattaccaccttagtagcactgtagtataggttgatgtgagttataagattataaaaagatctaagtgacttctagaatctatttgacaaaaaaaggtaaattttcgacagtcaaaagtcacaattatctgttgcttaaatagaactgttttgtcttcatgccctagtctgcagcccaggcattaagaagaaaccaaggaaatttaagaaattactcaaggttcttagaaaagaagtataaatacgtttatttacatgttcttagagtatttacattcttagtatctcttttatctcagtatttccttgaaaaagaaagcaagctaagattaaaagaaattgaaaccaaatcctcgcaggtagggacctcctctgtgaggctctgtgctggaccctgggaatgtgtgcttcccaaggtatgaaaccccttggggaactttacagcaggacctcagtgagctgtttggcaggtgaggaaactaagacccagagaggagagggactttcctaaggccctggtgagtgacctgccagtagccacttccaggggagagcagagcatctgcagccaaatcattgcagccccaggtagctttctagatagactgtggaccagatgggccacctgagctccctgctagggttacacattatagccctgtttgtgtagtagagaaatttcatgactctcaattgtggacttaagccgatgcctccagaccttggcatggtccacaggccctgggagcatgggctctgaatgtagcctttgatccccatagcggtcttacagcccctccaagttcattctgaagaaggaatggagtgagaatcctggctgcagatccagtcttgaatttagtcatatacttaaaattccaattcaactgttaacattccagcatccattttaagcatcagactttcttcatttagcactttttattataaaagggagatctgctggagggggatttctcctaccccacccccacccagggaaggaaaagctctttggcacttagaagtctgagccgtgagtgggactttggcattgtctgcatccatgtgctgctgtgttcacccggggtgaaaaggactcacttaggcaggcaccagcaagatgcacagggtctgtgtagaccttgagttttagagatgtaacggggacctagaaaacaagccaccaacatgcttgcatgattctgagcccctgaggcaaaacgctttgcaggtaataattcagttttcccatctgagctggacaccaagctcttataagcgtgtttacctggtagcattgaggacggtactggtcaaccttggaattcccataagggcttgttacaactcagactcgtgccgccactccagcgtttccggagtggagaatgtgcatttcttccaagtccccgggctgccgctgctcccgcgggtgggaggaccacacttggagttgactgcaaaatttctgagccggcgctgcagcagcctcccgtggctcaggtctgccccctgccggtggaagatgaagcatactgccttcacctactgaggggcactgaagcgtttgtctgccttctttagttgcagctacttaggaagagcacctgtcagattgactttcaaacagataacttcttgaggtagagcaaccaccatgtagtgagtagtatgatggaataatacttcatcgaggtatttaaaaaaaaaacctcacttggattgccaactaatattgtcatttacatgtgacctggttgcaacgttaagatttttacaagactgtgatagatattgatgactctcatgtgtttgtctctcttgggcgttttaaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatgtgagagcttttcctcttgttaaaggaggagggcaagacctgccaagcctgggtactcagagcctcttgagggcaattcttactcaacaaaccccagcgcctggctgatgggtgggcaacccctagcccctctgtgccctacctctctcctctccttacataaagaatattgacccttttggagaatcttatgaggatcaagctgaaataacactcttaaaagcatatgggatgtcataaagacctctgcagataatgaaaatattctcataaagatagttttatttacttcatcctctatgcttgttgacctgctattggttccatgccagcttctgtgccttactctgggaagagcaaaaaggagacagggagtgatggttagcttattcgggggactttcgtgctacatcagacataaggtatctgaggagcaaattacaggtcccacttttggtagttgtgcagcatcgtaagatttttaaagcacacattctagagtaaaaactgtgactctgttgctctggtccttcctgatccccagggtccctgccgtagagaaatggaagacacactgaatcacctgaagttcctcaatgtgctgagtcccaggggtgtacacattcccaactgtgacaagaagggattttataagaaaaagcaggtgagtgaggtcctcagtgtgttttcttcctcttctgttgacacagaggagaaacccatgtcaccagcgcccaggctcttgtggccatagctctaactctgagcctgtgcagcaccagtgcccaggacttggtgccagtctcaggaggtcagaccaagggctgctttgacttgttgctctgagtgctgctatattggccataatcctcaaccctagtgcctttccaccacccgcttcccactcctgtcctttcaatggttcacccacaggcggacaagatgctgcccagtggcaccctttataaactgcaagtggacatgttaacacatttgttaatgctgcgtcagggagtgacatttcaaacaactattatagtcagtttccaagaagtgtgacatgaggtcataccacaaaaaagcttaccctgaaatcccacaatcgtcccctttcctactgatgccttcccgatagtgagcaggttgcaatattaagattttgaaaaggctgttgctagatgttggtgactcgtgtgtctctgtctcccttgggcttttcaaggaaatgctagtgagtggggggatgactgcagcatggccagcttggagagcccagccatccccagcacataccaggtgtctgtcttggcgtggaggggatggaacttgaaatcagacactcggtccatgctggggatggccagtctctccaaactggcatgtggtcttcctccgagtcactggcatttccctagaaagtccaagtgagaagaaggcatgagagtcatcaacatcaaacaacagtcttttcaaaatctttatattgcaacatagtcccattcctggaaaaggaatggagtgagaatcctggctacacatcagccccaaatgtagtcattgcctaaaatcccaattaacctgaaaatgatcaaacaaatttaagatatagtaatattaagctgtaataaatatgcttctataggctttgtgttatgtgatggcactatttcaattggctttctaattggacaattgatactatgctatctacagaattggcctttggagacctaagtgagccacagtggcctcagggtgaccatatactaggattcatagcagtggccacagtcagaagcctaagctttcctccattgccattgctcgtttataccacgtttctgtcaaagtcatattcattcaacaaagtcatactgagaaggtgtcatgtgaggctggatgtgggctccaaagtcatagctgtgacattcgcaggcagcgggatgttctcagttccacatttggcagagaagtcagtcaagaggttctacaagggctggtgtccaccttatactcctagaaacacaaaactgcccccacccccgctttcttggagcaggaagttacacccacacgcatgcacaggcgcacactcagcgggcctaggcagcgtggctcttgtgttgccttagctgaaatttctgttgtgctttctcagcatagcagagtcacgctggcaaaccatcatgcgccctggccaccgacctgacaccagacccaggagcattcacttctctgtcttctgtttctctcccacagtgtcgcccttccaaaggcaggaagcggggcttctgctggtgtgtggataagtatgggcagcctctcccaggctacaccaccaaggggaaggaggacgtgcactgctacagcatgcagagcaagtagacgcctgccgcaagggtgagtactcaggaggggcagcctgggctccagggcctcactgtccttggaccagcctcaggggctgggcgtggccactggccttccccaggcttacagacccaggagctgcagctcagggccagaaagagcaaagcaaataggacagagccctcagaagggtgcagggagagggagaccccatcaacccaaccaaacaagtgtggggaaggaggccggccagtgcacctcagggacactctgctttatctcagatacctcacagcacctaagctatcattcatccacacacaaagtgaagattttcaaagttaggctttacccgtgagtctggaggtcatttatcttcacagagaacgtttatcgcagactgctaagatacatgttctaattaagatgtgatgtgagaacgctgaatgctcgttggagactcagttgaagtgcagctttttttctgtcaaatatataatgaatattctgttagtctgtggctaatataattttaataaagttaatttaaatctgatagaaaaatgaaattttaaacgataattttagagaatgctattatatccagtcttcttttttcttttaataaatgagggaactattgggggaaaggaataaatacattttctttcattttattaagacaaatttagtaagcagaagaaatttgcatgtttagttataagggtttcttttttccttacaagttggaaaaaataattctaatttaagggtaactctttgacaatgaacactgtgagcagcatctggtactcgttgctttgtttgaaaacatgagttgagaccccagccgcacttgcagcctagtgccattagcctgcaggctgtgctggatatctcagggcaagagtcgagcccttttgattttggggggattatttcaatatatttgctttttctttttgttttagttaatgtggagctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggctttatcgagaataggaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacgggagagtcagcctccacattcagaggcatcacaagtaatggcacaattcttcggatgactgcagaaaatagtgttttgtagttcaacaactcaagacgaagcttatttctgaggataagctctttaaaggcaaagctttattttcatctctcatcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatatgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagcttttctttatttttttaactttttggggggaaaagtatttttgagaagtttgtcttgcaatgtatttataaatagtaaataaagtttttaccattaaaaaaatatctttccctttgttattgaccatctctgggctttgtatcactaattattttattttattatataataattattttattaaaatgttccctgctttcccttttagcaa

SEQ ID NO: 33 - DNA, 8322 bp, Homo sapiens
  Genbank Accession No. D50030
gaattccgtgactccagcaccatcccaggccccagggtacctggcccagggttgtgctgccgcagacttggcctgtaccatccaggcggcggtggggagctggggttggaaaggcttcttggagtggactcctgggtctgtctgggagacggggaggaagggacactctgaacatcaccaggggctgctggggggccctggccacccccagagtcagaacaggcaggtggggcaggatctcaggtcatcctatgctacactcagccattgcgtggcccctctcctccctgtgcctggccttttggccagccctggggccaccgagaggatgcagcaccgaaccctccaggagcccccagtgctgccgtctgtgggacagggacaatcccatccccactgctactgtctgtgctgtgctgggcacagagctggacacctccaaggcccagcgcccgtagtggctctcatcatggacaattcacaggcagatggtggccagctctgtggcctgcagggactgggagcggcgccagaccatctaggccccaacctatctgcattatcctggaagacttcctggaggaggcttctaagctgaggcccaaggaccatgtcaggtctaggactaggaccagtgcaggccgaggccagagagacagctgggcttccaggtagggtcaaagtgaggtgggcagcaggtgtgggggccaggggactcggggacttcctctccggctgggcccgcctgacgtgggaggcagccagggttaatcatttccacgaagccttgaccccacctgccttggccgctctggagggcgctcccactgcccctcaggccagctcaggagccatggggcgctgggcctgggtccccagcccctggcccccaccggggctgggccccttcctcctcctcctcctgctgctgctgctgctgccacgggggttccagccccagcctggcggggtgagcactgaccttgtcgcagtgcgcacagaggttcccagtggctacttgggaggaggccagagggagggtcgccgcagagctggggatttgagggggcgggggtccgagcagggggcttacgttgaaagctggccctcacagggcaggtgggtgctggactggccttggaggctggggcctgggctcacggagccccacctgggggctgtcatccctgtgcggcctggcctgggcccctatgggtctgggggctgccccctctctctggacctctgttgggtctggatgcgtgaggttgggggaacctcccagggcctcctagctgctcccatcaccaaaaactgacgtcagggtttaagcttggtgggtagcagtgcctgggcctgggcagtgaccctgagcaggtgttaactaggggccgagggcggggaaggtgtcagggcctggctgaggcctcaggaatgtcgtgtgtctcctggaacctgctccgagatctgggggccccagtatggaaacaggctcagggctgtgacctcctgcccggcaggacctgagtgtgagggtctgtcccacactgacaccctttctgctcctcctagaaccgtacggagtccccagaacctaatgccacagcgacccctgcgatccccactatcctggtgacctctgtgacctctgagaccccagcaacaagtgctccagaggcagagggaccccaaagtggggggctcccgcccccgcccagggcagttccctcgagcagtagcccccaggcccaaggtgggtcaggtgggcctgggaggaggtgtcgtgcttcaccttagggctgggtgggaggagcatggctgcggctggaggtcctgaggggctcgggtgcccctcgagggagccctgaccctgccaccccctccccacagcactcaccgaggacgggaggccctgcaggttccccttccgctacgggggccgcatgctgcatgcctgcacttcggagggcagtgcacacaggaagtggtgggtccgggcagccggggcacccgagctggggtcacctgccccacgctgcctgcttttctgggagccgggcacacagtaggcgctcaccacgcagcaggcggacccagtgaacccagagaccctccagggtggtggggtggggtggggggcctctgcctgggacccccatgcacgcaggtgtcgccccccaggtgtgccacaactcacaactacgaccgggacagggcctggggctactgtgtggaggccaccccgcctccagggggcccaggtgggtgctgggttgggtagcctggggcgagcagggggcactgggccaggccagaagggccaaggggaggatggggcggacccgggcaggggtcctggctggaggggagcagaagaggagctctgggattaaccccggtgggtgccacttagggccagagcctctcccaggtgggacctgcgtctcatggtgcgtgcgcggccatcgctggggccaaggcgccgcctggtggtgaggccgggccctgcagccacacaggcgtgccccaggcaggggacaggtgggcagggtctgtggggtgcggtgccagccgccctgctcacatgggtcctgggacacatgggaggggtctgctgcctgacagtgtgggtgtcaggcttccaggaagccacgtgcacagctgggccagtggcccaggtgttgagtgggggacgctgggaggaaggggaggtctgtgcccctcccagggaccttgcaccccgaggggcgtagagagggcccctttgctctcagagcccctcactgacctgatggacgccttagcaagcagagaatgtcacagagggaccctgagcctcccagtccgcccctcacatcccctcccgcatgtccccagctgccctggatccctgtgcctccggcccctgcctcaatggaggctcctgctccaatacccaggacccccagtcctatcactgcagctgcccccgggccttcaccggcaaggactgcggcacaggtgagctgggcccggaggtccgcagggtccaggggccggagcaagtcccggaggatgggagaacaggtggctcccgagtgcagggcaggggccctgcacggggacagcaggtggcagggtgtgagggcttactgtgcacccctcagagaaatgctttgatgagacccgctacgagtacctggaggggggcgaccgctgggcccgcgtgcgccagggccacgtggaacagtgcgagtgcttcgggggccggacctggtgcgaaggcacccgacatacaggtgcgccacggggtgtgagccgtgccactgacccctgacgggtgtccctgtccacacccgagtgggaggaatggcctgaggtcacccagaaacaagggacaaggggtggaccccggccccgactccgctgtcgtggggcactgcgcggcccctggcccagctcctcggccctgcccccagcttgtctgagcagcccttgcctgaacgggggcacctgccacctgatcgtggccaccgggaccaccgtgtgtgcctgcccaccaggcttcgctggacggctctgcaacatcggtgagtgggtcagccccccggggtgccctggggcagtgccgggtggacccaccgtgggccggcctcactgcccctctgcccgcagagcctgatgagcgctgcttcttggggaacggcactgggtaccgtggcgtggccagcacctcagcctcgggcctcagctgcctggcctggaactccgatctgctctaccaggagctgcacgtggactccgtgggcgccgcggccctgctgggcctgggcccccatgcctactgccggtcagcaccaacgccgctccaggccgccgcacgcggggcaggcaggatttttcctggtgagaggccccccggctccctaggacccaggcggggccatctccgtccagacaggccccggaacctctgcctgggaggctgcccgagggagggccactctcttcccgctgctccaggtgcggggaaccgccctgctgggggttccgatgccccctccccatgccctcttcgaggggcagtgtgactccctgccagcccccacttatgcaccgcaggaatccggacaatgacgagaggccctggtgctacgtggtgaaggacagcgcgctctcctgggagtactgccgcctggaggcctgcggtgcgcggctggcggggggtgctgccttgggccccaccgaggtcacagcagttttccccgtgatcctcctagcccctctgcacacctggctactgcatctctgacaaatggggaaactggagctcacgggatcaggctggtccaaggtcacgcagtgggtcggtactggggtcacagtggggaagcagcccgctgcagggggctggtcaccaggcgacggcctcggggtggcacctgcccagcctggaccatgcccaggatggccgcagaagcctttcagggaggtgcagggagggcagcgtgcaggccccccagaggccacctgcttcctgccctggggagaggccccccggctccctaggaccccggcggggccacccggggctctcatgtgggggtctctgactgtgctggggctggctgtggggttccgggctgctgagggggccacaaaggcctccgtgtgtccagcccctctggccctcacagagcctcgggatcgggcatcaaaccccattctacagatttggaaactgagggcagaggtgagtgggcgccagggcctgagcagcgtggacagggggtccggccatggccctctgcagcgcctcctgccgggtagggcctgtgtgtggaagggggctggccctgtgaaccccaggggtgtgacccggtgacctttgctcccagaatccctcaccagagtccaactgtcaccggatctcctggcgaccctgcctgagccagcctccccggggcgccaggcctgcggcaggaggcacaagaagaggacgttcctgcggccacgtatcatcggcggctcctcctcgctgcccggctcgcacccctggctggccgccatctacatcggggacagcttctgcgccgggagcctggtccacacctgctgggtggtgtcggccgcccactgcttctcccacaggtgcacctcctctgggccccagtcacctgccctgaggccccacacaccatccagcgtcactatgcgcctgtccccacccgcttgcggaccccatcccggtgcctctgctgaccccttcccggggtccccggtaaccctctctgacccctctgggtcttggggctcagtcctgagctgggcatgcatggacactccagaaaccctgtccctattgagggaggccaggggtcagtggcatttccacagaggacccagcgcctgcggagagtgcagtgggccaggtcctgtcctccccgcagccccgcccctcccagagtcctggctctcagtagggagccagggagcgcagtacaatgcgtcagagccctaggagcccaagcacagccagggaaactgaggcccagcccactcagccggctcccctcagtgggggcgcacagccacccaggcccacactctgggaggcgggcctcgagtggggagaccctgagacggagcagcctgggccagacgcccttccaccttcgatgggcaccccagtgattaggggcaggggtccggccagccgagggtctggcggcctttcgaggaccacagctcccagggtggggtcctgcgtggccctgcagaacctgcagccgacgtctcctgggcctgcaaatggggccagagccttcctcagtgtggagaaatggggttccccacatggagcccagggcaggaggccgaggcagggtggagcgacctccacacagcagatgggagccaggctcaggggcccaggctctgtgcccagcaggagtgagggctgcatctggggaccccacaatgtggatggaagctggggcagggcctgagcaggccaggtctccacccactcgggtcggccttgcagccgatcctgcctgggcttggcctgagactccccccagggcttgtgaccagtgttgtcaggagggatggcaccgcacttcagctcccatgcctggatctgggggtgactggcaatagcccctgcttggtggtccctgtctccccagtatcaacagcccacacccaggcacctgctcagacccctccccgggaccagggtcccacaccattggcctccgtgggcctgacagggggtggggagaggtgagcccggtggctgggggagggctggtccatgcagcctccagccccccttgcacagcccccccagggacagcgtctccgtggtgctgggccagcacttcttcaaccgcacgacggacgtgacgcagaccttcggcatcgagaagtacatcccgtacaccctgtactcggtgttcaaccccagcgaccacgacctcggtgagctccggcgtgtcgtggctgcactctgggcaggtgggccctgtgctccccaggccaggcccagacaggggcaggagctgggcagacatgtggtgcgggggaagctgggggcagggcagggaggacagggaccgtcccttctggatctcccaggctgccctgtggaccccactggctaccctccctccaccacgggccccgacagcctcccgttcagcccgcacaccacaggctgaccctggccactcttctgatcagtcctgatccggctgaagaagaaaggggaccgctgtgccacacgctcgcagttcgtgcagcccatctgcctgcccgagcccggcagcaccttccccgcaggacacaagtgccagattgcgggctggggccacttggatgagagtgagttgggaggggggcgcccagcgtccgccaggtggacaggtggccagccacagtgtggtgtcacgctgagggcattgtgtccacagacgtgagcggctactccagctccctgcgggaggccctggtccccctggtcgccgaccacaagtgcagcagccctgaggtctacggcgccgacatcagccccaacatgctctgtgccggctacttcgactgcaagtccgacgcctgccaggtgagctggtgcccgccccaccaggacccgactggtgggggctcagctggtcctgagtctccgagatgcttgcccctggggagcccagagcctggcggcaccccaacctggcagggccagccagggacccctgggcagggagcacacttactgtcggtggccaggcaccccagggccacccagagccctgccggaagggcatcctcctcacacagaaggggagacccaggctctgagaggggcctggggtcccacagagcccggacagttcctggctgtccagcccaaggcagtgctttccgtgtgacactaggcaccaaaggcccctttgccccgagttctctcaagccagctcttcaaccttcaataagttcttcaaccttcaataagcccaacagcccaaccacgtcctgggctctgcagcctgtgtgctcagcgcacgcacctgcatgcgacggctctcttatgccccatttatagctggggagactgagccccaggcagtcaattcactcttccaaggccacaaagcccaggagtggtagagtgggtagggcttgtcactggacctcgataagggaagtcaggatggtttcctggaggagggagtgtttgcactggtcctgagtgatgcgtaggagttttccagggagtgaagaagtcatttcaggaggtgctgggacacagggtggctgtgtggtccacacagggttcatgctggtggggagagggggaccgtggcttcaggaaggggaataacgtggttggatgccctggcccagccagtgctgcccccagggaatgagacaccttttcatgaggttcaggagagtggatcagctcccttgctcagaaatgcctttgtcctctgtccccaagctgccacttggggtagaggggtgtccctgaaccaggcccctgggagggtggctctgaccaacgtctctgcccagggggactcaggggggcccctggcctgcgagaagaacggcgtggcttacctctacggcatcatcagctggggtgacggctgcgggcggctccacaagccgggggtctacacccgcgtggccaactatgtggactggatcaacgaccggatacggcctcccaggcggcttgtggctccctcctgaccctccagcgggacaccctggttcccaccattccctgccttgctgacaataaagatatttccaagaacccggcccacgctg

SEQ ID NO: 34 - DNA, 2576 bp, Homo sapiens
  Genbank Accession No. L04751
gaattccgcagagatccagcaggtgctgcaccatgagtgtctctgtgctgagccccagcagactcctgggtgatgtctctggaatcctccaagcggcctccctgctcattctgcttctgctgctgatcaaggcagttcagctctacctgcacaggcagtggctgctcaaagccctccagcagttcccgtgccctccctcccactggctcttcgggcacatccaggagctccaacaggaccaggagctacaacggattcagaaatgggtggagacattcccaagtgcctgtcctcattggctatggggaggcaaagttcgtgtccagctctatgaccctgactatatgaaggtgattctggggagatcagacccgaaatcccatggttcctacagattcctggctccatggattgggtacggcttgctcctgttgaatgggcagacatggttccagcatcgacggatgctgaccccagccttccactatgacatcctgaagccctatgtggggctcatggcagactctgtacgagtgatgctggacaaatgggaagagctccttggccaggattcccctctggaggtctttcagcacgtctccttgatgaccctggacaccatcatgaagtgtgccttcagccatcagggcagcatccaggtggacaggaattctcagtcctacatacaggccattagtgacctgaacaacctggttttttcccgtgtgaggaatgcctttcaccagaatgacaccatctacagcctgacctctgctggccgctggacacaccgcgcctgccagctggcccatcagcacacagaccaagtgatccaactgaggaaggctcaactacagaaggagggggagctggagaagatcaagaggaagaggcatttggattttctggatatcctcctcttggccaaaatggagaatgggagcatcttgtcagacaaggacctccgtgctgaggtggacacgttcatgtttgagggccacgacaccacagccagtgggatctcctggatcctctatgctctggccacacaccccaagcatcaggagaggtgccgggaggagatccacagcctcctgggtgatggagcctccatcacctggaaccacctggaccagatgccctacaccaccatgtgcattaaggaggcactgaggctctacccaccggtgccaggcattggcagagagctcagcactcccgtcaccttccctgatgggcgctccttgcccaaaggtatcatggtcctcctctccatttatggccttcaccacaacccaaaagtgtggcccaacccagaggtgtttgaccctttccgttttgcaccgggttctgctcaacacagccacgctttcctgcccttctcaggaggatcaaggaactgcattgggaaacaatttgccatgaacgagctgaaggtggccacggccctgaccctgctccgctttgagctgctgcctgatcccaccaggatccccatccccattgcacgacttgtgttgaaatccaaaaatggaatccacctgcgtctcaggaggctccctaacccttgtgaagacaaggaccagctttgagggcctccacctgccgtcctgtcttcctgacccccgcttctgtccccttcctgtctgcccatatcctgttttctgtctgcccaccttcccttcttcccacctgcctgctgtcccccagtctgcctgcccttctctctctcacctttctccaggctccctacctgcttgtctacctgtctcctacccacctgtatctcttgttgggagaaaagctgagtgttgggagaagctgaggccgagcttgcatgtctgacataatgtaaaagagtcttgaatcatgtccaggatccagggtctaaaaccccttgtggcctttggaacaccaagctctgtgctgaagggtggaaggctaccctgacgcaccataatctaagcccggggcataaaacccctcgtggcttggatagaatccagggctcgtggctctggaatgtgtctggacttgctggctccttgctccttgctctcccaggatcaattgtatcttgagttaaaagaacctgctctccattatctcaagtaacagagcagatgctaaaccgtcacagctgtaaattgtgtgcttaatgcaacatgccctttcgacccaccccccattctcaccacctgtttctttgtttgatcaccaataaataatctgcacttccagagctcggggccttcacagcctccatccttagctttggcgccctggacccactttctctctcaaactgtcttttctcactgctttgactctgccggactttgtcacccccacgacctggtgttgggtctgaacaccccaacatccctgaatctccacccacctcccaaactcctgcctgccctccagactgtctgcccatacacctgtctccttcttcctgcctggcttgtctgttcctatattagtttcctattactgctgtaataaactatcacaatctcagtgattttaaataaaaaaaaaaaaaaaaaaaaaaaaaaaaaaaaacggaattc

SEQ ID NO: 35 - DNA, 1719 bp, Homo sapiens
  Genbank Accession No. L27050
caaacacatacaggaagcgatcaaacctaccaaggcagtctcacttctcaatgactggactgtgtgggtactctgctccagacatgcgtggcctcagactcatcatgataccagttgagctgctactttgctacctcctgctgcaccctgtggatgccacttcatatggaaagcagacaaatgtcttgatgcactttcccttgtccttggaatcccagacaccctcctcagaccccttgtcctgccaatttctgcacccaaagtcactgcctggtttcagccacatggcccctctacccaagttcttggtaagcctggctctaaggaatgccctggaggaagctggttgtcaggctgatgtttgggctctacagctacagctctaccgccagggtggtgtgaatgctacacaggtcctcatccagcatcttcgagggctccagaaaggcagaagcacagagaggaacgtgtcagtggaagccctggcctctgctctgcagctgttagccagggagcagcaaagcacaggaagggtcgggcgctccctcccgacagaggactgtgagaatgagaaggagcaagctgtgcacaatgtagtccagctgctgccaggagtgggaaccttctacaacctgggcacagctttgtattatgctactcaaaactgcctgggcaaggccagggaacgaggccgagatggggccatagatctgggatatgaccttctgatgaccatggctgggatgtcaggggggcctatgggtctagcgatcagtgctgcacttaaacctgcattaaggtctggggttcagcagttgatccagtattaccaagatcagaaagacgcaaacatctctcagccggagaccaccaaggagggtttgagggccatctcagatgtgagtgacttggaagaaacaactactctggcttctttcatatcagaagtagtaagttcagctccctactgggggtgggccataatcaagagctatgacttagatcctggggctgggagtcttgagatataaaagaatgtggtaaccacagaattaataactgtctaccctgacaagctatatacatgtcttcaaaattttaatctgatttatccaggaggaaggctgtacagtaaaacgtaagaacgtaaatgtttgggtgttgaagtcacagggtttggtttcgaatctaggctccacttgttagagcctcggtgatcactgaatagtaacttctttcttgaactaagatcagttttgaagtttctaaaggagatagaatgattttaacctcaatgagttgccctgtaaatttaaaatgatacaatgaatctaaaatgcttatcacagtactttcaataaatagctattagccaggtgcggtggctcacgcctgtaatcccagcactgtgagaggctgaggcgggatgatcacctgaggtcaggagttcaagatcagcctgcgcaacatggcgaaaccccgtctctacaataaatagcaaaaaattatcctggcggagttatgcacgcttgtagtcccaactacctgggaggctgaggcgggagaatcacctgagcctgggaggctgaggcgggagaatcacctgagcctgggaggtcgaggctgcagcgagccgagatcgcgccgctgcattccagcctgggtgacagagcgagaccatgtctcaaaaaataaaaataaaaaaaaattgttttcatt

SEQ ID NO: 36 - DNA, 1337 bp, Homo sapiens
  Genbank Accession No. U37100
caaaaacagcaacagaaagcaggacgtgagacttctacctgctcactcagaatcatttctgcaccaaccatggccacgtttgtggagctcagtaccaaagccaagatgcccattgtgggcctgggcacttggaagtctcctctcggcaaagtgaaagaagcagtgaaggtggccattgatgcaggatatcggcacattgactgtgcctatgtctatcagaatgaacatgaagtgggggaagccatccaagagaagatccaagagaaggctgtgaagcgggaggacctgttcatcgtcagcaagttgtggcccactttctttgagagaccccttgtgaggaaagcctttgagaagaccctcaaggacctgaagctgagctatctggacgtctatcttattcactggccacagggattcaagtctggggatgaccttttccccaaagatgataaaggtaatgccatcggtggaaaagcaacgttcttggatgcctgggaggccatggaggagctggtggatgaggggctggtgaaagcccttggggtctccaatttcagccacttccagatcgagaagctcttgaacaaacctggactgaaatataaaccagtgactaaccaggttgagtgtcacccatacctcacgcaggagaaactgatccagtactgccactccaagggcatcaccgttacggcctacagccccctgggctctccggatagaccttgggccaagccagaagacccttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcgatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactgaggatgtaaagatcaataaaaaaaataataatcat

SEQ ID NO: 37 - DNA, 1517 bp, Homo sapiens
  Genbank Accession No. U02390
attctttggggaggcaactaggatggtgtggccgaccacggatttgcattgccgaggacgggaccccagggcagcgaagcagaatggccaacatgcagggactggtggaaagactggaacgagctgtcagccgcctggagtcgctgtctgcagagtcccacaggccccctgggaactgcggggaagtcaatggtgtcattgcaggtgtggcaccctccgtggaagcctttgacaagctgatggacagtatggtggccgagtttttaaagaacagtaggatccttgctggggacgtggagacccatgcagaaatggtgcacagtgctttccaggcccagcgggctttccttctgatggcctctcagtaccaacaaccccacgagaatgacgtggccgcacttctgaaacccatatcggaaaagattcaggaaatccaaactttcagagagagaaaccgggggagtaacatgtttaatcatctttcggccgtcagcgaaagcatccctgcccttggatggatagctgtgtctcccaaacctggtccttatgtcaaggagatgaatgacgctgccaccttttacactaacagggtcttaaaggactacaaacacagtgatttgcgtcatgtggattgggtgaagtcatatttgaacatttggagtgaacttcaagcatacatcaaggaacaccacaccacgggcctcacatggagcaaaacaggtcctgtagcatccacagtatcagcgttttctgtcctctcctctgggcctggccttcctccaccccctcctcctctgcctcctccagggccacctccacttttcgagaatgaaggcaaaaaagaggaatcttctccttcacgctcagctttatttgcccaacttaaccagggagaagcaattacaaaagggctccgccatgtcacagatgaccagaagacatacaaaaatcccagcctgcgggctcaaggagggcaaactcaatctcccaccaaaagtcacactccaagtcccacatctcctaaatcttatccttctcaaaaacatgccccagtgttggagttggaaggaaagaaatggagagtggagtaccaagaggacaggaatgaccttgtgatttcagagactgagctgaaacaagtggcttacattttcaaatgcgaaaaatcaactattcagataaaagggaaagtaaactccattataattgacaactgtaagaaactcggcctggtgtttgacaatgtggtgggcattgtggaagtgatcaactcccaggacattcaaatccaggtaatggggagagtgccaacaatttccattaataagacagaaggttgccacatatacctcagtgaagatgcattagactgtgagatcgtgagcgccaagtcatctgaaatgaacatacttatccctcaggatggtgattatagagaatttcccattcctgaacagttcaagacagcatgggatggatccaagttaatcactgaacctgcagaaattatggcctaa

SEQ ID NO: 38 - DNA, 536 bp, Homo sapiens
  Genbank Accession No. AW006831
tttttctggaattcttttttttttttttttttttgaggattttgtgtggctaatgtgttctagaagcagagactccagggagaaccagaatttatgaagcctcgtgcaacatggtgctttctcaccgaggtcatatgcctggctgctgctgttccactcagctccatgagccacgtttgttattttatgtttcttctgtgcttttgctcatttccacccatgtgtttatagaccttttttcagccctttttctttgttcctttccctcatctttttgcctcaggtagaatccatcagttttcctccccctccaaatgactgtgtacctccagctgctcaggactttggaggtgggggcggggcctggggatctacccttctaaaaaactctccaagtaattctgataccaataaagtgggagacctccatttttgaggcctatttgcaagcatgtctccctccctttccatggctttttcgtttctcgcatgttccactgctttgcactgtctagacaggaaacctaggaagatgt

SEQ ID NO: 39 - DNA, 2194 bp, Homo sapiens
  Genbank Accession No. U90441
ggggaaggaacactgtaggggatagctgtccacggacgctgtctacaagaccctggagtgagataacgtgcctggtactgtgccctgcatgtgtaagatgcccagttgaccttcgcagcaggagcctggatcaggcacttcctgcctcaggtattgctggacagcccagacacttccctctgtgaccatgaaactctgggtgtctgcattgctgatggcctggtttggtgtcctgagctgtgtgcaggccgaattcttcacctctattgggcacatgactgacctgatttatgcagagaaagagctggtgcagtctctgaaagagtacatccttgtggaggaagccaagctttccaagattaagagctgggccaacaaaatggaagccttgactagcaagtcagctgctgatgctgagggctacctggctcaccctgtgaatgcctacaaactggtgaagcggctaaacacagactggcctgcgctggaggaccttgtcctgcaggactcagctgcaggttttatcgccaacctctctgtgcagcggcagttcttccccactgatgaggacgagataggagctgccaaagccctgatgagacttcaggacacatacaggctggacccaggcacaatttccagaggggaacttccaggaaccaagtaccaggcaatgctgagtgtggatgactgctttgggatgggccgctcggcctacaatgaaggggactattatcatacggtgttgtggatggagcaggtgctaaagcagcttgatgccggggaggaggccaccacaaccaagtcacaggtgctggactacctcagctatgctgtcttccagttgggtgatctgcaccgtgccctggagctcacccgccgcctgctctcccttgacccaagccacgaacgagctggagggaatctgcggtactttgagcagttattggaggaagagagagaaaaaacgttaacaaatcagacagaagctgagctagcaaccccagaaggcatctatgagaggcctgtggactacctgcctgagagggatgtttacgagagcctctgtcgtggggagggtgtcaaactgacaccccgtagacagaagaggcttttctgtaggtaccaccatggcaacagggccccacagctgctcattgcccccttcaaagaggaggacgagtgggacagcccgcacatcgtcaggtactacgatgtcatgtctgatgaggaaatcgagaggatcaaggagatcgcaaaacctaaacttgcacgagccaccgttcgtgatcccaagacaggagtcctcactgtcgccagctaccgggtttccaaaagctcctggctagaggaagatgatgaccctgttgtggcccgagtaaatcgtcggatgcagcatatcacagggttaacagtaaagactgcagaattgttacaggttgcaaattatggagtgggaggacagtatgaaccgcacttcgacttctctaggaatgatgagcgagatactttcaagcatttagggacggggaatcgtgtggctactttcttaaactacatgagtgatgtagaagctggtggtgccaccgtcttccctgatctgggggctgcaatttggcctaagaagggtacagctgtgttctggtacaacctcttgcggagcggggaaggtgactaccgaacaagacatgctgcctgccctgtgcttgtgggctgcaagtgggtctccaataagtggttccatgaacgaggacaggagttcttgagaccttgtggatcaacagaagttgactgacatccttttctgtccttccccttcctggtccttcagcccatgtcaacgtgacagacacctttgtatgttccttgtatgttcctatcaggctgatttttggagaaatgaatgtttgtctggagcagagggagaccatactagggcgactcctgtgtgactgaagtcccagcccttccattcagcctgtgccatccctggccccaaggctaggatcaaagtggctgcagcagagttagctgtctagcgcctagcaaggtgcctttgtacctcaggtgttttaggtgtgagatgtttcagtgaaccaaagttctgataccttgtttacatgtttgtttttatggcatttctatctattgtggctttaccaaaaaataaaatgtccctaccagaagccttaaa

SEQ ID NO: 40 - DNA, 1324 bp, Homo sapiens
  Genbank Accession No. D43642
ctggtgaggggctgcaggtggcggcgcagtctcggtaggcggtatgagtttggctgggggccgggcaccccggaagaccgctgggaaccggctttctgggcttttggaggcagaggaggaagatgagttctaccagacgacttatgggggtttcacagaggaatccggagatgatgagtatcaaggggaccagtcagacacagaggacgaagtggactctgactttgacattgatgaaggggatgaaccatccagtgatggagaagcagaagagccaagaaggaagcgccgagtagtcaccaaggcctataaggaacctctcaagagcttaaggcctcgaaaggtcaacaccccggctggtagctctcagaaggcgcgagaagagaaggcactactgccattagaactacaagatgacggctctgacagtcggaagtctatgcgtcagtctacagctgagcatacacgacaaacgttccttcgggtacaggagaggcagggccagtcaagacggcgaaaggggccccactgtgagcggccactaacccaggaggaactgctccgggaggccaagatcacagaagagcttaatttacggtcactggagacatatgagcggctcgaggctgataaaaagaagcaggttcataagaagcggaagtgccccgggcccataatcacctatcattcagtgacagtgccacttgttggggagccaggccccaaggaagagaacgttgacatagaaggacttgatcctgctccctcggtgtctgcattgactcctcatgctgggactggacccgtcaacccccctgctcgctgctcacgtaccttcatcacttttagtgatgatgcaactttcgaggaatggttcccccaagggcggcccccaaaagtccctgttcgtgaggtctgtccagtgacccatcgtccagccctataccgggaccctgttacagacataccctatgccactgctcgagccttcaagatcattcgtgaggcttacaagaagtacattactgcccatggactgccgcccactgcctcagccctgggccccggcccgccacctcctgagcccctccctggctctgggccccgagccttgcgccagaaaattgtcattaaatgaagagatgtctagtcctcagaaacttctttcctgccctgattggggctcttgctgttccgtttcttctccctgcttctcccctttgtcatctctgatctttgcctaatctgtttctttttccttttcccctagttcttacaggtttcgttgtgttttttaatctaataaaatagaaagatccctttt

SEQ ID NO: 41 - DNA, 890 bp, Homo sapiens
  Genbank Accession No. M91670
ggcggaccgaagaacgcaggaagggggccggggggacccgcccccggccggccgcagccatgaactccaacgtggagaacctacccccgcacatcatccgcctggtgtacaaggaggtgacgacactgaccgcagacccacccgatggcatcaaggtctttcccaacgaggaggacctcaccgacctccaggtcaccatcgagggccctgaggggaccccatatgctggaggtctgttccgcatgaaactcctgctggggaaggacttccctgcctccccacccaagggctacttcctgaccaagatcttccacccgaacgtgggcgccaatggcgagatctgcgtcaacgtgctcaagagggactggacggctgagctgggcatccgacacgtactgctgaccatcaagtgcctgctgatccaccctaaccccgagtctgcactcaacgaggaggcgggccgcctgctcttggagaactacgaggagtatgcggctcgggcccgtctgctcacagagatccacgggggcgccggcgggcccagcggcagggccgaagccggtcgggccctggccagtggcactgaagcttcctccaccgaccctggggccccagggggcccgggaggggctgagggtcccatggccaagaagcatgctggcgagcgcgataagaagctggcggccaagaaaaagacggacaagaagcgggcgctgcgggcgctgcggcggctgtagtgggctctcttcctccttccaccgtgaccccaacctctcctgtcccctccctccaactctgtctctaagttatttaaattatggctggggtcggggagggtacagggggcactgggacctggatttgtttttctaaataaagttggaaaagca

SEQ ID NO: 42 - DNA, 457 bp, Homo sapiens
  Genbank Accession No. AI478190
tttttttttttttttttttttttttttttttggggatttatatgacttttattttaatatcattttaatatcgaatatgtcacaagatttaatgaccaaattagtcatttacatttttcaaagtatctaaatataaaattgctaaaaattcaaatacagatagcaagctacaaatatttcttttgtttttggggggggggtaggaatggaagacattaaaaaaggacgctcctgttggctgaacaaagatcagaatgaaagaagaattcatcagcntgaccccttgtgcaaagaaatacactcagctttaagatgctgttttagacacatctcttcctgtacaacaatttaaaaaatgtttctaatgcaggtcctcagtgaaacacccctctccctgaaacgtgagaaacagcagctttccgcctgcttgaaaaaggaggcaggcgctgtaaggaga

SEQ ID NO: 43 - DNA, 1330 bp, Homo sapiens
  Genbank Accession No. U51007
aattcccaaatgaccttttatttcatacagagatacaaaggcaactatgtgcagcaacaatctgatgggcagtccaaactcttgggaggaagtaaattcatggtaaatgtcatgatggcggtcgggagggaggaaggtggcaagatggtgttggaaagcactatggtgtgtgtggacaacagtgagtatatgcggaatggagacttcttacccaccaggctgcaggcccagcaggatgctgtcaacatagtttgtcattcaaagacccgcagcaaccctgagaacaacgtgggccttatcacactggctaatgactgtgaagtgctgaccacactcaccccagacactggccgtatcctgtccaagctacatactgtccaacccaagggcaagatcaccttctgcacgggcatccgcgtggcccatctggctctgaagcaccgacaaggcaagaatcacaagatgcgcatcattgcctttgtgggaagcccagtggaggacaatgagaaggatctggtgaaactggctaaacgcctcaagaaggagaaagtaaatgttgacattatcaattttggggaagaggaggtgaacacagaaaagctgacagcctttgtaaacacgttgaatggcaaagatggaaccggttctcatctggtgacagtgcctcctgggcccagtttggctgatgctctcatcagttctccgattttggctggtgaaggtggtgccatgctgggtcttggtgccagtgactttgaatttggagtagatcccagtgctgatcctgagctggccttggcccttcgtgtatctatggaagagcagcggcagcggcaggaggaggaggcccggcgggcagctgcagcttctgctgctgaggccgggattgctacgactgggactgaagactcagacgatgccctgctgaagatgaccatcagccagcaagagtttggccgcactgggcttcctgacctaagcagtatgactgaggaagagcagattgcttatgccatgcagatgtccctgcagggagcagagtttggccaggcggaatcagcagacattgatgccagctcagctatggacacatccgagccagccaaggaggaggatgattacgacgtgatgcaggaccccgagttccttcagagtgtcctagagaacctcccaggtgtggatcccaacaatgaagccattcgaaatgctatgggctccctggcctcccaggccaccaaggacggcaagaaggacaagaaggaggaagacaagaagtgagactggagggaaagggtagctgagtctgcttagggactgcatgggggaattc

SEQ ID NO: 44 - DNA, 776 bp, Homo sapiens
  Genbank Accession No. AI953020
gcggccgccatctcgccgtcgtcccgcggggtgcccggggcgttgctcaggccggccacggcgccgggggaactcttcggcaacccgtccatgtcgcccgagcccagggatccgttcacgtggtgaggctccatcgcgctcatggcggccatggggccctccgggccagggccgagcgggaaattagccctgccggcgccctgcccgatggggttcatgatagtgtacatgttttcgctggagttggtggaatctccagggctaggcatgatgggtgttccagggggcccacctcctcctgggggtccggtgtagctgccgggggatgaggaggagtaggggatcgagtttccactggggctggcccacgggccacgaactcctgggcccatgttcatggcaggcaggcctgggccggcgagggagttgggtgggggtcgcatgccacctccatagctctggggccccacgctggccatgccacgaggaggcgtcaccctctgcattgngccgcccatgctcggatgcccctgggctcgtggggagggctccatggcgccagggaggaggggctggggagccaggaggcctgcgggaggctgacnntcgcatccgcagggtggccgnangcccnctgngaagcgcggtgacatgaaggggctgggagagccangccgcatggagcctgcngncattgtgtcacctggggccatactccncataaccggggctgnggcggctgcagcactatagtnctggaaggncntggnctcngc

SEQ ID NO: 45 - DNA, 476 bp, Homo sapiens
  Genbank Accession No. AI971969
ttttttttttgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagcggtttagcacgaccacaacagcagagccatcgggatgcatcagtgccactgcgtccaggtcgttcttctgactggcaaccagccccactctctgggagccctcaggaatgaacttgctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggtttgttgatgaatgctgtg

SEQ ID NO: 46 - DNA, 2745 bp, Homo sapiens
  Genbank Accession No. AF046889
acaatcctcgccttgtctgtggcgccggcatctggagctttctgtagcctccggatacgcctttttttcagggcgtagcccctagccaagctgctccccgcggcggccgcacagcagcccgagcgccccctttccagagctcccctccggagctgggatccaggcgcgtagcggagatcccaggatcctgggtgctgtttgggcccgctccccaccatgacctcctcggggcctggaccccggttcctgctgctgctgccgctgctgctgccccctgcggcctcagcctccgaccggccccggggccgagacccggtcaacccagagaagctgctggtgatcactgtggccacagctgaaaccgaggggtacctgcgtttcctgcgctctgcggagttcttcaactacactgtgcggaccctgggcctgggagaggagtggcgagggggtgatgtggctcgaacagttggtggaggacagaaggtccggtggttaaagaaggaaatggagaaatacgctgaccgggaggatatgatcatcatgtttgtggatagctacgacgtgattctggccggcagccccacagagctgctgaagaagttcgtccagagtggcagccgcctgctcttctctgcagagagcttctgctggcccgagtgggggctggcggagcagtaccctgaggtgggcacggggaagcgcttcctcaattctggtggattcatcggttttgccaccaccatccaccaaatcgtgcgccagtggaagtacaaggatgatgacgacgaccagctgttctacacacggctctacctggacccaggactgagggagaaactcagccttaatctggatcataagtctcggatctttcagaacctcaacggggctttagatgaagtggttttaaagtttgatcggaaccgtgtgcgtatccggaacgtggcctacgacacgctccccattgtggtccatggaaacggtcccactaagctgcagctcaactacctgggaaactacgtccccaatggctggactcctgagggaggctgtggcttctgcaaccaggaccggaggacactcccgggggggcagcctcccccccgggtgtttctggccgtgtttgtggaacagcctactccgtttctgccccgcttcctgcagcggctgctactcctggactatccccccgacagggtcacccttttcctgcacaacaacgaggtcttccatgaaccccacatcgctgactcctggccgcagctccaggaccacttctcagctgtgaagctcgtggggccggaggaggctctgagcccaggcgaggccagggacatggccatggacctgtgtcggcaggaccccgagtgtgagttctacttcagcctggacgccgacgctgtcctcaccaacctgcagaccctgcgtatcctcattgaggagaacaggaaggtgatcgcccccatgctgtcccgccacggcaagctgtggtccaacttctggggcgccctgagccccgatgagtactacgcccgctccgaggactacgtggagctggtgcagcggaagcgagtgggtgtgtggaatgtaccatacatctcccaggcctatgtgatccggggtgataccctgcggatggagctgccccagagggatgtgttctcgggcagtgacacagacccggacatggccttctgtaagagctttcgagacaagggcatcttcctccatctgagcaatcagcatgaatttggccggctcctggccacttccagatacgacacggagcacctgcaccccgacctctggcagatcttcgacaaccccgtcgactggaaggagcagtacatccacgagaactacagccgggccctggaaggggaaggaatcgtggagcagccatgcccggacgtgtactggttcccactgctgtcagaacaaatgtgtgatgagctggtggcagagatggagcactacggccagtggtcaggcggccggcatgaggattcaaggctggctggaggctacgagaatgtgcccaccgtggacatccacatgaagcaggtggggtacgaggaccagtggctgcagctgctgcggacgtatgtgggccccatgaccgagagcctgtttcccggttaccacaccaaggcgcgggcggtgatgaactttgtggttcgctaccggccagacgagcagccgtctctgcggccacaccacgactcatccaccttcaccctcaacgttgccctcaaccacaagggcctggactatgagggaggtggctgccgcttcctgcgctacgactgtgtgatctcctccccgaggaagggctgggcactcctgcaccccggccgcctcacccactaccacgaggggctgccaacgacctggggcacacgctacatcatggtgtcctttgtcgacccctgacactcaaccactctgccaaacctgccctgccattgtgcctttttagggggcctggcccccgtcctgggagttgggggatgggtctctctgtctccccacttcctgagttcatgttccgcgtgcctgaactgaatatgtcaccttgctcccaagacacggccctctcaggaagctcccggagtccccgcctctctcctccgcccacaggggttcgtgggcacagggcttctggggactccccgcgtgataaattattaatgttccgcagtctcactctgaataaaggacagtttgtaaatcttaaaaaaaaaa

SEQ ID NO: 47 - DNA, 524 bp, Homo sapiens
  Genbank Accession No. AI937030
ggaagggaccactgcctttattgcctctgtgctggggtcccagcctggggttcagaggcctctgggggcaataggtgaccctggacccaaattattgctacttggccaggtcaccttggggcttcccatactgccctgagaatgggtgggatgagggcatgcaaacgatatgcaaatgacatgcaaaccaacccagaggcctctggcacatccatgggtgctggaagagtcaaagcctantggcctgggagggcgacggggcacccagctcaggcccagcctcggaggcaaggtttgagggttggggggtccctgagcatcagccctgcttctgccgctggctgaggaggagcagaagcctggtcccaagacagcaggaagaaagcaggaccctcgctaggcggccagatggcctgaggcccacccagacagggacgggacctgccctgngtcgctcagccaggcagggacagcagggaccagaatccaggtctcctgagccaggcagaagccaaagtagcttg

SEQ ID NO: 48 - DNA, 571 bp, Homo sapiens
  Genbank Accession No. AI829890
tttttttaacaggaaaaaattatttaatagtataacaaaatgcgaaataaagtacccaagttacaaaacataaattcctttggttcatgatcacaccactatttttaccttccacatagctacagacatcacaccctcaaagtgaagtcaaactgtccccctcatactgaagatgtcatgccaaaaccatcacataccccactgttcagtgaaactgttggcaacttacatggaacagagctgtggggtaggaaaaaggggaaagggttgcgttaaaaaaaatggggagactctacacatgcanaacaagttagtgggagggagtgctctgctgggtcaacacgccatgaaccacacccctattcgtgctacatgaggctgagtccttgctacaaccacacagaaatacagacaatcaagtgaacctgagcacccccagggataacagaagaaaaatacagagaagcagaggagagaaagaatggcagcaagaggcagatcacagaataccagggaacacctagtccaaaccctgttttacagatggggaaagtgagacctggagtagtga

SEQ ID NO: 49 - DNA, 1204 bp, Homo sapiens
  Genbank Accession No. D17793
ctctgaggagaagcagcagcaaacatttgctagtcagacaagtgacagggaatggattccaaacagcagtgtgtaaagctaaatgatggccacttcatgcctgtattgggatttggcacctatgcacctccagaggttccgagaagtaaagctttggaggtcacaaaattagcaatagaagctgggttccgccatatagattctgctcatttatacaataatgaggagcaggttggactggccatccgaagcaagattgcagatggcagtgtgaagagagaagacatattctacacttcaaagctttggtccacttttcatcgaccagagttggtccgaccagccttggaaaactcactgaaaaaagctcaattggactatgttgacctctatcttattcattctccaatgtctctaaagccaggtgaggaactttcaccaacagatgaaaatggaaaagtaatatttgacatagtggatctctgtaccacctgggaggccatggagaagtgtaaggatgcaggattggccaagtccattggggtgtcaaacttcaaccgcaggcagctggagatgatcctcaacaagccaggactcaagtacaagcctgtctgcaaccaggtagaatgtcatccgtatttcaaccggagtaaattgctagatttctgcaagtcgaaagatattgttctggttgcctatagtgctctgggatctcaacgagacaaacgatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtttttgagttccagttgactgcagaggacatgaaagccatagatggcctagacagaaatctccactattttaacagtgatagttttgctagccaccctaattatccatattcagatgaatattaacatggagagctttgcctgatgtctaccagaagccctgtgtgtggatggtgacgcagaggacgtctctatgccggtgactggacatatcacctctacttaaatccgtcctgtttagcgacttcagtcaactacagctgagtccataggccagaaagacaataaatttttatcattttgaaat

SEQ ID NO: 50 - DNA, 2436 bp, Homo sapiens
  Genbank Accession No. X62078
aaggcacctctgccgccacagaccttgcagttaactccgccctgacccacccttcccgatgcagtccctgatgcaggctcccctcctgatcgccctgggcttgcttctcgcgacccctgcgcaagcccacctgaaaaagccatcccagctcagtagcttttcctgggataactgtgatgaagggaaggaccctgcggtgatcagaagcctgactctggagcctgaccccatcgtcgttcctggaaatgtgaccctcagtgtcgtgggcagcaccagtgtccccctgagttctcctctgaaggtggatttagttttggagaaggaggtggctggcctctggatcaagatcccatgcacagactacattggcagctgtacctttgaacacttctgtgatgtgcttgacatgttaattcctactggggagccctgcccagagcccctgcgtacctatgggcttccttgccactgtcccttcaaagaaggaacctactcactgcccaagagcgaattcgttgtgcctgacctggagctgcccagttggctcaccaccgggaactaccgcatagagagcgtcctgagcagcagtgggaagcgtctgggctgcatcaagatcgctgcctctctaaagggcatataacatggcatctgccacagcagaatggagcggtgtgaggaaggtcccttttcctctgttttgtgtttgccaaggccaaactcccactctctgcccccctttaatcccctttctacagtgagtccactaccctcactgaaaatcattttgtaccacttacattttaggctggggcaagcagccctgacctaagggagaatgagttggacagttcttgatagcccagggcatctgctgggctgaccacgttactcatccccgttaacattctctctaaagagcctcgttcatttccaaagcagttaaggaatgggaaccagagtgttttaggacctgaagaatctttatgactctctctctttcactcttttttttttttgtcactaagttaaaagcgaagtgagagtattaacgtttttgttctcctccggccccctgttacaatgaaggggcaaaagtatttgctcttagtctattcctcccttaacttctgtgactaatttttatttcctttctagatttgcccaattaatactagggtgcagtgtatcctggagaggtagggtgtgtgggggaggaatcccttgggggagatattaggagtgctctgttgtttacaaactcacggtacccgcagggcctagcaagagacttaaatgactgataagaaccgtgagaaacatgttgcttccaggcttgatttcgatttttcgctttttttttttttgagacagaatctcactttgtcaccaggctggagtgcagtggtgcaatctcacctcactgcaacctccgcctcctgggttcaagcaattctcctgcctcagcctcccaagtagcttggactacaggccctgccaccacgcccggctaatttgtgtatttttagtagagatggggtttcaccatgttggccaggatggtctcgatctcttgacctcgtgatctgtccaccttggccttgcaaagcgctggattacaggcatgagccactacacccagccgatttttcctttttgattaaagatgctattacaatgtaaatatttcttacacagaaagtcacagcacatgtgcccattgatacaaggctgctgaggcctggtctccagttggaaatataattaagggtggcaaggactggagtcagttggagagtgcatagccagtctgtgaagacaactgccagatactggcaatactccagcctggtgacagagtgagactctgtctcaaaaaaaaagtttcaatgtttactcctagagaagccaaaaatccagatttgtatatgaaatcttaccattttaaaagattggcagctaattatttttttaaaaagctgtgcagtgtgatgtgtcccaaacggactggctcatgggtggccacgtcacaacctctgatctcagaccgtgcatgccttgtcctcttaagacaactcctgtggcaccgtttctccctccacagggccaaagccatagtgtccggtcccaaggacaaggctcttccagtgctaggagaggtatgagcagcctctcacctgtgagctgtggggatcacaaggctgcctgcctcagtcttggagtcctgttgggtgaatgaggcagatgggaaagagcctcaccagcagctgcttttggagcaggggtccaaggaagagagggtggcctcgacatcaaactgcctggatttttctaccaccctgttacatcataacaacttctgaaacacacaccagccctgagttctgggctcatttgaagcctggaatagcaataaatctttttaacttgcggacagtt

SEQ ID NO: 51 - DNA, 3590 bp, Homo sapiens
  Genbank Accession No. L76191
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggttccaagccctgcttccctgtggcctccaccgccatctccagccccttcttctaccaagccaggcccagagagctcagtgtccctcctgcagggagcccgcccctctccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcatttggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttccctaaaaaaaaaaaaaggaattc

SEQ ID NO: 52 - DNA, 3068 bp, Homo sapiens
  Genbank Accession No. U81802
gaagtccctatcagattacacttggttgactactccggagcagccactaagagggatgaacaggcctgcgtggaaattgaatgagattagttgagtccacgctccatgagaatgctgaacaccatccaaagcagcaaattgagattccttgatttggggaagaggtttgggaggaacccttcaataattggcatgggacaagaggggacccagtccaagtgtatttgggactcgcagtagggaggaacaattcagagagagcttggaagctcgaagtctggctgtggccatgggagatacagtagtggagcctgcccccttgaagccaacttctgagcccacttctggcccaccagggaataatggggggtccctgctaagtgtcatcacggagggggtcggggaactatcagtgattgaccctgaggtggcccagaaggcctgccaggaggtgttggagaaagtcaagcttttgcatggaggcgtggcagtctctagcagaggcaccccactggagttggtcaatggggatggtgtggacagtgagatccgttgcctagatgatccacctgcccagatcagggaggaggaagatgagatgggggccgctgtggcctcaggcacagccaaaggagcaagaagacggcggcagaacaactcagctaaacagtcttggctgctgaggctgtttgagtcaaaactgtttgacatctccatggccatttcatacctgtataactccaaggagcctggagtacaagcctacattggcaaccggctcttctgctttcgcaacgaggacgtggacttctatctgccccagttgcttaacatgtacatccacatggatgaggacgtgggtgatgccattaagccctacatagtccaccgttgccgccagagcattaacttttccctccagtgtgccctgttggttggggcctattcttcagacatgcacatttccactcaacgacactcccgtgggaccaagctacggaagctgatcctctcagatgagctaaagccagctcacaggaagagggagctgccctccttgagcccggcccctgatacagggctgtctccctccaaaaggactcaccagcgctctaagtcagatgccactgccagcataagtctcagcagcaacctgaaacgaacagccagcaaccctaaagtggagaatgaggatgagcctgttcgactggctcctgagagagaattcatcaagtccctgatggcgatcggcaagcgggtggtcacgctccccaccaaagagcagaaaacacagaggctgatctcagagctctccctgctcaaccataagctccctgcccgagtctggctgtccactgctgggtttgaccaccacgtggtccgtgtaccccacacacaggctgttgtcctcaactccaaggacaaggctccctacctgatttatgtggaagtccttgaatgtgaaaactttgacaccaccagtgtccctgcccggatccccgagaaccgaattcggagtacgaggtccgtagaaaacttgcccgaatgtggtattacccatgagcagcgagctggcagcttcagcactgtgcccaactatgacaacgatgatgaggcctggtcggtggatgacataggcgagctgcaagtggagctccccgaagtgcataccaacagctgtgacaacatctcccagttctctgtggacagcatcaccagccaggagagcaaggagcctgtgttcattgcagcaggggacatccgccggcgcctttcggaacagctggctcataccccgacagccttcaaacgagacccagaagatccttctgcagttgctctcaaagagccctggcaggagaaagtacggcggatcagagagggctccccctacggccatctccccaattggcggctcctgtcagtcattgtcaagtgtggggatgaccttcggcaagagcttctggcctttcaggtgttgaagcaactgcagtccatttgggaacaggagcgagtgcccctttggatcaagccatacaagattcttgtgatttcggctgatagtggcatgattgaaccagtggtcaatgctgtgtccatccatcaggtgaagaaacagtcacagctctccttgctcgattacttcctacaggagcacggcagttacaccactgaggcattcctcagtgcacagcgcaattttgtgcaaagttgtgctgggtactgcttggtctgctacctgctgcaagtcaaggacagacacaatgggaatatccttttggacgcagaaggccacatcatccacatcgacttcggcttcatcctctccagctcaccccgaaatctgggctttgagacgtcagcctttaagctgaccacagagtttgtggatgtgatgggcggcctggatggcgacatgttcaactactataagatgctgatgctgcaagggctgattgccgctcggaaacacatggacaaggtggtgcagatcgtggagatcatgcagcaaggttctcagcttccttgcttccatggctccagcaccattcgaaacctcaaagagaggttccacatgagcatgactgaggagcagctgcagctgctggtggagcagatggtggatggcagtatgcggtctatcaccaccaaactctatgacggcttccagtacctcaccaacggcatcatgtgacacgctcctcagcccaggagtggtggggggtccagggcaccctccctagagggcccttgtttgagaaaccccaaaccaggaaaccccacctacccaaccatccacccaagggaaatggaaggcaagaaacacgaaggatcatgtggtaactgcgagagcttgctgaggggtgggagagccagctgtggggtccagacttgttggggcttccctgcccctcctggtctgtgtcagtattaccaccagactgactccaggactcactgccctccagaaaacagaggtgacaaatgtgagggacactggggcctttcttctccttgtaggggtctctcagaggttctttccacaggccatcctcttattccgttct

SEQ ID NO: 53 - DNA, 662 bp, Homo sapiens
  Genbank Accession No. AW004018
tttttttttccaaacaattaatttataaaaataacaaaacatttgtctatgaaaaaaagatcaggattcactctcatcgacgtcctcatctggatggggctcagcatcctccttttcctgctgctgtttcttccacagtttggccatggccaggagtttgaggttaggttggttggcagcatcttctggaaagatgtaatcaaagtattcttcccagcctgcatcagacccatcatcagtctggacctttcttctcttcttgactttctctggcatgagtttgtctactctctccttatctgaagctgttccaaattcttcttcaaaacttcgccaagattccagcagcataagtctctcttccttttcttcacagtttcgcatggttttgttagcttcttcataaatttgtctgcatttagtcaaacttccttcttttcctgaagacaactcaaactgagcaaagctgatccataccttacatgctgcgtccgttgaagcaaccgccggtaaaggtttcgtgttctttctgtttcttcctgctcaatttcaaaatcaatatatgatttccaaagcacctctggcatgtctaaacgtggctgactgatggctaattcatagattgcccgggctctgtcaatatcaccaaggattgtctctaa

SEQ ID NO: 54 - DNA, 75270 bp, Homo sapiens
  Genbank Accession No. AF023268
tcccagggtcccgggttgggggggtggagcagcatttcgtcgccgcgggggtgccgggactccggccgcagtgtcgccgccatcacggacttcctgtgggacaagcgcacgggcctcgccgccagaacggtgagcgcgcgggttggctggccgcgcgaaaagatggcgaccgcggggcgggcggggttaggcttgggttgggggcaagggacgggggcgagatttggagaggaggagagttgagacctacgaagcgacggagtagggagatgagggggaaggaggtcggcctgcgttagatgtaccaagaagacctgtaggaagcttgggcggaaccagactagaagggatgatagagtaaggatgggagtggttccagagatgtgaaggagtggaatatcaaagttggactcgggtccaatcaaagctggaggggaggaatacatcaccgtgtcacttggaacatgcaggaagaatttgcctgtaggagttctggattttctgagtaagaaaggaaaaagcaaaagatttcaaacttgagaaaggtaggctagtgatgttttgaggggacagtgctgtgaggccgtttgatggtagtatgtggtgagatggcatttatgtggctatgctgattaggataactatgctgggaaaatcttgtgcttgcagcttggtatgctaccggccccagttacaccaagaatgcatcaaataccaccctcctcaatttggggaggttgggaagaagtgtatacagaatggtggtcagacatcggaatcattaaaccttttgcatacgcgattctcatacacccccaccaagacttatgtccctgggcctgtttcattttattgtgccttcctattctcatcttcctggattgtgacagtgatttttctcttttttccttgaaggaaaaaaaggatttgtgaaccatcccattccaattccattaactggaaggtggccagctgtcagctaagtagggctgatattaattaaaaaccacttactggccaggcgcggtggctcacatctgtaatcccagcactttgggaggccaaggcgggcggatcacctgaggttgggagttcgagaccagcctgaccaacatggagaaaccctgtctctactaaaaatacaaaattagctgggcgtggcggcgcatgcctgtaatcccagctactcgggaggctgaggcaggagaatcgcttgaaccctgcaggcggaggttatggtgagccgagattgcgccattgcactccagcctgggcaacaagagcaaaactccgttttcaaaaaaaaaaaaaaaaacacaagaaacagttgttatctacgtcttaagcacaagagtggaggggccagatgggatgctgactgtattttgtggcctactacattaaaacctctcaagggagagttggccaaagatgtgagagatgattaagttaattcaaggggcccgacccagatccctggagctgatagtatttggcgtttacctggtatatgtagaaacctgggagactggattcctaccctcaggacactgatagtctacttgaggagatgagatgtactcttaatgagtgtacaacatttatcataactgtaaaccaaagctttactctattgacttgtgaggaactcagcatcacacagatctatatcttagatcctgaacggggatacactgtggcagtctccatcggtcgtcatacactttgctctgtagtgaactgtgtcctagacctgaaggtcttcatggagctgtgtttgaagaaaataaggatagaacacttgaactggctgggtgcggtggctcacgcctgtaatcccagcactttgggaggccgaggcaggaggatcacgagtcaggagtttgagaccatcctggccaacatggtgaaaccccgtctctactaaaaatataaaaattagccaggagtggtggtgcgtgcctgtaatcccagctacacaggaggctgaggcaggagagttgcttgaacctgggaagtggaggttgcagtgagccgagattgtgccactgcactccagcctggccacacagcgagactctgtctcaagaataaaataagaacacttgaactgaggggtgggtaatgagggatgggtttgggggctttctagagtttcagccagagttagtaggtggctgtactccaataaggaggaattgtaagtacagaggaagactgaggaagttgtgcctggtaaaaagaggcagaaataagaggggaaagcagcttagcatctttggagcagccagtccttggaggagatttctgaaaactcgaaactttcctatttccgctttcccttgccttccagatgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaaggtgaggcctcaggaagagatactcaaacacagggtcactcgctcattcctttgttcaacaaatacttattggttatatgctaaaaccattgccttaagtagctggctgaaggtaatgtcgggatagataaccacagaagccatttgggctgcagtgttaagtgttagaatacataatgaggaactgaggacgtacccagtgcgtttggttggagtggggcaggatgttaattcagataaaacattgcagaaagcaacatctgtatatgtctggaggacaagtagagtcaggtgggggaagtgtctttgggcagagggaaagcatgtgcaaaggactgggagctacaggtaactaataagctaacagataagacaaagttagatctttgtgtttttaattttttaaaaattttaaaatatcatttatttatttttgagacagagtttcactctgttgcccaggctggagtgcagtggcaagatctcagctcactgcaaactctatggccccaggttcaagttgattcttgtgcctcagcctcccaagttacctgggactacaggcatgcgccatcatgcctggctaattttttttttttttttttgtatttttagtagagacaggatttcaccatgttggcctcaaactcctgacctcaagtgctccacctgccttggcctcccaaagtgctgggattacaggcgtgagccactgtgcccggcccagttttaatattttagtggtatgttcagtggaaagaatgggttaaggatggtttagagcaaggggggatttcattcaggaaagcttgagaaaaaaatggctctttgtttctttgtatcactgcagcgggaaaaaggccccaatagggagtggttctgcagatgccttttcagaggttttggtgacatggcagatgtgcttggttaacctcaacttcttctggtcccttggtcctctccagtgactcaaacaagtgtctgagaacagtttttcttagcattcactctttgtcctcactaaggcaggaccttgtcacagggtatggtagggaatgtgatctgaacagatacacctcattggtatttgttatccaacagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcggtgagtgccagcccaggcccttcctctccccactcttctgcaggccctctaggactctggtaagtgagcagtatccttgttctcagctgaacattggggcatgaacactgaggtgggcactgagtttgcctactttcttggaagctctccgactcttgaagggccctggatctgctttggagatggatgggcacggagcatttgtgacccccagtgctctccctggcatgttgggcttattgtgttgggagcagcttctccgccccagcggcctccactctttaatggggaccttgcttgttgaactgcctttttcccccaagccctgggctctgtagcccccttggcaggcgggcttgggtgggggcaaggggagatctgtgtctgcccggaagggcattgtgtagagcatggggttgtgggtgacattggcaacaacaccacttctctgctctgcccatgcctctccctgttcctgttttgggtttggggacttgggattatgcgccgctctctcttctcacaccgatccaccttactgcatctgacgtgttcccttccactgtcccccatcatcgtctgtcccccctggctgggcgcctgtgaccggtgacccctctaccacccaccccgcctccctccggcccccggctccgaacactgggcttggttcggaaccccccctgccccccgacagcagcacagcagccggagagccaagagtgtagaggacgacgctgagggccacctcatctaccacgtcggggactggctacaagagcgatgtacaagccaaatcgtaacaatcctatagcctgtaatggtcccatagccatcctaacgtcccaagccaacctgagtcacagcctcttgccttttgctcagtggtgttgtttatctgggtggtttgagttgctgttgagaattgacctctgttgtccactcccagctctcacggcccctgggttaaaggtggtgggaatcacgcaggggttttcttcccctgtgaccatctgtatctgttccccttccttcatctccaccccaggttgctgtccccttttttcttccaactcagctcattccccaccttctctccctccctctccccgaccctgctctctttcatttcagatgaaatcgttagcaccttaggagaggggaccttcggccgagttgtacaatgtgttgaccatcgcaggtaactgtcagtccctccctactatgtggggctaaagagatggttggggttatatggggcttttttgctaattaacctgaggtagaatttcttagtccccctacagccctgttcattttgagacattcttgagaacccagcaaaagcctctcctgccaacttacaggggtggggctcgagttgccctgaagatcattaagaatgtggagaagtacaaggaagcagctcgacttgagatcaacgtgctagagaaaatcaatgagaaagaccctgacaacaagaagtaagcaagcaaggaagtgtgtagggaggctgagagccccaacccctacacgggagagattcctagacctggtttaggcagacagggggagtcctagaccttctcatccattcatcttttgttcatcatcttagagtagtagaacatcagggtaggaaagggggggggcccatgacattccttaatccactccccttgttttcagggaagttagattgctctgtcgtttgaccccagcctagaatggcttctgtagagatataccctggacatagccctaggactgagcatcaacctcatcgaacaatgaattaagcttttataagtagaactatgctgataaggccacagacatttatgcagtaatattttgttcacatacattcacagtccaaaaatagaataaggactttaagccaagatatgaggccagtaggtataaataggagcccagtggtagctagtggtgaatgaatatcaggagttgggctggggtttgggatttaggatggacagtttgatgattccaggatctatactgtttggagcttggcactccacagctcttccagcaaatagtttttgaactatttaaaatgacgcaggaagatattttgaaacttgggctgggcatggtggctcacgcctgtaatcccagcactttggaggccgaggcgggtggatcacaatgtcaggagttcgagaccagcctggccaatatggtgaaaccccgtctgtactaaaaataaaaaaattagccgggcatggtggtggacaactgtagtcccagctacttggaaggctgaggcaggagaatcgcttgaacaggaggcagaggttgcagtgagccaagatcgcgccactgcactccagcctgggtgacagagtgagacgccatctcaaaaacaaaaaagaaaaaaacatggcagatagtaccatttctttcttgggtctggtagaggctactccttagctgaactgaattttggtgttagtactagctggcatggttttacacaagttatgtggaatcaacagctatgaagtacctccttgttggatggctgatgggcagatgggagctcatcagacaatcccccttcccccatctctcttctcagcctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagtgtgagtggggtgggccgaagtggactctggggcagtccctcccttcattggatctcttctgtcggttgtgcactggtgaagcccctaaacagtcagctgtctgttatctgcagttctttgatttactgtcatcttgaaacgtcttctgacttaactccttgactgatgtctttatcgtcactgattgctcttactctacacctagcctagcagcagctagaagagaaagcctttggaatcaaagcacttaattaccctccccttttcctttctccctttcttgggaaagcatcagtcagacagcaaacataaagagacaaaaatacactccttagggtaaaggcttacatttgtctgggatgagatgttcattcacagcaaaggagatgggaacacagagtatgtagttcagctaaggggcaaggtggggaattcaaagaaatatatcattcctctggagtcatcaaaataatacagtttcacagaattgagttaacataatgccagctagacacatgtcaaagtgtgtacacactacaactcaaagcaaactttttttttttttacatcagttgagctacatatgtatcttacattagaaagagcagagcttcttagaccaggcattccatttagacgtagagcggaaagcagccctagtgattctggacctgtctcctcactggctttgccctaggtaaccaggcctggggtcagctgataccagttagctctggccacctgcaccaagcctgacctggccttctcccctacagtcctccatgataacaagctgacacatacagacctcaagcctgaaaatattctgtttgtgaattcagactatgagctcacctacaacctagagaaggtaagatggatagggtctgcccttggttactgggggcaggcagctgcaccactttgccttctgccgagccctttgttttctcccttttatttcgtcctcccacattttctccctgactggctccaactgggtaaaactaaataggttgaaagggagaaatctcttaagaagacttaaattgggagataacttgtacaggggacttcagataactttccagtagagtgaaagtttttaaggttcttggtttgggctttatttatttatttatttatttatttatttatttatttgagacagagtctcactctgttgcccagactgatgtgcagtggcataatctcggctcactgcaacctctacctcccaggtttaagtgattctcctgcctcagcctctggagtagctggggttacaggcacccgccaccacgcctggctagtttttgtgtttttagtagagatggggttttgccatgttggccaggctggtctcgaacccctaacctcaggtgatctgcctgtctcgggctcccagaatgctgggattacgggtgtgagccacagtgcctggcccggacttcttttttgagatgtgtatttctgtgaggtaggaaagctcggctcctttgactaactggagataatgaagatctcagacctaaagaagctctccctcctttgacccctttgctacatgttacatatttttagagaaactcctttgctacatgttacattttttcagaaaaacccatttgctacatgttacattttttcagaaaaacccatttgctacatgttacatgttttcagaataactatatatctggcacttgagtgtagtcttcagatgcttacagatgtgcacgctgttctataatcatttcttaatccttttgctcccctacttctaaaagtattatgctggatgtggtggaagctgtaaaacaggaatttcccaggcttgtaaggccaggcaggagatcacagtaccttttttaaggctcagaagcaatgtagagaatactggtggaatctcagtctgggtgcagaggttcatcctctttctcctctgctcctagaagcgagatgagcgcagtgtgaagagcacagctgtgcgggtggtagactttggcagtgccacctttgaccatgagcaccatagcaccattgtctccactcgccattaccgagcaccagaagtcatccttggtaagggagggcaaggctgtccaagtgtgtgagatgatgtgagggtggggccacctaagcctcataacaccttttccctcccattctcacccagagttgggctggtcacagccttgtgatgtgtggagtataggctgcatcatctttgaatactatgtgggattcaccctcttccaggtaagtgatgggatgtcttacttgactgcctggcattcttctacctggttcctttgttttctgctgaggacctgcctactcagttctccacattgcctgccttcctggcagctgatccttagcatgcccttttcagtcccatgctcagtttctgtttttgtttcccagacccatgacaacagagagcatctagccatgatggaaaggatcttgggtcctatcccttcccggatgatccgaaagacaaggtgaaccttgagggggcactagttaactcttttccttttctctccacagaattggtctatttcacatcattttcttttttctttgatacctcctctccccccagttactttcagatggggaaataagggaattgtaacaagggtgaccttctgattcctcaacctccccttcccctctagaaagcagaaatatttttaccggggtcgcctggattgggatgagaacacatcagctgggcgctatgttcgtgagaactgcaaaccgctgcgggtgagctgggctcgggataaatagtgcccaccgtccagaagtcacttccttcttagggtggttgccccctggaatgctcttcaacaagccagagggttaggaaaggaggggaggaaagctgaaagaagacatctttggtcaacagaggaaacataagagggagtggttttgcggagggaaggaggttagacagcctaaccttgagacaaccagagatcaaagcaatgtcctggattctttaggtcagacagaaaagaataaactacccttgaagagcttacattttaatgaggaactaaagaagattcatgaagttgacaaggatatacaagtagaaagaactttcaaagattatggagtaattgtgctagagggaaggtaggttgagctataatatcagaaacgttggtcctggtgtggtcgtggttaggtagccttcaaattggttgcaagcagagccttggttctccaagaatgaaaggtagggtcttgaagaaggcagggtttgtaaggcatcctgcctcacctttttcctgccctcctccaccagcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtgacgatcaggccctgggcccccctgcatcttttatagcagtgggtgtccagtccaggacactggtgcttttttatacaagagaacgagccagagttcactccttcctcctggctctctatatacctgtgaatatgtgaaatagtgtaaatatgaaagaacttgtacctatcacttcaacccctgccttgtacataatactattccatccacacagtttccaccctcacctgccccctcatacggagttggatgggggccgagtgaggtaaccaggtggcatctaccccatgttttataaggaattttgtacagtctttgtgaaataaaataacgtgcttcatttgacccccatccctggagttggaggtttgggaatgctggggtggagggatgaaactattggcaaactttctgagtttgggtatgaagggagtcctccttaccctccaaaatgaagcacagccaggctaccatttatttcccctgtccaccttatcatatgggagggtagtgatgggtggggcagcatttctttcagattaaaacagagaagtgttatgaggtggcacttctcggatgtggaattatgagagttgggaagatctgactcctagagtcattaggccgcggcccagtatagagccagaaactcaggttgaaaacgtgctcaacctggctcctaggggggattgccaggaccggtcagaaggctcccgtcgtccatctcgggagactaggaaggccggattcctacgcgaggcctgctgggaagtgtagttcgttagtggaaggaagtcacatggaagaggggcggtagttggttgtgggcactgggttagaggtatcacgtgggggcactttcgtcttagcttttggacaagacgcaggcgcaaacccacggctgctgcgggggatccttgtggccctttccggtcggtggaaccaatccgtgcaacagagaagcggggcgaactgaggcgagtgaagtggactctgagggctaccgctaccgccactgctgcggcaggggcgtggagggcagagggccgcggaggccgcagttgcaaacatggctcagagcagagacggcggaaacccgttcgccgagcccagcgagcttgacaacccctttcaggtgacttgcgccagtcggcctcttttgggcggtcaggttgattcttcccggttctgtagggtcgggctaacttgtatccccatttgtgacatttgatcctgggaagagccgccacgtggggtgacagtgactccagaccagtgagcactctggggggcgggccctgccccttaatgggctggtgctggcagtgttggatgatggatttgaggtaaatgttgtcccagtcctgggacaacggcgtggccccgaaggtgcagtacttggagccacaggcagtttgggaatggtccctgggaattcccctaggtggcactgggtgccagctgagacccgggtctctgccctcaggacccagctgtgatccagcaccgacccagccggcagtatgccacgcgtgacgtctacaacccttttgagacccgggaggtgagctactggagagcataggagttcaaggaagggaagggtttgccagtgagacaagttagcttgggtacaggggacttttctgcatgcacaggaaggaagaggcactgtgatcttagttccctgagagaggctagtcagccatgggatgcctcatccttctagggccaccagggccaccaacccaaatggtttgagctctaaaagtcagtcctgggaagatctggaggctggcaaggccttaaccaatgttctgtatgggaaggctttcttgaagaaaataaaggttggggctggttggagtttaggttaggtgtgcagtttgaaagcaaggaatgaggaggactacacctgtttgtcccactggattagaagatggtgagttgtaagaatttaaggggttttcatggtacttaaaacttaaaaaaaaaagccaagcatggtgtgcatgcctgtaatcccacttgggaggctgaggtgggaggattgcttgagcccaggaatttgatggagaccatcctgggcaacagcgagacccccatctcttgagacgttttaaaaattggctaggtgtagtggcacatgcctgtagtccttgctacttgggaggctgggatggaaagactgcttgggcccaggagtttgaggcttcgatgagtgatgattgcaccactgcactccagcctgggtggcagagagacaaccccacactccatccccagaagaaaagagaaaaaaaaaacacagttaagtggggagatatggtaaatgagtgatttgagtcctcactaaggaattggatatgagggatgataaggctgctacaggtcatcatagagttctctgacagaaattcaacagaagaactagcttcctaacaaatgggggaaaggctgcttccttacatcatgagttctgggtctaaaggtattcaagcagactatagattcctgtgttatgagggactagatgatcactaaggtcttttcctcaacagccaccaccagcctatgagcctccagcccctgccccattgcctccaccctcagctccctccttgcagccctcgagaaagctcagccccacagaacctaagaactatggctcatacagcactcaggtacaggaggtgtgcaggtgagggctggggagaagggccagtcctggggcccagggctcacatctccgtctgctcacactgggcaggcctcagctgcagcagccacagctgagctgctgaagaaacaggaggagctcaaccggaaggcagaggagttggaccgaagggagcgagagctgcagcatgctgccctggggggcacagctagtaagtaatagagtggggaagagcatctgagagtttgggaggagcaaaaacaggtcttaagggcctgggctcggctgggtgcggtggctcacacctataatcccagcactttgggaggctgaggtgggcggatcacgaggtcacgaggtcaagaaatcgagaccatcctagccaacatggtgaaaccctgtgtctactaaaaatacacacacacaaaaattagctgggcatggtggcgcgcacctgtagtcccagctactcgggaggctgaggcagggagaatcgcttgaacccgggaggtggaggttgcagtgatccgaaatacgcgccactgcactccagcctggtgacagaaatgagactccatctcaaaaaaaaaaaaacacaaacctgggctctggagtcagactgctgagtttgaattttagagatactgcttactaaccatgagcttttagggaagttacttaatcttgaatgcctcagtttccctatttataaaatgaaaaccacgtttgcatccgtttgacagggttgttgtgaaggttaaacaaaatatatgtgaagtacttggtacagtgcttagatgttttaaataataataaacagtattaatttttccactctcacttgtctccttggaccccaaattggagataaaagagaggatccagggctgggtatggtgcctcacgcctgtaattccaacactttgggaggccgaggcaggcaggttgcttgagttcaggagttagagaccaacctggacaacatagtgagatcctgcctccaaaacattaatgaaaaaaattagcagggcatgatgtacctgtctgtagtcctagctcctcaagaagctgaggtggacggccaggcgccggggctcatgcctgtaatcccagcactctgggaggccaaggcgggtggatcacctgaggtcaggagttcaggaccagccaacatggtgaaaccctgtctctactaaaaatacaaaaattagctgggcgtggtggcatgtgcccataatcccagctactcaggagactgaggcaggagaattacttgaacccgggaggcggagattgcactgagccaagatcacaccactgcactccaacctgggcaacaagagcgaaactctatctcaggagaaaaaaaaaaaaaaaaaaaagctgaggtgggagggttgcttgagcccaggaggttgaggctacagtgaaccatgatcataccactaccttccagcctgaacaacagagaccctatctcaaaaaaaaaaaaaaaaaaaaaagagaggatccagggatggagagaagggggagtgattcctttgttggtctctgttttacttctggggtccacctgttttttggcgccttacagctcgacagaacaattggccccctctaccttctttttgtccagttcagccctgctttttccaggacatctccatggagatcccccaagaatttcagaagactgtatccaccatgtactacctctggatgtgtgagtagtgagaagccttttggaggaagttacaggtagatctcttaactgccctggggtccgctcaccaagaaccaaacacttcacctctatttagaactcaccagcctgctagcaaatgttcttgccctctccccactttttattgtccatatgcaggaatatattttgaattctttagatgtctttgggctgggtgcagtggtatacgcctgtaatcccagcactttgggaagctgaggtgggtggataacctgaggtcagaagtttgagactagcctgatcaacatggagaaaccccatctctactaaaaatacaaaattacctgggcgtggtggcacatgcctgtaatcctagctactcaggaggctgaagcaggagaatcacttgaacccgggaagtggaggttgcaatgagccaagatcatgccattgcactccagcctgggcaacaagagcaaaactccatctcaaaaaaaaaaaaaaaaaaaaaaaaaaaaaaaaaaaggccgggcacggtggctcacacctgtaatcccagcactttgggaggcccaggtgggcagaacatgaggttaggagatcaacaccatcctggctaacacggtgaaaccccgtctctactacaaatacaaaaaattagccgggcgtggtggcgggtgcctgtagtcccagctacttaggaggctgaggtaggagaatgggcgtgaacccaggaggcagagcttgcagtgagccgagatcctgccactgcactccagcctgggcgacagagcgagattccatctcaaaaaaaaaaaaagaaaaaaaaaaattagctgggcgtggtggcgggcgcctgtagtcccagctactctggaggctgaggcaggagaatggcgtgaaccggggaggcggagcttgcagtaagctgagattgcgccactgcactccagcctgcgcgacagagccagactccgtctcaaaaaaaaaaatgtctttgcaaggggatcacacattattgacatttgctttctccatctcccctgttgaggattccatgaaggcagcagctgccttcatttccttactctgccatgtttggtgaatattataggatgagcatagatgggaaggagccttcattgcagtccagaagggctcctcatcctgtccctctgccccttaggcagcacgctggctcttctcctgaacttcctcgcctgcctggccagcttctgtgtggaaaccaacaatggcgcaggctttgggctttctatcctctgggtcctccttttcactccctgctcctttgtctgctggtaccgccccatgtataaggctttccggtaagtgtgttagtggtgggagagtgatggagacctgggatgggccccacgtctgcccatccttcagctctaattcttctcccaccctccccatttttttcctctttgtaggagtgacagttcattcaatttcttcgctttcttcttcaatttcttcgaccaggatgtgctctttgtcctccaggccattggtatcccaggttggggattcaggtttgtgaggctgttatccaccctcacctttccctctagatccagccagcactgggtgctggataggagttgttcagaaaaaggaaatgtggttttaatccttgggaggtactagtttaatgagatacaagacatacttccaggatagagcgcagacagcactcttgacacatatagattgaagggaagaatgctgcatttggccaatctaaggtggcttcctggaggaggcatagaaccatggttgaaaggaggaagaagaatccccagatgagtgcctggaagagcttggagagctcagggctaatggttcagaaactggaattaaactatgaagagattagatacagtttgggattgtggtgcttggaatgctgctcgattgacagggagccactgctgatgggaagggtaggaacaggggatgctggtgacataaccagtggagaagctgaggagcccctcttcactggtacatccttccctttacagtggctggatctctgctctggtggtgccgaagggcaacacagcagtatccgtgctcatgctgctggtcgccctgctcttcactggcattgctgtgctaggaattgtcatgctgaaacgggtgagggctgtgtcgaaggtggggccgggatggtgagatcatgggtccccaggggcgtgggtggaacattcaggagcaactggcacaggtcaggctgctgggttgttctcagctaatggacctctggggtgtgtgtttctgtgtgtgagtgtgtgtgctgggcagcaggctgctgagtggtagtgatgctgttaggctgggggtggggaaccagtggctggaatgggcggtaatgtctttgtcctctacttgcagatccactccttataccgccgcacaggtgccagctttcagaaggcccagcaagaatttgctgctggtgtcttctccaaccctgcggtgcgaacccgagctgccaatgcagccgctggggctgctgaaaatgccttccgggccccgtgacccctgactgggatgccctggccctgctacttgagggagctgacttagctcccgtccctaaggtctctgggacttggagagacatcactaactgatggctcctccgtagtgctcccaatcctatggccatgactgctgaacctgacaggcgtgtggggagttcactgtgacctagtccccccatcaggccacactgctgccacctctcacacgccccaacccagcttccctctgctgtgccacggctgttgcttcggttatttaaataaaaagaaagaggaactggaactgacatccccgtttcctgaatcttcattgggaattaggccttatgaaagagaaaggagagtgtggagtgaggtgggaaggtcggacccggctttagtgtaaactgggagagagatgaggggcggggcggtggagcccgagtggctggcgcaggaaggaggtgggaagtccacggaaacgcgaaacccggagacgccagggagccctgctccccacccctctccattaatgacggggaagagccaccgcctctgccgggaacgccaaggaatacgcgggcctggagcctgaaaagctggatggggctcaagtggaggcccaaaggatcactagcagcctagccagggtccagagcgaggcagggactggaggagcgcttatccgactgcctcgccctgccgcgggatcccccaaccccgacagggtctcagtcccgaactacaactcccggggtgcaccgcgccggccctcgccgccatgcccctccttcccgcaccatccccattcccatcccccttctctagtccccgacctgcggcagccggagctcggggagcggagcgtggtggggaggggagcgggacaggcgacacaggagacagcggcgccgcggcctctccccaccaggcggccccggatcctactggacgccctgagggcacaccgaccgcgcctctagagtcaccccacgccgacccctcccctcttctctagacttatttccatccttcccgcttttaccctccccacccgtccctgggctccaggccgccgccccctcctcactcctggaccggcccttctcggtgcccctcttccctagggagatgcgatgagccggtgcccccgcgtcctcatcgtcgccccgggcacggtgcccgtccagtgcccgtggtggggagggagcactccgcggtccctccgtgacgcccctcgcttggccccccccacagctggcgtccctcggccatgccccaggggacccagccagggggtgggctctagagcgagtggggtggagaggagaaaggacggggccttgggggcctctgagatgctcccaagtgccagggagggccgagcgaggcgcaggcaaccgggcagcaggcatgatgccctcgcctagtgactccagccgctcgctgaccagccggcccagcaccaggggccttacccacctccgcctccaccgaccctggctgcaggccctgcttacgctggggctggtccaagtgctcctgggcatcctggtggtcaccttcagcatggtggcctcttccgtcaccaccaccgagagcatcaagaggtcctgcccgtcttgggctgggttctcggtgagttgggtgcacagttgttgggtgggggaggctcctcgggccccaccctccacaccagctgcaagtccacatgcttgcctctcctccctctcctggtcctctgcctccttacagggctgggtgcatcctgtggtgaggggctcactcaggagcctccccctgccaggctgagcctgtgcccactctgtccccagctggcgttctccggggtggttggcattgtgtcctggaagcggccattcactctagtggtaggtgccagggtccagtgcccactgggaggcaggtgcccagcacgcaaggggaagcccatttatgacctcaagaagggaactggctccccagttggtgccagccgggtgggcacgaagttctgtgaaggagggggactctgtccgtggcagaggagtactcatgagggtcccagccctgagtctgcaccctgttttccccagatctccttcttctccttgctttcggtgctctgtgtcatgcttagcatggctggctctgttctctcctgtaagaatgctcaactggcccgagacttccaacagtgctctctggtgagatttgaggagggagagctggaaagaactggctgggggaggtgtgcaggacacctcagtttgtgctgactcaggctgcctcaccctccctgctccactcaggaaggaaaggtctgtgtgtgctgtccctctgttcccctcctccggccctgtccagagtcggggcaggaactgaaagttgcccctaactccacctgtgatgaagcccgaggggccctcaaggtgagcttgcaccctgcaaacatcctcctggttctcactcttgcctcccctgctggggatctcacaggcccataatgtgtggaacttagccgtctcctgactcctctcctcctttcccttccccagaacctgctcttcagcgtctgtgggctcaccatttgtgccgctataatctgtacactctctgctattgtctgctgcatccaaatcttctccctggacctcgtgcatacggtgagaagggagcaggggccagggcacgcaggtatggtgggggcaggggtgtgtgtgctgagacttgcctgagggaacaatggctacagatctggcttttgagcaccaggggctatgggttatctattatcctcatctattggaggaatggatgtttagtggggaggatgagaaggggagatggcagggagtgagttaagtatgtgatgctggtctgggaccaggagagggtgctttagagaatgggactggatggtggggtagagtcaagaaggtcctatgctgggcacggtggctcattcctgtaatcccagcactttgagaggcccaggcaggcggatcacctgaggccaggagttcaagacccacctggccaacatggcgaaaccctgtctctattaaaaatacaaaaatcgggccgggcgcagtggctcgtgcctataatcccagcactttgggaggctgaggagggcagatcacctgagatcaggagatcgagactatcctggctaacatggtgaaaccccatctctactaaacatacaaaaaattagccaggcgtggtggcgggcgcctgtggtcccagctactcgggaggctgaggcaggagaatggcatgaacctgggaggcagagcttgcagtgagctgagatagtgccactgcactccagcctgggcgacagagcaagactccatctcaaaaaacaaacaaacgaaatacaaaaattagccaagtgtggtggcgggtgcctgtaatcccagctacttgggagtctgaggcaggaaaattgcttgaaccggggagtcagaggctgcagtgagctgagatggtgccactgcattccagcctgggcgacaagagcacagactccgtctcaaaaaaaaaaaaaaaaaaaaaaaaaaaatatatatatatatatatatatatatatatatacacacaaaattacaaaaattaggctgggctccgtggctcatgcctgtaatcccagcactttgggaggccaaggcaggaggatcaccagatatcaggagtttgacaccagcctggccaacatggcgaaaccctatctctactaaaaatacaaaaattatccgggtgtggtggcgggtgcctgtaatcccagctactcggaagactgaggctggagaatcgcttgaacctgggaggcagaggttgcagtgagctgagatgtagccattgtactccagcctgggcgacaagagtgaaacttcgtctcgaaataataataataataataattagctgggcatggttgcacacccttataattcctgctactcaggaggatgaggcatgggaattgcttgatcttgggaggtgggggttgcagtgagctgagatcgcgccactgcactccagcaacagagtcagactctgtctcaaaaaaaaaaagaaggtcctagatggaggtgaggctaaagggtggacattcctgtgaagacacagaatgtgtggagcttctggatggaggtggggccataaagaggaatttgtgggtgggcagggccagaggctagaggtacagggtgacggtggggttaaggagagctgattttgggtgagggaggggccagaataagagcttctctcaggggatagggccagaaaaaataatgtagatgagagaggagtttggggcccaggaggagctgtattcccagaatccagacttgctgacctgctcgcttccccagcagctggcccctgagcggtcagtctcaggcccactgggacctctgggctgcacgtccccgcccccagcccctctcctacacaccatgctggacctggaggaatttgtcccgcctgtgcccccaccgccctactatcccccagagtatacctgcagctcagaaacagatgcacagaggtaaggcctggtggggtcttgctgggggagctaaggaaggggactggggctgggcctggattgctggagagagggatctttgtggagggggaattatttctcctacattgaccctcttcctcatctgccagcatcacgtacaatggctccatggacagcccagtgcccttgtaccctaccgattgccccccttcttatgaggcagtcatgggactacgaggagacagccaggtgagagcacagcacggcttggggcgggctggggagccgggttgtagcctggaaagctgaacaggctgtagcctctggataaagatagtaacagtggtcaaggtctttacagcaccagcatgcccactgttgcctttgatcttccctagagcctggtgaggtgagtgggttggaagctatcatttctgccatacagacgtggaagctgaggctgcacaattaagtgacttgtacaaagggacaaggctggtctggaatctaagtctccagagctctgtctagcacatgggggtgttcagtgtgtagggctgaacccttgaccctgtgtcttctgcaggccactctctttgaccctcagcttcacgatggctcgtgcatctgtgaacgagtggcctccattgtagacggtgagcagggcgtaatgaggggtggacaagggcggggctgccagggatagctggggtgggtagagacaatagaaggggaaaacaaggcggagttggtggtttggggacataggagggctgtggcaacttggaaccctggacttatttttctcctctgagataaagctggaggcacagtggcccctagaggctgggcttgggagaagaggaactgcctgggcagggctaggccaggccaggctggtgctacacagcgccccctgccgcccacagtgtccatggacagcgggtctctggtgctgtcagccattggtgacctccctgggggctctagcccgtcggaggactcgtgcctgctggagctgcagggctccgtgcgctccgtggactacgttctctttcgctccatccagcgcagccgtgccggctactgcctcagcctggactgtggcctgcggggccccttcgaggaaagccccctgccacggcgccccccacgggctgcccgctcctattcctgctctgcccctgaagctccacccccactgggtgcccccacagctgcccgcagctgccaccggttggagggctggccgccctgggtgggaccctgcttccccgagctgaggcggcgggtcccccggggagggggccgcccagccgcagccccgcccacccgagccccgactcgtcgcttcagcgatagctcaggttccctcaccccaccggggcaccggcctcctcatccggcatccccaccaccgctgctgctgccacggtcccacagcgacccaggcatcacgacctccagtgacactggtgagccccctccccgactgcccaggctcaggagagggtaggcactgggagttaggtggccagtgatgcccaccaggattggggcacagttgaggtgggtgaggaggaagaaagggtgagttcactcctctggtagacattctcaggtactcacctcctagggacagaacatccatagcttagcagctaaaaaaggagaatttttttttttttttgagacggagtctctggctctgtcgcccaggctggagtgcagtggcatgatcttggctcactgcaacccgtttcccgggttcaagtgactctctcctgcttcagcctgccgagtagctgggactacaggtgtgcgccaccatgaccggctaatatttttttttttttttttttttgagacggtgtctcgctctgtcacccaggctggagtgcagccgcgcgatctgggctcactgcaactccgcctcccgggttcacgccattctcccccgggttcacgccattctccttcctcagcctcccgagtagtgagtagctgggactacagatgcctgccaccacgcccggctattttttgtatttttagtagagatggggtttcaccgtgttatccaggatggtctcaatctcctgacctcgtgatctgcccgcctcggcctcccaaagtgctgggattacaggcatgagtcaccgtgcccgggcaatttttgtattttttagtagagacaggtttcacccttttggccaggctggtcttgaactcctgacctcaagtgatccacccgcctcggcctcccaaaaggatttattttttgaaaccagttccacagctctcagcttggtccacttatctgtcctccccaagcttcagctgtcacttgttaacatgtataataatagtacttcacgccgggcacggtggcttgcacctgtaatcccagcactgtgggaagctgaggtgggtggatcacctaggtcgggagttcgagaccagcctggctaacatggtgaaaccctatctctactaaaaatacaaaaattagccaggtgtggtggagcgcgcctgtaattccagctactaacgagagaggctaaggcaggagaatcgcttgaacctggaaagcaggggttgccgtgagccaagatcatgccactgcactccagcctgggtgacagagacacactccatctcaaaaacacaaacaaacaaacaaaaaacatgtataataacagtacttcagccataggcattgtactcgaagatgctgagaaagaacagtggcgggcaaggctgttcacacctataatcccagcactttggaggccaaggcaggtggatcacctgaggtcaggagttcaagaccagcctagccaacatggtgaaacccccatctctactaaaaattgaaaaattagctgggcctggtggtggacgcctgtaatcccagctactagggaggctgaggcaggagaatcgcttgaacctgggaggcggaggttgcagtgagctgagatcgtaccactgcactccggcctgggcaacacagtgagactccatctcaaaaaaataagaaaggagatagtactggggaacgctcagcactgtgcgccaggtgctgaacaacaccactgcagtccttgttgtggtggattgtaccatctagttgctggctaatatggacagagatgctggccctttgattggggatggagcgtgggagctgtgaaagctcctctgggcttgagttcccacaggagggtgggcgtgtccacagaacacttccactcactccctgtctccctttctctcttctccccagctgacttcagggacctttataccaaagtgcttgaggaagaagctgcttctgtttcctctgcagatacaggtcaggcatgtggtttgcgccccagggatggggattgggcatggctgcccagccccctctccaccctacaataccattctcttatctctgtctctctgcagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtaggaattcggggagccaggaaagatgtttgggaaagcgtggagcttcagattgagccttattgatgatgccctttcttgtgtccctgtccaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttccctcctacctctggggtctcttgctgcttgcctctgctgctctggtctgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttccccttgttttcttgatcattctctgtcaccagtgaaatttgttctagtgtctctcatatttaagaaaactctttcaggactgggtatggtggctcacacctataatcctagcactttgggaggccgaagcaagaggatcgcctgagcctaggaattcaagaccagcctgggcaacatagtgagaccctgtctctacaaaaaacaaaaaattagccaggcatggtgggacacgcctgtagtcccaactactcaggtggctaaggtgagaggatcacttgagcttgggaagtccaggctgcagtaagctgtgattgagccactgcactacagcctgggcaacagagcaagaccatgtctcaaaaaaaaaaaaaaaagaaaaaagaaactttcaagacactctttccaaccactaattgtaactctgctcctccttttcacagcaataggttttctttttcttccctccactgttaaacatccattctctcctcacccacccccatcagactccttcccctatctttccacagccactgctctgaccaaactttccagtgaccacagtggtgtcagacccagtgaccatttctctgcctgcatctcacttgacctcgaggcagcaattaatacccataatcagcatcttcttgaatttgtccctttgaaaagggaaatattggctcttctactttgtcctgctgaactgcttaacattggagggccccagggccctcacctaagccctctttcctacctccactctttctataggtggccctactactaaagtccatggctttaaataccatctttctatgtgttaatccatgactccagccttgacctcccatgagcgccatccaactcagcatgtctgcttggatgtctaatgggcatttcagattcaacatggccacaactgaactcttgattcccaccccagcaccggttatttttccactgttcccatctcaatggcacctccattacccatttgcacattccaaaagctcaggaaccatggtgacttcttttcccatatccaacacaaccaatcctatcctgaattcatccacatcccaccacctccccagctacctagctccagccatcctctctccacaacctctgaatcagtctttcacttttcccagcaatccattctccactcagcaaaatgatgataaagcacgtcacatcaaggctctgcctcaatttaatggcttcccattgtatttagaatcatctccaaactcccagagactatggtcgagctacaatctggcccaccttctgttccagccaaatttcctcacagcacaaggacgtttgcacctgctgttttgccaagcatgaaacccttggcccctatatctggtgctatcacctaatatcaggttttagctccattctcaccatttcagtgagcacccaatccccatcgcagtcattctatcacatagccatgtttttttttgtttgtttgtttcattttgtctttttttgagacagggtcttgctttgttacccaggctggagtgcagtggtgtgatttgggctcactgcaaccttccacctcctgggtcaagcaattctcctgcctcagcctcccgagtagctgggattacaggcgcccgtccccatgcccgcccagctaaattttgtatttttagaagagatagggtttcaccatgttggccaggcgggtctcaaactcctgacctcaagtaatccgcctgcctcggtctcccaaagtgctgggattacaggtgtgactcaccgcgcctggccacatacccatggtttcagcatgtatcactatctaaaattattatttttgtttatatatctgtgtcgtcccatagaaggttaaggtcccaagatcagaaacttgctcattgcagtgggtctaacactcagtaggtcctcaacaaacattcgttaagatactaaagtggcagggtggggccctgtaaacagcttcaggagcttcaggaccctgtgcttgtaggggcaacgtggtgccctccaaggaagacagggaggtgggaggagcactgcccagagatggcgtcaggctgcaagacttcttgaataattcagcatcataacaacccagcctcaggaagggatagggcacggccaggacgaaacattaggaggcgatggacaatgggattcccacggggcagcttctgcgcactggacgttccctaacctgaggctctctaaagaggaaggttaggaatcctctgagcttcggtgggctggactcactgtgggaattcaatcgcccccatccaccaacagtgtgctggcgggaaaacgccgacacgcatgcgtagttctcgcgccggctcctctctctctctctctctctctcgctcgctctctcgctctctcgctctctctcgctcgctctctcgctctcgctctctctctctctccggctcgccagcgacacttgttcgttcaacttgaccaatgagacttgaggaagggctctgagtcccgcctctgcatgagtgaccgtctcttttccaatccaggtccgcccgactccccagggctgcttttctcgcggctgcggtgatcggtcgggctgcatcctgccttcagagtcttactgcgcggggccccagtctccagtcccgcccaggcgcctttgcaggctgcggtgggatttcgttttgcctccggttggggctgctgtttctcttcgccgacggtaggcgtaatgaatatttcgacctttggatcttagctgtcccctccctgcgttcgcacttaacctttttcaccattattattattattgttattattattattttttgagggagtctcgccctgtcgcccaggctggagtgtaatggcgccttcttggctcactgcaacctccgcctcccgggttcaggcgattctccgacctcagcctcccaagtacgtgggattacaggcacccgccaccacgcacggctaattttttgtatcttttagtagagacggggtttcaccatgttggtcaggctggtctccaattcctgacctcgtgatccgcccgcctcggcctgccaaacagctgtgattataggcgtgagccaccgcgcccggccaaccatcattattatttttaacggtaaggatggtcagattttactaatgaagaagagattataaaatcttcaagtctttatatccacttgctttttgaggggtggagtgggaagaaggttatgtaattcatacgttcttcagagatgtgacaaacattcacggagccggacgacgtcgggttggattcgcactggagctgcagatgggtgccaggatggactggtccctaccctccgcttgaacctaggaggcggaggttgcagtgaaccgagatcgtgccactgcactccagcctgggtgacagagatactccgtctcaaaaaaaaaaacaaaacaaaaaacaagcggactgggcgcagtgcctcaccctgtaatcccagcactttgcaaagccaaggcgggaggatcctttgagtttaggagtttgagaccaacctgcgcaacacagtaagaccccgtctctacaaaaaatacagaaattagccaggtgtggtggtgtgcgcctatagtcccagctattctggaggctgaggtgggaggattgcttattctggaggcagaggttgcactgagccgaaatcaagctactacactccatccagggcaacatacggagaccctgtctcaaacaaacaaacaaaaaattgctcagtacctggccaaaaaagaagaggctcactatgcagaggggaagtggaaggagatgtttggacttctaaactcaatagagcaggagaggcaaatgtagaatgtgctcaggaaatatctgtgagatgaatgaacttgagggaagtaaggtactagatattacctgccctacccagaacaaatcctgtgcaatgtttccttgaaaagtgagaagtctggaaggggtggctactgacatagtgaagcaactagttcaattctacaacttgacagctaccctgtgccaggctatctacgaggatacttagaatgcataagacattccttcaaggaactccaggaacagaggcctgacatgttgcaatgtttagtgtcaagcagtgtactagagacacattatcacactcaaacctcacaacaattctgtgaggtaggagttatcactccccttttatagatgaaacagaggcttagagtgattgatttattgaaagtcaaacagccagtaaatggtgtagccaggattccaaacttgctgtctcactgagactgtacttaattactggagggaccgggtgtggtggctcattgctataatcccaacaccttgggaggctgaggctggtggatcacctgaggtcaggggttcgagaccagcctggccaacatggtgaaaccccatctctactaaaaatacaaaaattagctgggcatggtggtgggctcctgtaatcccagctactcaggaggctgaggcagggcaattgcttgagccgagatcacactgcactccagcctgggcaacagggcaagactctgtctcaaaaccaaaaaaaaaaaaattactggaggaacctagaagaagaaatgatcaattttgcttggagtgtatctagaaagacttcactgagatcatttaaagaacaaaaaggatggctggggtccaggcagtggctcatgcctgtaatcccagcactttcggataccaaggcagcagatcacctgaggtccagagtttcagaccagcctggccaacatagtgaaaccccatctctactaaaaataaaaaaattagctgagcatgttggagggcacctgtaatcccagctacttgggaggctgaggcaggagaatcactcgaacccaggaggtggaggttgcagtgagccaagatcacgccactgcactccagcctgggcaacagagtgagactctgtctcaaaaaacaacaacaacaaaaaatacaaacaagagacaagtagttcccaggtgcctaccaagtggtcaggcactgcacttacctcactgactgcagtaaccaccctttgaggttgtggcattgctccattttccaggcaaggaaatgggctgagagctgggattagtcaggtcatgactgtgtgtgccactcccgctaaatctcatttgatgtggttcatgaggccacaccatggacagcttcctccttgtgtccactgaggatatggctttgtacaacactttggtttttgaacgactttacaaacctccctgtcttgtgaggaaggaagaacagttattaccatctgcatctgatgatgaaacaagggacgctgcagaggagccgcactgaccactccctccctccagtcctgtcatcccactgccagtgtcccaccctcttgtgccctgcacttcactggctaataacccccctcactttttcctctgtgaagccatcctggataattccccacccacgaatggtccctcctcatctcagagagctctccatgcacacctgttaccgtttctgtctttatctgtaaatatctgtgtgtctgacttccatgcctcacacacctctatagggcaaagactgtcttaaacatcttggtagtgtcagtattttgcacagtgaagtttttttttttaaattatatcagctttatttgtacctttttgacatttctatcaaaaaagaagtgtgcctgctgtggttcccatcctctgggatttaggagcctctaccccattctccatgcaaatctgtgttctaggctcttcctaaagttgtcacccatacatgccctccagagttttatagggcatataatctgtaacagatgagaggaagccaattgccctttagaaatatggctgtgattgcctcacttcctgtgtcatgtgacgctcctagtcatcacatgacccatccacatcgggaagccggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtttctgtgggccttgtcctaatgaatgtgggagaccgggccatgggcacccaaaggcagctaagccctgcccaggagagtagttgaggggtggagaggggcttgcttttcagtcattcctcattctgtcctcaggaatgtcccaagcctttgagtagggtaagcatcatggctggcagcctcacaggattgcttctacttcaggcagtgtcgtgggcatcaggtgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggattttgttttttttcttttgaaacagagtcttactctgttgcctgggctggagtgcagtggtgcaattttggctcattccaacctctgcctcccagattcaagcgattctcttgcttcagcttcccaggtagctgggattacaggcggccaccactacgcccagctaatttttgtatttttagtagagacggggtttcaccatgctggcaaggcaggtctcaaactcctcacctcaggtgatccgcccacctcggcctcctaaagtgctaggattacaggtgtgagcccctgcgcccggccaaggggtgaggaattttgaaaccgtgttcagtctctcctagcagatgtgtccattctccatgtcttcatcagacctcactctgcttgtactccctccctcccaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggtaaccattacacccctcaccccctgggccaggctgggtcctcctagaggtaaatggtgtcagtgatcaccatggagtttcccgctgggtactgatacccttattccctgtggatgtcctcaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggtgaggaggaaggggacaagatgacatagagccattgaaacttttcgtttttcttttctttttttaaaatttttttgaggcagaatctcactctgcccattctgtcggcgagacaggagtgcagtggtgtgatctcccctcacagcaacctctgcctcccaggctatagtgattctcctgcctcagcctcctgagtagctggaattataggcgtgcgccactaccacctggctaatttttgtatttttagtagagacagggtttcatcatgttgaccaggctagtcttaaactcctgacctcaaatgatatacctgccttggcctcccgaagtgctggaattacaagtgtgagccaccgagcccagcagacacttttcttttttctttttttttttttgagacagagtctcgcactgtcacccaggctggagtgcagtggcacaatctcagctcactgcaacctccacctcccgggttcaggtgattctcctgtctcagcctctcgagtacctgggattacaggtgcctgccaccacgcccggctaattttttgtatttttagtagagacagggtttcactatgttggccaggatgattgcgaactcctgacctcgtgatctgcccacatcggcctcccaaagtgctgggattacatgcgtgagccactgacacttttctttgccctttctttggaccctgacttctgcccatccctgacatttggttcctgttttaatgccctgtgaaataagatttcgccgcctatcatctgctaactgctacggactcaggctcagaaaggcctgcgcttcacccaggtgccagcctccacaggttccaacccaggagcccaagttccctttggccctgactcagacactattaggactggcaagtgataagcagagtcccatactctcctattgactcggactaccatatcttgatcatccttttctgtaggaatcggatataacatcatccgggtacccatggccagctgtgacttctccatccgcacctacacctatgcagacacccctgatgatttccagttgcacaacttcagcctcccagaggaagataccaagctcaaggtaggcattctagctttttcaggccctgagggccctgatgtctgggggttgagaaactgtagggtaggtctgcttgtacagacattttgtcccctgctgttttgtcctgggggtgggagggtggaggctaatggctgaaccggatgcactggttgggctagtatgtgttccaactctgggtgcttctctcttcactacctttgtctctagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagtaagggatcagcaaggatgtgggatcaggactggcctcccatttagccatgctgatctgtgtcccaaccctcaacctagttccacttccagatctgcctgtcctcagctcacctttctaccttctgggcctttcagccttgggcctgtcaatcttgcccactccatcaggcttcctgttctctcggtctggcccactttctttttatttttcttcttttttttttttttgagaaggagtctctctctctgtcacccaggctggagtgctgtggcgccatcttcactcactgtaacctctgcctcctgagttcaagcaattctcctgcctcagccttccaagtagctgggattataggcgcctgccaccaggcccagctgatttttctatttttagtagagacggggtttcgccaggctgttctcgaactcctgaactcaagtgatccacctgcctcggcttcccaaagtgctgggattacagtgtgagccaccacacccagctggtctggtccactttcttggccggatcattcatgacctttctcttgccaggttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtaaggcctggacctccatggtgctccagtgaccttcaaatccagcatccaaatgactggctcccaaacttagagcgatttctctacccaactatggattcctagagcaccattcccctggacctccagggtgccatggatcccacagttgtcgcttgaaacctttctaggggctgggcgaggtggctcactcatgcaaacccagcactttgggaagccgaggcgggtgatcacctgaggtcaggagtttaagaccaccctggccaacgtgttgaaaccctgtgtctactaaaatacaaaaaaaaaaaattatctgggcatgatggtgggtgtctgtaatcccagctactcaggaggctgagaagggagaatcagttgaacccgggagatggtggttgcggtgagccgagatcgcgccactgcactccagcctgggaggctgagcgagactccatctcgaaacaaaacaaaacaaaactatctaggctgggggtggtggttcatgtatgtatgtgtatatacatatatatgtgtttatatgtatatatatatacacacacacacatacatacacacacatacacacacaaattagctgggtgtggcacccgtgtagtcccagctactcaggaggctaatgtgggaggatcagttgaccctaggaagtcaaggctgcagtgagtcgtgattgcgccactgtactccagcccgagtgacagagtgacatcctgtctcaaaaacaaaaaaaaatctccccaaacctctctagttgcattcttcccgtcacccaactccaggattcctacaacaggaactagaagttccagaagcctgtgtgcaaggtccaggatcagttgctcttcctttgcaggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacggtaagccaccccagtctcccttcctgcaaagcagacctcagacctcttactagtttcaccaaagactgacagaagcccttcctgtccagctttccccagctagcctgcccttttgagcaactctggggaaccatgattccctatcttccctttccttcacaggtctgcacacctcattgccccttttgcaactactgaggcacttgcagctgcctcagacttctcagctccccttgagatgcctggatcttcacacccccaactccttagctactaaggaatgtgcccctcacagggctgacctacccacagctgcctctcccacatgtgacccttacctacactctctggggacccccagtgttgagcctttgtctctttgcctttgtccttaccctagaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcaggtgagtggagggcgggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctctgcaggagttatggggtgggtccgtgggtgggtgacttcttagatgagggtttcatgggaggtaccccgagggactctgaccatctgttcccacattcagcaagttcattcctgagggctcccagagagtggggctggttgccagtcagaagaacgacctggacgcagtggcactgatgcatcccgatggctctgctgttgtggtcgtgctaaaccggtgagggcaatggtgaggtctgggaagtgggctgaagacagcgttgggggccttggcaggatcacactctcagcttctcctccctgctccctagctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccagtcagtgtgagtggctttattctgggtggcagcaccccgtgtccggctgtaccaacaacgaggaggcacgggggcctctggaatgcatgagagtagaaaaaccagtcttgggagcgtgaggacaaatcattcctcttcatcctcctcagccatgcccagggtccgggtgcctggggcccgagcaggcgttgcccgctggatggagacaatgccgctgagcaaggcgtagcccaccatggctgccagtcctgccagcacagataggatctggttccggcgccggtatggctcctcctcagtctctgggcctgctggtgtctggcgttgcggtggtacctcagctgagggtcaaggaaggaaggtgtgttaggagaactagttcttggatccctgcccactctccccagggctgcccctcccatctgccccttacctccatcccaggggaagtagagactgagaatgtgggtacaataggcacagaggttgtgcagcccacgcaggtggacctgcagcttcccactgggcagctttgcctgcagcagcagggccaagtagctgaagacgaaggcgtccaaggaggcagggctggagcagagagagaagggtgggatggaggagaaccactggggtagaaggggtaaagatggagctggaggaagagtcagccttgggaggtgggctctgggcagcaggcggccaccagggaaggacaggacacacagttctagacctggtatggggagagatccccaggtggcgccagctggccctgaatagggctctatcccagggctgcataaagggcacactcagtgccccacagctcttcaggcccttcctgtgcctggctgccctcccaccctacccttttgtacctctgagaaggctctggccccacgcacagccccactgtcaccagggccagtatctgtctcagggacctcctatccagagcctgagccagccccagccccagccccagctccagctgctccatctgaacctgtatcttcttccaagccacccattaccctcttggagtcagactcacgcatctccaaagaagaacttttgagagcccaggcgctgagagagcagggtcagacactcccgagcctctcggtacagctgtaggggcgacacaggtaggcttgcagctgcgggaacagtgccacctccgcacctaagcactcccattcctggccagcatccttggggctcatctcatacaatagcccccggtctcagagctacctccttctccagctcttcctcgtcctcaggcctgtgctccccagtcagcagctgtagccgttccatgtactgccgctgcatgcggccaggcaggaagaagttgaggggaaagggcatagcctctgcataccacttccgggtcacttctacgtagttcttggtgtctatccaaaaagtatgtacctggattgggtgggcaggaagaaacaggcaggtctgagccagtgcacctgtctgattcaaggtgggcttctgacctccatgctctcctgagtctctgtgtgggtctgtgtgttcccgtcccctccccggctggccatggatgctgggaggtctgggcacactcaccagcaccgggatcaacttctcctccaggagagacatgaaggccagggtgtctgccccttgctgagctgacagatcataatcagcattgtacttctgtggaggaaatatccatggcgtggacgctggggagctgcaagggcacttcaccagggaggaaggagtcctgtctggtacccccctcactggcctctgagtgcagtggaggtacagcaaggaacttttcctgccaaggcccccttgcctgggcccagccagtagcctgttgctgttggcaaaaagcctgggccttggagcccgctggccgtcaaggtcctgggcccattgagaagaaggaagaaaggttgggccgcaaactaggagcagctcccagaatttccatggaaagctggaacaatgcctgctgacagcaactttctaacagtaactttcccgacccagacaccacaaagctagcacaacggagctcagatgcaggctaggactcggtccatgcctcaggaaccagggaaagccatcctcacactccctggatccagggaacccacgcccagggccccccagcttgttccctcagtgcccagctcttggctatttctttcacttcattccatcgcccagacaccattaccacatacacattccatccatacccccaggtctcagcctgccctaccttcccaggctccagtccctgttcctcagcatcccccaccacatcctgagtaagctttgtccccagataacctcttcagcatgatccttaaatctccctaagcctcagtttctcccctgtggaatgggggtaagaatctctttctctgaatgcccctgtgttaggaaataatttagaatacttcggaaacaaaaagctctgttcacacctaagcaatcagggcagtggcctggccttgccaggaacttaggcttttatctggatcctctttccaggcctctcaattaattccccaggtccttaacctttgggaaattagaaattaggaagagtgtcccacttctgacactgtgttccctcttggaacctgaccgtcaatgctagaagaacccttggaaaacatgctggcccagccctctagttttacaaataagggagtgcacagccctgagaggttacatggcctgcccaagatcacgcagtcaatggcagagtaaagagcatagcctaggcctccccactcctctagtaatgctctttcatcttctccaacctggctctaagccttgtccatcctgagccccatatctagcccaacctagtccctgaaaacaagaagtggcccttagaaatctctctccagtcccactatcagaggccaactgctgtcttccagtctccttcagcctgtgctcctctccctccctgactgacaggcagaaggtaccgtgcctctggatatccccacagtgccctgagctgcatctcttgccgactgctttaatacatcacagtgacattgtgtgtgtctctgccaccagactattgctccttgatgctctgggtcacctgcatctagcatggcatatatctagtgctcaataaatgtgtattgtacggaattgactgaacttctctcactggcagccccctctatccaaatcacccacctctttttgaaggtgggtgatgatcttgtgtggtactgaggtgacctctccatgactggtccaaagggcaggcagggttcctgatactgagagaaaagtataccaaccaggaccttagctgcctatccatacgtagttgcaacacattcctgcctatctcctgcttctcctcttgtacacacccttcctcacccccaagggatactgggtacctgaagggctctgccaggggttgctgatcttgtgctatatggtgcacaagaaacttttaagaaaaaagaaactttcaagacacactttccaaccacgaattctatctctgctccttttcatagcaataggttttctttttcttccctccacacttaaacatccattctcttatcacccacccccatcagactccttcccctgtgtttcttcagccactgctctgaccaaaatttgagtgaccaaaagtggtgtcagacccagtgaccatttctctgcctgcatttcacttgaccttgaagcagcaattaatctccataatcagcatcttcttgaatttttccctttgaggacattgctcttctactttgtgctctggttatcctttacaaacttttacacctctcctgaactggttaacagtagagggccccaagggtctcacgtaagccttctttgtttttgttttttttcttttcttttattttttatatatttttttgagacggagtcttgctctgttgcccgggctggagtgcagttgcacgatctcggctcactgcaagctctgcctcccaggttcatgctattctcctgcctcagcctcccgagtagctgggactacaggtgcctgccaccatgcctggctaattttctgtatttttaacagagacagggatacaccatgttagccaggatggtctcgatctcctgacctcgtgatctgcccacctcagcctcccaaaatgctgggattacaggcgtgagccactgctcccagccgtcatttttattttattttatttatttttttgagatggagtcttgctcttttgccaggctggagtgcatggtgcgatctcggctcattgcaacccccgcctcccaggttcaagcgattctcctgcctcagcctcctgagtggctgggactacaggtgcctgtcaccatgcctggctaattttctgtattttagcttgagacacggttttcaccttgttagccaggatggtctcgatctgtgagcctcgtgatctgcctgcgtcggcctcccaaagtgctgggattagcaggcagtgagccactgcacccggccatcaattttttttttttttaaatggagtcttgctctgtcacccaggttggagtgtaacagtgcaatcttggctcattgcaacctccgcctcttgggttcaagcgattctccagcctcagcctcctgagtagctgggactacaggtgcatgccaccacacccggctagtttttgtatttttagtagagacggggtttcaccatgttgtccaggatggtctcaaactcctgacctcaaatgatgtgcctgccttgaccttccaaagtgctgggattagaggcgtgaaccacattgcccaccgtaagccctcttttctacttccactctttctgtaggtggccctactactaacgtccatggctttaagtaccatctttctatgtgttaatgcataagtccagccttgacctctcttgagcgccatccaactcagcatatctggttggatgtctaatgagtatttcaaattcaacatggccacaactgaactaactgaactcttttttttttttaggcagagttttgctcttgttgcccaggctggagtgcaatggcgagatcttggctcactgcaacctctgcctccagggttcaagcgattctcctgcctcagcctcccgagtagatgggattataggccccgctacccggctaatgtttttgtatttttagtagagacagggtgttgccatattgaccaggctggtctgcaaatcctgacctcaggtgatccccctgcctcggcctcccaaagtgctggaattacaggcgtgagtcactgccccggccacacaactgagctcttcattcccaccccagcaccggttatttttccactgttcccatctcaatggcacctccattacccatttgcacattccaaaagcccaggaaccatggtgacttcttttcccatatccaacacaaccaatcctatcttgaattcatccacgtcccaccacctccccagctacctagttccagccaccctctctccacaacctctgaatcaatctttcacttttcccagcaatccattctccactcagcaaaatgatgataaagcacgtcacatcaaggctctgcctcaatttaatggcttcccattgtatttagaatcatctccaagctctcagagactatggtcagctacaatctggcccaccttctgttccagccaaatttcctcacagcacaaggacgtttgcacctgctattttccaagcacgaaaccctcgggcagatatatctggtgctgtcacctaatttcaggttttaactccactctcaccatttcagtgaggacctaatccccatcgcagtcattctatcacatagctttattttattttattttatttttttttgagatacagtctggctctctcacccaggctggagtgcagtggtgtgatctgggctcactgcaaacttccatctcctgcgttcaagcgattctcctgcctcagcttcccgagtagctgggattacaggtgtctgccatcacgcctgactaagttttgtattttcagtagagacggggttttgccatgttagccaggctggtctcgaactccttgacctcaagtgatccacctgcctcagcctcccacaatgttggatttacaggcgtgagccactgctcccggccacatagccatgttttaagcatgtatcactatctaaaattattttttgtttatacgtttgtgtcgtcctgtagaatgtaaggtcacaagatcagggacttgctcattgcactgggtctaacacacagtgcttcaacaaacactcgttaagatactaacgtggcagagtggggccttgtaaacagtttcaggaccctgtgcttgtaagagcaacgtggtgccctcccaaggaagacagggaggatgcaggagcactgcccagagatggcgtcaggctgcaagacatcttgaataattcaccatcgtaacaacccagcctcaggaagagatggggcaaggccagaacgaaacattaggtaagaggcggtggacaatgggattcccacagggcagcttttgggcactggacgttccctaacctgaggctctctgaagaggaaggttaggaatcctctcagcttcggtgggctggactcactgtgggaattcaatcgcccccccccacccactaagggtgtgctggcgggaaagcgctgagacgcatgcgtagttctcgcgtctggcacccgctccctttccaatacgcttgcgccccgtctgtgctacggatggtcagggagagttgtccgtcttcaaatggaccaatgagacttgtggaggggctctgagtcccgcctctggatgagtgaccgtctcttttccaagtgaggcccgcccgcctccccagggctgcttttctcgcggcacgggtggtggggctgcttcttgacttccgcgcctagtgcgcagggccccattctccagtcccgcccacgcgcctttggaggctgcggtgggatttccttttgccttcggttggggctgctgtttctcttcgccgacggtaggcattataaatatttcgccctttgaattttagctctcccctcccggcgttcgcacttagcctttttcatcattatcattatttttaatggtaaggatggtcaaattttactaatgaagcgattataaaatcttcaagtctttgtatccacttgctttttgagggctggagtgggaagaaaggtatataattcattcattcttcggacatgtgacaaacgttcacggagcgcggcaacgagcgccggtgtcgcgatgcgcactggggctgcacatgggagccaggatggactggactggtccctgccctgcccgctgacgattggcaggccactgcctttgatgagctgggcgctataactgccattaagccatttgtacaattaatcacaacagtgataagagcgacaaaggagctattcgggtggctgaggcggaggatcttttgagcctaggagttcgagaccagcctggggaatagagcgagatcctgtctcaaaaaaagggggagaaaaagtgacaaagaagttgtgagggctatgagtttgcgttagaggaggtgtgtgaggtggggaggcggcagggggcgcggttgttcttagagaaagtgacatccgggctaattttgaaagaataagtgttaactaggctaagcggtgggaaagagtagtgtgtgctgaaggaagggaagaggaacctggcaagttcatggcaggcctgtaattggatgctggtctccctccacacttgactcctacagtctgatttctacacagcacccaaagtgatctcttaaaaatacacatgtgatcttgtcacttcccagcaccaaactctacagtgaatttccatcttagaacaaaattcagactccttaccatggccaccaagaccctacacaatctggcctcccaatttccttttccaaggtcaccttttaccactatccatctcactcacactgctccagcactctctatgcttttatttttctttcttcctctttctttctttctttctttttctttttctttctttctttccttccttctctctctctctctttcttttcttttctctttcttttttttttttttgagatgcagtcttgctctgtcacccaggtgtgtgatcttggctcactgtcaacccagagcagtgggggtgatcttggctcactgcagcctccgccttccaggttcaagcaattctgcctcagcctcctgagtagcagggattagcgccatcagtcccagctaatttttgtatttttagtagagatttcatcatgttgtccaggctggtctcgaactcctgacttcaagtgatctgcccatctcagcctcctaaagtgctgagattacaggcaggagccaccacacctggctcagtatttgttttatttttgttgtgtttatttatttagagacaaggtcttgctctgttgctcaggctggagtgctgtggcaggaacacagctcactgcagcctccactacctcaattcaggcgatcctcccacctcagctgagactacaggtgtgcatcaccatgcctggttaattttttcgttttaccatgttggccaggtttgtctcgaactcttgggctctgctgtcctcctaccttagcctcccaaagtgctaggattgtaggcgtgagccactgtgcccagctggtgttcagtatttgaatccatatttcctgtagccgcaaccaaagttccactgttaggtctcactttgactttaataattgtgttcaggctgggcacactggctcaagcctgtaatctcagcagtttgggaggccgaggtgggtggatcacatgaggtcaggtgtttgagaccagcctgtccaacatggcgaaaccctgtctctactaaaaatacaaaacattctccgatcgtagtggcgggcgcctgtaatcccagctacttgggaggctgaaggaggagaattgcttgaacctgggaggcagaggttgcagtgagcggagatcacgtcattgcactccagcctgggctacagagcgagactctgtctcaataagtaaataaataaataaataaataaataaataaataaataattgtgttcggagtcagcatcattcttcctgaagttcaccactcctttgccaagaacacttcctgaaacactgacaagcaggaccttgaataatggggtatggttggtaacaactcactcattcaacaaacattgagcacctattttgtgcttgcctctaaatgaagagctggttgtattatttatttttttaggtgacagggctttccctatgttgcccaggttcgtctcaaactcctgggctcaaaagatcctcatcttctcaagtggttgaatatacacgctccagcgaccatgcctggctgaatgaagagctttgagattttgaagaaacaggaaccatgaaatttgctttgcaactgtttgcaacctttaaggaagactgaaaaggcattcctgaagcatgtgagaagcagtctgtgtgacctgatgactcagaactgcttggaatttagattaggacagatatgagcttaggcttcactctgccacatatttaacttctctaagtcttagttttcttttctttttttttttttttgagatggagtctcgctctgtcacccaggctggagtgcagtggcacgatcttggctcactgcaagctccgcctcccgggttcacgccatcctcctgcctcagccacccgagtagcggggactacagtcgcacaccgccacgcctggctaaatttttgtatttttagtagagacgggtttcaccgtgttagccaggatggtctcgatctcctaaccttgtgatccgcccgcttcggcctcccaaagtgttgggattacaggtgtgagccatcgcgtctggcctctaagtcttaattttcttatctgtaatgtagagttgtcaggctagtgcatgtaataagcactcatgaaagactgactattatcttgcgaaaaattggaagagatcatatgaggtacaaggaccttccatctgcactgggctgtacattgaatgttgtggttactgttgaagcattggtaaatcacatgaatccaatggtaaaaccacaccctaaggtcagactcagtggctcacgcctgtaattccagcactttgggaaaccaaggcaagaggattgcttgagctcaggagttcgataccagtctaggcaacctgtttctataaaaagttaaaaaattagcttggtgtggtggtgtgcacttctggtcccagctactcaggaggctaaggtgggaggatcccttgagcccaggtggtcgaggctgcagtgagccaggatcacaccattgcattccagcctggatgacagtgtgaggccctgtcttaaaaaagacaaaaacaaaccaaaaaaacccacaccctagtgggtaaggggcagcagaggtcccacccaagagtgaaatcatttttggtgtcagtaatcagggagagtaatataccctcaggccagaaactagaggtgagtgatggctcacgcctataatcccagcaccttgggaggctgaggcaggtggattgcttgacctcaggaattcgagaccagcctgggcaacataggaagaccccatctctaaaaataaatttcaaaaattagttgggcatggtggcatgtacctgtagtcccagctactatggaggctgaggtgggaggatccaattgagcctgagaggtcaaagctgcagtgaattgtgattgtgccaagaccctgtctctaaaataaaataaaattacaattaaaaactagagctgaaaggacaggttctaggttaactggtagtagttgttcattcatctgtataaaaagtacttttttgagcacctactctgtgctgtgcatggaatgaacaaatgtctttatttgacttgaacacagaccatagaagtaactaacagtggaactgggagtggcactctccatgggacctaaagacctaggagcctgtgatatgatacctgtcagtgtgaaagggctggagtttgctatctgagccagacctacactccaagacaggtcttgctgggatgggatcaaaattgctgctgggtcagccgaactatttttgtacttctgcttttcacttatatttatgtcttccctaggtatatagctgtctttttttgtttgcttgtttttctgagtcagagtcttgctctgtcactcaggctggagtgcagtggcgcgtctcggctcactgcaacatccacctcgggttcaaacgattctcctgcctcagattcccgagtagctaggattacaggtgcccaccaccatgcccggctaattttttgtatttttaatagagatggggtttagtagagacagggttcactgtgttatccaggatggtctccatctcctgacctcgtgatctgcccgcctcagcttcccaaagtgctgggattacaggcatgagccactgcgcccggccactggagaccccttttctaccaaaaaaaataaaaataaataaataaatatatatatatatttgtaaagacggagttctctatgttgcccaggctggtcttgaactcctggcctcaagggatcctcttgtctcagcttcttaaaatgatggggttacaggcatgagccaccgtaccaggccttagcaaactctttttcaagtgctatacaaaggggacagaggaacgtgattgtggccacctagtatgcccctattggccactgcagtgaggcctaggtgtttgaagagaggcacctagggtagtggctctcaggcacaccctaggggcattttggaaatttgtggggcatctttaatagagacaatgattaggaggcactttgagaatttagtggatggagcccaggaaaggtagacattctgcaacatgtagtacattcatacacaaggaagaattcactcatttcacctgacttacaaatataaaatcaaatataaatcagtaggactttattataaaatattgcagaaaagtttcacaagaattttttgtagaaaaagcccaatcagggccgggcatggtggctcatgcctgtaatctcaggactttgggaggccgaggcgggtgaatcacctgaggtcaggagtttgggacaagcctggccaacatggtaaaaccccgtcttgactaaaaatacaaaaatcagccaggcatggtggtgtgcgtctgtaatcccaggtactcgggaggctgaggcaggagaatcgcttgaacccaggaggcggggtttgcagtgagccaagatcaagccactgtgctccagcctgggtgaaagagcaagactccgtcgaaagaaagaaaggaagggaggaagggaggaagggagggagggagggagggagggaaagaaagaaaaaagagaagagaggggaggggagggaagaggagaaaagaaggaaggaaggcaggaaggaaggaatccagtcgagatcgttaaatttgttgtaattttgtctttggcacaataaacattagtccttattaaaactaactttacttagaaaataaaatagaaaatcctcaaggacaaatcacattgtacaggaaatatgaaatgtgaaataatatccaccttggaaattttttttttttgaaacagagtgttactctgtctcccaggctggagtgcagtggcacaatcttgggtctctgcaacctctgactcctgggttcaagtgattctcgtacctcagcttcccaagtagctgggattacaggcgtgcaccaccacacccagctaatttttgtattttcatttcagatgggattttgccatgttggccatgaacacctgtcctcaagctatccactgcctcagcctcccaaagtgctgggattataggcatgagctaccgtgcccagccaccctggaaaaattttacaaaatgaagtataaaaagaagaaaagaggggcgggcatggtgccttgtgcgtgtaatcctagaactctgcaaagccgaggcaggagatcctttgggtttaggagtttgagaccaacctgcacaacatagcaagaccctatttctacaaaaaatacagacactaggctgggcgtggtggctcacgtgtaatcccagcactttgggaagctgaggccagcagatcacgaggtcaggagatggagaccatcctggctaacatagtgaaaccctgtctctactaaaaatacaaaaaattagccgggtgtggtggcaggcgcctgtagtcccagctactcaggaggctgaggcaggagaatggcgtgaatccgggaggcggagcttgcagtgagccgagatcgtgccactgcactccagcctgggagacagagcaagactccgtctcaaaaaaaaaaaaattttgctcagtacctggccaaaaaagaagcagctcactccctgtacacagaggggtaagagaaaggagatggttgaacttctaaactcgctaaagcaggagaggcaaatgtggaatgtgctcaggaaatatctgtgagatgaatgaatttgagggaagtaaggtactagataattacctgccctacccagaacaaatcctgtgcaacgtttccttgaagagcaggaagtcaggccgggtgctgtgctcacgcctgtaatcccagccctttgggaggccaaagtgtgcgaatcacctgaggtcaggagattgagaccagtctggctaacatggtgaaaccccatctctactaaaatacaaaaattagccgggcgtggtggtgcgtgcctgtagtcccaactacttgggaggctgaggcaggagaattgcttgaacctgggaggcagaggttgcggtgagctgagatcggccactgcactccagactgggtgacagagtgagacaacatctcaaaaaaacaaaaaaaaaagagaaaagcaggaagtctggaaggggtggctactgacatagtgaagcaactagttcaattctacaacttgacaactacccctgtgccaggctgtctacaaggatatttagaatgtgtaagacattccttcaaggaactccaggaacagaggcctgacatgttgcaatgtttagtgtcaagcagtgtactagagacacattatcacactcaaacctcacaacagttctatgaggtaggagttatcactccccttttatagatgaaatagaggcttagagtgattgatttactgaaggtcaaacagccagtaaatggtgtagcaaggattccaaccttgccgtctcactaaaactgtacaaaaaaagatacaaacaacagacaaatagttcccaggcgcctcccaagttgccaggcactgcatttacctcactgacccctttgaggttgtggcattgcctccattttctaggtgaggaaataggctgagagctggggttagtctggtcatgactgtgtgtgccactcccaccaaatctcatttgatgtggttcatgaggcaaatggcatggacaacttccttcacatgtccactaagcatatggccttttacaacactttctggtttctgaactactttaaaacctcactgtcctgtgaggaaggaagaacagttattacaatctgcatctggaagccaattgccctttagagatatggctgcaattgcctcactgcctgtgtcatgtgactctccgaggcccttaatgagtaaatgaggggtgctgcaggggagccaagctgaccactcccctccctccagtcctgccaccccactgccagtgtcccaccctccttgcgccctacacttcactggctaataacccccctcactttttcctgtgttgaaggcatcctggataattccccacccacgaatggtccctcctcatctcagagagctctccatgcacacctgttactgtttctgtttttacctgtaaatatctgtgtctgacttccatgcttcatgcacctctatagggcaaagactgtgtcttaaacatcacggtagcctcagcatgttgtgcaatgaaggtttttttgtttttgttctttgttttttttttggtattagctttatttgtatcattttgaaatttttatcaaaaaagcagcgtgcctgctgtggttcccatcctctgggatttaggaatctttacccgattctccatccaagtctgtctttcgtattctaggctcttcctaaagttgtcattcacatataccctccagaattttatagggtgtataatctgtaacaactcggaggaagccaattgccctttagaaatatggctgcaattgcctcacttcctgtgtcatgtgactctcctagtcatcacatgacccatccacattgggaagccagaattacttgcaggagtaacctagtgcctatagctatggcaggtatcctgcatccttgttttttgtttagtggatcctctatccttcagagactctggaacccctgtgctcttctcctcatctagtgaccctgaggtgatggagttttcaagtccttccagagaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtgtctgtgggccttgtcctaatgaatgtgggagaccaggccatgggcacccaaaggcagctaagccctgcccgggagagtagttgaggggtggagagggacttgcttttcagtcattcctcattctgtcctcaggaatgtcccaagccttcgggtagggtaagcatcatggctggcagcctcacaggattgcttctacttcaggcatgtcgtgggcagtcagatgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggatttttgaaaccgtcttcagtctctccaaacagctgtgtccgttctccacatccttgtcagacctcacctctgcttgtgctccctccctcccaggtggtgcccctgcatccctaaaagcttcagtacagctcggtggtctgtgtctgcaatgccacatactgtgactcttgaccccccgacctttcctgccctaggtgccttcagccgctacaagagcagaagcagtgggcattggatggagctgagtacaggaccatacaggctaattgcaccggcacaggtaaccattacacccttcaccccccgggccaggctgggtcctcctagaggtaaacggtgtcagtgatcaccatggagtttctccctgggcactgataaccctgtggatgtcctcaggcctgctactgatcctgcagccagaagttccagaaagtgaagggatttggaggggccgtgacagatgcaggtgccctcaacatccttgccctgtcaccccctgcccagaatttgctacttaaatggtacttctctgaagaagatgaggaggaaggggacaggatgacatagagccactgacacttttctttgccaattcttgtggaccctgacttctgcccatccctgacatttggttcctgtcttaatgccagtgaaataagatttcgccgcctatcatctgctaactgctacggactcaggctcagaaaggcctgcgcttcacccaggtgccagcctccacaggttccaacccaggagcccaagttccttttggccctgactcagacactattaggactggcaagtgataagcagagtcccatactctcctattgactcggactaccatatcttgatcatccttttctgtaggaatcggatataacatcatctgggtacccatggccagctgtgacttctccatccgcacctacacctatgcagacacccctgatgatttccagttgcacaacttcagcctcccagaggaagataccaagctcaaggtaggcattctagctttttcaggccctgagggccctgatgtctgggggttgagaaactgtagggtaggtctgcttgtacagacattttgtcccctgctgttttgtcctgggggtgggagggtgggggctaatggctgaaccggatgcactggttgggctagtatgtgttccaactctgggtgcttctctcttcactacctttgtctctagatacccctgattcaccgagccctgcagttggcccagcgtcccgtttcactccttgccagcccctggacatcacccacttggctcaagaccaagggagcggggaatgggaaggggccactcaagggacagcccagagacatctaccaccagacctgggccagatacattgtgaagtaagggatcagcaaggatgtgggatcaggactggcctcccctttggccatgctgatctgtgtcccaaccctcaacctggttccacttccagatctgcctgtcctcagctcacctttctaccttctgggcctttcaaacttggatctgtcagtcttgcccactccatcaggcttcctgttctctcggtctggcccactttcttggctggatcactcatgacctttctcttgccaggttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctacccttgccaacggtactcaccacaatgtccgcctactcatgctggatgaccaacgcttgctgctgccccactgggcaaaggtggtaaggcctggacctccatggtgctccagtgaccttcaaatccagcatccaaatgattggctcccaaacttagagggatttttctacccaactatggatcctagagcaccattccccgggacctccagggtgccatggatcccacagttgggacttgaaacctctctaggctgggggtggtagctcatggctataattccagcactttgggaacccaaggtgggtggatcacttgaacctaaggagttcaagatgagcctgggaaacatggtgaaaccctaactctacaaaaaaaaaaatagaaaagttagccgggtgtggtggtggcacgcctatagtcccaagtattctggaggctaaggcgggaggtttagttgagcctagaatttcaggctgcagtgagctatgattgtgccactgtactccagcctgtgtgacagagggagaccctgtctcaaaaacaaaaacaaaaaatccctcccaaaacctctgtagttgcattcttcccaccacctaattcaggattcctacaagaggaactagaagttccagaagcctgtgggcagggtccagggtgacttgttcttcctttgcaggtactgacagacccagaagcagctaagtatgttcatggtattgctgtacattggtacctggactttctggctccagccaaagccaccctaagggagacacaccacctgttccccaacaccatgctctttgcctcagaggcctgtgtgggttccaagttctgggagcagagtgtgcggctaggctcctgggatcgagggatgcagtacagccacagcatcatcacagtaagccaccccagtctcccttcctgcaaaggaggacctcagacccattagtagtctcaccaaagactgatagaagcccttcctgtccagctttccccaggtagcctgcccttttgggcaactctggggaaccatgattccctgtcttgcctttccttcacaggtctgcacacctcattgccccttttgcaactactgaggcacttgcagctgcctcagacttctcagctccccttgagatgcctggatcttcacacccccaactccttagctactaaggaatgtgccctcacagggctgacctacccacagctgcctctcccacacgtgacccttacctacactctctggggacccccagtgttgcgcctttgtctctttgcctttgtccttaccctagaacctcctgtaccatgtggtcggctggaccgactggaacccatcattgtagacatcaccaagcacacgttttacaaacagcccatgttctaccaccttggccacttcaggtgagtggagggcggggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctttgcaggagttatggggtgggtccgtgggtgggcgacttcttagatgagggtttcatgggaggtaccccgagggactctgaccatctgttcccacattcagcaagttcattcctgagggctcccagagagtggggctggttgccagtcagaagaacgacccggacgcagtggcactgatgcatcccgatggctctcctgttgtggtcgtcctaaaccggtgagggcaatggtgaggtctgggaagtgggctgaagacagcgttgggggccttggcaggatcacactctcagcttctcctccctgctccctagctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacaacagggccagcgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccagtcagtgtgagtggctttattctgggtggcagcaccccgtgtccggctgtaccaacaacgaggaggcacgggggcctctggaatgcatgagagtagaaaaaccagtcttgggagcgtgaggacaaatcattcctcttcatcctcctcagccatgcccagggtccgggtgcctggggcccgagcaggcgttgcccgctggatggagacaatgccgctgagcaaggcgtagcccaccatggctgccagtcctgccagcacagataggatctggttccggcgccggtatggctcctcctcagtctctgggcctgctggtgtctggcgttgcggtggtacctcagctgagggtcaaggaaggaaggtgtgttaggagaactagttcttggatccctgcccactctccccagggctgcccctcccatctgccccttacctccatcccaggggaagtagagactgagaatgtgggtacaataggcacagaggttgtgcagcccacgcaggtggacctgcagcttcccactgggcagctttgcctgcagcagcagggccaagtagctgaagacgaaggcgtccaaggaggcagggctggagcagagagagaagggtgggatggaggagaaccactggggtagaaggggtaaagatggagctggaggaagagtcagccttgggaggtgggctctgggcagcaggcggccaccagggaaggacaggacacacagttctagacctggtatggggagagatccccaggtggcgccagctggccctgaatagggctctatcccagggctgcataaagggcacattcagtgccccacagctcttcaggcccctcctgtgcctggctgccctcccaccctacccttttgtacctctgagaaggctctggcccccacacagtcacactgtcactagggccagtttctatcccagggacctcctatccagagcctgagccagccccagccccagccccagctccagctgctccatctgaacctgtatcttcttccaagccacccattaccctcttggagtcagactcacgcatctccaaagaagaacttttgagagcccaggcgctgagagagcagggtcagacactcccgagcctctcggtacagctgtaggggcgacacaggtaggcttgcagctgtgggaacagtgccacctccccacctaagcactcccattcctggccagcatccttggggctcatctcatacaatagcccccggtctcagagctacctccttctccagctcttcctcgtcctcaggcctgtgctccccagtcagcagctgtagccgttccatgtactgccgctgcatgcggccaggcaggaagaagttgaggggaaagggcatagcctctgcataccacttccgggtcacttccacgtagttcttggtgtctatccaaaaagtatgtacctggattgggtgggcaggaagaaacaggtaggtctgagccagtgcacctgtctgattcaaggtgggcttctgacccccatgctttcctgagcctgtgtgtgggtctgtgtgttcccgaaccctccccggctggccatggatgctgggaggtctgggcacactcaccagcaccgggagcaacttctcctccaggagagacatgaaggccagggtgtctgccccttgccgagctgacagatcataatcagcattgtacttctgtggaggaaatatccatggcgtggacactagggagctgcaagggcacttcaccagggaggaaggagtcctgtctggtacccccctcactggcctctgagtgcagtggaggtacagcaaggaacttttcctgccaaggcccccttgcctgggcccagccagtagcctgttgctgttggcgaaaagcctgggccttggagcctcctggccgtgaaggtccagcgcccaatgcagggaaggaaggaaggctccgccgcaaactaggagcagctcccagaatttccatggaaagctggaacaacgcccgctgacggcaactttctaacagtaacttccccgacccagacaccacaaagctagcacaacggagctcagatgcaggctaggactcggtccatgcctcaggaaccagagaaagccatcctcacactccctggatccagggaacccacgcccagggccccccagcttgttccctcagtgcccagctcttggctatttctttcacttcattccatcgctcagacaccattaccacatacacattccacccatacccccaggtctcagcctgccctaccttcccaggctccagtccctgttcctcagcatccccctccacatcctgagtaagctttgtccccagataacctcttcagcatgatccttaaatctccctaagcctcagtttctcccctgtggaatgggggtaagaatctctttctctgaatgcccctgtgttaggaaataatttagaatactttggaaactggaaaagctctgttcacacctaagcaatcagggcagtggcctcggctctgccaggaactttggcttttatctggatcctctctttccaggcctctcaattaattccccaggtcctcaacctttgggaagttagaaatgaggaagagtgtcctacttctgacactgttccctcttggaacctgaccgtcaatgctagaagaacccttggaaaacatgctggcccagccctctagttttacaaataagggagtgcacagccctgagaggttacatggcctgcccgagatcacatagtcaatggcagagtaaagagcatagcctaggcctccccactcctctagtaatgctctttcatcttctccaacctggctctaagccttgtccatcctgagccccatatctagcccaacctagtccctgaaaacaggaagtggcccttagaaatctctctccagtcccaccatcagaggccaactgctgtcttccactctccttcagcctgtgctcctctccctccctgcctcacagtgccctaagtttcatctcttgccgactgctttaatacatcacagtgacattgtgtgtgtctctgccacaagactgttgctccttgatgctctgggtcacctgcatctagcatggcatatatctggtgctcaataaatgtgtattgtatagaattgactgaacttctctcactggcagccccctctatccaagtcacctacctcttttcgaaggtgggtgatgatcttgtgtggaactgagatgacctctccatgactggtccgaagggcaggcagagttcctgatattgagagggaagtataccaaccagacccttagctgcctagtcatacatagttgcaacacattcctgcctatttcttgcttctccccttgtacacacccttccttacccccaagggatactgggtacctgaagggctctgccaggggttgctgatcttgtgtaccttcagtggagcaccagtaaatctggcataggtctgcagggaaggaagcagaagtcagagaggcagagccatgtcccccacagtggtatcaagaagagaaataacatttattgaataccttatgtgccattccctatacttagtatcttagtctcgaaaaaaaagggggagtcactaccatttctattttacaaatggacaacacagggctcagagagattccggacatgtctgtgattacaggacagccaggaaaatcctttcctctatctcctctcttgccatctacctggtgaacatctatcctcagaagcttttcctaaccagtatccctcttcttggaagcactgataatgcccttctctttggcagtatctgtggtctgttcctactttaacttatatgtgaatattttattatatttatttgcttacctatctgcctttctgtaaactcgaagggaggaaccaaattttattcatctttgcctggcaatgtttaatatttggaagaaatctaatagatcctctttaaagcaaggagtcaatgaataaatgaagcaaaggaagtctgattgctaagctctttatacaaagcctggctgtgagtcgtttggtctaacaaacaatagcctcagtaaatgctgttaagtgaggaggaagaggagaagggggctgaggagaaggcaggaattccaagctagagtaggtccctgagacagtccagcctagtaaacctccgaatggagaaaaaaaatgcccaaagagaaaggatctccgcaaggtcacacagccagtgaatggatgagcaaggactagaacccatcacctgaactcccaaccaggcgtcttttcattgctgcattaagcctagaaaactacagcatagggcactgaggccatatcggccacgtacgctctttgactgcctcagtttcccccactgcgcccgtgctgagtagccctggcaaggtttggatgcttggctagttcggccccctccccagtcatcagggcacagcagagggcgccggcgccacccctcaccagcacggccaggctgtccaggtccactgacggcagcccccagccccctgaccagcagaacagctccatgggcgccgccatcttgcccaccctctgtcccggaaacacttccttgtgtgcttctgccctcccctgcctgggcccgccccccgccaccgcccctccgatcgttgcggtcagggggcctggggagatccccggggaggcgaggcttcttctggcccgactggcagctgaactgcgggggactgggccgcgggcctcgggggagggcggccgccggcccatccagaggtggcccacgtagcgggacagcgctgtcggcccggcgcgcctcggagtgtcacggcgcctcgtccaagtggagccccgaacccctgaaggcgcggcaggctctggagagcggggtcttgtgcgcctgggccaggtctgggggctcttgccaaactgcacgtggcctgtactgctccagggccccttggggctcgtccccgagcggggactgcgggggggtcccccgagcagcatgttttccacagcgcgttatgtttggagcgggccctgcgccgcctgtcgccatggaaacaaaacaggggcggtggcggcggccggagcggaggccgggctggggcttgggtgggggaggggaagagaggctcgcaggctgtcgcttaggtgacgggaactcaggcgcccctctgcttcatccgggtcacggcccgtccgctagtacccacagtgttccacagtctggtccttggctcctcgcctgtacccctggtcttctgcgcctgtccctggtgtccctttcctcttttttggtttcttcactctgacctcactgaccactgctttagattctcccttcagttcccgtcagacgcttccagactcccaagctttcctacgaatgagggaaaatggagaaacaggcacttgtcaggggacccccaccctaataaagagcacttgctccgccagaacagcaaaattcatgccatgtgggcatccctgggcactatagcaagctagttgcggccactcccttggcatcctttcctgccagctgtggaataatgcccactgtctagcactgcccctgccaggggttcttgccttccacaatcgtggcttccagaaaacagtggcattcggtagcgctgtgtgccgagacccccaacaatgatgactgcggagagggaggcacctggggggaggatcattagggagaggtagaaagcagggaggcctccaggattctttcccagtgcccctggttcccagagctgatgatgcctccagggtgattggcagctctttgtttcagccccccctcacccgcctgctggccccccctccaattctgtccctccgccccccggctcctgctctctccgcctagccttttcccctcccagctgcctgcctgccaggggtagtgagccggctgagaggcatggagacgcaggaacttcggggggccctggctcttctcctcctttgctttttcacatctgccagtcaggatctgcagggtaagcctgtctccatcctcttagaccgctctctgcttcttccccatttgccctcagcccaagtagcagagaacatgtgggcaaggggagaggggaagagtccagaaattgagccagaggaaaacttaagactgcctagagttggtgaaattatggcctggtggagaggagtgggcaccggagagtagtgggggagctggaacaggacagggtcaggcatgaggccagggcagaggactcaggaactggatgctcaggctgcctggctggggtggttcctgagcatctgtaggcaccctagggtctgggagagcagcctgaaggtgggcgtactgtaggtccctgggcttcgtccccctgggctcctggctgcccaaggacagggcgggggtggggaccaagaagcctgggctctcccggaggtctggtggtgggatgggccgatggatgtgggagaggtagctggagcagatgacacaggcatagattctttactggctggagggaaaaccacagattggccaggacacaggaggcagaggagctgggtcctacattcccgtgggagatagcccagatggtgggagggttagagtcctcagtgggcagctgttcttatccagagcgagtgtgctgcgcccctaggatgggaggaggggaactagggtgtggaacgagccaaacccaggagtggaaagagaagctgccttccatttctacgttgtggacaccaggtgccactcctgtgggggatcagcacagcatctcctttgcgccacctggtgggggcatctcaaatttgtggggtgctgtttctgtgtttccaagtgagtctcaaacctgcaggttcctggaaggtagtaataggcaataatggggaaaggaaggcacagtgcctctgtcctctgagagcaacaagggttgaggtttttttttgttttcgttttgtctttttgagatggagtttcactcttgttgcccaggctggagtgcaatggcgcgatctcggctcactgcaacctccacctctcaggttcaagcgagtctcctgcctcagcctcctgagtagctgggattacaggtgcccgccaccacgcccggctaggtggtttttttttcttttttgttgttgtttttgtatttttagtagacacggggtttcaccgtgttggccaggctggtctcgaactcctgaccttaggcgatccacctgcctcaacctcccaaggtgttgggattacaggtgcgagccaccatgcccagccaagggttgaattttgaggtgactaacgctgcctggtggtgggggtagggggtgatgtgggctcggggagggcataccttagcatttgatcccattctctaaccttgataacccctgcctgaccagtaattgacctgctgactgtgggcgagtctcggcagatggtagctgtggcagagaagatccggacagccttgctcactgctggggacatctacctcttatccaccttccgcctgccccccaagcagggtggtgtcctctttggcctctattctcgccaagacaacactcgatggctggaggcctctgttgtaggcaagatcaacaaaggtgactggtgggcatctcctttcttgcaatggtgacctccttaagcactctccctcatccctacttcccaagtgcttcctcattctccttggcctccattagggaccaaggtcccatcaggctgtgcccagacatcaggggcacctggtatggggtgcttataaggtgctttaggagcccatgtaccaaacaatagggtgaggcttgagtcctgatatttatagggatgggaggttgtacttggggatgcctggaaggagggtgaggcatgtctctgtgcctcagcggtgatacagaaaggtctttcagatccctggaacagtcagtggccacaaaaccagctgtcaccctaggagaggatacagtcatctgataccaatgaagactaagaaagtatttgtctctaaaccaattatttttttaaatggagtcttgctttgtcacctaggctggagtgcagtggtacaatctcagctcactgcaacctctgcctcctgggttcaagcaattctcctatctcagcctcctgagtagctgggactacaggtgcccaccaccatgcctggctaacttttgtatttttagtagaaacagggttttgccatgttgaccaggctgttctcgaacttctgacctcaagtgatctgcccaccttggccttctaaagtgctgggattacaggtgtgagccaccgtgcctggcctctaaagcaatttcaaaaacatttattgtgtacctattaaatacaaggtgttgagagccacatagtagaattagggataggggccctagagatgtggtatctagcagagttgatccaaggctcagctgggaggcagggggtgggggatagcgcccatactgatctcccctcctgggcagtactggtgcgataccagcgggaggatggcaaagtccacgccgtgaacctacagcaagcgggcctggctgatgggcgcacacacacagttctcctgcgactccgaggtccctccagacccagccctgccctacatctctacgtggactgcaaactgggtgaccaacatgcaggccttccagcactggcccccattcctccagcggaggtcgatgggctggagattaggactggacagaaggcgtatttgaggatgcaggtgagccgggaggagcctctgagttctgtggaaatagagtttgcaccagctggggaaggggttggtagaccttcccaccttactctgctgctatccccccagggctttgtggaatctatgaaaattattctgggtgggtccatggcccgggtaggagccctgagtgagtgtccattccaaggggacgagtccatccacagtgcaggtaacacagagacttgtttgctgacattggaccacggatcctgtggcctttggtgactcctgtcttcttgatctccctctcccttaaaacccattcctttggctcacctgttcctagggtttctaaccctgttattccaaatctttcacctgactccacaatctccaatacacccatccgagaaaaaaagtgatctaagagaagaattggttaattgcttggccttggctgaccaagagatactggtctcgagtattttttttttttttggtgatggagtcttgtcctgttgcccaggccggagtgcagtggtgcaatctgggtcactgcagtctccactgagttcaagtgattctcctgcctcagcctcccaagcagctgggattacaggcggccctccaccatgcctagctaattttgcatttttagtagagatgggatttcaccacgttggccaggctggtctcaaacacctaatctcaagtgatgcacccacctcggcctctcaaagtgctgggattacaggcgtaagccaccgcgcccggcctggtctcgagtcttttatagttctcactggcagctgtcaccagcaatttctctgagtgttgcccatctgcctggtctctctgacagggatgttcagaagctctcacctaaataaaagacccacctttcccagatatttgagggagagctcttgaagaagggaatgggatggcgggtgtggtggctcacacctgtaatcccagcactttgggaggctgaggtgggctgatccctcaaggtccggagttcaagaccagcctggccaacactgtgaaacctcctctctactaaaaaatacaaaaaattagctgggcatggtggtgggcacctgtagtcccagctacttgggaggcggaggcaggagagttgcttgaacccaggaggtggaggttgtggtgagcagagatcacgccactgcattccagcctgggaaacagagcaagactccgactcaaaaaaaaaaaaaatggggatatactggggccctggccctgctttgggtccatcccttctgccactaccatgcctaggaaccaggaggatttgggttctaacttcctgtgaagcaactcccttagagggccttttgccccatagaaggagctggcactgcttgtctgccagctctgccctcccagcatccagcaccccatctttattctggggctccagccctgtccctgtcctcaccttccttcctccttctcaccaaccaggcctctcttcacttcacctcacccctctgactatgttttcttctcctctccagtgaccaatgcactgcactccattctaggtgagtaggccacactgaagcggaagcggggagcggggaggaggccccaggctctggcagctgcctgaaactaagtcctcttcagtcaggaatgtagtaggtttaaaggcaggggtgggcagccgtggcaggtactggcttattgcccatggagggcccaggactggtgctccagtactgaacccctcacaccctgggtcccgacaggggagcagaccaaggcgctggtcacccaactcaccctcttcaaccagatcctggtggagctgcgggatgatatacgagaccaggtttgggtgggctggcgaagggtggcactgattctggggtagggtggcagatgtcaagtgctgactcctccccatccttctccaggtgaaggaaatgtccctgatccgaaacaccattatggagtgtcaggtgtgcggtgagtgggagagcaggggaggctccacatgaccgtgccacgttcccaccattggctttggctttcccttctggtggcttaaatagtgaccaccgggtagctctgactgtgtccacccctcaggcttccatgagcagcgttcccactgcagccccaatccctgcttccgaggtgtggactgcatggaagtgtacgagtacccaggctaccgctgtgggccctgcccccctggcctgcagggcaacggcacccactgcagtgacatcaatgaggtgagggaggtcagagcccagaagggtacagaaaactggggtgaggatgtcaggaggcacccaagagggtgggataaatgctggtccggaggagaggaatctggagtttaggagaggtcagaggcaagagaaatgcaagatgggagagacagaaggcctggggcaaagactgaaggccatacagggaaggagctggggaaactgcaggggaggcttgagatggcgaccagtggcatggggagggagagagggaaccccgagggaagtggggtggggaccaggagcacaaggcagttgtgtggggagagctgcacaaaggggagacctggagcaatggttcctggatcacaggcagggacctgagtttcccagagggcggcctgacctctgccttctcatctggtccccagtgtgctcacgctgacccctgtttcccgggctccagctgcatcaacaccatgcccggcttccactgtgaggcctgtcctcgagggtacaagggcacacaggtgtctggtgtgggcattgactatgcccgggccagcaaacaggtcagactgggtagtgtgtgtggacaagggatcttggccttgtagaggccaggggcttctgggttggacatgggacccttgtgatgaatgggacatggatggctttgcatcggctttgggtctgggcttaatgttgatgttcacagataaggccataccagtgtcctctgctgtatctgaggagacccaccataggtctggttccacccaaagtctgccccttcaggtctgcaatgacatcgatgaatgcaacgatggcaacaatggtggctgtgacccaaactccatctgcaccaacactgtggtgagctgaatatcctgagtgtattctggggtggtgggaatggtaaaaccccaacccccgccccttcttaccttcaaatttcctgctgcctttcctcctccccagggcactacacagattaggtgttcacatggacttggtttggaatactggtttgcctcttgctagctctttagcaggttatttaacttttctgagcctcagcttcctcatctgaaaactgaggctgttatccaccctgaaaggttgtcatgataacctaaaggagatactgatatgcctggcataactgagtgcccagcccacactccgcaggtggagcagtgctggtcctgggtgcctggtgggctcaccctccttcctaagatgctgcctctccactcttagggctctttcaagtgtggtccctgccgcctgggtttcctgggcaaccagagccagggctgcctcccagcccggacctgccacagcccagcccacagcccctgccacatccatgctcactgtctctttgaacgcaatggtgcagtgtcctgccaggtgagctaggcttcaggcgtggaaggaaaagggagggtctggggaaaggagtagggctatgtttagggcctgggttgggggtcttcataggagagaaggtgggcctgggcccaggaactgtttggtggggagaataggacctgaagcagggaaaatatagggaggaggggagccagaccaaactgctagctcctaccctttgtgttgccctagtgtaacgtgggctgggctgggaatgggaacgtgtgtgggactgacacagacatcgatggctacccagaccaagcactgccctgcatggacaacaacaaacactgcaaacaggtgcagggagcaggcgggcaagggggcgtagtggggagcccaagctgggtcaggccagaactcatccatcctcttcccctgaactttaggacaactgccttttgacacccaactctgggcaggaagatgctgataatgatggtgtgggggaccagtgtgatgatgatgctgatggggatgggatcaagaatgttgaggtgactcccagactgccctgccccttgaagctcctctcccctcctcctgtcctctctgtgcctcacttacctcatctggcagctctctagtaagggccaaatactccaaatcagggaggcaaaaacctcgtgcccaggacaaggaggctgggtgggtgggactgtactgagcagtttgtccatcacaagggtgtgatcttagaaaaggatacagagacaaggtaggtgcaagatgacaagtgatttaggggagacctgacccttcccctcccaccctctgcccaggacaactgccggctgttccccaacaaagaccagcagaactcagatacagattcatttggtgatgcctgtgacaattgccccaacgttcccaacaatgaccagaaggacacagatggcaatggggaaggagatgcctgtgacaacgacgtggatggggatggtgcaggcctggggctgaaggggtggctgggggacctgtgagaatttggatcaggtggggatgaagcagggaagctaggaagtctctgtgaaatagggaggcaggcttgtggacgttggcctgggtgaggagagattacctgcagcagatgtcaataggaatgtgaggtagggcgtagtgttaggcagagtgtggactagagggtgagacaagaaacaggcagatttcctggccagttgtcctctgggtggggagacaaagttcgggactttcaccaacctagaagagagaatatggcatgttctagtaacaaccttgtgctacccatgtcttctaggcatccccaatggattggacaattgccctaaagtccccaacccactacagacagacagggatgaggacggggtgggagatgcttgcgacagctgccctgaaatgagcaatcctacccaggtacagggagatggtaaggacaggggagggatgagggtactgatggatgaagccccagccctttggatggaaagtggtcagatcaccctcttcagagttatcaagaggagatggtgagaacaggtccctctctctcagacagatgcagacagcgacctggtgggggatgtctgtgatactaatgaagacaggtaaggtcttggtcaagagacgcaaggtctttcttttttttgtctttctgagacggcttgctctgtcacctaggctggagtacagtggcacgatcttggctcactgcaacctccgtctcccagggtcaagtgattctcatgcctcaacctccctgagtggctaggattacaggcatgtgctaccaagcccagctaattcttgtattttcagtagagacagggtttcaccatgttggccaggctggtctcgaactccttacctcaggtgatccgccagcctctgcctcccaaagtgctgtgattacaggtgtgagccactgtgccagcgaaatgcaaggtcttataggggatattttactttcctctagtatatccttttttttttttttttgagacggagtcttgctctgtcgcccaggctggagtacagtggcatgatctcggctcactgcaagctccacctcccgagttcacgccattcttctgcctcagcctcccaagtagctgggactacaggcgcctgccaccacgcccggctaattttttgtgtttttagtagagacggggtttcactgtgttaaccaggatggtctcgatctcctgatctcgtgatttgcctgcctcagcctcccaaagtgctgggattacaggcgtgagccaccgtgcccggcctcctttttttttttgagatggagtcttgctctgtcactcaagctggagtgcagtgatatcggctcactgcgacctccacctccagggttcaagcgattttcctgcctcagcctcccagtagctggattacaggcgcgtgctaccaagctcagctaatttttgtgtttttagtagagacagggtttcatcgtgttggccaggctggtctcgaactcctgacctcaggtgatctgcccgccttggcctcccaaagtactgggaatacaggcatgagccactgtgcccaggccatagtatattctaaattccttctatgatttagccttaattctctattgctattcagcaggaatttattcttacagtcatccctccattcctactgccagaccttcacctcacctggctgactgccaggggtctgattgtatggagagcaggctccagagactcccaggcagaagcgaagggaaggacaaggagtacctttaaatcctttatttggtacctgttcttctgattagcgatggggatgggcatcaggacaccaaggacaactgcccacagctgccaaatagctcccagctggactctgataacgatggacttggagatgagtgtgatggggatgatgacaatgatggcatcccagattatgtgcctcctggtcccgataactgccgcctggtacccaatcccaatcagaaggactcagatggtaagcctgcggacccagagcacgttagactggtgttgcctttgcccaggtggaggcagcaagccctgttgggaagtgaggaagggcaaggtgggaaagatgtcaggaatgcaggcccaacagatggtattgttgcctaatggcagtggccagggccttcctgagcacccagcctcactctgcccaggcaatggcgttggtgatgtgtgtgaggatgactttgacaatgatgctgtggtcgaccccctggatgtgtgtcctgaaagtgcagaggtaacgcttacggattttcgggcctatcagaccgtcgtcctggatcc

SEQ ID NO: 55 - DNA, 2385 bp, Homo sapiens
  Genbank Accession No. X86809
gccagagcgcgcggggcagtgtgcgcgggagccgaggaggaggttccggacgctgcttaggaaccggggactcaggagtgcccgcgccctgagcgctcagctccagaggcgtcatggttgagtacgggaccctctttcaagacctgaccaacaacatcacccttgaagatctagaacagctcaagtcggcctgcaaggaagacatccccagcgaaaagagtgaggagatcactactggcagtgcctggtttagcttcctggagagccacaacaagctggacaaagacaacctctccatcattgagcacatctttgagatctcccgccgtcctgacctactcactatggtggttgactacagaacccgtgtgctgaagatctctgaggaggatgagctggacaccaagctaacccgtatccccagtgccaagaagtacaaagacattatccggcagccctctgaggaagagatcatcaaattgggtcccccaccgaagaaggcctgagcaagggggaggaagaggaggaaggttggaccttcatcagaccacttccttcccccatcctccaggagagggggcaagggcaacccaccatctacccacttactaacctggtcctaacccccttactgtgcgcgtgtgtgtgcgtgtgcgcagctctggctgtttgtctatatgtctagctcatctagttcctcttcttaaggggatgggggtcaggggctaggggagggggctgagtttccccactttaggaggaggtgggggctatttctatgcaaatagaaatcagcacattcctcctacttccctttcctccactccccccatatctttaaagtgtggaagcagaaaggacctgcattttcctacattgaggagctgacataggggtaaggtatgggagaggtaggtggatccagggaaaagcagtggggacggaaggcaaagagaccacttaacccccacctggaaggggcaaagaaaagccagagttccatgtttgtactcctgtgctggactgtttcctgagtaccagcaggtccctttttgtctctcatgggcctagcataggtatgagccagggatcctttcctggtccctaagatcaaaccccatggagcagccagcgttagatgcccccacccacctgtactctggagagactgtgctgggaacatgtaccactgagcctgagatggggatgagggcagagagaggggagccccctcttccactcagttgttcctactcagactgttgcactctaaaccttagggaggttgaaagaattgagacccttaggttttaacaacgaatcctgacaacaccatctattagggtcccaaattggttattgtaggcaaccttccctcttttcttggtgaagaacatcccaagccagaaagaagttaactacagtgttttcctttgcaccgatccccaccccaattcaatcccggaagggacttacttaggaaacccttctttactagatatcctggccccctgggcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgcccttttttgtggtgtctagcattaacttattgtctaggccagagcgggggtgggaggggaatgccacagtgaagggagtggcagaatcaaattgctacatagtccaaacaaaaaagaaggctttttcaaaaaacattaaattcacatgcagtctcagagactttttagacaaagttcaagttaggagcttttaggatgtgggagtaaaactttaatgggaggggagggctggctgctggaagaaggaagaagccagactggttagacagtactcttaactcctagcccagcctagcgtgccctgcccctctggccactgctgcagacacctgccttaacacacacacctctaggactccacagttttgccttaaaggaccttcccaagtctccctttccctgtctggcttctcccttaagaagagagagatacttgtagaattgggtggggggaatgagcatgaactgtccttccatttgggatatgttacattagagtgagagagagaataaggagcctttcttatggaagaaatgggagaagagagacagggttcttttcagcagagtctagtagtttctctgtaaggcaaaataatctaaaaagactaacctgcccacccactccttatattgctgtgagattgcccctatcttgtgctcttctgtctgcagtgtgcacggccttgttctaacccggaataaaggtgattga

SEQ ID NO: 56 - DNA, 1689 bp, Homo sapiens
  Genbank Accession No. U32680
cccctagacaagccggagctgggaccggcaatcgggcgttgatccttgtcacctgtcgcagaccctcatccctcccgtgggagccccctttggacactctatgaccctggaccctcgggggacctgaacttgatgcgatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtattcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttccttgcaggggggccagccattgtctcccacttggggagtttcttcctggcatcatgccttctgaataaatgccgattttgtccatgg

SEQ ID NO: 57 - DNA, 925 bp, Homo sapiens
  Genbank Accession No. D26600
ttttttctgctaccgtgactaagatggaagcgtttttggggtcgcggtccggactttgggcggggggtccggccccaggacagttttaccgcattccgtccactcccgattccttcatggatccggcgtctgcactttacagaggtccaatcacgcggacccagaaccccatggtgaccgggacctcagtcctcggcgttaagttcgagggcggagtggtgattgccgcagacatgctgggatcctacggctccttggctcgtttccgcaacatctctcgcattatgcgagtcaacaacagtaccatgctgggtgcctctggcgactacgctgatttccagtatttgaagcaagttctcggccagatggtgattgatgaggagcttctgggagatggacacagctatagtcctagagctattcattcatggctgaccagggccatgtacagccggcgctcgaagatgaaccctttgtggaacaccatggtcatcggaggctatgctgatggagagagcttcctcggttatgtggacatgcttggtgtagcctatgaagccccttcgctggccactggttatggtgcatacttggctcagcctctgctgcgagaagttctggagaagcagccagtgctaagccagaccgaggcccgcgacttagtagaacgctgcatgcgagtgctgtactaccgagatgcccgttcttacaaccggtttcaaaccgccactgtcaccgaaaaaggtgttgaaatagagggaccattgtctacagagaccaactgggatattgcccacatgatcagtggctttgaatgaaatacagatgcattatccagaactgaagttgccctacttttaactttgaacttggctagttcaaagatagactcttcttttgtaaagtaaataaattcttcaaaatg

SEQ ID NO: 58 - DNA, 86080 bp, Homo sapiens
  Genbank Accession No. AL008726
gatcagagtagtaaagggtgaggcagcatggtaaaatggggagagtacaagccttgaagtccaaaacccaagatccaaggccagcctctgcgagttgtgtgaccttgggccggtagcagtgcctctctgagccatttcttcatctgtgggattggacctgtctgtggggtctccctcacagagagaccatggtaaatgggaacccgggtataagtcacctagcttgtgctgggtacacagcgttggtgcttggcaagaggaatgtcccgtctttgaaccatggtaaggggcaggggataggaggagaaagtgacaggtaggccactgtttactgaaccccatgacgtgccacacactgtgctaagcattttaacatgatctcatctagtgctcctcctatgaggtagcaacgattatccccacttgactgcagagctttgtaaattataaaggtctatactcactctgtcaccaaggctgatgtgcagtggtgctattatagctcactgcagcctctatctcctgggctcaagtgatcctcccacttcagcctcccgagtagctgggactagaggtgcaccaccacccctggctttttttttttttgtatttttatagagatggggtttcaccatgttgcccaggctggtctcgaactcctaggctcaggggatccttccaccttggtttcccaaagtgctagaattacagtcatgaggcaccatgcccacccatgttgatgattcttattattaatcctgactggagggaagttgggacagacagtccttggggcacctgtccagatggaaaaatgtatggagggatggaggagaggaggatgtgcatttggaggagaaataatcattgaaagcaaagatccttctctgaaagttaccttctccagggaaagggtgtttggattcactttttttgaagtaaggagtaggcaggggaaggttctttaagtggtaaaaggacagctggccccttcctagagctcagtgaagaaatctggtcatctggggagtaaaggggtgggcagaccaagtccagagggagcccaacctgagaatccacaaggtgcaggctgggcactggggagtaggcaaggcttgtagacagtggctacgtggacagaaagagcaccaagacaaccgatagcaggctctggggccaggaaaggggggctggacaccaaggaatgcagatggacagtggcaactgggctgcccccttcccctatatatagacattcccaaggaaagctcagcagggctgggccaagctaaataaggcctcccccaggcaggcagggtgccaaagggtgagggagacccatgggattccaatttagtttttattgttttttggttttttggggttttgtttttgagacagactctcactctgtcacccaggctggaatgcagtggcacaatctcggctcactgcaccctccgcctcctaggttcaagtgattctcatgcctcagccttctgagtagctaggattacaggcacataccactacgcccagctaatttttgtttttggtttttttttttttttttttttgagacagagttttgctcttgttgcccaggctggagtacaatggtgcaatctcggctcaccacaacctccgcctcttgggttcaagcaattctcctgcctcagcctcccgagtagccggaattacaggcatgagccaccatgcccggctaattttgtatttttagtagagacagggtttctccatgttggtcaagctggtctcaaactcccaacctcaggtgatccgcccacctcagcctcccaaagcactgggattacaggtgtgagctaccacacccggcaaattttttactttttgtagagacaaggtttcatcgaccgggcacggtggctcaggcctgtaatcccaccactttgggaggctgagatgggcggatcacgaggtcaggagatcgagaccatcctggctaacacagtgaaaccccatctctactaaaaatacaaaaaaattagccgggcatggtggtgggcgcctgtagtcccagctacttgggaggctgaggctgaggcaggagaatggtgtgaacctgggaggcagagcttgcagtgagccgagatcacaccactgcactccagcctgggcaacagagcaagaccccgtctcaaaaaaaaaaaaagtttcatcatgttggccaggctggtctcaaactcctgacctcaagtgatccgcctgcctcagcctcctaaagtgctaggattacaggcatgagcccctgagcccagcctttgttcttttgtttattcaagggattaggtaaggcccaaattgcttaatgtagccaagcaaggtcccattggcccttctaatctcactccttccctccacatcatgttgctgtctgctctgatcagactttaaaatactgctctaggctgggcacggtggctcatgcctgtaatcccagcattttgggaggtgcaggctggcagatcacgaggtcaggagttcgagaccatcctggccagcatggtgaaaccctgtctctactaaaaatacaaaaaattgctgctgggtgtggtggtgggcgcctgtagtcccagctacttgggaagctgaggcagaagaatcacttgaacctggcaggcgaaggttgcactgagccaagatcatgccactgctctccagcctgggcgacaaaaggagactgcatctcaaaaaaaaaaaaaaaaaaaaaaagactactctaaacaccaccttctatgaaccttcaagcctttgcacatgctgtttttttctgccagaaactcccatctttcctctcttcatctgtctacttccctatcatctttctaagctctaacatcacttcctcggggatccttcccccactgtgtccagaccagggagtccaaggtggtctctagtagtttcctcaatcatagcactgagcataccttatgctcacaacccattctatgttctacagatcatcacatttcccagtgctcagcacttgctatgtagcaggtgttcaacagatattatattctacataagaataactgaactggatggtatttagttcaatataagggggaacttcctaatagtcatagttgaacaataatgggaacaggtggcctcaggaggtaatgagctccgtatcactgagtatacccaaacaggagctagatgaccatttctcagggaggctgtagggagatatccatacagaatttaagaatgggggcagtggctcacacttttaatcctagcacattgggaagctgaggcaggtggatctcttgagcccaggagttcaataccagcctgggcaacatggcgagacgctgtctccaattaaaaaattaaaataaaacaaagaatggtgggtggtagctcatacttgtaatcccagcacattgggaggcagaggtaggaggatctcttcaggccaagagttcaagatcagcctgggcaacatgcaaccccacctttacaaaaaataaaatactagccaggcatggtgggcacatatctgtagtcccagatacttgggaggctgaggtgggagaatcacttgagcccaggagtttgaggctgcagtgagctatgatcgcacccctgcactccagcctcacaacagagtgagactctgtctctgaaagaagaggccgggcacggtggctcatgtctctaatcccagcactttgggaggctgaggcaggtggatcatttgaggtcagcagttcgagaccagcctggccaacatggtgaaaccccatctctactaaaaatacaaaaattagccgggtgtggtggcacatgcctgtaattccagctacttgggattacttgtaattccagctacttgggattacttgaggcaggaaaatcgcttgaacccaggagatggaggttgtagtgagccaagagtgtgccattggactccagcctgggtgatggagtaagactctgtctcaaaaaaaataaaataaataaaaataaataaaaaaggaagaagaagcaggaggaggaagaaggaagcaaaaagaagaagaaataataatagtggagaatgcgcaaccttcagatctcctagttctgactgtttatatgacccagcataagcccttcccctcactaagcctcagactacctatctgtaaaatggaggtgataggccagatttctgaggggccctcctgctctaacaacccatggtttcatgaagaccagctttgtgcccagacatgtaaggcatataccgaaggacaagttattttttaaccaatggttggtgaatgagtgagtgttgaattaatgaatgaagtatgccttttttgtcccaatatcttgcccataagccaaatgaagagtaattcgaggtttgaaattgtgcaaactggccaggcacagtggctcacgcctgtaatcccagcactttgggaggtagaggcaggtggatcacttgaggccaggtgttccagatcagcctggccaatatggtgaaaccccacctctactaaaaatacaaaaattagccgcatggtggcacatgcctgtagtcccagccactcaggaggctgaggcaggagaattgcttgaacctgggaggcagaggttacagtgagccaagattgcatcactgtactccagcctgggcgacagagcaagactctgtctccctctctaaaaataaataaataaataaataaaattgcacaaacttaggcttaaaatcccagcccctctatttcccagatgtggatcttggggaagtcactttgcctcagtttcttcatcttgaaaatggaccattaatgtccagttacagcctcttccccaggacgctgtgagggagagatgtgacagtaaagtgcctagagcaggatgtagaaagtggaccctcagaaaataagaaccccagcctttctgctcacatgggagggcagggagctgaaataccttctgcttatcccaaggcattttcccccacccttctctcctgtgccactcagaaagggtaggagctactttggagagattttgattttatttattgtattgtataactatcaaaatctgccctaacaactgatacctaggctttgtgacaggaagccacaataactttttcttgacctgcagctgccctgcggataaaagataaaacaaagctggggagccactgagattatcaggcctgctgcacaggaaaagggcagtatgcagcaggggacctgggctccaggccccatactcagaaacctctgagggaggcagaggcaggtggatcacttgagcccaggaattcgagaccagcctgggcaacatggccaaatcccatctctacaaaaaatacaaaaattagctgggtgtggtggcgaacgcctctattcctagctactggggaggctgaggtgggagggatgacctgagcccagggaggcggaggttgcagtaagcagagatggtgccactgcactccagcctaggtgacagagtgagactccatctcaaaagagaaagaaagaaagaaagagagagagagaaagaaagaaagagagagagagaaagaaagaaagaaacctctggcaagctgtgtggttaagccaagtaactcaccctctctggtcctcagtttccctgtttgcctacttcccatgtgtgaggttcaaatcagattgtgcaagcctgtgaacactgtagaaacaataatcattatgtaagtgatcatttcgtcagcctttgctctatgccaggcacgggacgagcgttcttacaagagctgcaaaattagttttagcagtattgttcctgttttacatatgagaaaacctaggctcagcgaggtcagattccaaccaacccaaatcacgcatcttctcctggcccttcgttttctctcgggagacatggcctccctactattgcataattactttgtgaagttaaagagcgttgggtggaaatgagtgggacagattggagatgacgtgggtggggtgggaggattcaaggagggtacgcgcctggccggactgggatgactctgcggcaggcctgggactccctcaggccgacccaggcttcccaagtccctcctggagccaagggcccgggattctcgggcggctcgggcgagcgggcggggcaggaggcggggcggcggcaggaagctgcccgagcggcgctctgagcggcctgagcccggcggagccctgcagaacccggccgacctccatgggctgcggggggctgcacccggacccctggggcgcggcgcgcccctgaatgcaccgtgggacgcaggaggtagaggcgcacgaggcggcgcaagagacatcgggagggtcctgagcccctccggggaggaacgcggtcctaggctgagtgggttggggggaaagcgatgctggctgggtcccctggttcgcaagacctcggactgccagggggtggggcctcaccgcgcccctccctctccggttcgggccccgcccctcctcctcctgcgccgggctggggtacccctcctccgggatccggtacacagcgtcgcgcgctccccccagggtgccatggcctcccggctcctgcaccggctgcggcacgccttggccggcgacggccccggggaggcggcggccagtccagaggccgagcagtttccggagagctcagagctggaggacgacgacgccgagggcctgtcctcccgactcagcggcaccctcagcttcaccagcgccgaggacgacgaggacgacgaggacgaggacgacgaggaggctggccctgaccagctgcccctcggggatgggacgtcaggagaagacgcaggcgagtgcaggaggacggaggcgggaaccctggggctcgattaggcctccatcccagccgcgttcccagagcatccagggcgctaagttgatccagctgaaactcccacacttcttaaagcgcctgggccacctcttggctgtgtgaccttgagagagtcttaacccccctctgagccaagttttcccttgtgtcaagtgggagaactcactctcatctcagaattgctgcaggcattaaatgtacagggcttgggatccgaatggaaatgatgtattaacgatattttgagggtggagctattaatttagcccctactgtctgccaggcacggtgtttggctcctcggtgcctctctgtccctcagtttccatttctgtaaaaagggggtaagtcctgcctcacactgggaggatgagtgaggccacgtcagtggttgatgtcgaccctggccctaggcccattgatatgactggtcatgtgtctgcagaacggagccccccacctgatgggcagtggggcagtcagctcctggcgcggcagctgcaggatttctggaagaagtcccggaacaccttggcaccccagcggctgctcttcgaagtgaccagcgctaacgttgtcaaggacccgccctccaagtacgtggtgagtgagggtctagaacccctgggctgtgagtccagaagtatggctaggagcaagtagggaagaccccaacttcctgaggggtctatgtagttataaatggttaccttgtttacaaatggggaaactgaggtcagaaaggggatgtaacttgagcaaatcctcacacaagacttagtttccttgagactgactgaagtggggattaaggaccttcctcttaacctggttttgagactaagagaactggtgtgtgcacttccccacccagcccacaaggtttttgcaaattgtagcaagatgtgagatacagagagacagatgttatctgggtcaaacttgagatccaagttgcatgggctggtagttaaggaaataggctttcgatccagaccaatcttgactcccacttatttccagttgtttgactctgggtgagttgtttaaccttggtcatcttccgtttcctcattatagcaatggggaaaatgcagaagattacagcaggagttaaatgagacaatggacaaaaagcccttagcagtgtgcctggcattatgacgtggtcaatgaaatggtagctatgtcatcattatttaactttgggttgaaaatgtttgtcatctagggcctgctgtgtccttataacgcacaggtcaatcagttctcaagcacccgaccttgtccctaagggctagacttagagggttggtggaggttaggatgaagagcaatgttggggaggaccttggagtcccttgctgttttccttgctgttgagagactgacttggagaatgaggtctgctcctttaatgcccacagcgactcctgctccagtcccttttcaggccaggcctgaaagccagctgagctggccactcctgtcagtggcagctctccatctctcactcccctcctcttcaccacccttctccacctctgactcagcaggagggggcctggagcccagtcccacatctgggcctctccacccttttctgttgctgttagtaagcaagggccacacccatggggtgactactccaatttcagcagggaccgactccgattattgagtcttttaaaaaaatttttgaggccgggcgtggtggctcacgcctgtaatcccagcactttgggaagccgaggcgggcggatcacgaggtcacgagaccatcctggataacacggtgaaaccccgtctctactaaaaatacaaaaaaatagccgggcatggtggcaggcgcctgtagtcccagttacttgggaggctgaggcaggagaatggcgtgaacccaggaggcagagtttgcagtgagccgagatcgagccactgcagtccggcctgggcaacagagcgagactccgtctcaaaaaaaaaaaaaaaaaaaaaaaaaaattttgagacagggtctcgttctgtcactcaggctggagtgcagtggtgcagtcataactcactacaacctccaactgggctcaagctatcctcccacctcagcctcccaagtagctaggactacagctgtgcaccaccacgtgtggccaatgatgaagtctgaagtggcaaaggtctcagaactctgggttcatacactggaaggggacaccaaggaatagttcagaggctgagaggaatgggggtgactgtgggtacctggatgggaagggatgacaagatagagcaggatggccaagtatgccccggcctgtgtctggagaagcaaggcacctcctgtgtacaatggggaccctaattccccactcaacttgtgggggtgaagagcacaggctttggaatcagatcaacctgagtttaaatcccagctgggctgttttccagctgtgtgacttacggcaagtctttgagcccttttccccctctttgagcctgcttcctcagctgtcagagataaggaccttccttgcagtgctgttgtgatgattaaatgaaatcacagatgtaaagagtgcttggtacaacaggaggcacagggtaaatgcttgggtaatggaactactgtcattatcacccatctggctgcatgttacaaggtcaatgagataatggatacaaaatgtaaactagatgaacataaaggcttattatatagaccagtgtgtcctgaagaaaactggtgtcctaaagtgtgtcctgcagaaaactagttccctggaatgttaatagctgtttctaaataaaggttttgcaggcaatatgttttgagaatgcagagttaaatgagaatacaataagtttctttattgttaaagtatttaatccctaatagtgatatacactgtgaatcttcaagaggacagagtaagcaaacctaactgaccaggagtggctagagtattgtcagttccacagaacaaatgtgagagaaagatctggatttggtgttagggtgcctttaacatctaacactggttaactctccttttaaacctgttggcttggcattataggttaatccatattcaggttctcagacctgggaggcggggaacttcgtgatcatcttgttcagtgttttccaaacctcagtcattccaatacctccctgattcttgtcctatctaaaaatgccacttgtgtccttttcttcttaacatttttctttaaatgaacctatttttttttctttatctttttttttttcttttttgagacagagtcttgctctgttgcccaggctggagtgcagtggtgcaatctcagctcactgcaacctccacctcctaggttcaagcaatcctcccgcctcagcctcctgagtagctaggattacaggtgcccaccaccatgcctggctaacttttgcattttcagtagagatggggtttcactgttggccaggctggtcttgaactcctgacctcaagtgatctgccgcctcagcctcccaaagtgttgggattacaggtgtgagccactacgcccagccgaacctattttttttcttaaataagtttatcgagacggagtcttgctctgtcgcctaggctggagtgcagtggcgtgatcatagctcactgcagccttgaactcgtgggctcacatgattctctgcctcagcctcctgagtagctgggaccacaagtgcccaactaattctttttctttttctttttcttttttttagagacagggtcttgccatgttgcccaggctggtctcgaactcctggccttaagctgttctcctgcctcagcctcccaaagtactgggattacaggtatgagccactgtgcccatcctttaagtaattttaaaagacaacttcatagcactaccgtacgtggaaagtctcacttgccaccgagagaagctagccataaaaataaaaggtgaggcacggtggctcacacctgtaatccccgcactttgggaggctgaggtgggcagatcacctgaggtcaggagttcaagaccagcctggccaacacggtgaaaccccgacactactaaaaatacaaaaattagccggatgtggtggtaggcgcctgtaatcccagctactcgggagattgaggcaggagaattgcttgaacccaggaggcagaggttgcagtgagcggagattgtgccactgcatgccagcctgggcgacagggcgagactaaataaataaataaagtaaataagtaagtagtgaaaatgaaacgtcattaaatcttgcctagatgttgttgcctgaaggaggccctgacctgagatccactttctgactcttttctaaaaatggagatgagaagtgttagagaagtactttttttttttttttttgagagagagtttcactcttggtgcccaggctcgagtgccatgagcaatctcggctcaccacaacctccacctcccaggttcaagcaattctcctgcctcagcctcccaagtagctgggattacaggcatgtgccaccacgcctggctaattttgcatttttagtagagacagggtttctccatgttggtcagcctggtctcgaactctcaacctcaggtgatccacctgcctcagcctcccaaagtgctgagattacaggtgtgtgtgtgagccactgcaacctgcccaagaagtcctttttttaaaaccaaactgagagctggccaggcgcggtggctcacacctataatcccagcactctgggaggcaaaggcgggcggatcacaaggtcaggagatcgagaccatcctggctaacacagtgaaaccccgtctctactaaaaaatacaaaaaattagccgggcgtagtggcaggcgcctgtagtcccagctactcgggaggctgaggcaggagaatggagtgaacccaggaggcggagcttgcagtgagccgagatcgtgccactgtactccagcactccagcctgggcaacagagcgagactccgtctcaaaaaaaataaaaaaataaaaaaaccccaacaaaaccaaactgagagcttctcaatttgtagtcagaaagattaaaaaacaattaggctgggtgtggtgctcatgcctgtaatcttagcactttgggaagcctagctaggaagattgttcaaggccaggagttcaaggccagccctagcaacatagtgagaccccccctcatctccatacaaaaaaaaaaaaaaaaatttttttaaatagctgagtgtggtggtgcatgcctatagtcctagctacttgggaggctgaggcaggaggaatgcttcagcccaggaggttgaagctgcagtaagctatgattgtaccagtgtactccaacctggccaacagtgagaccctgtctcaaaaaataataattaaaaggggaccaacctcctgatacagtgatatttactctagtgcacaccacctaaaaacctcaggtaccaatacttgtacatgtctcatttttggacaaacactgacttgaccgagcacttccttcactagaaacagaagtggacccagagggggactcccttgcctgggtcacacagagaaggagtagctgacctggggttagaacttgagtcctaggccagtatacttttcacagttatggcagttgtggctatctacagacctgtcctctgggtctagtttggaaaagcctgaccagggatgtggaggccaggtgtccaaagtggaacaagctccaagctggtcctaaaggagccccaagagattggctggggtttcagcttatgtctggttttccagagggggctgtcagaagaactaatgctctatgggaagggagtacaaaaaaaggggcagcagccccgggcactgaagctcatctctgtctttctctcttcttgctgccttgggcctgggtcatttcagacaaacctcaggtaagatgggactgggcttccccgccactgcgcagcagctcccgccctcctgggtgctgtcccaggggtgaaaggaagaggtggggaggtacagctgggagtgtgggggatggggaaggatggggagggaacgggcccgtggacaacttgcatttgccctgacacccaccctccctgcagctctacaccctcgccgtgatcggcccaggaccgccagattgccagccagcccagatctctcgccgttactcggactttgagcggctgcaccgaaacctgcagcggcaattccggggcccaatggctgccatctccttcccccgtaagcggctgcgccggaattttactgcagagaccattgcccgccgtagccgggcctttgagcagtttttgggtcacctgcaggcagtgcctgagctgcgccatgccccggacctgcaggacttcttcgtgctgccggagctgcggcgggcacagagcctcacctgtactggcctctatcgtgaggctctggcactctgggccaatgcctggcagctgcaagcccagctgggcaccccctctggcccagaccgccccctgctgaccctggctgggctggccgtgtgccaccaggagctggaagaccctggagaggcccgggcatgctgtgagaaggccctgcagctgcttggggacaagagcctccaccctttgctggcaccctttctggaggcccatgtccggctctcctggcgcctgggcctggacaaacgtcaatcagaggctcggctccaagccctgcaggaggcaggccttacccccacaccaccccccagtctcaaagaattgctcatcaaggaggtgctggactaacccttgcctagatttaaggccactgtgaggagaggggttgccccagaaggcaggggaaggacctgatgagaacagaatagctgggaggctgcagagggtgctgggagcccctagaagttccaaaagagaatgtgaagcagatcaaggaaacttctgttgagctaggctcagggtgagctttggctggggttgcccttgtgtagtacagggaagtctgacacagcctctccagcctataaacagccggggggctgtggcacaggttggggcaatgttcccttgttggtgggcccccaagctggcaaggcctcttggctgaaggccagggactctgcccctggagtcctggagttaagggatgaaggcaaggctgcaggtctggcccaggggaattaaaagccagccactccagtggtatcagtctctttattggatgtgagggccaaaagggactgtaactcctgtctcaggaatggggatagatgggaggttcttgaagccccaggcgaagctggtacctctggctacagcttgctctctgagacctggggcttcactcggatcacgccctcctgggcacaggtcacagctaggactccatcctgacgccacagccgcccatggaccagcccccgagagccacctgtgggtgaggtgaagggtgatgatggcctgcttcagaacagccaaatacactttttttttttttcctgaaacagagtctcactaagttgccaggctggtctcaagcgcctgggttcaagggatcctcccgcctcagcctcctgagcagctgggattacaggcgcacatcaccatgcccaacctccaagtggacttcttgcaaagggtctggcccagggcagggctgccccacacaagggtgcactgagtgtcgtggctgctccaaatgccccttcatgagcttattatggaccgtcattgaggggtaactcctcccacaggaaccccagttgacagtttaaaagcactttcacacctctcctcgcttcctcaaaaagatcacagagggaggagctctgagaacagtctccttcaacagttcggccaagcagaactgctgtacctctgaccacttgtgttaggaaaactatcggctccctgtataataaatcaagccaggtcctccaagtggtaattcatgaaaaatatccccactaccaccaccaaggggaagaaaggactcagaagagaggacttgaggccatgaggtctggcctcttccctccccatctggagactctttctcccttgctggctttgggcccaagtttgatgtttatgaggatgattgctggtttcccttacacatagcagagctcactcagttctcacagtagccaaatgaagcagttatgtgttgtccagttttccagaccaggactgaaatccagagagggtcaggaatttggccccatgatgaagccaaatctgaacccatgtcctcacttcaaggttatcaatcttggattgtgatgctattggtacttgagaataccccttatctgagtataaagaatccttgagttttgtccttggtttatcaagcaaagcttttcttcatttgaaatgtactcccttggaaaggaggtcagggttctgaagctagacattgatgaacgagtcttgtttctctcccctgcaaggaaggtccaagcaggcccttagggaccactgaatgccccgatcccaatcaggttaatcagaatcacttagagaacttaaaaatacagtttcctggaccttatccaagacctactgagtgagaatcttgagggtggaatcagaatctattttgaaaaggcatccccaaatggcagtctgatggactgcgggtttggataccactgctgtaatgtaaccctctttgtttagatgaggaaactgaggttcagatggaagatatgatttgcctattgtaatacaaagaatctgaactggttttgggactagaactgggtccctgggagaaggtgaatcacatttccagcacattctagaaatttagagcagagcaggtgtcccggcctcaccctctcattttgaagatgaagatgccaaagcctgggagggaaggaaaggaagtgacctgtgcagcgccacacaggaagtcgggaaatcagcatcacccaagaaagcaggctcctgccactcaaggtcagtgcccttgtcccacacggccccacactcaccggcccaggggctctcgcattcatagagcatccagtggtcagctcggaagggggcgtggaaccacatggaatggtccagtgagaccatgaagtgcaccttgtgctgccactggtgaggcagcagtgcagtgcccaagaaggcatagtcggagatataggcggccacgcagcagtgcatcttcatgtcgccctcgcctgcaacaggtccccatcagccctgggctcctgggctttccagctcaggacctgcacggaggtagggggaaaagggactcccgtgcatgggtgtgcagactggttgctgcacaagagcgcttggcctacagtgtgtgtgtgtgataggggagaagtgagatggcgacttggggagggcaggggtgagcagggactgaaattcagtcttcaccacccaagtctcccactctaggccccaaaaggggtgtcttcttccaatttgcacaaagatactttatagttagcagcaaagctggacaacaaccccagcgaggtgccttttgccttcctcagaaaggccttgtgtcgggattatagtcatctccaccttctccccatgcaaagggctgagagggcccaggtgagaagctaagccaacttccccatcatgcagataaggaaacaggcccagagaagggaactcatgtgcccaaggccacacagcaaatgagtggtagaccccttggagagtggtttcctcccatccatggggtactcttaccaatatagccccgggctcgcacccagaacatctgtttgggctccattctctgcagctggctcaggggggatgggtttactggcttgatctcaatggggacctcctgagcagcaattcggttgagcgccaatgggtacctcttttggaggttagggtccctgaaagtaaagggaagagggtcggggtgagacccaggaagagagggagtcacatctgcattgcctcttctgactcccctcattcttccacagggacccctgatatccccaaagctcccctcccctgtggcaggcaggatagtgcagcgtttgaatcccttcttggccacttacgagccatgtggtctcgtgctaattagtaatctgagtctattttcccatctgcaaaatgtggataatgatagtaacctacctcataaccaacactgtgaagacaagatcatgaatatctaaccaacacatcatagtaataaggcataccctgtaatatgcactgttctaagcattgtacatacaatagtattcctcacaacaaccctgtgagataggtactattatcacccccatttcatcagtgaaataagcacagagtccaggtagcttgcttaaaaattggagagacaggatttggactcaggcaatctgggtccacagtatgattattttctatttttttagagacaaggtcgtcttctcacccaggctggagtacagtggcgcaatcagctcactgcagtctccaactcccaggctcaagccatcctcttgccttggcctcccgagtagctgggactagaggtgtacgccactgtgcctggctaacatttttattatttctttattttgagacggagtctagctctgtcacccaggcttgagtgcggtggtgcaatctcagctgactgcaacctccgcctcccaggttcaagtgattctcctgcctcagcctcccgagtagctgggattacaggcgcctgccaccatgcccagctaatttttgtatgtttagtagagatggggtttcactgtgttggccagggtggtctcgaattcctgacctcatgatccgcccgcctcagcctcccaaaatgctgggattatttttacttttttttgtagagatggggtctctttatgttgcccaggctggtctcaaaatcctggccttaagtgatcctcctacttcagcctcccaaagtgctgggattacaggagtgagctgctgtgcccagccacttcattattttaaacactcaagaaacaggctgcctgacttggcctgagttaccaaaccctacacagtagggaacattgcatccccccagggggaggagcccattccaaggaactcccttcaatacagtgacaaccacccatagtgctctgcacaggtcctgccattttcacccctatcctctggagtgctgttggacagaacctcctatgatgatggaatgcctatctgcacagcacactatggtagccactagccacatgtgactatggagcacctgaaatgtggctaatgcaactgaggaacagaattgttcattctactgactttggtttttttttgtttttttttttttaagacggaatctcgctctgttgcccagtcaggagtgcagtgactcaatcttgcttactgcaacctctgccccctgggtccaagcgactctcctgcctcagcctcctgagtagctgggattacaggcacgggccaccacgcctggctaatttttttgtgtgtgtatttttaatagagatgaggtttcaccatgttggccaggctggtctcaaactcctgaccttgggtgagccacccgccttggcctcccaaagtactgggattacaggcatgaaccactgcgcccggacctatttaattttaattaatgtaatatagccacgagtggctagagactaccttattaacagcacagatctaaattcaaggtcctttaacaatgtcttcctttcatttcagcaaacatttgtcgaactgcaaccacacaccaggccctgtgttttttctgttaatagtgatataactacaattcactgccattgtgctagttagtactagcacgctaagtgccttttctctcttatttaatcctcacagcagccttatgaggtaggaatatgattagcaccattttataatggtgagtactggctcagagtgggaaagcaacttgcccaaactcacacagctagtagggacaaaatctagaattgaatccaggcctttcaggttcccaaacccacatccatcccgtcatactctgctgcctccccagtatctaacacgagatagagacattctctccttatgatacagctaagaagtaggcagggcagaaatgagcattcccattttttagaagaagaaactagggccctggggagttgggcactgtcccacagcaggtgagcattcacagaggcacagccctggacagaatccatgtgtctatcctttgtctggacccagagctctttcccctgtaccagcatttttcaaacttgattaggtgtgatccaaataaatatattttaccaggtattcaaaatatctaacatgctccctggtggtctaatggttaggggaaaatacatatttgtatttttgtacatatgttttatatacataaaacatgtggccgggcacggtggctcacgcctgtaatcccagcactttgagagaccaaggcaggtcgatcacttgaggtcaggagttcaagaccagcctggtcaacatagcgaaacctggtctctactaaaaaaatacaaaaattagccaggcatggtggcatgtgcctgtaatcacagctgcttgggaggctgaggcacgggaattgcttgaacctgggaggcagaggttgcagtgagccgagatcatgccactgcactccagcctaggcaacagggcgagactctgtctcaaaacaaacaaacaaacaaaaacatgcactaatgcaaaggcagtgaccaaacataggccatagcgctgcagctctccagaggacactcactcctggtgtgcattaagtcctaagttgctggaccaggggcctgggggagagagggaatcacttagggggctggggaggtggtacttaacaagtggttacggaagtatttctatatacatatatatataaaatatatatatggttttttttcccaggtatggtggctcatgcctataatcccagcactttaggaggtcaaggtgggtggatcacctgagatcaagagtttgagaccagcctggatagcatggtgaaacgccatctctactaaaaatacaaaattagctgggcatggtagcaggtgcctgtaatcccagctacttgggaggctgaggcgggaggatcacttgagtctgggagtcagaggctgcagtgagccgagatggtgtcactgcactccagcctgggcaacaagagcaagactccatctcaaaaacaaacaaacatgtatatatataaacaacaacaaaatatatattctttttttcttttttcagacagagtcttgctcttgttgcccaggctggagcacagtaacacgatctcggctcactgcagcctccacctcccaggctcaagtgatccccccgcctcagcctcccaagtagctgggactacagacacatgccccgacacccggctaatttttgtattttttatagagacagggtttcaccatgttgcccaggctggtcttgaactcctgggctcaggtgatcctcccacttgggcttcccaaagtgttattataggtatgagcccccacacccagccccagccttatttttaaattagagatggagtcttgctatgttggcgaggctagtatccaactcctggtctcaagcaatcctcctgccttggcttcccaaagtgctgggattataggtatgcgccactgcacccggccaagcccttcattttataccatgacccagtacagacacacaccagaaacacacatgactaaacctcaagtttcacaaaaggattcttgcccttaatacatacatattattcactccagtatcttcttttctaatctgtattttaaaaatgctggtcaagacccactaaaggattctatgacccacaaacagatagcagctgcattttgaaaagtctgccatggagcctccaggtctctcaccttaaatactggtcaatgagggtctcacagtcaagcagctcttctggtggtggcacagtgggcatggagaactggtgctgcatggggctgggctgggcctgctggaaggaggcctggcagatgaagatgggcttcccatgttgcacggccttcacagagcgcaccgagaagctcgaccctgttcgtgtccgctccacttggtacagtactggcagcttcgggtcccctgcagtgggcacaaggacacagtgggtccacaggccctgtcatcatcacaacgcctgacaggcactctactactcatcttccattccatttcatctcagtgactactaactgaggtctgctaaggtccaggcccagggatacagagaccaccaggccctgtcatcaggcttctccctgtgcaggtggagcttttctctaacagcacaaacagcagagacattgacagattccttgcattcaaatcctggctccaccactcaccagccgtgtgacccttcacttctctgagcagtgtgcttatttgcaaactggggatcatcatggtgctcatcgcttacaggattttaatgataatgaaatgaatccgtagttgtaaagtgtttagaataatatctggcacatagtgagggctctatatgtgtttgttaaataaaattaatactccttgcttctatcttgctccatctatgcctgggatcttgtcatcagtccaaaaccattattctagatcagggagcagtaaatctagcctgcagcctgtttactgtttattctgttgttgttttcttttttgagaacaaacaaaggctatttattcagcgcttgttcttttttttgtttgtttgagatggagtcttgctctgtcacccaggctggagtacagtggtgtaatctcggctcactgcaacctccacctctcaggttcaagcgattcctgtgactcagcctcctgagtggggggggactacaggggtgcatgaccatacccagctaattttttttttttttttgagagacagtttcgctgggtcaccgaggctgaagtgcagtggcgctatctgggctcactgcaacctctgcctcccaggttcaagccatgctcatgcctcagcctcctaagtagctgggactacaggcgtgcgccgctacacccaacaaatttttgtattttttgtagagatggggtttcactgtgttgcccaggctggtctcaaactcctgacctcaactgatccacccaccttggcctcccaaagtgttgagattacaggcatgagccactgcgcccagccaatcagagcttgttgtagcaggaagtcaaccaccatcacttgcttttggcagagacttagaggcaggcaaaggaatgggaatgctctgtagtggaaaaaaaggcttcagggatgctcccattgcaggctgttggcatggggaagctgttggtgggctaactagaagcatgacacctggctgggcacggtggctcacacctgtaatcccaacactttgggaggctgaggcgggcagatcttctgagaccaggagttcaagaccagcctggccaacatggcaaaaccctgtctctactaaaaatacaaaaaattagccaggcctggtggcacacacctgtaatcctagctactcgggaggctgcagcaggagaattgcttgaagctgggaggtggaggctatggcaggagaactgcttgaacctgggaggtggaggctgcagtgagccgaaattgcaccactgcactccagcctggggaacaagagtgaaactctgtctcaaaaaagagtgggacatcctatgtgattggttaggagtacatatttggctttctccggttggtcctaagttggaagcaggggcaaaaattagggcagctatcagttattaatcaagtcctggctttggaggccaattgttacagtggtcattatttggcttgctggactggttggtaaagatagtggtctgacttcctacaagtctgacttgtggatagtaggctggcttcctgtgctggttgctgctattgtgcgtcagagttctgtttttctacatggtctagccattgtctgtttgtatattcagcctctcccaggcaagggccactgtcagtcacttcacccctgatgtatatggaagccatttggtccttcactgtcaggtgtctctgctccaaagcagagtggaagagcctaggcaacacagggagaccctgtttctacaaaaaataaaaaataaaaattagctggacatggtggcatgtgcctatagtcccagctaatcaagaggctgaggcggaaggatcacttgaggccaggagatcaaggctgcagtgagccatgactgcgtcactgtgctccagcctaggtgacagagcaagaccttgtctcaaaaacaacaagaccaccaccaccagagtagaaggcactggccagggataaggcagccaacaacaagcaggtgagggaagatttggagaggaagagaacttccctggtttccctgaagttctcactttgtctgagtcaggctgaccttgaggaaccctgacatctgggccatgagacatcttggggatgactttcaaccttcagatgaaagaagcaggtctagagaagtgttgcttccttcactctgatccaaatggggagtcaaccaggcaccagggctcaatcaacagcaataaacatcactgtctgaggtcaactggcttgctcagctctctgttatcaggcactgccaccagcagcacttctatcagagtgcgagctcctcaaggttaaaggcatttgagaagggcttaccaagagtccagacactagacaaagttacttgtgcagtctgctttcagacactgggaatgaagtcttcctccctaccaccaaacttggggggaaaaaacaaaaaaaaacctctaatccaatccttggtctgcaggtaagctaaagtgcagattcctgaggaggatcacagaaacttcagtactctggtctacctttcagtgtgaatgagttccagccctattcttgaaaattgttattggaacagagcaggtaggccctgaggcatattgaagggagttaatgattatggagtgatgcagggtgcctgacagcattggacaggagggtatgttttctcatgttggtctcacaataactatgtagagccactcttacccccattgtacagatgaggaaactgttgccttaaaatacaatttttttgtaataaagtcatgctaatttgtttacatattgtctatagttgctttcactggcaaaacctaaaatgtttactatctggccctttctagaatgtttgctgaccttgttctgaattcttcaagctctaaatttacctctgtagagaaaaactaaaaacaaaacaaaaacccaactaattcacattctttcctgggcttttagaaaaagaactgatttgataaatggccaaggaccaaccctccccctcccccataacatcttttatttttaaaatgtaattgaaacatctcttttttgttttgtttttttgagatggagtctcgatctgtcgcccaggtgggagtgcagtggcgcagttttagctcactgcgacctccagccgccgggttcaagcaattctccttgcctcagcctcagcctcagcctcccgagtagctgggatcacgggctaaggtcaccacgcccagctaattttcctttttttttttagatggagtcttgctctgtcacccaggctggagtacagtagcacgatctcagctcactgcaacctccacctccagggttcaagcgattctcctgactcagcctcctgagtagctaggactacaggtgtgcaccaccacacctggctaatttttgtgtgtgttttttttttttagtagagatggagtttcgccacattgtccaggctggtctcgaactcttgacctcagccgatctgcccgcctcggcctcccaaagtcctgggattacaggcataagccacatgcccagccaaaaaacagcattggttttaatatctgatatagctggcagagtcacaccacaaacagataagagctctcccctttttgccttgcagccaggaagctcagaactaaaccttgtatgaaactgcaacaatatggcttagccaaattttttgacatgatcgcttctcccaaatcccattactactggcctaatgtcctttgacttttaatattcccataataactaccaattattttagctatcttgcagcctttcaagacacaagcaagggcacaaataattatttttatttatttatttatttttgatggagacaagagtctcgctctgtcgcccaggctggagtacagtagcatgatctcagctcactgcaacctccgcctcctgagttcaagcaattctcctacctcagcctcctgagtagctgggactacaggcacatgccgccatgcctggctaatttttagtattttagtagagactgggtttcaccatgttgcccaggctggtctcgaactcctgagctcaggtgatccgcccgcctcggcttcccaaagtgctaggattacaggcatgagccaccacgcccggccaaataattatttattttgaaatggtggcttggacacagctcatttcacgctgctcctggatgaagactttccaagaaagaggatcttttccccagaacctgggatgggtaccctggcagctctctcattgtttagaattattgcccaagagcgaagcaaagaagcaggaaagagtttggcactcaaagacacataaaccaggttcaaatcctggctctgcctccttgggcactgtgggagctggttccaaacctcttcaggtcccagtttccttaggtaagaatgatggcaggaatgcctgccacccatgtttacttatgaagactaaacaagataatgcacacaactatgctctgtatcatgcaaattttaattattcccataagttaatcatcaaaaactaataatatttatggaaaactagattatgccaagccctgcactagatactttactggcaaagactcattgaattcttccatcacaggatgaagagattcagagttcaagtaatgcccccacccacacaaaagaaaacagccaggtatttcactaggctggaaggattacaatatcaaaaggctggtacttacaaacaaattaaagaaacaaagcacatatttactcccaactagcataaagataagaacctggctgagcccaaggtaagtattcaaatactttatagcagtgactgggggccgagaagaggggggccaatcaatactcctcagggatgaggagtaagagacacgggacagtatgaaaaagtgtatttaccattacagggataactttcatatttacaaaatgaagggggaaaccccatggttttcttaatacaaatcacatactgtaaaggtccacaaactcttctcagctggggatctacgcagaacacatgagggcatctgctctgggggtaacagggaaggaaagaaaacagtgagggttttttttgtttgttttttagcataggttttgcttttattttccttttatttatttttttgtagagacaaggtctcgatttattgccctggctggtcttgaattcctgggttccaacaattctccgcattggcctcccaatgtgcggggattataggcatgagccaccacacataggttttttgtttttgagacagagtctcgctctgttgcccaggctggagtgctcgcccaggctggagtgcagtggcacgatcttggctcactgcaagctccgccttccgggttcacaccattctcctgcctcagcctcccgagtagctgggactacaggcacccaccaccacacctggctaatttttttatatttttagtagagacggggtttcaccgtgttagctaggatggtcccgatctcctgacctcgtgatccgcccgccttggcctccaaagtgctgggattacaggcgtgagccaccgcgcccggcctcacacataggttttaagttacattatctgaccccagagctgtaagaagctgtagagatcatcccactaaaatacccttggtcccacccttctccattttacagctgaagtaaaccaaactgtagggcggggaggtaacctcacaaggctgcatggtaaattatggacaaaactgaagctaggccctacatctttggtttgtttttattgaccctatgctcagttgacaaaagaagccttagatcccttgactctcaggctggtgctctcttctccaacccaagctgccctacctccacacctctagcacagaggtcacagcacactgagaacctagcacagcgctccgcaccaggagctatgttaagaatggaagaacaaaagcccacaaggagcagttaagcaacaaagtcttgtgtcattatcagccagacatcaggtacccagtgagtcccagcaggtgtgaggtccaaggctcattgttagccccagacctcgttccaccctagacacctcttagggtggtagtgtccgccacccctccccttgtcttccagtctccctctcttcccttttgacctcagcccccaaggccagaaatggctgctgggccaccagggttgggttggggcaggaagtggggggtttacctgcccgaacaaagtagcagtgcagggagtgcacgtggacgtcttcactcacagacttggctgcagccaccagggcctggcccacgatctgaccaccaaacagcctcttggccggtacccagtaatgccttcctctgtagggagagggggaaagagggaaagacttgggaactgggccaaattctactaccctcactaagactctaatgcatgatctcttcaccatattggggtttaagatccttttgagaatatggtaaaagccataggtcctttccccaggcaaatatccttatgcataaaaatcctctgggccgggcgcagtggcttacatctgtaatcccagcactttgggaggccgaggcaggtggatcatttgagatcaggagttcaagagcagcctggccaacatggtgaaactccatctctactaaaaatacaaaaattagctggacgtggtggcatgcacctgtaatcccagctactcaggaggctgaggcaggagaatcacttgaacccaggaggcagaggttgaggtgagccaagatcgcaccactgcactccagcctgggtgacagagcaagactccatctcaaaaaataaaattaaataaatgtataaatgaataaataacaagagccctcagacatcaagtctaactccttcagtgtacagatgagaaaactgcagcttggccccagagatgaaaatgcttgcccagaatgtaggcccattggaagctgaataattatgtctctagcctcccacgtcccagctgtttggcattttctgtaacaatgagttatgaacagccctccatgacctggcaggtggctctcgcctgtaatcaatcccagcactttgggaagccgaggtgggaggatcgcctgagcccatgagttcaaaaccagcctgggctccaaagtggggcctcatgtctactaaaaataaaaaagttagctggatgtggtggcaagcgcttatggtcccagctaaacgggagactgaggcagaagggtcacttgggcccaggaggtcgaggttgcagtgagccatgttcgtgccactgcactccagcctggacaacagggccagatcttgtcttcaaaaacaaaaatcaaatagccctccaagaagttatttacaattccttaagtggaaggaagggagctcaaagtaagataacttccagtaaagccaggagaagccttggaggtcaaagctgttagaagccttgctgcagggggctgggcgcggtggctcatgcctgtaatcctagcactttggcagaggtgggcagattacaaggtcaggagttcgagaccagcctggccaacacggtgaaaccccgtctctactaaaagaagttaaaaaattagctgggctgtggcgcgcctgtatgtcccagctaatcaggaggctgaggcaggagaaatgcttgaacccgggaggcggaggttgcactgagccgagatcgcaccactgcactccagcctgggcaacagagcaagactccgcctcaaagaaagaaagaaacccttgctgcagtcaagaacttcagcgatgctgggaaagaaaaaaacatgcttaggcctcagaataagtaatgagaaaccagacccaccaaatggtactgggggcgggatattgtgagaagaaaaggatactgaagacacaaggagctgcggcgtcggttttaaggctgatccatcagctgtgtgcccttgggcaaatcactgccctctctgggctagctcccccgtgaaggcttagggcaggctgccgagcagggtctctcaggtcaccactatatttgctcagctgtgcagacctcgcagcgcggagaggaagggaggaagccagagcagagaggacagctctgttgggggcgagttctctcctaatcgtggcagagggtgcatcccacgcagagccatactagtcccggagccaggggccccgggcggcggcagtcaagatcaagtttctgccctaaaggaggggatgctgggcaggagctgccggctacctgaagagatcctcgtccagcggctcgaggttgagcacggtcgtgaccaagacgctacggaggtccccagggggatcgccgcggtcgccacagccctgcccatcttctggggcctgcggggacgacatctagttcaatgctgcaggccctgcacacccgctccgcggaagacgcggagacatacacagaacctgactcttccggcagattgccctagtaaccggaagtctcctgcagaccccgggaaggaggaatctaggttatgattggtcaggactcctgtcagtccgctggtggctgtggccctctttctctagcaattggagaaaatgaatgccaatctgcgaatcctgtgtgtgactggatatgtcgctgaggaggcggaacctcgggactggataatatggactgagtgtcatttcccctgtcagatagcaaatacacccccttcctcttgtaacccggtcaggcctagggttcctccctgagttccagaataggccacccagttggggcggacccttaaggcattctgggcccacgcctgcctataaaccctcatagtgtgacctttgacccctggtgtgatcttgggcccgggctgggaccagcccctagtgtgggttgtgggggcggccatggagctgggcagctgcttcaagacctatgaggacttcaaggagtgcttcagcgcctacaaaagggagaacaggtgctccttcattctcagggactgcgtctccgtccgcttccacaacctcaaccatggcacctccatccgcgaagacatcctgtaagggcgggcggggcggggcgggccaagagggtggggaggaggctggacctccggagggctgcctggaggaggaggggtgcataggcacgcattgggctgcgtggccattcattcaacaggcactcattgagcacctgctgtgtgccttgtgaagagagctaagcctccacggccattggtttttgaccacccactccactttcactccattcacaggcttttctggaatttataactgcagtttctcccattgttttgagcagctgcagcaacccaaatacaaagtatggctttcataaacggataaaaacaccttggcgataatttccttcatcagtctcctctttacaaaacagtcacatggtcgttggaatgttcgttcattcattcattcagtttttagttcttatatatttgttgaaacagggtctcactctgtcccccaggctggagtgcagtggcacaatcagctcactgagccttcaactgctgggctcaagcgatcctcccacctcagcctctcaagtagttggaactggaggtgcacgccaccatgcctggctaattatttgattttgtacagactgggtcttgttatgttggctaggctgttcccagactcctggactaagcgatctttcggcgtctcaaagtgttaggattacaggcatgagccactatgcctggtctattcattcattttgtaaatggtttttgagctccttatattaaccgttttcaaacactttccatgtgccaggcagtgttctctatgcatgctttaccctgcagggcttttttttcagaattatagataaatgttatttttttgcatttgacagatgagaagatttgacagatgagaagtcaggcttagaggttaagacaggagttgaaaactcaaatgccttcagggattaggtgaaacaacccaatgtaagaaagttttaactttgaattttactttaagaaaaagaatgatgaaatagcaaccaacaccaggtctttgtgctagggacaacagaagtgtgaaatgggccaaatggagagtagatgccatctaatcagctccagctgatggctgccatatgaaaataaagatctagtgttgccagaattttgggcaagagttgtcagaaatccacactttatgtggattttcccatttaaaaaaattgaatcagttcaaattttaactcagagcatgcgggccaactcagagttaaagcatatccgagtttgagacttggattgagatgctcaaccaaagctcctcaattagacagccaggctatattctttccactgttctgaagtccagtgctatgctgggtataggtgacataaagatgaataagtgttcaaactatccccagagaactaggataaacttgaactcaagcaaactggtgtgtgataaatgctttaaaaggaatgtggaaaaaaaaaaaaaaaaagaaatgtacggagaaagaaatgaatgacaaatgcctgggaggtgggggaagtcttcacactggaattgatggtttccctgggctgtgaatgaatgtagagtgctagagtagatctgtggttaattactgagtaccatggaggcatttgaccagaagttgctgtgcagaaggacctacaggagcaaagacaaagatacatgaaagaaggttggcatgttagagtaacagtgagggataccatgtttggcaggagatgaagtcatttgcagtctaagggtctttagggaccattgtgaggtgtttggaccttgtcctttatgggtaggaccttgctaagcaatgagaggaatacaaaaaaaaaaaaaaaaaaaaaaaaccacagttgcccgtaaacacccttcaggaagaccatctcaggtggctagcacaaggtgtggcccatagcaggcattaaataattattgaatgagtgagttatcaagtactaaccatgtgattcagggaaataggtgctgcgggaactcagaagagaaaaatcactgcgagctagagacgtaagagtgaaagggaggcttgagctgggcccccaaagatggagagctgtcagaaggtgtattggttttctgtttgccactctagtgaattaccacaagtttaatggtgcaaagcaacacaaatttattatcttagagttatttaggttagaagtcaggcacaggtcttagcaggctaaatcaagatgtctgcagggctgtatttctttctggagtccctgggtaagaatccatttcccgccgggtgcagtggctgatgcctgtaatcccagcactttgggaggccaaggtgggcagatcacttgaggtcagcagttcaagaccatcctggccaacatggtgaaaccccgtctctactaaaaatacaaaaattagctaggcgaggtggcacatgcccgtaatcccagctactcaggaggctgaggcagcagaattgcagccgggaggtggaggttgcagtgagccaagatcgtgccattgcactccagcctcggcaacagagcgagactccatctcaaaaaaaaaaaaaaaaaaaaaaaaagaatccttttcctttccttttccacctcctagaggctgcccaacacaccttgggtcatggcccccttcctccatcttcaaagccagcagtagcaggttgagtcatcacatcactctgaccttgcttctgtcatcacgtctctgactccctcttctgcctccctcttccacttttaaggatccttgtgattatattgggcccacccagataatcccagataatttccctattttgaagtcagctgatagccaccttaattctatctgcaaccttaattctcctttgccatgtaacctaacgtattcacagagtctaggattaggacaaggacatcttcaagggaccattctgcctgccacaagaagattaataacctggaggtaaaaatggtgtgagtcagtacagacgtaaacatgacgtgcttctattagaaaacagtgatgggctgggcacggtggctcacacttgtaatcctaatactttgggaggctaagacagggagattgcttgaaatgaagagtttgagaccagcccagactataatcctgggcaacaaaatgagatccccgtctccacaaaacaaaatttaaaattaggggccaggttgtggcagctcacacgtataattccagcactttggtaggctgaggcaggcagatggcttgaggccaggcattctagaccagcctgggcaacatgccaaaacctcatctgtacaaaaaatacaaaaagtagccaggcatagtggtgtgtgcctgtagtaccagctactctggaggctgcggcaggaggattgcttgatcccaggagtcagaggttgcagtgagctatgatcgcaccactgcacactacattttagcctgggcaacaacagagtaagatcctgtctctaaaaaaaaaaaaaaaagcaagcagtgatgtatgtttgcacgcacatacaaaagccatgaggagacctgcttagctagaaggaaggatttatgctggagcagcaggaaaggaaattggagggttgataagcaggttggtgggttgggatttgttttgtttgttttaggagacaaggtctcgctctattgcccaggctagagtacagtgggagggtcatagtttactgtaaccttgaaatcctggcctcaagtaatcttactacctcagcctcctaagtagctaggactacaggtatgcacccccatgccaggctaatttttttttttcaattttttgtgtagagacagggtcttgccatgttgcccaggctggtcttgaactcctaggctcaagctgtcctcctgcctcagcctcccaaagtgttgggattacagatatgaaccaccatgctggacctgtttttgtttttgaaggtgaacaattttaaaagtgtttttgggccaggcacaattcaatcacttgaacccaggaggcggaggttgcagtgagtcaagatcatgccactacactccagcctgggcgacacagcgagactctgtctcaaaataaataaataaaaagtgtttttgtgtttgagtggttaacataattaaaaatattttcaaaaagtacagaagggactaaaagggtcacctccactgcattagcttttggttttttttggttttttttttttttgagagatggagtttcgctctgtcgccaggctggagtgcagtggcgcgatcttggctccccgcaacctttgactccctggttcaagcgattctcctgcctcagcctcccaagtagctgagattacaggcacgcaccaccccacccagctaatttttgtatttttagtagagacagggtttcaccatgtttgcgaggaaggtctcaatctcctgacccggtgatccacccgcctcagcctcccctgctgggattacagatgtgagccaccgtgcccagtctgcattatcttttttatctatttatttatttcatttttatttatttacttacttacttacttattttgagatggagtcttgctctgtcacccaggctggagtgcagtggtgcaatcttggctcactgcaacctctgcctcccgggttcaagtgattctcctgtctcagcctcccaaggagctgagactacaggcatgcaccaccacgcctggctaattgttgtatttttagtagagacagggttttgccatgttggctaggctggtgttgaactcctgacctcaagttatacacccatctcagccttccaaagggctgcgattacagctgtgaggtgccacgcccagcctatgtatttatttatttttatttttgtagaggtgaggtctcactatgttgcccaaactgatattgaactcctaggctcaagtgatcctccctcctcggccttggaaagtgctaggattacaggcatgagccactgtgcctggcccactgcattattatatagaggcaatcagcgttaccttcctgagagatactgaggctgaacaaacatgtcttgtatatgcatttgcatctttttgttgttttaacacaagtggcagcatgctatttagacatagccttctgtacctgctgtacctggctcttctcacttaccaatatatcttggaaattatgccaccgtatgggcaatttgatttgactttcattgatttccttgtttttttttttgtttgtttgtttgtttttttgtttgttttttgcagttgttaactaaaaagttactccatgctggtgtctggagatacgttgttgaaaaatataaattgaatctctgctcttgtggagacaaatactaatctataatcacagaaataataatttccttaaagtggtagtagagctccaaaggaaaaacccagggtgccctgtggaggggtgatggtgacacttattctgattgtcaggaacagggtgggggtgctggttgacaccctaaaccaatgcaggctcttgctcagaaaaaactgtcagccctgagattttgcagatactttacaaagcacattcacctttattacctgacctggttctctgaataaccttgtcagaggcaaattttgctgttcccactaaattagttaatacatgtaaagtgcttacagcagtgccttgtagatggaaaatagtccacaaatattgtattgttttacagatggggagacagagccaggagaagtcaaacattaagagacaaatagtttagggccaggcgcagtggctcatgcctgtaatcccagcactttgggagaccagggcagacagatcacctgaggtcaggagttcgagaccagccaggccaacatggtgaaaccctttctctactaaaaataaaaaaattagccaggcatggtggtacgtgcctgtaatcccagctactcaggaggctgaggcaggagaatcactttaacccaggaagaggaggctgcagtgagctgagatggcaccactgcactccagcctgagcagcagagcgagactctgtctcaaaacaacagcataaataactgaaaattagacccagcctatttactgacagagctcttaattgttgtacttagccactgggctctaatagttttctaatgtcacttgtccatttttccctcaacaaacttgtggtaagaggctaagtgtttaccatgggccaggagtacaaagatgagctggagtacaaagatgaggaaattgtaggctccacccctgaggacaatagcggactgagatagcttaggaggcggtgaattcatgagcagcacaggtctttagcagaaacaagttgactgctggttgtgatgttataaaggacattcttggatcatttattgcaaactggctctgtgcagagctcactgtcttgggctttttgagtgatacgtgggtgtcttgggggactggagggttggtgggggaaatgtttttaggaggtaatttagtacatgtttctgcagccaaatacacatattactgggtggtcaggtgagatgatattaatacttatgtgaaaatgtagacataaagttccttgataagcaaagtaaagcaagtgaactaatttaaagaaaaccatttacagctggatgttgctcacgcctataatcccaacactttgggaggtcgaggcgggcggatcacttaaagtcgggagtttgagaccagcctgcccgacatggtgaaaccaactctactaaaaatacataaattagccaggcacggtgatgggcatctataatcccagctactcagggggctgaggcaggaggatcccttgaacccgagaggcggaggctgcagtgagctgagatcacgccactgcactccagcctgggtgacagagtgagactccatctcaaaaaaatttttgaaaataatggtgcagggccgggcgtggtggctcacgcctgtaatcccagcactttgggaggcccaggtgggtggatcatgaggtcaggagatcaagaccatcctggctaacacggagaaaccctgtctctactaaaaatacaaaaaatttatccaggcatgtagccggggcgcagtggctcacacctgtaatcccagcaatttgggaggccgaggcgggcggatcacttgaggtcaggagttcaagaccagcctggccaacatggcgaaaccccatctctactaaaaatacaaagaatagctgggtgtggtggcgcacacctgcaatcccagctacttgggaggctgaggcaggagaatcgcttgaacctgggaggcagaggttgcagtgagccaagatcatgtcattgcactccagcctaggcaacaagagcaaaactctgtctcaaaaaaaaaaaaaaaaaaattatccaggcctggtggcgggcgcctgtagtcccagctactcagaaggctgaggcaggagaatggcgtgaacccgggaggcggagcttgcagtgagccaagatcgtgccactgtactccagcctgggtgacagagtgagactccatttcaaaaaataaaaagaaaagaatagtgcaaatattatatgggtggctatggcagatgtaaaggtgattggcaattctttttctggaaaagctggcccaagggttaaaaggaagagctctggagtgaaagagactgcctttgcccttgaccttgggtaaaacttaacctcttcacatagtttcatcctttacaaaatgagaagggtgtggcaatgattaagaaaaattcagggctgggcgtggtggctcatgcttgtaatcccagcactttgagaggccgaggcgggcagatcacgaggtcaggagattgagaccatcctagctaacacggtgaaaccccatctctactaaaaatacaattagctgggcgtggtggcaggcgcctgtaatcccagctactccggagggtgaggcaggagaattgctggaacctgggaggcagaggttgcagtgagccgagatcacgccattgcacttcagcctggatgacagagtgagactctgtctcaaaaaaaaaaaaaaaagaaaaagaaaaaaaaaattcaggctagggggcatgcctgttatcccagcacttgggaggccaaagtgggaggattgcttgagcccaggagttcaagaccagcctggccaacatggtgaaaccgtctctactaaaaatacacaaattagtcgggtgtggtggtgggcacctataatcccagctactcagggggctgaggcaggaggatcatttgaacccggcagaggttgcagtgagctgagatctcaccactgcactccagtcttggtgacagagcgagactccatctcaaaaaataaaattgaaaataatagtgcaaatattatatgggtggctatggcagatgtaaaggtcattgggaattctttttctacaaaactggcctaacggttaaaaggaagagctctggagtaaaaaagacccccccttgcccttgaccttgggcaaaacttaacctcttgacacagtttcatcctttacaaaatgagaagggtgtggggatgattaaggaaattcaggctaggggcagtggctcatgcctgtaatcccagcacttgggagaccaaggtgggaggattgcttgagcccagaagttcaagaccggcctgggcaacatagcgagaccccatttctacaaaaaataaattaactggtcgtggtgacatgttcttgtggtcccagctactcaggaggctgaagccggaggatcacttgagcccaggagtttgaagctacagtgagccatgcttgcaccactgcactcctacctgagcaacagagaccctgtctcaaaaaaagagagaaagaagaaaaggaaaggaaaatgctcgtagcacctaaaacagtgctcagtaaatgtttgctgaagctgtaactatagtatgtagtagaaagtggtagttatttgaaagagagtcaaagatacgtgctctgggaattaccactaaatgaccaaaagttcacgtcaaaaaaataaaatcagatcaagttagaaatttttttttttttttttttttgagacagaatctcactcttgtcacccaggctggagtgcagtgacgctatctctgctcactgcaacctccgccttccgagttcaagcaattctcgtgcctcagcctcctgagtagctgggattacaggtgcccgtcaccacgcccagctaatttttgtagttttagtagagacagggtttcaccatgttgggcaggctggtctcgaactcctgacctcaagtgatccacccacctcagcctcccaaagttctgggattacaggtgtgagccaccgcacctggccagaaaaaattttattatgcacacaaaagaacagttggagattagtcagatgctccagtcactgtagtgagggcagtgttaataaagcctaaggaaggattttgaccttttttgtgattggctgttatacattaacaatctgtttacagccagtagagctgttcaaactgttttgtctatagttgattcctttaattttactgaattatgctgacaaggatgtaaagctttgtgttctgtttatgattgattagagctacatttcagggaatcaggatatcttaaattccggctatgtggctgtgggcagttggccttggagtatatctaaactgtggcctccactttcatttttcttgaaacattgctatcaactgggaagagtatgtgactttatgcccagtttcccctctcagatttttatgacggttgtttttctttttgttatgccatttgagggattgatgtttcttaaactatgaagtacttggctgtctctctccattgctgttcaggttaacagccaccatttgtaaacactttgttggagctcattccttctctgctaggaactttacaagaattgtctcaatcctaccacacctttggcaggacccttgttatcagagttttcattatccatgtatccatagttacaaggctatatagaaaagcaaaagtcatcacacaagaatcttgctaacaaactacttataaaattagtcactacttaaccatccctctgcttatccctgccagtcccattaacatgtttggtagtcaatttctggctgatgaagagatacattaagcatcaaggtaatgcaaatgtaaataaaactgactgtagactgaaatggcacatctcccaaaacttgtaggactaggcgaaatccctgcaagtgatgatagaaaaccttcagccttataccagttaacaaaggaagaagggaaatcaacaaagatgatgagatggtaaaaacttgaaaagtagaggatttgcctaggaaaaattaagcccttcggaaacttagtaaagctaaggaatattttggcaaaaccatcttttgtatgttgcaaaagtgagacaggaaatgaagtatagtgtttgggaattttttttttttgagatagagtcttgctctcctgtccaggccggagtgcagtggtgcgaccttggctcactgcagcctcagcctcctgagtagctgggattacaggcatgcgccaccacacccggctaatttttgtatttttagtagagacagggtttcaccatgttggccaggctggtcttgaactcctgacctcaggtgatccactcgcctcggtctcccaaagtgttgggattacaggcatgaaccactgcacctggcctagtgtttgggaaaactatactaggaaaagaatagttgctttaagtcattctttgattattctgagaattggcatatagctgccattataacctacttttgctaaatataataataataatcattatttttattttttgagacagggtcttgttttgtcaccccggctggagtgaagtggcgcaatctcggctcactgcaacctccacctccgggtgcaagcaattctcctgcctcagcctcttgagtagctaggattacaggcacaagccatcatgcccagctaatttttgtatttttagtagagacagggtttcaccatgttggtcaggctggtcttgaactcctgacctcaggtgatccacccgcctcggcctcccaaagtgctgggattacaggcgtcagccactgtgcccggccctgctaaatataatattaactttataattttagtctcattttccacccagatcttgtttatcactatctgggtggtagtgataaagttccacccagatcttgtttatcactattccctatgaaatgtttctgggtggccgggcgcagtggctcatgcctataatcctagcaatttgggaggccaagacaggtggattgcttgaggcccggagttcaaaaccagcctggccacatggtgaaaccctgtcactactaaaaatacaaaaaattagccaggcatgatggtgggcgcctgtaatcccagctactcgggaggctgaggcaggagaatcacttgaatctgggaggcaggggttgcagtgaggcgagatcatgccacggcactccagactgggcaataagagcaaaactccgtctcaaaagaaaaaaaaaagaaatgtgtctgggtatgtttttggctgcaattagctgaaaaccctgactcaaaatggcttattgcagatacttagaagcccagaggtgggcctccaaaggtcattgtctcagtggcctggactctgcatttttccactctgccatgttcaggtctcagctttgtccttaggaaggctccccatgtggcacaaatgcaggagtcacatccagaagaagaagcctttcttgccgggcgtggtggctcacacctgtaatcccagtgctttgggaggctgaggtgggtggatcacgaggtcaggagttcaagaccagcctggccaacatgatgaaacccccatctctactaaaactacaaaaattaaccaggcgtggtggcgggcacctgtaatcccagctactcgggaggctaagccaggagaatcgctggaacccaggaggcggaggttacagtgagtcgagatcttgccactgcactccagactgggcaacagggcaaaactccgtctcaagaaaaaaaaaaaaaaaaagaaaaggccaagcgtggtggctgtaatcctagcactttgggaggctaaggcaggcagatcacgaggtcaggagtttgagaccagcctgaccaacatggtaaaaccctgtctctactaaaaatacaaaaattagccgggcacggtagcatgcctgtagtcccagctactcgggaggctgaggcaggagaatcgcttgaacccgggaggcggaggttgcagtgagccaagaccgtgccagtgcattccaacctgggtgacagagggagactccgtctcaaaaaaaaaaaagccttccttggtttcctgccaatttcctgtcatgtttctttgtcccgaattgggccaatatccatgcccattcctaatacaattaccttaattgacttagattagccctgaagcatgtagtgatgtgggggaggaatgacaactgaagaaaatagaaaagaagggacaaaggttgttgagttggcaaccaacaattcctgttatagctttttaaattaattcactttaagtggtactgattattcatattgttatttccatgtatagagagagaagaaattggaggcttagaggaagtgaagtgacttgtccaaggtcacatagctaagtggaatttattttttttttttttacttttctgagacaaagtctcactctgtcgcccaggctggagtgcagtggcgtgatcttgactcactgcaacctctgcctcctgggttcaagtgattcttctgcctcagccacccgagtagctgagattacaggtgtgcaccagcatgcccggctaattttttgtatttttagtagagatggagtttctccatgttggccaggctggtcatgaactcttggcctcaagtgatccacctaccttggcctcccaaaatgctaggattacaggtgtgagccacggcgcccagctactggaagtcatgatgcaagctgttgtttgaatcatgaattccaaacttaatgagtgctaggttgaagttattttttttctgtttcccttaatcttctattgagatataacatgtattgaagcacagacatcttttgcaagaattcattgagcactatagttatattcatctgtacttactgtctccctggtaaaggcataaactcccttcaagatggggacatggttttatactttcctgaatctcctttagtgcttatatctaacagcacttaaccaggatctggagggggtcaaccagtcttgcttccttccaatctactctgtagatgtttgtgtttttctactgtaactatagaggtaaaggagccaatggtgtttccagtgtctggcacataataggtgctctatgaatgtcaaatgaatgactgaggcataaaaagttcttcctatgacataagtagtataataggaagatcactgggatagaattttcttagaagacgtggctcctagtcctagcactattactcttactgactataactttggacaagtcatttgtttaatttcaaacatttactgaattctgtgtaccaaatactgtacaagttgttcaaaaggctacagagtctctactctcaaagtctagtcaggggagagattacacaggagtgaaaccccatctctactaaaaaaaaaaaaaagaaagaaaaaaatagccaggcatggtggcaggcacctgtaatcccagctcttgggaggctgaggcagaagaattgcttgaacccaggaggcagaggttgcagtgagtcaagatcacactactgcactccagcctgggcaacagaacgaatatctgtctcaaagaaaaaaaaaaaaagaaaaaaaaagtatgtttagagatccttaaggaagttttgaacctatccaatatgttgggagtcaagatgaggagggtaaaagtaggaattcagagaaaggataaatattttgaaggaaggcagcataatggttaactagtcagactgcagacaaacaagcagtacaaatcccatgttcccaggtcttagctggaggattaaatcccagattctcatccttgagaacctctttaagcttgttttcccatctgtaaaatgtagatgataatagaatccttttcctactgtaaaatgatgcacattgggggcttcatgtagccagatatataataaatcctcaatacatgtagcagtggtaggggagaactaaaagacttgttaacatattggatgtaggtgagaaagaagaatcaaaaagaatatccaggttcccgccctgggaggggcagacataaatggagccaggaatggtacaaggagctttgtggtgggcactcactagtacctttctttggaatagctcaacaaatgttcactaagtacacataaagtcaatataacttgatgagctagagttaggctaagatgttgagttagcagaagagtagtacccaactaagggcttgaggaaggcttcctagaaaagataatgggtgcacgtagtcctgaaggatgagcaaggcttgcgaggagggaaggtaggaagggcattccagacaaagggacaaacatgcacaaaggtgagacagagcatggtgaacacacaaagttgtaggcattttgtctttctaggacacaaaaaacaaggtgatgaaaacaaggctggagagacaagcggagaccacaagtggtcacccctcactggcctcgctgtactactggactgctccagcccttcctggcccagagctagagatggcaccacccccgcactgtgcacacaataggtacagacactctcagccccaaacccctccagcctgttactgtgagactcacacatcctgttttcccaagcttgccccacagggtgagcaaactggaccagctggattagagtatttggaaaagtgcaggtggagtttctaggctatgccacgtcaggccagggggtgagatttttttcccttcccctggtctttgggctggggcactccctgaggagggaggggtgtgacccatcccacccattggtggtccatgatccattcatccagtacatcatccataagcatctgctgagccgggcgcagttgctcacgcctgtaatcccagcactttgggaggccgaggtgggcggatcatctgaggtcaggagttcaagaccagcctggccaacatggtgaaaccccgtctctactaaaaatacaaaatttgccgggcatggtggcgcatgcctgtaatcccagctactcgggaggctgacacaggagaatcgcttgaactcaggaggcagaggttgcagtgagtcgagatcgagccatcgcactccagcctgggcaacaagagtgaaacgccatctcaaaaaaaaaagcatctgctgaaggcacagtattgtgttagaggcttggactaccctgaggaataatagtcctgacacttgaggacatccccatccaattggaaaggcactggataaacatctaagaaaatgtcacaaggcataaaagctgtaataataatagctaacttgtatttagggtaaacaaaaggctgaatcaggctttggactaaatattttgcctgtggctcatttaatcttaataacaaccataagattatctcattttatgatgaggaaactgtatacagagaggtgattgacttgcctaaggttgaacagcttatgagtagtagagccagtattcaaatccagtcagcctccagtgtcccctctctttagtcattagatatagtgctacccagagaatctgctaagccttatagataaatgctatgaattcagaggcaggagcaattactgtggcttgggggcatccacaaagactttctaaggtggtgacacctgatttgggccttgaaactggttgaatagagataagaaaagaacagataatcaatttaactgccgttgtgttttgaatcatcactttaagcctagaatgacaaagctggaaaagaccctaaatatcatctagcccagtggttatcaaattttcttaggatgtggacatttttctttaaatgaaacatgagtagaagccagatgcagtggctcatgcctgtaatcccaactacttgggaggctaaggtgagaggattgctcaagcccagaagttggacaccaacctgggcaacggtgagaccttgtctcaaaaaagaaagaaagaggccgagggcggtggctcacgcctgtaatctcagcactttgggaggctgacgcgggtggatcacttgaggtcaggggttcgagaccagcctggccgacatggtgaaaccctgactctactgaaaatacaaaaattagtcaggtgcagtgctgcatgcctgtcatcccagctactcgagaggctgaggcaggagaatcgcttgaaccccagaggtggaggttgcagtgagctgagattacaccactacactccagcctgggtgacagagtgagactttaatttcaaaaagaaagatgagcagtaatcttgtctcttaaaaaggaggaccaaacattccctagccccaaaacattccctgaaaagttcagttgagaaccactgatctagtccaatgtcctgggatcctcgcttttatctttgctatctggagaaggaagagacagcggtagttcccaaaagacacaaacaatggggacattttaaacgggagtaaccagctaagcactctgctgctgctgcttgtctatttgccctcttgtcctgcctcagggtgaattgttggcagctacaaatggcaaccaagagagaatggagttggggcttagaagtgcggtagaggaatatgaagggggctggagttggagaggctcttaaagaaagagctgactatattgggactggagagttaatgtaaatctcttcttggttcatggggattttgttgttattgtttttttccccatgacacaactctcaggagatcctgagaacacgtgccctgatttggggggggttttaattttaactttttttttttttttttttttttgacagagtctcactctgtcacccaggctggagtgcggtggcgccatcttggctcactgcaagcaagctccgcctcccgggttcacgccattctcctgcctcagcctcccaagtagctgggactacaggcgcctgccaccacacccagctaattttttgtatttttagtagagacggggtttcactatgttagccagaatggtctcgatctcctgatctcgtgattcacccgcctcggcctcccaaagtggtgggattacaggcgtgagccactgtacctggccaaaatttttttttaattagagacagtctcttgctgtgttgcccaggctgatctcaagcagtcctcccaccttggcctcccaaagtgctgggattacaggcgtaagctatagcacccagccttccttctttccttaattctatttttttttttgtttgtttgtttatttgtttgcttttgagatggggtcttgcactgttgcctgggctggagtacaatagcgtgatctcggctccctgcaacctctgcctcccaggttcaagggattctcctgcctcagcctcccgagtagctgggattacaggtgcctgccaccatgcctggctaattttttgtatttttaacagagacagggtttcactatgttggccaggctagtctcaaactcctgaccttgtgatctgccctccttggcctcccaaagtgcggggattacaggcatgagccaccatgcccagccttccttaattctattaatatatgaatccttttcttttttttaaaaaaaagagatagactcttgatctgttgcttaggctggagtgcagtggcacaaatcagagctaactgcagcctcaaactcctgagttcaagcaatcctccagcctcagcctcccgagtagttgggaccacagttgggaaccactacacttggccctactacgggtttttcctgtttgtttgttttttgtttttttgtttttttgtttttttgttttgagacggaagtttgctctcgttgcccaggctggagtgcaatggcgcgatctcagctcactgcagcctccgcctcccgggttcaagcgattctcctgcctcagcctcccgagtagctgggactacaggcatgcactaccatgcccggctaattttgtatttttagtagagtcggggtttctccatgttggtagtcaggctggtcttcaactcctgacctctggtgatccgcccatctcggtctcccaaagtgctgggattacaggtgtgagccaccgcgcccagctgtttgtttttttgagatagggtctcactctgttgcccaggctggagtgcagtggcatgaacatggcttactgcagccttaacttcccaggctcaagcgatcctcccttctctgcctcctgaatagctgggaccacaggaatacaccaccacacctggctgatttttaaattttttgtagagccagggtctctccatgttgcccaggctggtctcagactcctgggctcaagtgatcctcccacctcagcctcccaaagtgctggggttataggcatgagccactgtacccagcccccttcagggtcttatctttataaggggtggggggtggtcagcacctttctcattgttacctctaccttccctaggtatgtgcaggtgaaatttgtctgcattcggacccaatcaaacaggaagagaacgcgggaggcagacatgtgcccagcgtacttgctcctaaggtacaacgagagactagatagactatttatcagtgaactaaacacacagcacatacatggtgactctaaagtggctagtcctggaggagacaccactggcaaatctcaaaagacaatgtgcctgcagagactccagcctgtgcagcccacaaccaaaaaagaccttgacactgccgagaagtccctggttgagccatcgttttgcctagataaggtacaagtgtcctcaaagccagagcaggaaggcatcactccttctgacctggccaagatagcaaaagtgatgaagaactttcttaaggtagatgagggttccatggcttccttcagtgtgggtgacagccagcacctggaccggctcagcttccagagcagtaagatgaccgacctgttcatccgcttcccagagaatctcttgctacaccgggtggagaacacccagggccacatcctctatgctttcttggtggagaacaaggaacgagaaagtcgagtggtgcactttgctgtgctcaaggcggagacagtcacctctgtggccaagatgctgagcatcttcacagagttcaactccgattggcccaaggtcaaggtggtctttgtggacccttcattccattaccgggctatcctgcaggagatctttcctgctgcccgcatcctcctttccatctaccacacaacccgactcttggagaagaagttgcatcgtagttcagcaaatccatcctttaaaaggctcatgaaggaagccctgcgggaggccgtgtttgtcacttctgaagccagcctgaaaaatctctgccagatgtcccaggccgtactggatgaggatctcttcaacttcctgcaggcccactggttcacctgtgaactgctgtggtacatgcatgttaggaagggcctgcttgcgtgtaacacctacatggacagcctagacattgtcaccagcaaggtgtcaagcctctttcgggaacagcagtcgctgctggactgcatcctctgctttgtggattacatagacttctttaataccaaaggcttgaagaacttgcccacacctcctcccaaattaaagagagctcggccggcaagcatgccactgaagtccaagaaggcttttggaatctgtggagagagccttaccagcctccctgcagaagagaccaagccagacgcacagcaggtacaggtacagcagcagtcacaagtgccgccctcgcaggttggcatgctggacaccttgcaccagagtggctctgaactagcctacaagctgtgccacaatgagtgggaggtggtacagaactccacccacctggtggacatggctggctcttcagtggacgttcagctgctagaggactctcaccaggttagcaaagatggctgtagctgcagctgttcctttcaacaatggtaccacctgccatgccgacacattttggctctgctgcacaccagccagcagccggttggtgaagccatggtgtgccgccggtggcagaagaagtaccagtacctccttgggcccaatggggagctccaggatcgtggtatggtcccaaacacaggccagcctgagaagcaaggacggaacgacatgattcaggacctaagcagggagttagcaaacctgctcatgcagaccgaggggccagagctggaggaacgctactccaccctgcgcaagattgtggatatctgggctggcccctcccagccatctgagctctttcagcagccaggagactttaaggacgtgggccgcctccctttcctctggggaaagcaagaagaaggggagggattccctcctgctacagctgtgatgcattattgaagcactttagctgaagcattggaccacaaacacttctccttggaagtgtgagagtttaaagtgggcaggacatactagggtttagcattttagccaatgtcttcctaggtggggctaggaatattgttacagtagagaggaagggaactccactgtgtgacagtcctttcaatctgccccttttcagccctacttttggcattccttgggagcctcagttgttgttcaaggccaaagttatctccgtgctgcaaggtcaccctcttcctcccccagcccctgagatcagatcttatttgctctgcaaagatgaatccctgcctcaggttagggtgagacaaaatcggtctggtaaaagggcctgttttcagggacaaagggaatgaggatgatctttggctgttgctgctctttatgaagaatcttatttgtaactttttattcatattaaagttgtttttaataaaattaaacaagggaaatgtgttctgcttgatcagggaaggaagaggaattgcatagcaataggactcgactttacagttaggaagtggctgctctaggtggatttattaattttaaagaaaattgaggccaggcgcagtggctcacactcataatcccagcactttgggatgtcgaggcgggtggatggatcatctgaggtcaggagttcgagaccagcctggccaacatggcgaaaccctgttctactaaaaatacaaacattagccaggcgtggtggcgcacctgtaatcccagcttcttgggaggctgaggcaggagaatcgcttgaacccaggaggcggaggttgcagtgagcctagatcgtgccaccgcactccagcctgggtgacagagtgagactctatctcaagcaaaaaacaacaacaaaaaaaaaagaaaagaaaattaagtattagcaacctaatggggttgtggagccaaatagaacctgatgcctcacattcaaggatttggcaatctgcgtcttaatttgcctccttcctacaactaagccttcaaggttgatttcaggtttccttatattctagcgggaagaggccaaatagactggagggtgtgggggaaaaacctgaatcttagacttgactgccagcaacttgctggggaactgtgggcaatttctttgacctcagtttcctctgtaaaaattagcatgttggcctgttctaatcccagatttcccctctaggtttacataagcacctttttgttttttgttttgagatggagtttcgctcttgttgcttgttgcccaggctagagtgcaatggcagaatctcggctcactgcaacctctgcctactgggttcaagcgattctgcctcagcctcttgagtagctgggattataagcatgtgccaccacgcctgcctaattttttgtatttagagatggggtttcaccatgttggtcgggctggtcttgaactcccgatctcaggcaatccacctgcctcagcctctcaaagtgctgggattaaagggatgacctactgcacccagcccaataacattttcgatgcgtaagtaccaacactgatttgatgttgctagttggtaatcttaatcctccaaggcctggtaaacacccagttttctaagtctgccatcttcaagggaactgaggacaagagaccagttagtaggcccttgcactactgtagtaggaccctaaataagtcatggcatcattctgtagaatactggagggggttgggctagagaatgccaagttttggcttgtttaaggggatctggatttcctcagctctaaaagttgggctcctgatccccgaaggaggagtagaatttgagacctggaactcagtttccccataaggtagaatatgcgttatgactatatatgcactgtttcctaaattccaggggctttagagtttcacacacacacacacaacaaaacaaaacccgcggtggctcacgcctgtaatcccagcactttgggaggctgaggtgggcgaatcacgaggccaggagttcgagaccagcctggccaacagggtgaaaccccgtctctactaaaaatacaaaaaattaactgggcgtggtggcgggtgcctgtaatcccagctactcaggaggctgaggcaggagaatcatcgcttgaacctgggaggcggaggttgctgtgagtcgagatcttgccactgcactccatcccaggcgacagtgcgagactccatctcaaaaaaagaaaaagaaaaaatatatatataactgctatttattttccttttcattttctgcttttgagggagtttccatagtaacgtgtccttccagtcgcctagcctgaagggaagccccggagctccagcttcaaggctcagctctcccagatgcaagtatctttacctccagccagctccttgtaaccctggcaacacggcccgcccgcgaatcgtcacgctacgattggtccgtggtgctaaagcctgaaagcgaattcctcagacattggtccgagtcactgcgatgctgacgctgtggttggtgagtttggatgtcagtcagagtcggaggcagggtcaaatagggagaaatggcgacggagcctggctgtgggtgagtgcttcctgaaggggtgaaagtgtgagacagcggatcaccgcagttagccgtcacacagctcccaaagggatgggagggaggttttcttctcatccggctccagctggactctgggaatgtcagacatccacatctcccacccaggttagaggtgagaggtcaaaggccgtattccctctgttggtttttggaccccaggtactgtggacagcagtcaggatcccgaaccaaagactcaactttcagctacttacagagtaatggtgggcaagtcacttagcttctttgagccttagtttatcatctgtaaaatgaggacagtaactcggtctggggccaaatagaggaaacaatggaatagagtattgctgctaaaagagggatatagtagaggcaggagaagctatgcacatttgttagccatattttatggtggaagggattgaggctcagagagggtcattgacttgcccaagataacacagcaagtttggagcaattgcagttctcttatacataccaggaccaaaggaaagaatggatgccaatcagtcatttataaatataaaatcgtatttttattttattttattttttgagacagagtttcactctgtcacccagagctggagtgcagtggcgcaatctcggctcactgcaacctcagcctcctgggttcaagcaggtctcctgcctcagcctcccgagtagctgagactacaggcatgggccaccatacccggctaattctttttgtatttttagtagagacgggttttcaccatgttggccaggctggtctcgaactcccaacctcaagttatctgcccaccttggcctcccaaagtactgggattacaggtgtgagccactgcacctggctagaatgctatttttttaaaaggccatatgcagctctggccatacctatttaaatcccctggaaccatatctttccatagctaacttcttatcatttagatttcagctccaatgtcatccttctcccctgagaggccttccattaccactttaactaaagtatccctcccttccccagcttactctctgacatgttactcaggtttatggtctttgtaagcttcattattggatatatatttgcttatttgtttacatgtttattgtgtatcttcccctagaacagtgctgacatatagtaagtgttcaacaaatattgtgtggatgaataattgaagagccaggtccctgctgtctccttaaacctctgttactctccataggcccatctttggaaaaaagatctgggaatgattgtctagcctccagcctcaacttacttgatgcttgagagactcaaagccccgtggtcagctgccctgcaaagaaagtattttgaccttggcatttggacagctcccatctctcccatggccctgacaatgctgaatgggctcctgattaaggactcaagcccacctatgctgctgcaccaggttaacaagactgcccagttagataccttcaactaccagagctgctttatgcaaagtgtctttgaccatttccctgagatcttatttatccaccggacctataacccaaggggtaaggtcttatataccttcctggtggatggacctcgggtgcagctggagggtcatcttgcccgagcagtctactttgccatccctgccaaggaggacactgaaggcctggcccagatgttccaagtattcaagaagtttaatccagcatgggagagagtctgtaccatcctggtggatcctcatttccttccactgcctatcctagctatggagttccccacagctgaggtccttctctcagccttccacatttgtaagttcctccaggccaagttctatcagctgtcccttgaacggcccgtggaaaggctgctcctgacctccctgcagagcacaatgtgctcagccacagcaggcaacctgagaaagttgtatacactcctgagcaactgcatccctccagccaagctgcccgagcttcactcacactggctgctcaacgaccgcatctggctggctcaccgctggagaagccgagctgagagcagccactacttccagagcctcgaggtcaccacccacatcctcagccagttctttggtaccaccccatctgagaaacaaggtatggcttctctgttccgttacatgcagcagaactctgcagacaaggcaaacttcaaccagggcctgtgtgcccagaacaatcatgctccctcagacaccatccccgaaagccccaaactggagcagctggtagaatcccacatccagcactccctcaatgccatctgcacagggccagcagcccaactgtgcctgggcgagcttgctgtggtccagaaatccacacacctcattggctctggctcagaaaagatgaacatacagatcctggaagatacccataaggtgcagccccagccccctgccagctgcagctgctactttaaccaggccttccacctgccctgccgccacatcctagccatgctcagtgcccgccgccaggtgctccagcccgacatgctgccggctcagtggacggcaggctgtgctaccagtctagacagcatcctgggcagcaagtggagtgagaccctggataagcacctggcagtgactcacctcaccgaggaggtgggtcagctgttgcagcactgcaccaaggaggagtttgagcggaggtatagcaccctgcgggaactggccgacagctggattgggccttatgagcaggtccaactctgattattctcgatgcccagagatgctcatgcacctgtgcacactcacatccacccatacacacacacacacacacacacacacacacacacacactcccttacactgttgtacttccgtgggccctccttccagaacaaggacaacaaggacaaggttgaagggtcttctcatctaccatggcctgctacctagcatgtgtctagctcaatgagacaggagtcagcaaatcttaatctgtttagtttactcaggtggccacatacagtctctgttgtatattcttggttttgttttaatattttttttctttttttttttttttttttttttgagaaggagtctgtctccgtcacccaggctggagtgcagtggcgcaatctctgctcactgcaagctccacctcctgggttcacaccattctcctgcctcagcctcccgagtatctgggactacaggcgcccgccacaatgccgggctaattttttgtatttttagtagagacggggtttcaccgtgttagccaggatggtctcgatctcctgacttcgtgatctgcccgcctcggcctcccaaagtgctgggattacaggcgtgagccatcgcacccggcctgttttaacattttcataaggtaaaaacctttcttagctcaaaggtctttaaaaaagcaggcaatccagctgggcatggtggctcacgcctgtaatcccagcattttgggaggctgaggtggacagatgacttgaggtcaggagttcaagaccagcctggccaacatagtgaaaccctgtctctactaaaaatacacaaattagccaggtgtggtggcgggggccctgtaatcccagctactcgggaggctgaggcaggagaatcgcttgaacctgggaggcagaggttgcagtaggccaagatcctgctgccactgcactccagcctgggagggtgagactccatctaaaaaaaataaaataaatggcaacccctggtctaagataagagataaaacatcaggtggtgaggttgaggtttggggcttggtagcagttgccccagtcatgagatgactcacttaacccgtctcctttaagtgagctgggctgggaggcttcctacaggggaagaggcccctctggggagctgactcagccaggctccctgaacttttttccttgtcccatcctggggtcaataaaactgaatgttgcatattctagcacttgtctagttttttttttgttccatagaaggcagtttagggtatatcatggagagaatagactttagagtgttatacaacatgtgaatcctggttggttccttccctgcttgatttttgtgcctggttctgccttttactagctatgagacttattaggataagttacccctctaaacctcaacctgattatctgtaaaaatggggatctccacagggtatgttcacagagcaggcatacctagtgggtgctcaattaagtattaattttccttccttgcctatggtcctatgacctgccttcaacatgctgggaaatttaaggcaagaggagaattcaaatacctaggacttaatataagaaattctggccaggcatggtggctcacacctgtaatcccagcactttgggaggccgaggcaggcggatcacctgaggtcgggagtttgagaccagcctgaccaacatggtgaaaccgtgtctctactaaaaatacaaaaattagccgggcgtagtggcaggcacctgtaatcccagctattcgggaagctgaggcaggagaatcacttgaacctgggagacggaggttgcagtgagccaagatcatgccattgcactccagcctaggtgacagagcaagactctgtctaaaagataaaaattaaaaaaaaaaaattctgggtaggccaggtacggcgtctcacgcctctaatcccaacaccttgggaggccaaaacaggcagattggagcagcctgggcaacatggcaaaaccccttctgtacaaaaaatacaaaaattagctggatgtagcacgtgcctgtagtcccagctactcaggaaggctgaggtaggaagatcacttgagcccaggaggttgaggctgcagtgagctgtgattgtgccactgcactccagcattggtgacacagtgagaccctatctcaaaaaaaaaaaaaattctgatcaagtcttcaaaacaacataaaagcagccttaactctccaagcaggggcccctacctcagataatttccattgtatctacctgacaaaacctatctcctgtcttagtggtatctggagaaactttagggtctggaagtccctgatgaaggactggaatttgtcagaatggctcaaccaccagctctatccccatccctgccataggccctcctcccaaccctgtctgcccaagaaccatacggagaaaccaccctacatatctggtgctatttcatccatctacaagcaggagccaggaggctggccccttttagatctcactaggggcatcttcccagaggctgtgtgggcatgtgcctgctcccaccgcgccccctccacagcaccctctggctccggatgggccatcatgaaagcttgagcggcccagatcaggtcctcttcccgaactcctgggacgggcacggtggggatgccagcgatcatcatggcgagggtgaggatgcagatatcaggctgggagctggcctccttccccactgcctcagagggtaccggtagaacccgtggtgacagcccacacagcatcaggggcctaggcctgctggggccacccaggtcaggggctctgcttctggagtagccattgtcccagcccaagctctccagctgcctcacatgcgggaatttgtggcagtttcggctgtattccttccgtagtgtctcccagctagtccccccagggcagcccctggcttgtggcattgccagggctgggccccaggcctgggcagcaggtgccacctgctcagctttctggctcagtgggcctgttccaccagaggccaccaccactggctctacaggactacagaggccaagggacaagcctggagaagcagccccacctgcagagacaggaatgaaatggaacgttattctcagttctttgaggagggggccctgaggggcagggcatgggagcagagcttgggggcgtaactaagaccagccccagcggactgtctagaggttgggaactaggtgttactcatatccacactcccagtccccaacacggagtaggtgatggagaagcgtttgttgaattaatgaatagtgagcaagcgggtagttattgattggcaaggatgtttggaatggcttttcttctaaaagaattgattctagggccagcccccaaatgcccagcagaaccgccacctccccagagccagaaagcaacttctcaggggaagctgccgcactccctccaattgcaggtgccccccgctcaccttggccggaaggcagaggacctggatgagtttgtggagtcctgaagtaggacatggcttccccaaagccccggtttgtgagggaatagtgatctgccgcctgtcctcccttcttcacccttctagaatgtcacctcatagaccctccccttcacacttggaatccactttacagcgttgacctattcattctagaacctggacatgacaataggatgaggttaaaagagcaccagaccaggagctgggtgttctaggttcttgccctaaccttgaacgagtcacccagatagttttgttccttgatatccttatctgtaaagtgtgtctgatgactgcccactacctcattaggagttttgaggatgaaatgagatcatttgtatggaagcagatgaccagagatggaaacacaggagccaagtgcttcgtgccttgtagagtaagacttcgggtcagacccagtattggtatttttctttttctttttttgagacagagtctcactttgtcgccatgactggaaggctggagtgcaggagcaagacctcagctcactgcaacctctgcctcctgggttcatgcaattctcctgcctcagcctcccgagtagctgggactacaggaacctgccaccacacctggctaacttttgtatttttagtagagacagggtttcaccatgttggccaggctggtctcaaactcctgacctcaagtgatctgcccacctcggcctcccaaagtgctgggattacaggccaggtattggtatttttcaaaattcctcaagtgattataatatatgacaaagttggaaccttttttctagggacatacttctcaaccctggctgtacatgagaatctcttgagaagcttttacaaaacacaatgtctaggcctaatttcccaacaaatcaaatacgaatccctagggatgggatgtgggacaccagcactctttacatatcctaagatgactctaacaagcagccagcctagggacccctgctcttttgctccgacctgctgacctgcagtgattctaaacgaagtgactctcataggctgtaggggcaaggagaccttccagtgatcaagatgtcagcatcggctgtttatttctggtcttggctggcagcaatgtggccaacttcggaccagccagccacaggtctggggctccaggatgcagctgtgctctggtgcactgtgggtcttcactcatacttgcagaaatccttaagttctttgggcaggtggagcccatcaatggccagccggtgcaccatgctcctttgtatcactaggcggcacagagtctgcagggatggcactgtgggaagaagactgtgaaaggcaaactgaactggagggtgggtctgaggccaagatccagaaggtctaggtcatggtccccactttgtcattaaatgtgggcaagtccttactgctctctgggcccagcctgcttcatggggctgagagaagcacctcttcctgagagctgccacaaagctgcagccctgtttatatcataacacttagcacaatcccttccccattcacttagcagcttctgggggcaggggtagtgtctgatttgcctgtgcaactgaagcctccagcacaggcctggcacaaagagggttctggtgcattgagttcatgaaatgtgatgagaggtataactgcaatgcttaccagggctataatgaatcatactcagcattaagaagctctcagtctggggtttgttatgcctcgaagccaatagccaatatgcccaagtctatgagcatgtttctttcttttctttctttttttttttttatttaaagagagagggtcttgctctgtcacccaggctgaagtgcagtgctcagtgaactgcagccttcaattcttgggctcaagcgatcctcctgccttagcctcccaaatagctgggactacaggcatgcaccaccatgcccagataattatttaattttttgtacagatggggtctcactttgttgcccaggcttgagctagctagcttgcttgcttgcttgctttctctctcttcctctctctctctctttcttttttttttcagagtcttgctctgttgcctaggctggagtgcactggtgagatctcggctcattgcaacctccacctctcgggttcaagtgactctcctgcctcagccttctgaatagctgggattattggcacatgccaccacacccagctactttttgtatttttagtagagacaggctttcaccatgttggccaggctggtctcgaactcctggcctcaagtgatctgcctgcctccccctcccaaagtgctgggattacaggtgtgagccactgtgcccggccaagcatgtttctaagaaaagggcaaacaattctcaaagaggcccctaatatctaaaattggtttagaatccctgccctaggtgaatacaggaaacactatcaaaactaaaagggaacttgtagggcatcttaatcaatcaaccaagataggtaccaaggcccaaaaccatgtagaggcttgtgcaagatcacaagcagaggtgcaatttgaagctagattctcccactcttaaactagtgccggacagacgaaggcctagcacatgggctacggagccctagtagattccagtccccacactgagccaggaggggtcgctgcccagggatacctacagccatactcgagctggacaaggcgcacgctctttgtggaagcaaacacgtccaccaccgcgtagaggggctgcgcagctggcagtccccgggcgctcgggcccatgtcctcgccgttgatgatgatgtgcatgtcggccgtgccatcggggcgcgggcaaaagaggacacccaggcggctacggcgcgcggtcggaggcagcacgttcagctcatagagctggtccaagagatggctgtagagccctggccggctgcggcccaccaggcggtcccggggaatgcgaaactgctcaatgcgcagatatggttccacgaggagggttggaggtcggctgggggccgctgcctccgcctccgggcggccctcccggggcacgcggttgtggtggcgcgtgatggcgaagacccaggtgtggcccaggttgaccagatcgggcagagaaaactcgggaacgggggccagactggcggggtccagcgcggtcagaccgagacgcagatgtccgcaccagcccagctctttctcctcgatctcgaccaggaagacctggcccggggccagcggctcgcggctgaagcacacgccgtgggcgaagctctccacgcgtgtggcccgcgtcccagaggggtccacgcggatgttggcaccgtgcacccgatggaagcgggtgggagggggctccgggcgctcgagtccccagagtgcacccgaatccacgggctcggaggcagcagccatctctcggccatagggcaggccagctggcgccgggggctattttgggcggcgggcaatgatggtgaccgcaaggcgaccttgtaaggcatttcccccctgactcccttccccgagcctctgcccgggggtcctagcgccgctttctcagccatcccgcctacaacttagccgtccacaacaggatcatctgatcgcgtgcgcccgggctacgatctgcgaggcccgcggaccttgacccggcattgaccgccaccgccccccaggtccgtagggaccaaagaaggggcgggaggaagactgtcacgtggcgccggagttcacgtgactcgtacacatgacttccagtccccgggcgcctcctggagagcaaggacgcgggggagcagaggtgagctggcaccggaggctggaggggatccccgagcccgggatcggtgcgcggcagaggaggctcgcgggtgggagctggcgctggggccggggcttccctcgcggaggcgccgccagcaactccccgggggctgctgcacggaagcgctgaggagcgagtcaacagcccctctgctgcctcccgtagatgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaggctccggctccaagcacctccactactggtctgccgccctgccttctgggcgggattgggagaagagatgacggatgagggatggggggtagttctgcagacccctgaggatgcctgggagccgggagggctggaaagggcccctccaactgcgccaaccctgccctgacccccctcccaggtttgtggagtcccagaaggatcccgagaacagccctgtggtgctttggctcaatgggggtcccggctgcagctcactagatgggctcctcacagagcatggccccttcctggtgagtggacagcagggggaaagcacagttcccaaagtaaaaggctggggaaagcgagagaggggctttgtgattttccaaaaagtttccttccttctaagcctcgggattttcctctatgccattggctttgctgcctgtacctctcagaggttttacccacatgtaaagtgcctctcatggtggccctttccctccacccagctggcccttgggacttactcagccatctctttcctcctcaggtccagccagatggtgtcaccctggagtacaacccctattcttggaatctggtatagctggagctgtgggtgtgtctgggcacttggatggggtggcatttagctaatttttccctcccctctagattgccaatgtgttatacctggagtccccagctggggtgggcttctcctactccgatgacaagttttatgcaactaatgacactgaggtgagtctggtgcctgcccatctgccccaggctccccggtcctccatccatggcatgatgctgggagagagagcatggccttggagtctacttttcgctctgtcatatgctattgtggtatggtggccagtcacttcctcttgcacaaatagggtgggttaatgtcattatctctgaagttctttccagttcaaataccaaagcttcctgattccctctgtcttgctctgccatccccaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggtgcagggtagctgcaggagggaagggaggtagcttgaggctgtggccttacagttagcaaggtcagactgactggtcaatgtccccatccaacccagcttcctgcataacctccccaccaccggctgccctaggtctgtctgtgtgcctcccacacaggaaactcacctgtcaggctgccagctctgtgttttgcattgagtggcctctcatacccaccggcctacataaaccaccctgggtttcagcttccggattgctgaaggtctgggtggtagggtgagggaggctcttcctttttgccctccacatgagctgagcaccctgggtgtttcacaggggctggctgtgggcaatggactctcctcctatgagcagaatgacaactccctggtctactttgcctactaccatggccttctggggaacaggtatgggatagggcagttgggcaatctctggggtgaggcaggtcacatgatctcaggtctgtcctccaggcacatgatatgacaggcccagcccaggtttatgagcagcaggtgggttgcagaaagaacctagtgctgaaaggccaaagaatagagatcagtttttctttctttcttttttttttttttgagacagattcttactttgtcacccaggctggagtgcagtggcacgatcttagctcactgcaacctccacctcccaggttcaagcaattctcatgcctcagactcccaggcagctgggactacaggcgcgtaccaccacacctgcctaattttttgtatttttagtagagatggggttttgctgtgttggccaggctggacttgaactcctggcctcaaatgatcttcccaaagtgctgggattacaggcatgagctacagcgcccagccagaaatcagattttaaaggaactaaggaggcctgtctgtatgaccctggggcacctctttgggagctagcttagatgacatacagaccacaccctgtgatatctttcccagtctcctagggtcagggaggttggtgggaggtggggggtatgctcgcctcctctgccttcctcttcccaccagcagctgatgcgcttttcactctcatctcctacaggctttggtcttctctccagacccactgctgctctcaaaacaagtgtaacttctatgacaacaaagacctggaatgcgtgaccaatgtgaggttctgccatcactttgcatgagctctcccatccctaatcctgagaacaggaccacattccctccttggggactccttgttctccgtccctcattgctttcttgcacctttaagcaatttagtgttctctgggacatgtgtctaagtgtgtattcccaccaccacctcctatggtggcaaatcccataacctccgtgaggagaaaggacttcttcacatttatcaaacacctgcaacgtggcaggcactgtgctgaactgtgtctatgtagattatttaagcttcaagattccccctcaaatgagatcatactacccacattttccagataaggaaactgagggctatgaattttgaaatcactgtctgaagtcacagtgttgggaggcagtgatgctaggatttggacccaggtctgggaagattcctgccactgagcctcagtttccctacctgtgaaaaaaagtgggtgaagaggtgaaatccaagagcagtaaatcttgggacaacttggtggggtcgtggagaggctggggatctgtaaagcatggtggcttggtgtatcattgcagcttcaggaagtggcccgcatcgtgggcaactctggcctcaacatctacaatctctatgccccgtgtgctggaggggtgcccagccattttaggtaggtgctgctgggtgcccctggagccaaccccagccccatctggaggctccacacccattcccccacctcacattgcaggtatgagaaggacactgttgtggtccaggatttgggcaacatcttcactcgcctgccactcaagcggatgtggcatcaggtgtgcgagggcgtgggcttcctcctggtgaggtgggggcagggggaggggcagggaagcagaggccctgacccactgtctgtgccttccaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaagtgaggttccgtggccacctgtgacttggggtggtgggttgctggggcttgtgggcatcggcaggtttctcagggatcttctgtgtagagtttctcaatgagatgagctgtagaggatgtttaacatccatccctgacccctcagtggattgagagtcagccaattcattcttttatcttttttaaatttaaaatttttttgtttgagacaaggtctcactctgtcacctaggctggagtgcagtggtgtgatcatagctcactgcagcctcaaactcatgggctcaagcaatcctcccacctccccagtagctgggactacagacgcaccaccacccccaacaagtttttgtattttttgtacagatggtgttttaccacattgctcaggctggttttgaactctggggctcaagtgatctgcccatttcagcctcccaaagtactgggattacaggcatgagccaccccgcccagccccatttcattacgtagccacattggttgttgtttcacagttcactacagcctcaacctcctaggctcaagcgatcctcccaccttagtctactgagtagctgggtctacaggcacatgccaccattcctggctaatttttaaaaaatttttttgtagagacagggtctcactatgttgtccaggttggtctcaaactcttggtctcaagcaatcctccctccttggcctcccaaagtgctgggattacaggcgagggtccccacacccggccatcacattggttaataactattacttccgactgggcgcagtggttcacgcctgtaatcctagcacttttggaggctgaggtgggggaatcacctgaggtcaggagttcgagaccagcctggccaacatgaagaaaccccatctctactaaaaatacaacaattagccaggtgtgctgttgggcacctgtaatcccagctattcaggaggctgagggaggagaatcgcttgaacccaggaggcagaggttgcagtgagctgagatcatgctactgcactccagcctgggcaacaaaagtgaaactccatctcaaacaacaataacaaactgttaacttccatgtccagtctcctgtggccacccccccccacaaccccaagtaaaagggtaacactgggcccagcagggctgctcaggtccattttgattggtcagtgctcacactctaccctgttgtatgttgtgactcccacacctgctggcccctagacactaaatgccagatgcatcccagtcctcgtaacaaccaaaatgcgtcccttgtacatttctttttctttttttttttgagatggagtcttgctgtcactcaggctggagtgcagtgctgcgatcttggctcactgcaacctccgcctcccgggttcaagtgattctcctgcctcagccttctgagtagctgggactacaggcacgcaccaccacgcctggctaatttttgtatttttttttttagtagagacggggtttcaccatgttggtcaggctggtctcgaactcctgacctcgtgatccactcacctcggtctcccaaagtgctaggattacaggcgtgagccaccacgcccagcatccctgtacctttctaaatggtggtgagttgaggctgtctgaatcccagcccatccagcccaacatccaagtcagtcctagagaggtggcccccccccaaaaaggggagtggaacccagctgtctgccttctgggttggagcttggagataggagagaaggtctgatctgttgactacttttcgccccgacctggtcttcctggggcctgctcgtatgttcccggcagctttctggtaaacttacagtaccgccgtctctaccgaagcatgaactcccagtatctgaagctgcttagctcacaggtgagtggggagagcacagctggatcaccagcagccttaggaccccagagtagcacagcaagctgggggcccttgagacttcctgtcaggacaagggaagctgaaactccgaaaggcgaagcccagggtcctgtgattggtggggtcagaatttgaacctgggtttgtccccacccatgctgtcctgctataggaggcctaggggtctgcatcagccacggagtcttaagggtgagaagagcttcattcatgccaaaaatgggccagcagaggtccagggtgggaggagaccttgcacaaggtgatataggaagcactggcagggccaagttaggactaggttctgtccagcacagtggttaatagcatgggctctggagtcacatggcctgggtgcaagtccctgtactagccacataatcttgggcaaatttcttaaatatgagcctcaggttctccatctgtaaaatggggataataatgatatctaccttcacagataaaacatttagtagaatactggacatgcagtaagtcgttagaaaacatgaaaattccctggttcgtgttatctagcttggccccttgaatttcctgaggggtaggccttgaactagggaagggctggggactgggcttgttccacacccctcatttttaccccatcctgctttagaaataccagatcctattatataatggagatgtagacatggcctgcaatttcatgggggatgagtggtttgtggattccctcaaccagaaggtaaggtagagattcctggccctgggataggggctgctgtggaggattgggagcgggtatgcctgggagtggccagctggctgcctggctctggcccacagagggcaagtgtggaggaattcccagccctaaggtcttgctggtagctgtgagcaaggatgcagctgctgtaggctgatgtctttcctggtggggcagatggaggtgcagcgccggccctggttagtgaagtacggggacagcggggagcagattgccggcttcgtgaaggagttctcccacatcgcctttctcacgatcaaggtagggactgggcctgctgagagataactgggccggaggcaaaggagcaggacccacccgtcctttccctctggctgccttttaggctgggtcatgggtcaccatctggcccctgtatgggcaagtttttttggagaggggtggggagggttctgggaagaataaagggtttgggatgaaggaattgcccccgagtgagcagttatatggggaggagggaatggtggggtcaggagctcacgaacattgctcctcagggcgccggccacatggttcccaccgacaagcccctcgctgccttcaccatgttctcccgcttcctgaacaagcagccatactgatgaccacagcaaccagctccacggcctgatgcagcccctcccagcctctcccgctaggagagtcctcttctaagcaaagtgcccctgcaggccgggttctgccgccaggactgcccccttcccagagccctgtacatcccagactgggcccagggtctcccatagacagcctgggggcaagttagcactttattcccgcagcagttcctgaatggggtggcctggccccttctctgcttaaagaatgccctttatgatgcactgattccatcccaggaacccaacagagctcaggacagcccacagggaggtggtggacggactgtaattgatagattgattatggaattaaattgggtacagcttcaaatcccgtcttctctgtggcactgggggttagagggggcactacaggctatgaatgtgggaaaagaggggctgagaggggttggggtcctgaatgacagctgccagcttggggattgagggctcagacagctgctgtggacggtgtgggggcagtggacgccctgacatcagcaggccggttcttctcaatcacctctcgcagccctttggcaaagtggagatcagccccgatggtgaggaatccctgtgttggggagaggggagcctcatttattcattcagttattgaggccagagtttagagatcaagagacagagtgtttgtctttcaggaactcaatctaatggataattccatgtcaaggtgttaaaatgaggctgtaggagtgtagactatgggtgtcaaggagggcttcttggaggaggtgacctctgagctaagacctgaacaattagtaggtattgggatgggagaagagtgctccaagaagtcacagtgtgtgcaacggcttttaggagtctaagattaaaatgggaggggcgactggtaatgggggagctggggtcaggtaatggagggcactggggagctatagagagaggggaaggccctccctccttccccatcctgcccccactcaccgcatggttcgtcaccacctcatgcacaaagttgatgccctcaggtagtgggatctgcaccccacgccaggtccgctctgtgggtgggagcaccccgctcagtctgggcccgcccagttcacccctcctttcttcctccatcctcacaccccagccttaccattgagcatgggcatcaccccaatctgcagcatggtcttcagaggggcctgtaatgggatcagctgggaggggcgagggcggaaacagggcagtgagtcagggttgggtttgcctttagctgcccgcaggtgagcagttatatgaggagggggggatggtgagggtcaggatctcacgaacccagcccagcccacccacccttccccactcacagccagcgactccagtgcagaatggttggaatagattcggaacctgcgggaaagaaaggagccctgtgggcaggaagcctggaagctccccttcctcaggccacccaggcctacctgccagaggtgtctgagttgcaggctcagatgcccacctaggcacctttccaagtgctacaactttccaggtatcctcggggcagtcattgctgccacttgggtctccttttctccaagactgctactttttatttattgagcacctcttttttttttttttttttttttttttttgagacagagtctcgctgtgtcgcccacgctggagtgcagtggcgcgatctcggctcactgctccgtctcccgggttcacgccattctcctggctcagcctcccgagtaactgggactataggtgcccgccaccaagcccagctaattttttgtgtttttagtagagacggggtttcaccgtgttagccaggatggtctcggatttcctgaccttgtgatccgcccgtctcggcctcccaaagtgctgggattacaggcgtgagccaccacgcccggcctttattgagcacttctaatgtgccagacttctgctggtagctttgcaggtgttatttattttatgtaacaaaacttcacattgcttagttcctgtcagacactaagcatatttattttctttttttatttttatttatttttgagacagcgtctcactgtgttgcccaggctggagggcagtggtgtaattttactcactgtggctttgacctccagggctcaagtcagcctcccacctcaggcttccaagtagctgggaccacaggcgtgcatcactatgaccaactaacttttaaatttttttgtagagacaggatctcactttgtagctcaggctggtatcgaactcctggcctcaagcgatctacccgccttggtctcccaaagtgttgagattacaggtgtgagccactgcacccagccaacgtgtttaaaaagttaatattagtaaactgttttaaaatacagtaccaactggtttagtcctcaaaacaagcccaaggagtgggcactattaggatttgcactaatttatagatgaggaaaatgaggccctgcaaggttaagtaatttgcctgaagtcacacagctggaaagtgtcagaagctgcctctagaacctagggtcctaacctacattatctcctttaaccttagaacagcactgtccagtcagattttctgtgaaacgttttgtatctgcaccgtctactatggtagtcactagccacatgtggctaatgaaatgtggcttagtgcaactgaaaaactatttaattataattaatttaaatgtaaatagtcacatgtggctagtatctaccatattggatagcacagccctagaaggcagatgctacttttgcctgtattaaacacatggagaaaatgaggcctagaaagacaaaatgggctgggtgcggtggctcacgcctgtaatcctagcactttgggaggctaaggtgggacgatcacttgagcccgagaattggagaccagcctggacaacaaagtgagaccccatctctacaaaaaataaaatagtcaggcatggtagtgcatgcatgcagtcccagttactcgggaggctgaggctggacgatcacttgagcccaggaggtcaaggctgcagtgagctatgattgcaccactgcactccagcttgggcaacagagtgagactctgtctcaaaaataaataaatacaataaaataaggccaagcacagtggctcatacctataatcccagcactttgggaggccggggcaggtgaatcacctgaggtcaggagtttgagaccagcctgaccaatatggtgaaaccctgtctctactaaaaatgcaaaaattagccgggcgtggtggcgcacacctgtaatcccagctactcaggaagctgaggcagaattgcctaaacccgggaggcagaggtggcagtgagtcaagaccacgctattgcactccagcctgggcaacaagagcgaaactccttctcaaaaaaaaaaaaaaaagaataataacatttttaaaaaagataaaatggcctcaaggtcacaaagctgtgttgtgctgaggtttttgtcaaaacccatgttctgatctttgctgtgtgactgtgggcatgttatttaacattgctggacccatttgttcatctctcataatacctgccctgcctgcctctgggacatgtgagaatgcagaggagtcagactttaccaaagcactttggcatgcattaacaaagacagaccccagctctcagtggcttacaggtatcactgtactttaagcgcaacatccctgtggacaggtgtgattgtcctccaatcactgaggtgcagggaagtgcgcctgcctacctgcgcaggtccagctgcgtgcgcagggccttcccccggagagccatcttggcgctgagacgggcgtcctgcaggaacatggggaggaggatgttactgacccagaaggtcagtaacccacagcccatttttccctgacccccagccagcagcccccactctgggacccgtaccatagtcatgctggacagctggacctcaggctggtctggtgggaccagggcaatggtgacgctagcagtgacagagatggtggtgccagagggcttgatggtgcagcgcggtggggccaggacccgcagctccagcttcaatggggagtcaatcactgctgggctctgggggatgagcagcaggggcgggtcaagtccctgccatttcctttggagcccccagggaagaaggggaaggaaggcaggtgcagcttctgtcctctggggactctcatattgcaaatacagagggagctctggcttccaaaactctcacatctgcatctagccttcatgcctttgccacacaaagaaacaggaatgtgttcttctctttctgcttcctaaatacatctcaatattcaggtctatctccaatccagactctttgtggatgtcctttttgcattagtatctcagcccttgccatgtcttctgtttttgatatggagtctcactttgttgcccaggctggagtgcagtggcacaatctcggctcactgcaactctgtctcccaggttcaggcgattctcccgcctcagcctcccaagtagctgagattacaggcacctgccgccatgcccggctaagttttgtatttttaatagaaacggggtttcgccatattggccaggctagtgattcgcccacctcggcctcccaaagtgctgggattacaggcatgagccaccacacccaaaccccttgccatatctttttgctctctctctcctttttttttgagacagggtctctgttgcccaggctggagtgcagtggcatgatcacggctcactgcagcctcaacctcctcagctcaaattatcctgccacatcagcctcctgaacagctgagaccacaggttcatgccatcacgcctggcttatttttgtatttgttttttgttttggtttttgcttttgtgttttcgtagagacggggtttggccatgttgcccatgctggtctcaaactcctgggctcaagtgatctgccggcttcagcctcccacagtgttgggattacaggcgtgagccaccgtgcctggccatatcttctttctctaaactgctatgccttcttctataggcagtgaaggaatactataagatgcaactgcctaatcttcaccaaacatgattctaaacactgtatatgtgttggttcctttaaacctcataataaccatgtgaggccgatattattactgccaattttgttgttgttattttttacaaatttatttttcaggctgagtcgtcatggcagagaattgcttgaacccgggaggttcaattgcagtgagccaagattgcgccactgcactccagcctgggtgacagagtgagactttgtccccccaccgaaaaaaaaagatctgactaaaaatttatttttcaatagagacagtgtctcactgtgttgcccaggctggtctcaaactcctgggctcaagcaattttcctaccttgacctctcaaagttctcagagtataggcgtgagccatcatgcccagccatatccccatttttgagatggggaaattgaggcacagataggttaagtaacttgtagaggttatgtagttactcagtttcagaactaggcttgaagcccaggcagtctgacaatagagaccctgccctgccataaattggatgatgataatgactgaattatttcttttaaaatttgtaattctgctaggtgcggtggctcacacctgtaatcctagcactttgggaggccaaaaatgggtgatcacttgagcccaggagttcaaggacagcctgagcaacataatgaaactctgtctcaacaaaatataaaaattagcggggtgtggtggcacaagcctgtaattccagctactcaagaggctgaagcaggaggatggcttgagccctgggaggcagaggttgcagtgagcctagattgtgccactgcattccaacttgggtgacaaagtgagactcttgtctcaaaaaaaaaaataaataaaatgtgtaactcttactctcctccccaatacacaaataaacaaaagtatatgtgaattattattaaagccctcacacttgcaaagcttgggttctcactaatcctcacagcagctctgcgaaggagatggggcaggaaggagcaggaaggagcagaccgcttcacagacaggaacagccatgacaaggcgagggggattggccacagacaagaggccagggccctgtctgcgtggctgccactgggggccccacctgcacccctccttggtctcactggtgtgcaggtggccctatccctgcccccgccagcactcaccagcaggacaatgctcccaaagtaggtggccctcagcagcatgtccaggtcgtggggcacctgaacaggggaatcaggagtgagggagtgagctctgtcacagaccttctcttgcccatcccacaaacaggccatgacataccttgtcccccaccagcaacagctgcagggcccccgcccggaagtagctctccatggcagagtcgaagaagaactcagagaaggccacatacaccatccgctcttcctcctgcagctggggctccactgcccggttggggaggctccagttcctctcagtcagggggaagaaggccccctggggtggggcattcacagtcagggtcaaggcagactggcctggcacctggactgacagcaggtgtctcccagccccgtgctaggcactgtggggggatggtgggaacccagctgctgttcttggaggattcacaagcacatctcttaaaaggcattggaaggaggacacctgctaccagggggcgagcccgtcagactggaggagggagggtcaggaaaggtttcagggaagaggtgacatctgagtgggccttgaaagctagaggcagtaacaacagctactgtttacatatattgtgcatactcatgcttctttgccaagtactctaagctcttcacatagatcgacttctttaatctgcataacaacgtgtgttttcttactagattatttccatttacatgttaggaaactggccaactgaaaacttatgcttagggccgtgcagctagtcagtggcagagctaggatttgaacttcaggcattctttttctctttgtagagaaggaggtctcactatgatgcccaagctggtctcaaactcctgggctcaagcgatcctctcgcctgggcctcccaaagtattgggattacaggcatgagccactgcacccgactgattctagctcccgagtctgcactctgaaactgctatactcctctgggagaccaaaggtagtggctacacactggctatgtggccttgggcaagtgacgtcacctctatgagtaagtctccacattcctaaaattaggatgatgttagtacctgactcacagggttgtgagagaatgaaatgagattatttgtatagaaatttatgtccagctccttcgggggagttggggaaaggccgtgcatgagcaaaggccctgaggtgggaagccagggtccaatttgggaaagagcagctgcaaagaaacaggatgagatataaaaggtggggccaggtaagattaacaggggctcaaatgccagacccaggaggctgaacctaattctgtagtaacagggagccatagcattctcagtaatgggagtggcctcatcagagctgtgctttaagaagtaaaatcttgcaacttagcagagaaagaggtcttgttaggaagtcagaggtcatacaccagcccaagcagctcacccggaagtccatgtccaggttgctggtggaagccacaggatccttcatgagggaatagtcaatgccaacaagctcgtccacagaactgcgcactgcaggaaggggctcaagtcactcacggctgtgtgatgtgggataaggtgcttaacctctccaggctcagaggcccacccataaaatgaggatacatccttcacagggttgtcatgagggtcaaattagttttatgtggaaagcacttagggcctggtgtgtagttaggtgctcattaatgttagttattattattactaccactatagaggtttcaaaaaaataactaaacaagatggcatgggggggccgcgcatggtggctcacacctgtaatcccagcacttagggaggtcaaggcgggcggatcacctgaggtcaggtgttcgagaccagcctggtccaccacggtgaaaccccgtctctactaaaaatacaaaaattagttgggcatggtggtgggcgcctgtaatcccagctactcaggaggctgaggcaggagaatcgcttgatcctgggaggtggaggttgcagggagccaggattgcaccactgcactccagcctgggtgacagagcgagactccatctcaaaaaaaaaaaaaaaaaaaaaagatggcatgaggttcctttcccttggtttctcctacctcctccttcgcttcccatgcctgtgtgttaggtgtgatgggaaaatacctttgccccacagtagacagaggtcatggcttagaaaaagggaattcatggccaggcacagtggctcatgcctataatcccagcactttgggaggccaaggtgggcagatcaggaggtcaggagatcgagaccatcccggccaacatggtgaaaccccgtctctactaaaacacaaaaaactagttgggcatggtggtgcacgcctgtagtcccagctactcgagatgctgaggcaggagaattgcttgaacccgggaggtggagattgcagtgagctgagatcgcgccactggactccagcctggcaacagagtgagactgtaagactccgtgcaaaaaaaaaaaaaaaagaaagaaaaagaaaaagggaattcatgttaattgagcacctattagatgctaggtgatgaagacaaatgggatgccagtgatcagcatgcacatattgaggaccttgacaactattctctcagataatcctcacagcaaccccaggaggaggcactgccagccaagtgaggaaacaagcactttgcccagacagaaatgagcacttcacccaaaatcgcacaacaagtcagctccaggggccacatttgaacccaggactgttcaacagcagggctcggaaggggcctgctctgaggtgctgcatgacagctgcacacccagactcacccgccaccaactcaccaggcacggtgtccaggagggagttgagcaggaccgtccctgcgtggtagaggacagggcagatctgcgaggtgggagccagagtcagggccttctcaaagtgagagcatgcccacccttgccaccctgcgacctgtcgctgcccacacctgctggttgaggaggaagcgcatccctgaggtgatgaacgtggagagaaaatcatacaccttcctgtgaggagaggagaggccggatgagcccagaacctgcctccaccccagtccaggtccaggagaaatcagtgactagggaaatagtggcagtaggagtcctcagcttcagcctgaataacagctcctccacgtaggttttccaagaaccaggcccaaattccctctccagtctaattctcaagacctcctgacctactctctggtcccatcagtcaaatgaatccacccacattgcctcaaacccactctgagcatccccacatccatcggcttactgatgccctcccttccgccctctgcattcctactctgtattttcccatctgagctcagatgcttcctcttccaggcagtcttcccagattcccctcctctgaatccccaaagcattttcttagtccttctctccaggccctgatatcaaatgttttttgctccagataggctctgagctccttcctgcatcctcagggactggcatacactttgtttgatgaataaataatactgacagtgtaccaacaccaggttataatccatcatcttctctcccccatcctcttccagaacaagaacaatatccccttctcagcagcaaaaataaaaaaataggccgggcacagttgctcaggcctgtaatcccagcactttggtaggccgaggcggacagaccacctgagatcaggagtttgagaccagcgtggccaatatggcgaaaccctgtctctactaaaaatacaaaaaaagtagttgggcatggtggtgcatgcctgtagtcccagctacttgggaggctgaggcaggagaatcgtttgaacccaggaggtggaggttgcagtaaaccgagatggcgccactgcactccaggctgggtgacacagtgagactccctctcaaaaaaaaaattattatttttgttattattatttagagataccaattattgagaatccagtacatggccaaatgtgcatatcagatagtcctcacagttactcttatctaagtggccagcacttaaatatacatttatcatatgccaggtgctgttctaaatttttttttttttttttttttgagatggaatcttgctctgtcaccaggctggagtgcagtggcacgatctcagctcactgcaacctccgcctcccgggttcaagcgattctcctgcctcagcctcccacgtagctgggactacaggcacgtgccaccacgcccagctaatttttgtatttttagtagagatggggtttcaccatgttggccaggatgatctcgatctgttaaccttgtgatccgcccgcccagacctcccaaattgctgggatttcaggcatgagccaccacagccagccttgttctaaatattttttacatataccaacacaaataatcctgacaacgatcttataaaataggatcttataaaatgaggatattgaggcaccaagcttcagtgacttggccacgttcacacagccaggaagtgacagagctgagctttggacccagttggtctgattcccaggcctatgcactttgcattgcgctaggcagatgagctgtggggctcgaaaagggtgagctggggttggggctggggcttacttgaaggttcccccgaaggccgcgtgcattctggagacagaggcctggcaggagacattggacactttcatccgtccagcgggatcccgggagagctccagaccagtgcggatggacacaccctcagctgaggcgttgatgtagcccccatcatagctgccaggggggttaatattcactccaggtaggagctcaattgagtcttcagtgcccccatgcatcaccatacctcttaataaggtttcacctctttccacacatcctaccaaaccttcctatcaactcgtatgaaatgcatatgcctgtttctgttacacagatgaagaaacggaaactcaggaaggctaagggggctgcccacagccccacagcacctccagccctcctgacccactctccactcccctgagggtgctgggtccttacaagaaccagtagagcagctgtctccggaagcgcagccccaaggaggcattggtgatttgaagcatcagctcctgctgtggctggaaatcgagctcggaagatgtcagttgcagctctgtgaccttcaccctacaggaggcctcagggtcagatccagggccaagaagagggaacttcccccaacgacaggaaatttgtctgctcccctttcaagtccaggcctggggaagcgggagtgggtggaatgaattcttccattgtccctcaagtgcccaacttagacttgggtggctcaaagttgaacttgggtggctgagagaggaggtcttcctgatccaggtgttaaaaatgagcttcaccccatcgcttaaaagtcttgtttgtttgtttgccaggtgcggtggtggctcatgcctgtaatcccagcactttaggaggccgaggagggcggatcacttgaggtcaggagttcgagaccagtctggccaacatagtgaaacccgtctctaccaaaaatacaaaacttagcagaccgtggtggcacgcgcctgtagtcctagctactcgggaggctgaggcaggaaaatcgcttgaactcgggatgcggaggttgcagtgagccaagatcgcgccactgcactctagcctgggcaacagaacaagactccctctcagaaaaaaaaaaaaaaaaagtcttgtttgcttgcaccccctcccctcccacaccctggggttgctacacccggtcttttaatagagtccgttcactttgcctccatcacctaagctccgtctcacatctcaattccttattccccctccctcatcctaaaccatacttagagccttcagtttctccacccctccgattaactccaccccgtgcccgagactccactctcatctattgggaagttccttgactccacctttctgttgttcttcgaccacgcctcacatccactggtcctgcttccttcttggccccctctacattttcaaggccacgcccatctgcctggtcccgcctatgatgactggcttggcctttatcttttcggccccatccggtttcttaagtcttgcctcatcgatttggccacgccccatcccactcagcctccagtctcccgagaccctgagatttctcgggccccgcctccaccgccgaggccccgccccggatcgcgaggccccgccccccacttactcagagatgttgtagtagaagtggccttctttgccccgcaggtccggaatggtgatagtctccagctcttgctccagaaagcgcagcccctcctgcttcactgaagcagcagagaaacccttacttagctcccgtgcccactgttggggcgaccccaaccccgtccgctcccgtccgtccctgtctgcccctgcgccttacccagctccagcgccttggaggtgacgcggatcttgcagcctgggaactctgcatgtgcgcctgccagcagcgctaggaagagggccccgaagagggccatggcgagcgggcctgggggtggggtggggtcgcaggagttatctgagccgcttaaacccattccttggacgtccaaccataagtgggaactagcctggatcgttaccctccagataactcttcggggaacttggaaccaataatcttgcctccatatggcagatgtggaaactgaggctcagagaggcgatgcgacttgcgttctggctcccagaggatttgggacacgggggaggggacgttcccagttccctcgtgctaccgccgcgtgatgagtttggacctccaggggaccagcccccacacccccaacaacctcccctttcctttagcggtgggcccatgcaggcttgcacgttgccacggcaacgactgcacgcgcattcttggagggtggcttgcggtctcataggcctcgcgcccggggaggagaggaggtggccgaccagaccgtgggctcccgggtctcccggctccctcaggtcctaaatctctcccattagagattctgcctggagcccccatgttagcgttctccttcatcggctctcctccccacgccggcttcagtgcaccacgcggctcggcgagggactgggaacgggatagggacgcgccccaactcactcagcggtggagctggggggcggcgcggtcacgtgggatggcgggcagctcaggggatccgggcgacggggtctggcgggcagggttgttccagccgcctttataaagccggcctccccgacccctcctcctctctgcagctctcccctcctcccttgggggctgggaggggacggggcgggacgctggcagccgggagagtggaaaaggctgcacagagccccgtgacaagcccggccgctgtctggacaagtgggagacccaagcccagggagagggaaggaaatgcaggagccggcccactcagccccaacaatcccactttgctgggaccttggttcaaattacaattatccatgtcttcctgcatgatcttgggcaagtcacttcacttgggtcactgaccctcagtttcttccttcagcgaaatgtgcacgatgataggaactacctcttgggcctttattacaaccaggtccaggcacaaagtcaaggaatattttttctttccctgccacccacaatcatctcgggacctccttgcctggctttgctatgggcccccctacctgtctctgtgtcaccacctggggaccttaaggtcccagagacctttcccacttgggaaagcaagactagacaagtgctgtcagcccaggaacataccacaccggctgaaatcagaaggaggtctgccatttatcgtggctggtggattgttttttctattttttttatttttattttttgagacggagtctcgctctgtcccccaggctggagtgcagtggcgcgatctcggctccctgcaagccccgcctcccgggttcacgccattctcctgcctcagcctccccagcagctggggctacaggcgcacgctgccacgcccggctaattttttttgtatttttagtagagacggggtttcaccgtgttagccaggatggtctcgatctcctgaccttgtgatccgcccgcctcggcctcccaaagtgctgggattaaaggcatgagccaccgcgcccggtccactggtggattttttaatgaacccttggagcaaggtgctctgaacagcatccccgcccccacccccacccccacctccatctactgcttacagggttcccggaccctaggacagggcacagagtgggtgcttgccacctttttgttgccaaatggacgaaagaaaggggaagcaataatcatgtctgctgagcatgtatttagcaccaaacactcatctaggaaggatgactaactcatcctgatttgcctaggactttccccattgtgtcttttttttctgatataatccacagattcacaaaaatagttccatttccttttttatttaattaattaattaatttttttgagacacagtcgtgctctgtcaccaggctgcagtgcggtggtgtgatcttggctcactgcaacctctgcctcctgggttcaagtgattctcctgcctcagcctcccaagtagctgggactacagccatgtgccaccacacctggctaatttttgtatttttagtggagacagggttttgccattttggccaggctggtcttgaactcctgccctcaagtgatc

SEQ ID NO: 59 - DNA, 804 bp, Homo sapiens
  Genbank Accession No. W22366
tttnggccagcngtgctggggagggaggaagggtgatactccagggttagccgtcttcttttgggggtgtgtaccagccgnttttttcgtggatctgcaccaaggacttgtaggactgctgtgctcttgtcagactgtattgagatttgttggctccaaactgcactcgtgctttccccttcaccagtgtggcactgatctgcatgatcactgactctatggagtaggcactgctccagccctgtttggtgagaagttccatgcagatggcccctccgcccagaacataccctccagagaggactggagacacaaccctgacaaatggggggtcaaagggaaagttatctttaaaggaaaaagttaagtaggaattgaagtcgggntccttctttctctttgganggatctttggagatcgnnggnggcaaaaggngnttgtcccnggggncaaactttngggggggagggttgggnnaaatcccaantcaaatncaaagacntttgccaantnccaaacggagntttccgggcnttttaaatttgggntttccccnccccctnngggggaaaacntccnttngggnaaccccgggggatttttttttttttccccccgaaaagnntttccccttnnccaaaanaaaaaanccccgggnccaaaaaagggggggccccccnnnaaacccccngggggccccaaagagnnannattttnnaaacnccccaaannnnnnnnggggggnaaanncnntttggggcccccccntcggggggnccccccctttnnnnnnaaannctctn

SEQ ID NO: 60 - DNA, 691 bp, Homo sapiens
  Genbank Accession No. AI433892
cacaatatgcatttattaatgaatgtatttatacacaatacaaacgtgcggggacaccgtccccttcacagcccagaacccagggtcagaagatgagggatccagcctcagaggggagatatgcgacttcccaagagcagttcttggcctggaggggccatgagagtgcaagacacggggccgtggccggggcggggctacgggagcggggcgtggccggcccctgaggttactataggggaatgggccccggccaggtcccctctccttggggcagtgggaagacagcggggcccacgggccagagctgctacacgtaggcgtttctgccgtatttgccaaagctgctgtcgccttcttggtccgaggtggcgacgggcatcacggacacgggagcctggtagggccgccgggcgctggcggctgggtcctcgtcagagccgcagcagcaggcggagcagaggcagaggccacccaggantgagatcagtgaggcgctccaccccaggtagagggcggtgcccagctcgtacttggttnccgggtacaaggggtccgagaagtccccgggtgatgtgnagggcgtacangaggatggcaccatcccgcagatacccggcaagattgtgaggggcccctgcgtggcccgcagcttggctttcctgagagctcaggcccccaatgtggtgca

SEQ ID NO: 61 - DNA, 537 bp, Homo sapiens
  Genbank Accession No. AA029831
ctggtgatagtcacagtggccgagtgagtttcttgggggcccagcttcctccagaggtggcagcaatggcccggctactaggggacctagacaggagcacgttcagaaagttgctgaagtttgtggtcagcagcctgcagggggaggactgccgagantgttgcagcgtcttggggtcagcgccaacctgccggagagcagctgggtgccctgctggcaggcatgcacacactgctccagcaggccctccgtctgccccccaccagcctgaagcctgacaccttcagggaccagctccaggagctctgcatcccccaagacctggtcggggacttggccagcgtggtatttgggagccagcggccctccttgattctgtggccagcagcagggggctggctgccgcatgttgcttgactttcggttggcgggtggatgtaacaatctccaccagtgccctggctcgctncctgcagccgagcgtnctgatgcagctgaagctttcagatgggtcagataccgctttgaggtccccac

SEQ ID NO: 62 - DNA, 592 bp, Homo sapiens
  Genbank Accession No. AI279571
tcgtaaatcaaagtactattttattaaatgagttattttacacaatatgaacatcaatataattacaattgtaaaaaaattttttataacaaggatggactgattttcatatttccaaatcagagtcaactgtacatttacacagaattgtctttgcatgaagcccaagagggaacagcataaaaatgagtgtttctgtagcccctttatttttgctgatcaacagtttgttagaaaagcagctgcaggtatgttacctaaggtctgagacagtagaagagtcaaaggtgtcatgaattcacctgtaaacaaccttatgcctgaacatcagctaattctggaggaagtggagtcttaggatgcttgctctcaaagtgctgcttgaaggtcttagggtctggcatttgtgtcctacagacagtgcaggtatatattaaggcagctttggcagcagccttttggtcatgtccttgtttcttcttttgtccagcttgctttttggcatttttctgctgagactgaattttctgctgtccacgagccatatccgggccgngacagtcttgcgtcggagagacagtgcagcgcgaga

SEQ ID NO: 63 - DNA, 460 bp, Homo sapiens
  Genbank Accession No. AI982714
tcatttttatttatttatttatatttaattttttttcagacatcagtgttacttgccaagaccctcatttttaaactccaacgagttattacgttcctttccactcttgctttccaattcctacaccaagacaactctgtattttgagtatatgagtcaagtatggcaaaaagtacagtaccttaaaaacttttattttagtccttcctggcccatgctaaaacttatatctagaaatacggcaaaacaaaatgaaacagatctctccttgtacataaaacagctaaaaatttggcctctttattgatcttatgtcatgtatttggcttgaaaaaaaaaacaacaacaaaacaaacagttacaagggttaaacaaactaaatttttcttctaaatctctaaatgctcatagacagccaacttgcagggcatacacagtgccttgagagtcggtcccacag

SEQ ID NO: 64 - DNA, 2805 bp, Homo sapiens
  Genbank Accession No. D87442
ggctacggcagggggtggctctggggctgacccgggaagtcggggtctccttcgccttctgtctttctgcgtcctactagcaggtttgtgcaggggaaactcagtggagaggaagatatatatccccttaaataaaacagctccctgtgttcgcctgctcaacgccactcatcagattggctgccagtcttcaattagtggagacacaggggttatccacgtagtagagaaagaggaggacctacagtgggtattgactgatggccccaaccccccttacatggttctgctggagagcaagcattttaccagggatttaatggagaagctgaaagggagaaccagccgaattgctggtcttgcagtgtccttgaccaagcccagtcctgcctcaggcttctctcctagtgtacagtgcccaaatgatgggtttggtgtttactccaattcctatgggccagagtttgctcactgcagagaaatacagtggaattcgctgggcaatggtttggcttatgaagactttagtttccccatctttcttcttgaagatgaaaatgaaaccaaagtcatcaagcagtgctatcaagatcacaacctgagtcagaatggctcagcaccaaccttcccactatgtgccatgcagctcttttcacacatgcatgctgtcatcagcactgccacctgcatgcggcgcagctccatccaaagcaccttcagcatcaacccagaaatcgtctgtgaccccctgtctgattacaatgtgtggagcatgctaaagcctataaatacaactgggacattaaagcctgacgacagggttgtggttgctgccacccggctggatagtcgttcctttttctggaatgtggccccaggggctgaaagcgcagtggcttcctttgtcacccagctggctgctgctgaagctttgcaaaaggcacctgatgtgaccaccctgccccgcaatgtcatgtttgtcttctttcaaggggaaacttttgactacattggcagctcgaggatggtctacgatatggagaagggcaagtttcccgtgcagttagagaatgttgactcatttgtggagctgggacaggtggccttaagaacttcattagagctttggatgcacacagatcctgtttctcagaaaaatgagtctgtacggaaccaggtggaggatctcctggccacattggagaagagtggtgctggtgtccctgctgtcatcctcaggaggccaaatcagtcccagcctctcccaccatcttccctgcagcgatttcttcgagctcgaaacatctctggcgttgttctggctgaccactctggtgccttccataacaaatattaccagagtatttacgacactgctgagaacattaatgtgagctatcccgaatggctgagccctgaagaggacctgaactttgtaacagacactgccaaggccctggcagatgtggccacggtgctgggacgtgctctgtatgagcttgcaggaggaaccaacttcagcgacacagttcaggctgatccccaaacggttacccgcctgctctatgggttcctgattaaagccaacaactcatggttccagtctatcctcaggcaggacctaaggtcctacttgggtgacgggcctcttcaacattacatcgctgtctccagccccaccaacaccacttatgttgtacagtatgccttggcaaatttgactggcacagtggtcaacctcacccgagagcagtgccaggatccaagtaaagtcccaagtgaaaacaaggatctgtatgagtactcatgggtccagggccctttgcattctaatgagacggaccgactcccccggtgtgtgcgttctactgcacgattagccagggccttgtctcctgcctttgaactgagtcagtggagctctactgaatactctacatggactgagagccgctggaaagatatccgtgcccggatatttctcatcgccagcaaagagcttgagttgatcaccctgacagtgggcttcggcatcctcatcttctccctcatcgtcacctactgcatcaatgccaaagctgatgtccttttcattgctccccgggagccaggagctgtgtcatactgaggaggaccccagcttttcttgccagctcagcagttcacttcctagagcatctgtcccactgggacacaaccactaatttgtcactggaacctccctgggcctgtctcagattgggattaacataaaagagtggaactatccaaaagagacagggagaaataaataaattgcctcccttcctccgctcccctttcccatcaccccttccccatttcctcttccttctctactcatgccagattttgggattacaaatagaagcttcttgctcctgtttaactccctagttacccaccctaatttgcccttcaggacccttctactttttccttcctgccctgtacctctctctgctcctcacccccacccctgtacccagccaccttcctgactgggaaggacataaaaggtttaatgtcagggtcaaactacattgagcccctgaggacaggggcatctctgggctgagcctactgtctccttcccactgtcctttctccaggccctcagatggcacattagggtgggcgtgctgcgggtgggtatcccacctccagcccacagtgctcagttgtactttttattaagctgtaatatctatttttgtttttgtctttttcctttattctttttgtaaatatatatataatgagtttcattaaaatagattatcccac

SEQ ID NO: 65 - DNA, 737 bp, Homo sapiens
  Genbank Accession No. AI816034
tttttttttttagtcttatcatttactttccccaccccacattcttggaacagcctttagttctacaggaaatggcactgatggacagaagactagcattaccttcatgaaagggctgttagagctgcctgggaagaaggcgtgccttggggaactgggaagatgccgtcagtgtgggtgggcaggaggacagccagtcgtcctgctgccagcccaatagcttccagcggcaggtgcccaggtgctaccggagcccctcataggggtaggggcagggactgcacctcctccaggcactcatcgtaagcctcctggtactcctcatggggcttgaccattatcacacaggtggggcgcttggagcctgcggctgcacccaggtccgtcttagaggggatatagacatagggcaaatttcggtcctcacacatgactgggagatggcagtatacctcaatgggcagtgtgtctcctgccaaaaccatgatccctttttctcctttgttgacaaatttctgaacctctttcaccccgcgccgaatctgcttctgcttcaccgctttcttgatgcatttgtagagctttcgcgtgaggcggcgagaagcccagggctgcgccgatgggttctggttgacagcagcttcctgtaggtgcgcttccccgaacacgcctncgcctgagcctngggcccccgtcgatctggcctttaatttgtcatcgcagcggccgctgaaacc

SEQ ID NO: 66 - DNA, 505 bp, Homo sapiens
  Genbank Accession No. AI634570
tttttttaattttgtaaaaaatggtttatcaattccatttttgtataaaacacacgggagaaacctagccaatcaacacacaactgtcaccacatgaaaaggtacttttatcaaacttcgagtctaagaacatacaaatgtttcttttatcatgtctacagtaattgtctatgcttttccatttaactgttgttaaaaattccacatatccccattatttcttctgtcccagttacagtacaatgacggggaggaagagggttggttaaagcatccctctaagcagttttctgctgtcccttctttccaatcagagatttgtggatgtgagggatcacacctctgcagtgaggtactccagaatcgcagcactgtacacggcagcagtggcacccacccttccatggcttgtggtgcgagtcttcaagtgtctgtggatgcggcccacaggaaactgtagcccagctctctgtgagcgagatactgccttagccttggcctttcc

SEQ ID NO: 67 - DNA, 805 bp, Homo sapiens
  Genbank Accession No. AI023344
ttcattaaagtccatggatcatcacaaaaacccaggaaatgcaactaaggagaaaacaaacgtccaaccaagatctaagaacccagagctatggaggagacgttgcactggactgctgggtgtgcacaagggggcaggaggggcgatccccatggggcatggccactggccatgggaaacacaggagggaggccaggcagctggctgggcggttatgttaaccgctgcacgatgacagcattgagcaggttggcttccttcagggtctggctctcatcagccagctctttgttcgggaaagtagtcatgaggataaagctggtggcagccatggctggccgggcatccacgatgaagagtcggatgtcgctgatcctgtggctgtggttaaatttctgcaccagcctcccgccgtctgcaagccgaatttggatgtttgtggtaggctctgattcgtcgattaagatggaagagctggctttggcttcattttctgcctgttgggctggagagctggtactcaacacctggggggcagtgctgcccagtttctgaccctcgccagtgaaggctttgaaggctcctttgggcttcacaaagtcctcgtcccgatggtcctccatatccaagttcacctgtccaccgtgagctagcctccgaagctctgctggcacctcccctctgcggatagactccagaaactgggcattggatgggncttgtagcttctgaggtctccattatcccagctggatccactcttccaaaggttcatactacatgaaccatcttgcttgaaatgctg

SEQ ID NO: 68 - DNA, 1529 bp, Homo sapiens
  Genbank Accession No. Z14000
ggctgctgtttctaaaacccctttccctctaacccacaccacctttctactcactgatgccttcaggaagccataatggatggcacagagattgctgtttcccctcggtcactgcattcagaactcatgtgccctatctgcctggacatgctgaagaatacgatgaccaccaaggagtgcctccacagattctgctctgactgcattgtcacagccctacggagcgggaacaaggagtgtcctacctgccgaaagaagctggtgtccaagcgatccctacggccagaccccaactttgatgccctgatctctaagatctatcctagccgggaggaatacgaggcccatcaagaccgagtgcttatccgcctgagccgcctgcacaaccagcaggcattgagctccagcattgaggaggggctacgcatgcaggccatgcacagggcccagcgtgtgaggcggccgataccagggtcagatcagaccacaacgatgagtgggggggaaggagagcccggggagggagaaggggatggagaagatgtgagctcagactccgcccctgactctgccccaggccctgctcccaagcgaccccgtggagggggcgcaggggggagcagtgtagggacggggggaggcggcactggtggggtgggtgggggtgccggttcggaagactctggtgaccggggagggactctgggagggggaacgctgggccccccaagccctcctggggcccccagccccccagagccaggtggagaaattgagctcgtgttccggccccaccccctgctcgtggagaagggagaatactgccagacgaggtatgtgaagacaactgggaatgccacagtggaccacctctccaagtacttggccctgcgcattgccctcgagcggaggcaacagcaggaagcaggggagccaggagggcctggagggggcgcctctgacaccggaggacctgatgggtgtggcggggagggtgggggtgccggaggaggtgacggtcctgaggagcctgctttgcccagcctggagggcgtcagtgaaaagcagtacaccatctacatcgcacctggaggcggggcgttcacgacgttgaatggctcgctgaccctggagctggtgaatgagaaattctggaaggtgtcccggccactggagctgtgctatgctcccaccaaggatccaaagtgaccccaccaggggacagccagaggaaggggaccatggggtatccctgtgtcctggtctatcaccccagcttctttgtcccccagtacccccagcccagccagccaataagaggacacaaatgaggacacgtggcttttatacaaagtatctatatgagattcttctatattgtacagagtggggcaaaacacgcccccatctgctgccttttccattgccctgcaacgtcccatctatacgaggtgttggagaaggtgaagaaccctcccattcacgcccgcctaccaacaacaaacgtgcttttttcctctttgaaaaaa

SEQ ID NO: 69 - DNA, 549 bp, Homo sapiens
  Genbank Accession No. AA632300
tagaaaacaaaaatggaacatttattcgcaactcaaatactacgcatatacggtaagagattaaatataaacacagcaagttccaccccagtcctatttgtccaaggctgcatggtcaaatggaatcttgaagagaacacctggacaacagaggacctgtcagcgacgtctccggtctggacttctgctgcgtcttcggccacctctcctcttgccttttggtggaccccgaacaaaacaccagtcaacgctgatgggctgtcccatcaaatcctggccattgagtccctccatagcagcctgggcttccttgtatgtttcatattcaactagagtataccccttcagatatcctgttcgcctgtcgaagttgagatgaatgtttttaatttccccatattctgcgaatttgtcgtgtatgtcttcttcggtggcttcctcatggactccagttacaaagagaatccagccttcaacagagcgttgtggtccgggttcatcgccatcctgctccacgctgtcataatcctcacgcatccgcgctcggga

SEQ ID NO: 70 - DNA, 460 bp, Homo sapiens
  Genbank Accession No. AI798743
gagaatccagctttgatctttattcaagagaccagatgggtgggcgcaggatccggctgccagccctgaggccaagcacggctggagacccacgacctggcctgccgttgccctgagctgcagcctcggccccaggatcctgctcacagtcaccgcaggtgcaggcaggaagcagccctgggggactggacgctgctattgattcattaaaaaaagaaaagaaaaatacacccaggcctcaagtttccccgtaacagtagggcctgaggggtcggcggtcttgaggatctggaaagagcacatgagggtctcgtccacactcatcatggcgccagcattgtcaaactcgccacagtagttgggagctgagaaaagtgtcaccagctgccgcttggcaaagaactcgtagccgtcttctaccacctggtgtgctcggcagatgaggtccaagtcgtgcttg

SEQ ID NO: 71 - DNA, 635 bp, Homo sapiens
  Genbank Accession No. H98166
cctaaagcaagtaactttattatcattcctttaaaaagaaccaaggaaaattcacaacatatgtgaaacacaaacagctgtggtttaggaggtaaacaaaggaccaacatagccctgaaatgcaacagcctctgagtgacttgagccgcatgtgactggggttctgttaaaagggcaggcncctccctcctagccctgaagccccaggaacctgccttgaaagacaagctctctaatactcaacttgcagggtctcgccctaacatccaagacttggtagcatctcctttctcccaaaacccagctggaactcaactaatcctaaaccgaaaactcaagaacagcacaccagatgccacctgttgtttgtcagggtcttcaaacttccagaggaaatggcatttgcctgtcatggttttcctctctaagggcacctgtctgaacttggagctgtgcttccactggtgcctcatggggcctggggctactctccacctgaattcacacttttcnctataagggatcaggtggggaataagtccagaaggtgacaagagggtcccgggcaaagagcttgncattcctggnagggatttcccaaatggccccganggttggcgaggcgggaagnaaaa

SEQ ID NO: 72 - DNA, 534 bp, Homo sapiens
  Genbank Accession No. AA039699
ggtgagggcgggcggagatgtagacccggtagtgttgtgccttgtggtgacaactggcggcacgcgccggggcccgagacttagtctcgcacnantnggccaagcagtccacgagagcctctacttcaatcccatgatgnnccaatggggttgtgcacgccaatgtgttcggcatcaaggactgggtgacngccgtacaagatcgcggtgctggtgctgctgaacgagatgagccgcacaggcaagnggcgccgtccagcctcatggangcggcggagnctcaaccagctgctcctgcccctgcttgcagggcccaggatattacacttgtcaaaactttacaagttaattgaagagtcttgtccacagcttggcaaattcagtgcagatcagaatcaaacttgatggcttgaaagcgagtttgaangatattggaacagtttttttgatgacctttcagattctttctctggaactgaaccagaagtttcacaaaacaagtgtagtaaggttgttttctgcgtcaaatgatct

SEQ ID NO: 73 - DNA, 981 bp, Homo sapiens
  Genbank Accession No. D78132
ctgaggaggccgccaagatgccgcagtccaagtcccggaagatcgcgatcctgggctaccggtctgtggggaaatcctcattgacgattcaatttgttgaaggccaatttgtggactcctacgatccaaccatagaaaacacttttacaaagttgatcacagtaaatggacaagaatatcatcttcaacttgtagacacagccgggcaagatgaatattctatctttcctcagacatactccatagatattaatggctatattcttgtgtattctgttacatcaatcaaaagttttgaagtgattaaagttatccatggcaaattgttggatatggtggggaaagtacaaatacctattatgttggtttggaataagaaagacctgcatatggaaagggtgatcagttatgaagaagggaaagctttggcagaatcttggaatgcagcttttttggaatcttctgctaaagaaaatcagactgctgtggatgtttttcgaaggataattttggaggcagaaaaaatggacggggcagcttcacaaggcaagtcttcatgctcggtgatgtgattctgctgcaaagcctgaggacactgggaatatattctacctgaagaagcaaactgcccgttctccttgaagataaactatgcttcttttttcttctgttaacctgaaagatatcatttgggtcagagctcccctcccttcagattatgttaactctgagtctgtccaaatgagttcacttccattttcaaattttaagcaatcatattttcaatttatatattgtatttcttaatattatgaccaagaattttatcggcattaatttttcagtgtagtttgttgtttaaaataatgtaatcatcaaaatgatgcatattgttacactactattaactaggcttcagtatatcagtgtttatttcattgtgttaaatgtatacttgtaaataaaatagctgcaaacctc

SEQ ID NO: 74 - DNA, 2263 bp, Homo sapiens
  Genbank Accession No. U03985
gagccggacgtgtgcgcgaagatggcgggccggagcatgcaagcggcaagatgtcctacagatgaattatctttaaccaattgttcagttgtgaatgaaaaggatttccagtctggccagcatgtgattgtgaggacctctcccaatcacaggtacacatttacactgaagacacatccatcggtggttccagggagcattgcattcagtttacctcagagaaaatgggctgggctttctattgggcaagaaatagaagtctccttatatacatttgacaaagccaaacagtgtattggcacaatgaccatcgagattgatttcctgcagaaaaaaagcaatgactccaacccttatgacaccgacaagatggcagcagaatttattcagcaattcaacaaccaggcctactcagtgggacaacagcttgtctttagcttcaatgaaaagctttttggcttactggtgaaggacattgaatccatggatcctagcatcctgaagggagagcctgcgacagggaaaaggcagaagattgaagtaggactggttgttggaaacagtcaagttgcatttgaaaaagcagaaaattcgtcacttaatcttattggcaaagctaaaaccaaggaaaatcgccaatcaattatcaatcctgactggaactttgaaaaaatgggaataggaggtctagacaaggaattttcagatattttccgacgagcatttgccttccgagtatttcctccagagattgtggagcagatgggttgtatacatgttaaaggcatcctgttatatggacccccaggttgtggtaagactctcttggctcgacagattggcaagatgttgaatgcaagagagcccaaagtggtcaatgggccagaaatccttaacaaatatgtgggagaatcagaggctaacattcgcaaactttttgctgatgctgaagaggagcaaaggaggcttggtgctaacagtggtttgcacatcatcatctttgatgaaattgatgccatctgcaagcagagagggagcatggctggtagcacgggagttcatgacactgttgtcaaccagttgctgtccaaaattgatggcgtggagcagctaaacaacatcctagtcattggaatgaccaatagaccagatctgatagatgaggctcttcttagacctggaagactggaagttaaaatggagataggcttgccagatgagaaaggccgactacagattcttcacatccacacagcaagaatgagagggcatcagttactctctgctgatgtagacattaaagaactggccgtggagaccaagaatttcagtggtgctgaattggagggtctggtgcgagcagcccagtccactgctatgaatagacacataaaggccagtactaaagtggaagtggacatggagaaagcagaaagcctgcaagtgacgagaggagacttccttgcttctttggagaatgatatcaaaccagcctttggcacaaaccaagaagattatgcaagttacattatgaacggtatcatcaaatggggtgacccagttactcgagttctagatgatggggagctgctggtgcagcagactaagaacagtgaccgcacaccattggtcagcgtgcttctggaaggccctcctcacagtgggaagactgctttagctgcaaaaattgcagaggaatccaacttcccgttcatcaagatctgttctcctgataaaatgattggcttttctgaaacagccaaatgtcaggccatgaagaagatctttgatgatgcgtacaaatcccagctcagttgtgtggttgtggatgacattgagagattgcttgattacgtccctattggccctcgattttcaaatcttgtattacaggctcttctcgttttactgaaaaaggcacctcctcagggccgcaagcttcttatcattgggaccactagccgcaaagatgtccttcaggagatggaaatgcttaacgctttcagcaccaccatccacgtgcccaacattgccacaggagagcagctgttggaagctttggagcttttgggcaacttaaaggataaggaacgcaccacaattgcacagcaagtcaaagggaagaaggtctggataggaatcaagaagttactaatgctgatcgagatgtccctacagatggatcctgaataccgtgtgagaaaattcttggccctcttaagagaagaaggagctagcccccttgattttgattgaaaatgaa

SEQ ID NO: 75 - DNA, 1476 bp, Homo sapiens
  Genbank Accession No. U16799
gccacccaccctccggaccgcggcagctgctgacccgccatcgccatggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccaggattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcgatgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattaaattttaagtgacactacagaaaaacacaaaaaggtgatgggttgtgttatgcttgtattgaatgctgtcttgacatctcttgccttgtcctccggtatgttctaaagctgtgtctgagatctggatctgcccatcactttggctagtgacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaaagcaatgcagtaccccatagactggtgttaaatgttgtctacagtgcaaaatccatgttctaacatatgtaataattgccaggagtacagtgctcttgttgatcttgtattcagtcaggttaaaacaacggtcaataaaagaatgaacac

SEQ ID NO: 76 - DNA, 825 bp, Homo sapiens
  Genbank Accession No. AI354351
tgaagttcatcatctttagacttaaggaattaacaagggtcagggagactacaccaggctgagggcttcttggttcctggagacatgcccagctacagcaaacacagggaaacacgaagggggcagctggaagatttggtcttgaacttggggggtgggtaagtgatgatccccacgactggagcagcaggaagaagttgtgtctgaggaagtgctgggccgcccagagggacagccctgccctggagcttgtcgccgggagggaagggaaacaagccccctccctcagtgctgaggaaaaggcacttggcctgggtctcctcctgccctctccccatccgtgggagagacggggtccttgcctccttgcccctttcagccgcccagaagccggtcctggttcagcctctggaagaagcttttgccgaactcataggtgctgatcatgatggcacaggagggggcagccttgatgatccgaggaaggaagcctgcaaagagtcccttgttgcccgactcggaccggatccttcgcagcagcagccaggtggagtccacatgccaggagttcacttttccagcctccatcgctccccagacgacctggccgttggtctttaccacgtcaaagggtagaagtaagaactgcgcccaccgccctgagatgccaccagcccacaaagctcaggcccacagaagtcttgttctttcggctgagcccattgacccagtttttcaccagctctaggttaacccagaacgggcttgaaaagccacatttccgaaggccagtggggcctagcccagccacagggaacgccagcc

SEQ ID NO: 77 - DNA, 692 bp, Homo sapiens
  Genbank Accession No. D26598
cctagtacaccgcaatcatgtctattatgtcctataacggaggggccgtcatggccatgaaggggaagaactgtgtggccatcgctgcagacaggcgcttcgggatccaggcccagttggtgaccacggacttccagaagatctttcccatgggtgaccggctgtacatcggtctggccgggctcgccactgacgtccagacagttgcccagcgcctcaagttccggctgaacctgtatgagttgaaggaaggtcggcagatcaaaccttataccctcatgagcatggtggccaacctcttgtatgagaaacggtttggcccttactacactgagccagtcattgccgggttggacccgaagacctttaagcccttcatttgctctctagacctcatcggctgccccatggtgactgatgactttgtggtcagtggcacctgcgccgaacaaatgtacggaatgtgtgagtccctctgggagcccaacatggatccggatcacctgtttgaaaccatctcccaagccatgctgaatgctgtggaccgggatgcagtgtcaggcatgggagtcattgtccacatcatcgagaaggacaaaatcaccaccaggacactgaaggcccgaatggactaaccctgttcccagagcccacttttttttctttttttgaaataaaatagcctgtctttc

SEQ ID NO: 78 - DNA, 1059 bp, Homo sapiens
  Genbank Accession No. X95404
gctctcgtcttctgcggctctcggtgccctctccttttcgtttccggaaacatggcctccggtgtggctgtctctgatggtgtcatcaaggtgttcaacgacatgaaggtgcgtaagtcttcaacgccagaggaggtgaagaagcgcaagaaggcggtgctcttctgcctgagtgaggacaagaagaacatcatcctggaggagggcaaggagatcctggtgggcgatgtgggccagactgtcgacgatccctacgccacctttgtcaagatgctgccagataaggactgccgctatgccctctatgatgcaacctatgagaccaaggagagcaagaaggaggatctggtgtttatcttctgggcccccgagtctgcgccccttaagagcaaaatgatttatgccagctccaaggacgccatcaagaagaagctgacagggatcaagcatgaattgcaagcaaactgctacgaggaggtcaaggaccgctgcaccctggcagagaagctggggggcagtgcggtcatctccctggagggcaagcctttgtgagccccttctggccccctgcctggagcatctggcagccccacacctgcccttgggggttgcaggctgcccccttcctgccagaccggaggggctggggggatcccagcagggggaggcaatcccttcaccccagttgccaaacagaccccccaccccctggattttccttctccctccatcccttgacggttctggccttcccaaactgcttttgatcttttgattcctcttgggctgaagcagaccaagttccccccaggcaccccagttgtgggggagcctgtattttttttaacaacatccccattccccacctggtcctcccccttcccatgctgccaacttctaaccgcaatagtgactctgtgcttgtctgtttagttctgtgtataaatggaatgttgtggagatgacccctccctgtgccggctggttcctctcccttttcccctggtcacggctactcatggaagcaggaccagtaagggaccttcgattaaaaaaaaa

SEQ ID NO: 79 - DNA, 3309 bp, Homo sapiens
  Genbank Accession No. M15036
ctgcagggggggggggggggggggggggggggggggggcgcagcacggctcagaccgaggcgcacaggctcgcagctccgggcgcctagcgcccggtccccgccgcgacgcgccaccgtccctgccggcgcctccgcgccttcgaaatgagggtcctgggtgggcgctgcggggcgccgctggcgtgtctcctcctagtgcttcccgtctcagaggcaaaccttctgtcaaagcaacaggcttcacaagtcctggttaggaagcgtcgtgcaaattctttacttgaagaaaccaaacagggtaatcttgaaagagaatgcatcgaagaactgtgcaataaagaagaagccagggaggtctttgaaaatgacccggaaacggattatttttatccaaaatacttagtttgtcttcgctcttttcaaactgggttattcactgctgcacgtcagtcaactaatgcttatcctgacctaagaagctgtgtcaatgccattccagaccagtgtagtcctctgccatgcaatgaagatggatatatgagctgcaaagatggaaaagcttcttttacttgcacttgtaaaccaggttggcaaggagaaaagtgtgaatttgacataaatgaatgcaaagatccctcaaatataaatggaggttgcagtcaaatttgtgataatacacctggaagttaccactgttcctgtaaaaatggttttgttatgctttcaaataagaaagattgtaaagatgtggatgaatgctctttgaagccaagcatttgtggcacagctgtgtgcaagaacatcccaggagattttgaatgtgaatgccccgaaggctacagatataatctcaaatcaaagtcttgtgaagatatagatgaatgctctgagaacatgtgtgctcagctttgtgtcaattaccctggaggttacacttgctattgtgatgggaagaaaggattcaaacttgcccaagatcagaagagttgtgaggttgtttcagtgtgccttcccttgaaccttgacacaaagtatgaattactttacttggcggagcagtttgcaggggttgttttatatttaaaatttcgtttgccagaaatcagcagattttcagcagaatttgatttccggacatatgattcagaaggcgtgatactgtacgcagaatctatcgatcactcagcgtggctcctgattgcacttcgtggtggaaagattgaagttcagcttaagaatgaacatacatccaaaatcacaactggaggtgatgttattaataatggtctatggaatatggtgtctgtggaagaattagaacatagtattagcattaaaatagctaaagaagctgtgatggatataaataaacctggacccctttttaagccggaaaatggattgctggaaaccaaagtatactttgcaggattccctcggaaagtggaaagtgaactcattaaaccgattaaccctcgtctagatggatgtatacgaagctggaatttgatgaagcaaggagcttctggaataaaggaaattattcaagaaaaacaaaataagcattgcctggttactgtggagaagggctcctactatcctggttctggaattgctcaatttcacatagattataataatgtatccagtgctgagggttggcatgtaaatgtgaccttgaatattcgtccatccacgggcactggtgttatgcttgccttggtttctggtaacaacacagtgccctttgctgtgtccttggtggactccacctctgaaaaatcacaggatattctgttatctgttgaaaatactgtaatatatcggatacaggccctaagtctatgttccgatcaacaatctcatctggaatttagagtcaacagaaacaatctggagttgtcgacaccacttaaaatagaaaccatctcccatgaagaccttcaaagacaacttgccgtcttggacaaagcaatgaaagcaaaagtggccacatacctgggtggccttccagatgttccattcagtgccacaccagtgaatgccttttataatggctgcatggaagtgaatattaatggtgtacagttggatctggatgaagccatttctaaacataatgatattagagctcactcatgtccatcagtttggaaaaagacaaagaattcttaaggcatcttttctctgcttataataccttttccttgtgtgtaattatacttatgtttcaataacagctgaagggttttatttacaatgtgcagtctttgattattttgtggtcctttcctgggatttttaaaaggtcctttgtcaaggaaaaaaattctgttgtgatataaatcacagtaaagaaattcttacttctcttgctatctaagaatagtgaaaaataacaattttaaatttgaatttttttcctacaaatgacagtttcaatttttgtttgtaaaactaaattttaattttatcatcatgaactagtgtctaaatacctatgtttttttcagaaagcaaggaagtaaactcaaacaaaagtgcgtgtaattaaatactattaatcataggcagatactattttgtttatgtttttgtttttttcctgatgaaggcagaagagatggtggtctattaaatatgaattgaatggagggtcctaatgccttatttcaaaacaattcctcagggggaccagctttggcttcatctttctcttgtgtggcttcacatttaaaccagtatctttattgaattagaaaacaagtgggacatattttcctgagagcagcacaggaatcttcttcttggcagctgcagtctgtcaggatgagatatcagattaggttggataggtggggaaatctgaagtgggtacattttttaaattttgctgtgtgggtcacacaaggtctacattacaaaagacagaattcagggatggaaaggagaatgaacaaatgtgggagttcatagttttccttgaatccaacttttaattaccagagtaagttgccaaaatgtgattgttgaagtacaaaaggaactatgaaaaccagaacaaattttaacaaaaggacaaccacagagggatatagtgaatatcgtatcattgtaatcaaagaagtaaggaggtaagattgccacgtgcctgctggtactgtgatgcatttcaagtggcagttttatcacgtttgaatctaccattcatagccagatgtgtatcagatgtttcactgacagtttttaacaataaattcttttcactgtattttatatcacttataataaatcggtgtataatttt

SEQ ID NO: 80 - DNA, 604 bp, Homo sapiens
  Genbank Accession No. AA219354
gaatacaatgatgtatttctttattttcacatacactctagctaaaagagcaagagtacacatcaacaaaaatggaaacaaggctttggctgaaaaaaacatgcatttgacaaatcatgttaatagctagacaagaagaaagttagctttgtaaacttctacttcatttgattcagagaaacagagcatgagttttcttaaaagtaacaagaaaaggaacaaaaaaaatgaggtttgaaatcttttaccatggcaaaacattaacatctttctcaaaaacatagagaaatctggaaaaatcaagaagataaaattctggaccagttagtgacattctttcaagcatacttgtaaaatgtttccttaaagtgttcttgggatgaaaatgattgtcatgtctccaacaacagtgaactgatgttgttccttggaataaaagtcaatccccaccttaaaaaatgtatggcttctttgaggaattcttatggcttaaagactttttacattctagaccattaaattgattgaggnctaaattaagaagtgaatagttacnctacacggtaaggtagcagcctgaagcattggnaccaanagggtgtgg

SEQ ID NO: 81 - DNA, 1550 bp, Homo sapiens
  Genbank Accession No. M16447
gtggcctaccatggtttcaacgggtaacggggaataagggttcgattcggagctgcgggagccgggctggcaggagcaggatggcggcggcggcggctgcaggcgaggcgcgccgggtgctggtgtacggcggcaggggcgctctgggttctcgatgcgtgcaggcttttcgggcccgcaactggtgggttgccagcgttgatgtggtggagaatgaagaggccagcgctacgatcattgttaaaatgacagactcgttcactgagcaggctgaccaggtgactgctgaggttggaaagctcttgggtgaagagaaggtggatgcaattctttgcgttgctggaggatgggccgggggcaatgccaaatccaagtctctctttaagaactgtgacctgatgtggaagcagagcatatggacatcgaccatctccagccatctggctaccaagcatctcaaggaaggaggcctcctgaccttggctggcgcaaaggctgccctggatgggactcctggtatgatcgggtacggcatggccaagggtgctgttcaccagctctgccagagcctggctgggaagaacagcggcatgccgcccggggcagccgccatcgctgtgctcccggttaccctggataccccgatgaacaggaaatcaatgcctgaggctgacttcagctcctggacacccttagaattcctagttgaaactttccatgactggatcacagggaaaaaccgaccgagctcaggaagcctaatccaggtggtaaccacagaaggaaggacggaactcaccccagcatatttttaggcctcatctcagtgcctatgaggggcctgccagaaaagtcactaacctgtctcagtgtggccttgtccagccttgtgttttctgtaacccctgtttgtggtacgagataatgagtcctatttttctctcacataatatgcatttgctctcctagggacaagtgtaatacatttatgtgaagtaagacaatgcgagactggtggccgtcaaatagcatccgtcaatctgtgttaactgcataggaggctctcgatagcacctgctatagcggtgtcatgttggatcgctttgtgactgttcatctgtccttgacagtggctgtcatcttgactactttgttgatttgttggtattggggacattttaaaggctgagttatttttgaatgtcatgtttatgtcatagacgtagaaaacgcatccttgaattaaactgccttaactccttttgtggtataagcaaactacatggactctgtccctggtatccttttcctgtgtggttgcccctgtgcctgtggtctggcctaggttaagtgtgcaagataactactcgtgagttattcagaatgttgttcctaataaatgcacttgttgtctgtcttctttaatcaaatcacatcttatatacagcagtcagagatgagtatactagaatcatggattgctggaggtcttttaatctgatgttctcagaagggggtggatttaaatcctgaaataaatatttcaacac

SEQ ID NO: 82 - DNA, 3634 bp, Homo sapiens
  Genbank Accession No. D10040
tcaacacaggacaatgcaagcccatgagctgttccggtattttcgaatgccagagctggttgacttccgacagtacgtgcgtactcttccgaccaacacgcttatgggcttcggagcttttgcagcactcaccaccttctggtacgccacgagacccaaacccctgaagccgccatgcgacctctccatgcagtcagtggaagtggcgggtagtggtggtgcacgaagatccgcactacttgacagcgacgagcccttggtgtatttctatgatgatgtcacaacattatacgaaggtttccagaggggaatacaggtgtcaaataatggcccttgtttaggctctcggaaaccagaccaaccctatgaatggctttcatataaacaggttgcagaattgtcggagtgcataggctcagcactgatccagaagggcttcaagactgccccagatcagttcattggcatctttgctcaaaatagacctgagtgggtgattattgaacaaggatgctttgcttattcgatggtgatcgttccactttatgatacccttggaaatgaagccatcacgtacatagtcaacaaagctgaactctctctggtttttgttgacaagccagagaaggccaaactcttattagagggtgtagaaaataagttaataccaggccttaaaatcatagttgtcatggatgcctacggcagtgaactggtggaacgaggccagaggtgtggggtggaagtcaccagcatgaaggcgatggaggacctgggaagagccaacagacggaagcccaagcctccagcacctgaagatcttgcagtaatttgtttcacaagtggaactacaggcaaccccaaaggagcaatggtcactcaccgaaacatagtgagcgattgttcagcttttgtgaaagcaacagagaatacagtcaatccttgcccagatgatactttgatatctttcttgcctctcgcccatatgtttgagagagttgtagagtgtgtaatgctgtgtcatggagctaaaatcggatttttccaaggagatatcaggctgctcatggatgacctcaaggtgcttcaacccactgtcttccccgtggttccaagactgctgaaccggatgtttgaccgaattttcggacaagcaaacaccacgctgaagcgatggctcttggactttgcctccaagaggaaagaagcagagcttcgcagcggcatcatcagaaacaacagcctgtgggaccggctgatcttccacaaagtacagtcgagcctgggcggaagagtccggctgatggtgacaggagccgccccggtgtctgccactgtgctgacgttcctcagagcagccctgggctgtcagttttatgaaggatacggacagacagagtgcactgccgggtgctgcctaaccatgcctggagactggaccgcaggccatgttggggccccgatgccgtgcaatttgataaaacttgttgatgtggaagaaatgaattacatggctgccgagggcgagggcgaggtgtgtgtgaaagggccaaatgtatttcagggctacttgaaggacccagcgaaaacagcagaagctttggacaaagacggctggttacacacaggggacattggaaaatggttaccaaatggcaccttgaaaattatcgaccggaaaaagcacatatttaagctggcacaaggagaatacatagcccctgaaaagattgaaaatatctacatgcgaagtgagcctgttgctcaggtgtttgtccacggagaaagcctgcaggcatttctcattgcaattgtggtaccagatgttgagacattatgttcctgggcccaaaagagaggatttgaagggtcgtttgaggaactgtgcagaaataaggatgtcaaaaaagctatcctcgaagatatggtgagacttgggaaggattctggtctgaaaccatttgaacaggtcaaaggcatcacattgcaccctgaattattttctatcgacaatggccttctgactccaacaatgaaggcgaaaaggccagagctgcggaactatttcaggtcgcagatagatgacctctattccactatcaaggtttagtgtgaagaagaaagctcagaggaaatggcacagttccacaatctcttctcctgctgatggccttcatgttgttaattttgaatacagcaagtgtagggaaggaagcgttcgtgtttgacttgtccattcggggttcttctcataggaatgctagaggaaacagaacaccgccttacagtcacctcatgttgcagaccatgtttatggtaatacacactttccaaaatgagccttaaaaattgtaaaggggatactataaatgtgctaagttatttgagacttcctcagtttaaaaagtgggttttaaatcttctgtctccctgcttttctaatcaaggggttaggactttgctatctctgagatgtctgctacttgctgcaaattctgcagctgtctgctgctctaaagagtacagtgcactagagggaagtgttccctttaaaaataagaacaactgtcctggctggagaatctcacaagcggaccagagatctttttaaatccctgctactgtcccttctcacaggcattcacagaacccttctgattcgtaagggttacgaaactcatgttcttctccagtcccctgtggtttctgttggagcataaggtttccagtaagcgggagggcagatccaactcagaaccatgcagataaggagcctctggcaaatgggtgctcatcagaacgcgtggattctctttcatggcagaatgctcttggactcggttctccaggcctgattccccgactccatcctttttcaggggttatttaaaaatctgccttagattctatagtgaagacaagcatttcaagaaagagttacctggatcagccatgctcagctgtgacgcctgaataactgtctactttatcttcactgaaccactcactctgtgtaaaggccaacagatttttaatgtggttttcatatcaaaagatcatgttgggattaacttgcctttttccccaaaaaataaactctcaggcaagcatttctttaaagctattaagggagtatatacttgagtacttattgaaatggacagtaataagcaaatgttcttataatgctacctgatttctatgaaatgtgtttgacaagccaaaattctaggatgtagaaatctggaaagttcatttcctgggattcacttctccagggattttttaaagttaatttgggaaattaacagcagttcactttattgtgagtctttgccacatttgactgaattgagctgtcatttgtacatttaaagcagctgttttggggtctgtgagagtacatgtattatatacaagcacaacagggcttgcactaaagaattgtcattgtaataacactacttggtagcctaacttcatatatgtattcttaattgcacaaaaagtcaataatttgtcaccttggggttttgaatgtttgctttaagtgttggctatttctatgttttataaaccaaaacaaaatttccaaaaacaatgaaggaaaccaaaataaatatttctgcatttc

SEQ ID NO: 83 - DNA, 377 bp, Homo sapiens
  Genbank Accession No. W42636
cactatgtacaaaaacattttaattgaaatacctgtataaaaaaatatgatctccagacatctcacttttgaactgaaagaacccccatctgcgatgcctgcacacaccgcattcacacaaacacaggtactgaataaattaaacgctcaggctctggccccaccccagctttcagagcccacaagcagactgtacaaagtcaataatttaaaacccaaaccctgggcacagtgcctggaagtgtcagggtcacccactccccttaagttagccactatacatgttcatcttctgacaggcggggccaggacagacgccaggcacagggaatcaggggctgggggtcccttggaccacaggccaaccccctcccctt

SEQ ID NO: 84 - DNA, 418 bp, Homo sapiens
  Genbank Accession No. T66157
tttttagaaagaaatagatttattctcatgtacaaagcggtcacccacgggaccatatacgacagttgcacagagtcctagaaaaacgcatctntctaaaggcaactcagaaaggtaaggcaggtggaccccctcccccaccccacaacgcacacagantgaaacggagaaaaagagagaagccagtgncngggntgacccaagattcccggccctatggggtttcccaagccccagggcacaggtggntatggccttnaagagagagccctgccaggttnanggccaggtttttcattggctgcaggnttgggtaaggggntcaggccaaggggaacantcagggggcctntnagacantcaggnttgggntctctgtgccttaggggtttgcagaaacattccatagcctctgctg

SEQ ID NO: 85 - DNA, 1811 bp, Homo sapiens
  Genbank Accession No. M62831
ggtttgtgtagagaggcgtgcagagcccgttgtccggagtgcacctgctgcctgttctgtccctcccgggagcccccgccgctgtcgccgtcgagtcgccatggaagtgcagaaagaggcacagcgcatcatgaccctgtcggtgtggaagatgtatcactcccgcatgcagcgcggtggcctgcggctgcaccggagtctgcagctgtcgctggtcatgcgcagcgcccgggagctctacctctcggccaaggtggaggccctcgagcccgaggtgtcgttgccggccgccctcccctctgaccctcgcctgcacccgccccgagaagccgagtccacggccgagacagcgacccccgacggtgagcacccgtttccggagccaatggacacgcaggaggcgccgacagccgaggagacctccgcctgctgtgccccgcgccccgccaaagtcagccgcaaacgacgcagcagcagcctgagcgacggcggggacgttggactggtcccgagcaagaaagcccgtctggaagaaaaggaagaagaggagggagcgtcatccgaagtcgccgatcgcctgcagccccctccgggccaagcggagggcgcctttcccaacctggcccgcgtcctgcagaggcgcttctccggcctcctgaactgcagccccgcggcccctccgacggcgccgcccgcgtgcgaggcaaagcccgcttgccgcccggcggacagcatgctcaacgtgctcgtgcgggccgtggtggccttctgaggaccccgagcggcgctgccggagcccagagcgcgcgtcgaaccgtcggcccgagggcgcagacctgaggcgaggccacccccctccatcctgggggaagcgcccgcgaaaaccgtggagagaagccgccgcccgggctgctgagaggcccggagaggactctgtccccggggagccatcgccttcagtgtgcagggacggcaccgaggagtctgagccgggcgcgggcgccttccgcagagacctgcgcccacaggtgctgtcttagtggactgggacgtgaacctttcgctctccttctggactgggagaagggaggcttgggtgttgtgttttttgttttgtttgtttgtttgtttttaaagatctcctcagggtcggacttcattttgtactgtgggctgtgctggccctttcaaggtttttcaagagttggttttgcgtttccaacctcggagaattccaggcactccccttccccctccgctgacatacttgtataagcggtcatcgttgcgtcatggggcaggcgtggggagcttcctgtcgccttggctgggtgtgggcctggaggaaggtcctggggcgtgcactcgcctgggcagtggggaggagagtggcctgagttacttcacccccgcgtgctgctggttaatgtcccgcgtctctgcaccttcgggtgggagcggggactgatctactttcacattctcaagtttttctcatctgcattagaggtccccagtaggttcccaggttccagcgtgcccctccctcagacacacggacacaatcagccgagaagttcctggtctgaatcacgagaatgtggaggggtggggggtgtcagtggaaaggcataaggctgagctgagaccagttgctggtgaaactgggccaatctggggaggggaacatccttgccagggagtttctgagggtctgctttgtttacctttcgtgcggtggattctttttaactccgtctacctggcgttttgttagaaatgtcagataggaaaataaaaaccatttgagt

SEQ ID NO: 86 - DNA, 372 bp, Homo sapiens
  Genbank Accession No. T98839
gattcaaaagaatggagatggagaagaaggggaggggagggaggggagagataaacagagagagacagagaattatatagcagtatgcaaaaaaccagtttaaaacctgtgaagcaaagagaaatgggtgtatgagctagacaggcatgaggagtgacagagtttcctcttcaaggagacggtatcccttctgttgacatacacaggtgatccagaactnccgtgagtgtccttcccgacgagatttccctctggggtgaattttgtgcggggtggccctcaaagctcctctctaccacccgtgaggaccctccccaaagactttttattctttttaaagtcttgggaaaagccctgcttttcctcctttta

SEQ ID NO: 87 - DNA, 924 bp, Homo sapiens
  Genbank Accession No. U41344
agttcagagagaagtctacagtccttgtctcggggtgtcttcccccttccccttcctcatcttccccgccagcctccactcccttctgatttctcgtcttctttttccgtagaaatcaacggaacccagatttgccccaacgacctagtggcgttccatgacttctcctcggacctggagaacgtgccacacctgcgctacctgcggctggatggaaactacttgaagccgcccatcccgctggacctcatgatgtgcttccgcctcctgcagtccgtggtcatctaggccctactccgccaccggatctgctctgaccgcacttgaaggctggggcccaggacctgtgccggccattcgttttctctctctccctttctttctcccagctttgcctccttatgccacctcgaggcagggaaaagccatctatacttctgcagcctaaggagcgagacttcaaggactcagtttggttccacccagttgaaagacacccagtgcacacccaaactcctggccttctgtggtttccctttgctccagaaacacagatgtgtctaaagacttggtgtccccttctctctcctcccccacccgccactcctgggtacatctgggccgtggactatctgatcttggtcctggccgagaaacagcccggagtaaggttcagaaagtccccctggggactcctccatcttcggcagctctggctcaaagaggccgagaaacccagttctcctgggccaaggatcccttctggcaaagctgctgctccgccgcttggttccccggcaccagacatcgccctggccctgctggcccgggtatcggaaggaggagcgagggaatgacggaggtagggtgcagggtaagggctcccaggtcttcatctggccagcaggacacagggatctaagg

SEQ ID NO: 88 - DNA, 511 bp, Homo sapiens
  Genbank Accession No. AI765890
tgcttttgaattattgatttattgcacatcaggagaaacacaagattactgctataataaattcactcttacatgctttagcaaaaatcagtaaaaatagaaaacatggtaacaattaaagtgaaaaaattggggtcattaaagaatgtctgactgattagcttgcagtttttgagacggctgagaactaccatcaatgagatcaccttaaacaaacactcttaatgacttgaaaaagtccccatcccaaagtcaactataggtattcaatattgtatatttaactgaatttaaagttatgttaaaacccattttattgaagtaagagaagaattccttttacattatcattctcaagccatatatcaagaaaaagattgaaattacattcctttttttttttttaatagagacaagttctcgctgtgttgcccaggctggtctcgaactcctaggttcaagtgatcctcctgccttgtcctgccaaagtgctgggattacaggcatgagc

SEQ ID NO: 89 - DNA, 318 bp, Homo sapiens
  Genbank Accession No. AL043089
gtgggttggagactcagcaggctccgtgcagcccttgggaacagtgagaggttgaaggtcataacgagagtgggaactcaacccagatcccgcccctcctgtcctctgtgttcccgcggaaaccaaccaaaccgtgcgctgtgacccattgctgttctctgtatcgtgatctatcctcaacaacaacagaaaaaaggaataaaatatcctttgtttcctaaaaaaaaaaaaaaaaaaaaaaaaaaaaaaaaaaaaaaaaaaaaaaaaaaaaaaaaaaaaaaaaggggcggccgntntaaaggatccaagcttacgtnc

SEQ ID NO: 90 - DNA, 716 bp, Homo sapiens
  Genbank Accession No. M11433
gggggggggcggagggcgctcatttccgggccgcccaccacccgcgtagcaccggcagccgctgtcccggcagtctccagccgtcccgcccgcttgtggccaaactggctccagtcactcccgaaatgccagtcgacttcactgggtactggaagatgttggtcaacgagaatttcgaggagtacctgcgcgccctcgacgtcaatgtggccttgcgcaaaatcgccaacttgctgaagccagacaaagagatcgtgcaggacggtgaccatatgatcatccgcacgctgagcacttttaggaactacatcatggacttccaagttgggaaggagtttgaggaggatctgacaggcatagatgaccgcaagtgcatgacaacagtgagctgggacggagacaagctccagtgtgtgcagaagggtgagaaggaggggcgtggctggacccagtggatcgagggtgatgagctgcacctagagatgagagtggaaggtgtggtctgcaagcaagtattcaagaaggtgcagtgaggcccaagcagacaaccttgtcccaaccaatcagcaggatgtgtgagccaggatccctctttgcacagcatgaggcaaaaatgtccagccacccctaggcatctgttagcagagtctgtctcttggctttgtcacttttccttttcttaaaacaaagccatgccaataaagtgacctgtgttc

SEQ ID NO: 91 - DNA, 2052 bp, Homo sapiens
  Genbank Accession No. Y11307
gcggccgcgtcgacgcgcccccgagcagcgcccgcgccctccgcgccttctccgccgggacctcgagcgaaagacgcccgcccgccgcccagccctcgcctccctgcccaccgggcacaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgaccaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttcctttggggcttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacctaaaaaaaagtcgacgcggccgc

SEQ ID NO: 92 - DNA, 748 bp, Homo sapiens
  Genbank Accession No. AI078144
ttttgtggattttccttttaatgcaaaatgttgcaatacaaaacaatgtggagaaagtcctgttcctcaggacactgaagggaggagtgaggaagagaggacagagctggacgtctcctcctatttctccctccccaagtcactctgaggggaagaacactgctgcctgctccctgggcctgcccgcatacaaggttagagccctgggtctggggcatccttagcctgaaatttgttgacatggggcaggagagcaggagggaacattgagggttttgactcttcgggctctaaaaggattactcaggatctggagttccgtatgaaacaaaggagctgaaagaatttgattgccattggctaaaaatatagaggatttgagccacaactggcccacatttgaagaatgaggaacaagaaatttagtggggatataatataaatgtatgggagtgagaaagatgcaaaaaacaaggctagctcctcaaacctcctcctctttcttcctcatctccagcttatagacaatctggtagccatcatcccaggcatagagctggcgttctcgggggttatagcggaggctggcatgggcaccatatctgcggngaaaataagggagtgctgcccgttcaggggtcagggtgccgctggcatcaaaggagcactggatgcgggcccgactggcaggacgggtgttatagacgacatagaaggtcccacagatgacaaaggcagcctcagcatt

SEQ ID NO: 93 - DNA, 602 bp, Homo sapiens
  Genbank Accession No. AA122235
gggagattaaaaccttggattctaggtggtagaggataatttttttttgaatgaatttctttttatttatttatttatttttttcctttttaggatagaatttttgcggcatttgaagagctttttccagattatgtttaaaattgaaaccaagccatgtggtgaagaactcaagggtggggataaagtgctgatgacctgtgttggcattggtttctccaaccttagcaagaccctcaagtgataaccatcacaagataaggccccaatgcctacagacaaagcagaagctgccacggacaccaatgggaccaagtccaaatggattaatccaggacagaatagccacttgcttaattttctgtgaagaaatatcaatatacaaataaaagacatccctgtagcatatggtttccagctgtttctccagtggcattgcattgcccaggagggnccagtcaccatgagagctcccttgccttacctggaggaagaatgtgcttcaggcacagtcgtgctgctagaacagtctcatagctgcagttcantgtcttcctcagctactatcatangttcctcagccctgtcatatgctgtttcca

SEQ ID NO: 94 - DNA, 674 bp, Homo sapiens
  Genbank Accession No. AI342377
gagatttcccaggactggctttaatttgaaaaatctgattggggtctcttcccgtatcagagaaggaacagcccaagctatgaccccagggccagggaattcagtccccaccagaccctgtcattccatcactagggggtaattccaggctccccctgccagccctgagacaggaggacggatgtgaagttgcccaggactagattctgtctctccaaagtggcccaagccctgttctctgtactagggaagccagctgtgtcttttcgaggacagttggtccagccagcaggctcagttcagataccagacaaccattccagcacgagggctcagcgccctggccccggcggtcgctccagtgcctgtgtgcccaccagcacatccatgaggtagtccaattcggcctcgtccagctccggagcttcctccttgcccggcccatcctcagggcctggtttgaggccctcagaggctggtgcccanagttcattgtcatacatagaggtgtcaatatcctcaaacaggccctcaagcccatcgtccagtagacagccagtggctgggcccagcaggtccaaggcacccaggctgggcgctgctcccccgatgctactggcctgtgggcccctcgtctgccaagggttgggagcctgactcangcccctcgtgcc

SEQ ID NO: 95 - DNA, 1155 bp, Homo sapiens
  Genbank Accession No. U86529
aagacacgggcctgattcgtcgagtctcactgagccttagtcgtcggcaggtcccaggcgcgaagtttctcggcctggaggagggggtcgcgcgaagtgccagatgcaggcggggaagcccatcctctattcctatttccgaagctcctgctcatggagagttcgaattgctctggccttgaaaggcatcgactacaagacggtgcccatcaatctcataaaggataggggccaacagttttctaaggacttccaggcactgaatcctatgaagcaggtgccaaccctgaagattgatggaatcaccattcaccagtcactggccatcattgagtatctagaggagacgcgtcccactccgcgacttctgcctcaggacccaaagaagagggccagcgtgcgtatgatttctgacctcatcgctggtggcatccagcccctgcagaacctgtctgtcctgaagcaagtgggagaggagatgcagctgacctgggcccagaacgccatcacttgtggctttaacgccctggagcagatcctacagagcacagcgggcatatactgtgtaggagacgaggtgaccatggctgatctgtgcttggtgcctcaggtggcaaatgctgaaagattcaaggtggatctcaccccctaccctaccatcagctccatcaacaagaggctgctggtcttggaggccttccaggtgtctcacccctgccggcagccagatacacccactgagctgagggcctagctcccaaatcctgccccgttggcacagggccacaggagcagaagctgggtgggctgaagaggcctggaaacgagagtcttaattgaggagatgggagactcgaactctagccctggatctgccttcctgctgaaacttgttccacctcagtcccctcatctgtcacacgcatgtggggtggagtagggagatgcggggagcagggtgggcaggaatactgttatctatgtgacggggcagtcgtgaggctgagatgagaatgcggattaaaatgcctggcgtgctcaccgtaacaccacggggaaggctgtgtgccttttctcatccgcttttgttgtgtgtgactccaaagaatgcccgcgctgaaatttggcgtgaattaaactgaagcccaggcctct

SEQ ID NO: 96 - DNA, 391 bp, Homo sapiens
  Genbank Accession No. AI680350
tttttcattaaaaaatagggctctttattataaattactgaaatgtttcttttctgaatataaatataaatatgtgcaaagtttgacttggattgggattttgttgagttcttcaagcatctcctaatagcctcaagggcctgagtaggggggaggagagaggactggaggtggaatctttataaaagacagagtgattgaggcagattgtaaacattattaaaaaacaagaaacaaacaaaaaaatagagaaaaaaaccaccccaacacacaactgccctgtccagcccaatacctgacacagaatactntgtgtttgtttagttgcccccccccacaaaaacaaaaacaaaaacaaaaacaaaaaactgttccaggtaattccatcgct

SEQ ID NO: 97 - DNA, 620 bp, Homo sapiens
  Genbank Accession No. AA576961
gttttttttttttttaatatcagagttttaatttcaaccagctggcacaacaatgaaagtgtcagactttctgaaagtactcgagaaataatgaataaattcttaatgttttcccctccaccgcccttttttattctccaagattaggaattactacggattaggtttttgaaaataaagtttcctttttggaaaatggtctacattcagaaatgtcttagaacaagcatttaaaaaaaactaataaataatcataaatcaaaatacattaaaataaaattacagtacatcatcgctcctagaaaattcaccatacaagacgatcctttcaaaggttcataaataaaagtcttcttgactcgaaatcgtttcctgcatcgtgatgaaaagtatgcagaaaactaagaagaatcgcaagttttcagtagggtgatgtccaaactacttgatctggtgcggggcggagagactgttttgcttttgatccaagtgaagacaatagaaatgtgctcgtcccacttcctcaagtcctcaaaaccttgtcttgcccgggagctgcccctttcaggcagagttggaggtgctgcggagaagccggtgcccgtgcggctgcgagtgcg

SEQ ID NO: 98 - DNA, 1108 bp, Homo sapiens
  Genbank Accession No. U75744
ggcagagcaggggtagacttagcatgtaagggagggtcagttggtcctgcaacaggtaggacatccctcgggtacaatggagcagcaccaggatgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacgtacaactatgtgattagctctcggcttggaagaaaaacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcctagacccaagggtctcatcttattaaccatttcttgcctctaaataaaatgtctctaacagatatgaactgctccctgtacttaggaaaaaaaaaaaaaaa

SEQ ID NO: 99 - DNA, 3921 bp, Homo sapiens
  Genbank Accession No. M59371
cggaagttgcgcgcaggccggcgggcgggagcggacaccgaggccggcgtgcaggcgtgcgggtgtgcgggagccgggctcggggggatcggaccgagagcgagaagcgcggcatggagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtaacaactttgagctcaactttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccttccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctgagcctcgacagggcctggagccccatcggccaagaatacttgaagaaacagagtggcctccctgctgtgccatgctgggccactggggactttatttatttctagttctttcctccccctgcaacttccgctgaggggtctcggatgacaccctggcctgaactgaggagatgaccagggatgctgggctgggccctctttccctgcgagacgcacacagctgagcacttagcaggcaccgccacgtcccagcatccctggagcaggagccccgccacagccttcggacagacatataggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgttaaagattttgttttgttggacatttttttcgaatcttaatttattattttttttatatttattgttagaaaatgacttatttctgctctggaataaagttgcagatgattcaaaccg

SEQ ID NO: 100 - DNA, 1247 bp, Homo sapiens
  Genbank Accession No. AF040639
ggaattcgccgcccgctccgagcctggtgcttccgaccgctgcgcgcggctcctgggctgtcacagtctcccgttgccgccgtcatgtcccggcagctgtcgcgggcccggccagccacggtgctgggcgccatggagatggggcgccgcatggacgcgcccaccagcgccgcagtcacgcgcgccttcctggagcgcggccacaccgagatagacacggccttcgtgtacagcgacggccagtccgagaccatccttggcggcctggggctccggctgggcggcagcgactgcagagtgaaaattgataccaaggccattccactgtttgggaactccctgaagcctgacagtctccggttccagctggagacgtcactgaagcggctgcagtgtccccgagtggacctcttctacctgcatatgccagaccacagcaccccggtggaagagacactgcgtgcctgccaccagctgcaccaggagggcaagttcatggagcttggcctctccaactatgcagcctgggaagtggccgagatctgtaccctctgcaagagcaacggctggatcctgcccactgtgtaccagggcatgtacaatgccatcacccggcaggtggaaacggagctcttcccctgcctcaggcactttggactgaggttctatgccttcaaccctctggctgggggcctgctgaccggcaagtacaagtatgaggacaaggatgggaaacagcctgtgggccgcttctttgggaatacctgggcagagatgtacaggaatcgctactggaaggagcaccactttgagggcattgccctggtggagaaggccctgcaggccgcgtatggcgccagcgcccccagcatgacctcggccaccctccggtggatgtaccaccactcacagctgcagggtgcccacggggacgcggtcatcctgggcatgtccagcctggagcagctggagcagaacttggcagcggcagaggaagggcccctggagccggctgtcgtggacgcctttaatcaagcctggcatttggttgctcacgaatgtcccaactacttccgctaggcccatcgtttctcaggctgcccaaggctcttctgtaacatcttttgttactcacattttctttaatttagaactgcctcagtaaattcttagggatggaagtntttggacaaaaacctaatagaagagtcaccaccaaatgaagaataaaactcccaaaaaaaaaa

SEQ ID NO: 101 - DNA, 1450 bp, Homo sapiens
  Genbank Accession No. M12963
gatgcacttgagcagggaagaaatccacaaggactcaccagtctcctggtctgcagagaagacagaatcaacatgagcacagcaggaaaagtaatcaaatgcaaagcagctgtgctatgggagttaaagaaacccttttccattgaggaggtggaggttgcacctcctaaggcccatgaagttcgtattaagatggtggctgtaggaatctgtggcacagatgaccacgtggttagtggtaccatggtgaccccacttcctgtgattttaggccatgaggcagccggcatcgtggagagtgttggagaaggggtgactacagtcaaaccaggtgataaagtcatcccactcgctattcctcagtgtggaaaatgcagaatttgtaaaaacccggagagcaactactgcttgaaaaacgatgtaagcaatcctcaggggaccctgcaggatggcaccagcaggttcacctgcaggaggaagcccatccaccacttccttggcatcagcaccttctcacagtacacagtggtggatgaaaatgcagtagccaaaattgatgcagcctcgcctctagagaaagtctgtctcattggctgtggattttcaactggttatgggtctgcagtcaatgttgccaaggtcaccccaggctctacctgtgctgtgtttggcctgggaggggtcggcctatctgctattatgggctgtaaagcagctggggcagccagaatcattgcggtggacatcaacaaggacaaatttgcaaaggccaaagagttgggtgccactgaatgcatcaaccctcaagactacaagaaacccatccaggaggtgctaaaggaaatgactgatggaggtgtggatttttcatttgaagtcatcggtcggcttgacaccatgatggcttccctgttatgttgtcatgaggcatgtggcacaagtgtcatcgtaggggtacctcctgattcccaaaacctctcaatgaaccctatgctgctactgactggacgtacctggaagggagctattcttggtggctttaaaagtaaagaatgtgtcccaaaacttgtggctgattttatggctaagaagttttcattggatgcattaataacccatgttttaccttttgaaaaaataaatgaaggatttgacctgcttcactctgggaaaagtatccgtaccattctgatgttttgagacaatacagatgttttcccttgtggcagtcttcagcctcctctaccctacatgatctggagcaacagctgggaaatatcattaattctgctcatcacagattttatcaataaattacatttgggggctttccaaagaaatggaaattgatgtaaaattatttttcaagcaaatgtttaaaatccaaatgagaactaaataaagtgttgaacatcagctggggaattgaagccaataaaccttccttctt

SEQ ID NO: 102 - DNA, 38059 bp, Homo sapiens
  Genbank Accession No. K02402
gtatatctagaaaaccccattgtctcattccaaaatcaccttaagatggataggcaacttcagcaaagtctcaggataacaaaatcaatgtgcaaaaatcacaggcattcttatacaccaatagcagacaaacagacagccaaatcatgagtgaactcccattcacaattgcttcaaagagaataaaatacctaggaatcctacttacaagggatgtgaaggacctcttcaaggagaactacaaaccactgctcaatgaaataaaagaggatacaaacaaatggaagaacattacatgctcatgggtaggaagaatcaatatcatgaaaatggccataatgcccaaggtaatttatagattcaatgccatccccatcaagctaccaatgactttcttcacagaattggaaaaaactactttaaagttcatatggaaccaaaaaagagcccgcatcgccaagtcaatcctaagccaaaagaacaaagctggaggcatcatgctacctgacttcaaactatactacaaggctacagtaaccaaaacagcatggtactggtaccaaaacagagatacagaccaatggaacagaacagagccctcagaaataatgccacatatctacaactatctgatctttgaaaaacctgacaaaaacaagaaatggggaaaggaatccctaattaataaatggtgctgggaaaactggctagccatatgtagaaagctgaaactggatcccttccttataccttatacaaaaattaattcaagatggattaaagacttcattgttagacctaaaaccataaaaaccctagaagaaaacctaggcaataccattcaggacataggcatgggcttggacttcatgtctaaaacaccaaaagcaatggcaacaaaagccaaaattgacaaatgggatcaaatgaaactaaagagcttctgcacagcaaaagaaactaccatcagagtgaacaggcaacctaaagaatgggagaaaatttttgcaatctactcatctgtccaagggctaatatctagaatctaaaatgaactcaaacaaatttacagaaaaaaacaaacaaccccatcaacaagtgggtgaaggatatgaacagacacttctcaaaagaagacatttatgcagccaacagacacatgaaaaaatgctcagcatcaccggccatcagagaaatgcaaatcaaaaccacaatgagataccatctcacacaagttagaatggcgatcatcaaaaactcaggaagcaacaggtgctggagaggatgtggagaaataggaacactttgacactgttggtgggactgtaaactagttcaaccattgtggaagtcagtgtggcgattcctcagggatctagacctagaaataccatctgacccagccatcccattattgggtatataccaaagtattataaatcatgctgctataaagacacatgcacacgtatgtttattgcggcacttttcacaatagcaatgacttggaaccaacccaaatgtccaacaatgatagactggattaagaaaatgtggcacatatacacctaggaatactaggcagccataaaaagaaaatgagttcatgtcctttgtagggcatggatgaagctagaaaccatcattctcagcaaactatcgcaaggacaaaaaaccaaacaccgcatgttctcactcataggtgggaactgaacaatgagaacacttggacacaggaaggggaacatcacacaccggggcctgttgtggggtggggggcgaggggagggatagcattaggggatatacctaatgctaaatgacgagttaatgggtacagcacaccaacatggcacatgtatacatatgtaacaaacctgctcgttgtgcacatgtaccctaaaacttaaagtataataataaaaaaaagatcattctaaaatttatacaagcccttagaacagttaaaaatatcttaccaaaagaagaataaagttggaggaatcactctacctaatataaagtcttactacatagctacagtaattatgacagtgttatattggcagagggataaatacatcaatggcacaaagaatagatagagaaactggaagtagacccaaaacaatatggttaactgacttacgaaaaaatttcagaagccattcagtcgaggaaggatagggtggtattgttgttttttgttttaacaaattgtgctggataaattggacatacctatggaaaaaaaaatgaagtttgacctaaacatcatactttacacaaatattaactcaaaatggagcatgggcataaatctaaaacttcaaactgtaaaacatttagaaaaaaataggaaaaaaactatcaggatctagtgttagtggaagagttctaaatgtgatccataaaacaaaaacaaataaactggactacatcaaaactaaaaaattctactctgtgaaagacctaattaagaggacaaaagacaagctacaggctggagacaatatatttaatccacgtatctatgaaaggattcatatctagaatatataaacaaccttaagaatctgacagtaaaaaaaaaaaatcagactaactggaccactcatacattgctgatggaaatgtaaagtggtacagccattttggtaaacatcattgctctctgacaaagatacggtgggtcccactgatgaactgtgctgccacagtaaatgtagccactatgcctatctccattctgaagatgtgtcacttcctgtttcagactcaaatcagccacagtggcagaagcccacgaaatcagaggtgaaatttaataatgaccactgcccattctcttcacttgtcccaagaggccattggaaatagtccaaagacccattgagggagatggacattatttcccagaagtaaatacagctcagcttgtactttggtacaactaatcgaccttaccactttcacaatctgctagcaaaggttatgcagcgcgtgaacatgatcatggcagaatcaccaggcctcatcaccatctgccttttaggatatctactcagtgctgaatgtacaggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagtttgattatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcagactgaaatgctgaaataacgataaaaaaaaatacagaggttaaactagcaaagtgagtaaagtcaagggataaagaaaatttgttggaaaactcacaaagcaggacataaagcaaggccattagatatatctcattagtgtgacatctgggaggacaaagcatccaaaccctttcttctatataagtggtgagatgatgaaggttgtaagaggcttctgcccccttgaagacttcagatgctggggaaaggatagataagaataaggatgaacctggcttttggagcctgggaaataatgactagcgataaacctgaagggaagttaagtatacgatccccagataatactaaggagaaaggcaatgtgattctgcagccattgtagccagagataataagcccttgaggaaggggccaggggaatttttctaaggatagacagtattaatgcagcactctcttctgctattaaactctcattggcttctaaaaggagtttcggtgagtgatttgctgagatgtttgcattttcatgctgctgcctttaggttattattgcaacagtttggaattttgaaattaaaacagttctgtaaaaccagtttagttttgtaaagtgtatgcatcaaagatgtccttcattcagacattactgagttacaactacggtgccaggtactgtgtcagggtactaggggtatggggataaaccagactccctctttgatctaaagcagcatgaggccaggtgagaggtttcaatatatgtgataaaatgtgcactaggtactaagggatcatagagaaaggaacacattaaatggggaaacaattgatagagagagaatattttcatctgggtcttaaaagatgagtaggcgttctctctctttaaatgtctgatataagggcattttatgcaaagaaggatcactcgtgcaaagactcagctttgcaagaacgtgaggtatttcaggagttttgtatggttccatatggactatgacaagtgagacaggtaaactaggcagagctggtcatcagataatgaagtcattaacctaaggagattggacaataaaatgcaatatggaggtatcgaagtataaacataaggagtaccactgatggctgatttaggatgcccagtctggcaacacgctaatgaaatgatagtgggggagggggccgtaccaagactaggagagagcagtcctgagactattgcaattatctgcgggagacataaaggctagaacctgaacagtagcagtacaaaaaaagaggggagttcaaatgatattaaggaagtagaagtggtatgacttaaccatctgggtatggaaggggaaatggctagagtcttggggactttgtgtttgatgtgattatggaccacagaataatgtctaagagaactggctctttagtctgactgccagagtctgaatcctgaatgttttagtatgttaccttgcaaagcccttagcctctatgaatctatcttcctcatttataaaaataagatgacagtgcctatctcgtgggacttttgtgaggatgaagtgagataatggatgcaaagttactgagcacagtgtccaacacagcagaagcattacatatacattagctattactggctacattatgatatacagttagggagttggaaagataatctgaaattcaggagacgtatctgactataggtgagtatttggaactcattgttctgtaaacagtagttacagcacgtgtgtgggcatctggagagtgagcatggatattgtgatacctagtacagtgcctggcagtagtggttgtatgctcagtaaattttgttgacagggtcagggccggactagactgtggtaagcaaggcctgtagggcataaatatacttgtatgccccgagaagtgaggacctcttaaatattgtgccctacatgccttgtttggttcactcttgtcccagccctagcaagtatatataaggtgaaaaaggaaaaagctgaggctggagcctgggagaaccctggacatttaagggccatggagaggaacaggagttaatcaattcaagtgctggatggataacaggagttagagcaaagcggggaaccagaatagagtgattattataaaaagagtttcctaaaaagggagagatcaacaattagaaattatttagagcagccagtaaatacataaactcaaaattattctttaggtcattcctgattgtgacaatagtcatttcattatataaatgtgattaaggaaggaaaagagctacacagaagttattaaagagctaaagagaattgagaaatttaaaacagaagaaagtagggccaacatgaaaggagtagggagaaaaagagataaccagcatattgtctcactgatcctgccaacacctgtgagatagatattcttattatactactgttaaacctaatttacatttgacaaagttaaggttcagagcttgtgtgacttgtccaaggtcacaggtctagaggaggcagatacttgattcaaacctatttctgtctgatctgattctaaagtctgttttttcactcaaccacactgtacagtcagctctccttgtgagttccacagccacagattcaattaactgcagatcaaaaatattcaagaaaaaaatggatggttcgatctctactgaacatgtacagactcttttatctttcattattccctaaacaatacagcataacaactatttacatagcatttacattgtattagctattaagagaaacctagagatgatttaaagtacaaaggaggatgtgtttaggttatatgcaaatagtaagccatttttatatcggagacttgagcatccacagatcttgatatttgcagggggtcttgccaccaattttccatggatactgaggaacgactgtaaatggatgcaggcatggatgctatttaggagtgtccagggccaagtaaatgagttgctgagcagagaggtgggtggaggctgtgaggcatcaatatgtggtggcatccatctgcattttggtgatttttttccttcacagtcctcggctgtctgggaagagaaggatgaaggcagatggctgctccaatttaggggctaggattgcagggtgggcacagcattgcaaacgagtgaaggaaattgagaaatatggccaatgaagagttgaagagaggcctggcatggtggctcacacctataatcccagcactttcagaggcccaggcaggcagatcacttgaggtcaggagttcgacaccagcctggccaacaaggtgaaatggtgaaaccccggctttactaaaaatacaaaaattagctgggcatggtggcgggtgcctgtaatcccagctacttgggaggctgaggcaggagaatagcttgaacctgggagatggaggttgcagtgagctgagatcgcaccactgcactccagcctgggcgacagagcaagactctgtcaaaaaaaaaagagttgaagagaaaaagtctaggctaaattcaaagaaaaaaagtgagcccaaaaggaacttgcagagcaagggaaaagcagggatgtcaagggactagaacactccataaagtgaacagctgcaatgaaaataagggaagaaagtttagttcatctccgtttctttcctttcctttttactttcctttctcttcctttttggagttagtcaggaagtagtcccaaataccccagaaagttcatcttataagcccttggtcctcttgagatggtatcagatatattgctagacccttgaagaaaggaacaactccaggcaacttcttgagtccctgttattaattttatacatacacacacatatatgtatatacatgaaaacacacaaacacatgtgtgtgtatacagccatgcattccttaacaatggggatatattctgagaaatgtgtcattaagcaatttcatcattgtgcgaacataatagagtgtacttacctaaacctaaatggtatagcttactacatacctaggttgtattgatgtggcctattgctcctaggctcctgggctgcaaacctgtacagcatgtgactgtactgaacactgtaggcaatggtaacagtggtatttgtgtatctaaacatagaaaaggtacagtgaaaatacagtattataaccttatgggaccactgtcgtataatgtggtccatcattgaccaaaatgtcattgtgcagcaaatgattatctcatatatatatatatatgatatgatatatatgatatatatgtgtgtgtatatatgtatacatatatatgtgtacatatatgtatacatatatacacacacatatatatgtacacacatatatgtatatatatgtacacacatatatgtatatatatgtacacacatatatgtatatatatatgtacacacatatatgtatatatatatgtacacacacacacatagagagagagagagaggagaggagaggaaggagggagggaaggagaaatatgattcagatagagacatctatcctccagagttcaggagtgtctcttcagactaggtagatgtagcttaaaaaaaacatatcctggaattctagagagatgcttaaatcactgcaattcctataacacttgccaaccaaaggtgctgttgatctgaaattgcttttttaaattaatgcagtgatttttctttaacatctagtgacagacactggggtcacatttgcagctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcaatttgagttctttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacagtgagtatttccacataatacccttcagatgcagagcatagaatagaaaatctttaaaaagacacttctctttaaaattttaaagcatccatatatatttatgtatgttaaatgttataaaagataggaaatcaataccaaaacactttagatattaccgttaatttgtcttcttttattctttatagactgaattttggaagcagtatgttggtaagcaattcattttatcctctagctaatatatgaaacatatgagaattatgtgggttttttctctgcataaatagataatatattaaactttgtcaaaaggactcagaaagatcagtccaaccctctaacccatattggatggtgatatactacagggttatgccagtgtgggaactatcgctggtaaataagtttaatcctccctagggcttcacaaagaacattgttccaccccaggagggtggaaggaagaaactgaaatgattgtgtcttagaacctaatgaaagtttgcattcctcagtaaaatcagagactgctgattgacttaaatgtttatagcttcaaagtcctcctcattatcatggcccagaagcccttccatgattgtccttccccaccctccccattacccttcttgcctcctctgctacttctctcctcgcacactgggctccagccaccctggccttcctgtcacttcttgcacactctaggaatgctcccactttggaggctttatctggctgtttctcttatttggctgttcccaacttcctgtgggctgactccctcacctccttcgggtctttgcccaaatgttaccatcttaatgaggcctaccttcaccatctattaatacttcaacctgccccagtagccttaccactctagacacctgtacagaactccactctactttttaacagagcttttcaccatctaatgtatcatataatttcttactcatactatttatcatttattttctcctactccactaaaatgcaagtttcatgttggcagggatattcaattgttttgtttattgatatattcctagcacctagaacagtatctggaaaagaggtactcagtaaatatttatcaaatgaattgaccaaaagaaggaaaactcaaaactttaatgacaactaactttaaagctacaataacttaaaattcagagtaggattttgagggagggtaagtttcaaagattgacttacctaagactatctgcataaataaaaagaaattaatccagacaacaaattcaccaacttccatcaattggaaatccaattctattttctacagtttatgttctggagacactactggacactcttttactctcataactcataactcctccacttttgtttttaaatcatgagagaaaaagagttgactctgttatattgttttatctacctttccttgatcttagaaacgaatactaccataccagcttctactgaggtgccccctaaagttagtccaaataggtctttgcaatctccattcccgcagaatttagaactttgaatcacatgatttatttctaaaagtaaatccatgccgattttccccaccaaaaaattcctgactattaaactcctacaatcccttcattgctcactccccacccccaggatcatattttaaagttgggcccttgccttttgggtcacataggtacactgtttgctataccacaggtatagctatctggaaaacatggagggtattattctgttactactgcttcgtcaaccaaaaaataaaacaaaacaagaacaaaaaagaaacaaactccctgcctcttttcacttgcagtcaaggttcctaaccactacaaaattagcctatgtttcttcttgcacatagtagaaacccaagcttctcactgctgtgctattctgtaccatcaactcatcacataaagagcctggttgaagaatgattgtccaaccacattactagcatctgtcaagactttccagtttacaaaaggcctatcacatttaaccctcacaccatccttgtgaccaaagcattattaactccattttacaggagagtaaactgaagcttagggaagttaaaagaactgccaaaggtctcccagttggggagtcatgaagcccagaagagaagccaaattctctgctgctcaaccccttgctttcactattacacctcagggccttcaaatctaaatgcagttattcattaaacaggaacctggtagtcttaaacaggaatctctcacttggtaagatcttgtctcttgttgtatttgaccccaactgtctatggctttgcctgaacccaaagtacacacagcctagaaaccaaaggagaaccaaatgtgggataaaatgacactcattttaacgacatgtctcagcaaatgagttcctgtgtagctggctgaaagcccagaccctttcagtaaaacatcctgaataattcacatttgttggtctataatataaagggcaaatgtagctcatttttagaccagttctgaacatcaatagtaacaaaccagagataaccgattttgttttcatagaattggaacaaattagagtatctgtgcaaaagcatatcagatctaggagcagaggggacaaggtctaatttttaaataagcaaattttccagggagggactacttatgataaagggatattagtctcttagtcaacggaacctggatacacgcttctgacagagaagagggagaataggcaggaatctacacaccagatgtcaaggagatttgctttaaaatacgactgataattagaaatttctcagtttcccccttttccctcattctttgattcttattgttatctttatctcttactcctttgtttctcatatattgagtcttacagatcaagctcccatttttttcttcaggggtatttttctagttcaaagtgcctaccatctcccttctggttctattcatccttctctcccaaagctcctttagaagtgtggattaaggcagagcactaagaaaccagacttaaagattcccttctcattctgacttttctcctttcacctattccttcctcctgttttcttaccatcagtgtcttcaaaggctttcaagtacacggtaaatgcagaaacttcaagaaaggcagaatggaaacataaccaatgcatacataaataaagcacactgtagaatctttttaaattctgtatgatatatcgaatgctgtctctcacattacctagaccatttgaaaccgaatttgtaaaacatagactatctttaagtagtaacagatgcttctgacatgttttctattgtcttgaaccattactgcatatgatacatcaaagttaagtgacaatacaagaaagcagattcatttgctccctgcctaggccgtcagttcctaaagtggaaacgccatatattatctagctcagtttgctctacaagacctgcaatagagccttgtgtgacatagagataatatttgttgaagcaattaaatttgacttggaattaactctgccatcattctataaggaaggattgaaaatccttctcaccctgtgctgatatagtacctttctatacaaaaacgtccttctccctcttcccttggattgcataaactatgtacatgccttcctcaggggcacttttctaggacagtgtcagcctaaggatctttgtttgggtggcttttagaaactcaggaagacaggagcatcatatgcctataggcagctggcttccaggtcagtagttttgctctgaccctaaaatcagactcccatcccaatgagtatctacaggggaggaccgggcattctaagcagtttacgtgccaattcaatttcttaacctatctcaaagatggagatcagtgtgagtccaatccatgtttaaatggcggcagttgcaaggatgacattaattcctatgaatgttggtgtccctttggatttgaaggaaagaactgtgaattaggtaagtaactattttttgaatactcatggttcaaagtttccctctgaaacaagttgaaactggaaaatgcaatattggtgtatcataatttttcttaaaaacatacctttgatgcttataaacatttcatttgtagtgatagttttcaggatatgagttcaagaagctacattaaaatcaataacaatatttggtaactaatattaagtaataatgatgttccgactcaccttattaatctttaatacaaccgtatgtggttagtactatcattatgcgcattctatgcagatgagaaaaccgcaactccaacggccaaaaattacagaggcataaatggtttagacaggacttaaacttcagtgtgaccaaaacccatgcttctaactactatattcaaaactcagagaaaactgaacccagaaaattgaaatcatgactaaattgctatcaacataggtgaaagtcaattaagtacagaactggagtatgactggccaattatcccatataatgggaattctccacatgtacaaaccacttcatatgctaaacttgttgacaacattcaaagctcatccctgaatttgactatattgattacatcgaaaatgttacatagcaaccttagaatccttgtgtaccttttcttctcaaagcctagattatttctttttccgacgttttcagtaattggagcagtaaaccccagtgtcccttacctacttgtttattacctccagatgcaatattactggtactgtgattgagaaacgcacacagtgctaatgaggaattcactttctactctgacactctggaagaatagagatgcaatcctaaggaagaatttaacaccacaggctacatgactaaggataaagagtagaaaattagcaggactctattaaccgattacagcaatccacctgacagatgaaaaaggcatgaaatgaaatgaaatgtagcagctacactcgtcctattgagaaaggaaaaaagtcacctgtaatgttgttcagaaatcctttcagtactaaaaaattcattgaccatcttcctttagtctcgaaaatttcttagaaggtaaaaaaaggaaaaggtgacagggcaaagacatttgaaaagaaagaaaagagtgaatgaacttgcacacctggcttggactccccattccccttaggtttccattgtgggggacaaactaatgcctgggttacctttcttgagagtgtgttaattgattcaatatctctgaagtgctactttcatctgaaaggttataatttgaaattcagatttacctggataaatttgatcttgctattatggaaacctctagaaatccttggagtagttactcattatcagcttaaataatatagccggtggagctgagggaatgagtaactcaattagtctcagttacaactgaagggcacattgttgtaaactataattgaaaacataaatatctttacctagtttaaaaaataaagatgctttaaaaggaggaagggaatagccctgaggaatgtaaatataagcacaaaacttctacaacagagtttgctacgtgtgtggctgtgttccacccagcaaaaatgctaagtctacaactgacacaacttggatactctcatgttcccacattttggtttggtcaaggctgtgcagttgtactgcaggccaccaccactcctggcctctacagtatattgatctgacccaccaatctgatcaaggtttagaaaaatattttcagcccagttagctcacaaacaaaatgagaattcccacaaattgctctttatctcagacaacagaggaaagctacagcaaaagcataaacaaattaccatttaagtttgttgcttcaaattaaagactaattgcaacagctactagatagcacagtttatggggcatctcggccccaagtcttttgtcttataaggtcttgaaaaaaagaaaggagattttcatcaataagagttttttgttatctttttcccttgttcatcaggcccttcactgcgagagagaggtgtaaacgttcagggcatgcattctagttaaagaatattaattggctattgggtccctttggttagaataaagacctctgtatgatgtccctagctgtacatcaaacccaaatatctctcagataaatgaaggtctgtaagaatttggtcattcctgtctcttctaaagagtaacagaggcattttcccgcagtaaagtagaatggaaagaaaacaaaaatcacaagcctataaacaccttcttcaattttcccagcatgtcacagacactactgtcttatttactacgtatttctgaggagtaaaaaaaggaaatatgttgagtttagctgaagcacagcatattttgtggtaaacttgttaaataaaacatcttttgtccaagctttggttgtcacacaagtggatatatcaggaaatataaaggcagaataaactaaagcagaacatactaacatttgtagtaggcatgaagggaattagaaagtgtttgtgttaacatggagggagggagaacagatgctttgagatgttcttcaacagatattctaggcactgagacccccttcgggaccagagagagcccatatccaccacagtacctgacacataaatgctcagtaattgataaatgagtcccattctaactgttccttagccctgctctatggaactctcccctgaattccttgtgccattattttatttctggaatcttcagccttttagctgagggcaaaagattgctgattaggaagcaatatttcccacctcctgcgcaaaacaagccaaagatcaacagcagcagcaacatactgagccctaaagggcaatgacaaatgtggagaatgatacagaggtctggttacttcttagccaatgacacagaatcacaattgagaaaacacagagtttattcattcccattgtgcatgccctggacaaaccaagctgcacctttcgtaacttatcacaatctcatattgacggaacactttctacaggtaatgtttgatttggctgaacactttagcattgcttcgtagcaacaaaatgatagctagtaacagaaaaagatccagggatattaccactgttagtgaggagaaaggccttttaattaattaattaattaattaataggaccaagtgccatctttttggatcatgcccttagtggattattggtagcaaaggttaaagctcaagctggttcctttgtccccctggcaacagttgatttgcctcccttatctcctgaagtaccgtaaggactaagagccaattattacatttggctatgctagcatatgtaaaatagagtttaaaagtttagattcatcactcaaaaattcatattctccaaaaccatacagtcactctgttagcctgtgttcccccagaaaaaaagtcacaagcttattattaacatgtgcaatccaggggcaagagaaaggaactgaagatgaggcagaaaggaaaagaaagccaataagaggatgagttatcaaactactcgtttcttaacagcaactgattgcttaacttcctgggactgtctccaataagtcaaattggcctcaggttagtccacctgagtgggaagaagcggtgaaagaatttgtctgtcagtatctgtctctcattggttagaagttcgacttatggggaattaactccctcacatttcctagttggatagcttgggtaccagaggcatatggcatccatgctcagcatgaacagggaagcttcaaggcaaaagacacatagtgcagctatgagccaaggcaattcaaggatacacccataggaggctggttgacatccacccagagctaatcaccaccatgctggaaaaagacacaggtgaagctgagaagaatgaaggtggtgcataggaggtatctaatacagtcactcattttcaaactttccatgttatgattgcactgaccactgaggatttctattgaaagttttactgttgtcaaacacgtacacaaggggaaaggtgtcttacattgtttatgttcctgtgctgctctagaaacagaaataggctcaagagcagagcctgtttttcttaattcagcaggtctaagctaacaagtcctgaaacatggtacttcctgttattggtattgcataggagaaacaaagggaaagcacagtaattagaaaatacaaacaagatggcaggaataagccaaaaatatcaggaaacacaattattgtgaattgggattaaactaatctattaataatgacaactttcagcttggagttaaaaatttaattgtatactgttaacgaaagtgatacctaaaataaaattacactgggaggccaaaatgaagggatgtgaaaagaactatcaggtaaaaactaacaaaaagaaactagcaaagcaatcttaatatcagacaaaatagaatccaagaggaaaatcatttcaaaagacaagagattttttttattaataaggggaattgcataggagagtaaagaaaatgtgggccactggaatgcttagcactaatgacatattggtctttggtcttcagttaccttacaggaccctatttcattctcttatgtttgatatgtaaccacctcagccagcttcaagttgctttttggccctaatggacttcctagcactataatttctttttttttaaatgttttattttaggtttaggggtacatgtgaaggtttgttacatagataaacatgtgtcacaggggtttgttgtacatattattacatgacgcagatattcagctcagtaccaaatagtgatcttttctgctcctctgcctcatcccaccctcctccctcaagtagactccagtatctgttgtttccttctttgtgtttataagttcttaacacttagctcccgcttacaagtgagaacctgcagtatttgatttttgttcctacgctagtttcctaaggatgatagcctccagctccattcatattcccacaaaagacataatctccttcttttctatggctgcataatattccatggtatatatgaaccacattttctttatccagtctgtcattgatgggcatttaggttgattccatgtctgctattctaacactgtaatttctaaagacttccagattctacttttataggtaacctgttaaacagtctagctctggaagccaagcaatttctagaataactaagcaatagaaattacacttcaatgcagaaaggcagtatctacatgagattatgaaattgcggttgctttttgtgttcactgaaaaaaataagtaaaactgtaactttcagaaaaaatgattgtacatatagaaaacccaaagcatctaaacaattaaaataaataagtatagaaagattactggatacagagtcaacatacaaatatcaattgtatgtctatataccagcaacgattcaaaaatgatttttataatagcattaaaaattagacgcttagtaataaatgtgagaaagatgtgcaagaactctacataaaaaattatgagacgttattgagaaaaattaaggaaaacctaaataaatgaatgaataggcaatgtttatcattaaaggatacaatatagtaaatatatcaaatgtttactaatggattcaatgcaataccaaagtgccagcaggcttttttggtggtgggaggtcgggcaggattcataagctaattataaaatgcatatggaaatgcaaagagccaaggatagccaagacagttttgaggaagaataaacttgtactacttacactaccagatgtcaagacttattatcgagttacatttattaagacagtgtggtactgacacaaggatagacaaatagatcagtgaaacacactagagtgctcagaagcacacctgtacatatataaaggcttgatttatgatagaggtgccagtgcagtagagaaggaaattattggtgttttcaataaaaagtgataggtcaattagatattcatatggcatgaagtatgaaacaataacaatttatattcataacttgcagaaagcaaaaatttcttaaaatacaaaaagtgatcaccataaaggaaaagattgataaactggactatattaaaactaaggactcctgttcagcaaaagacactacttcgactgaaaagacaagtcacagagtgagacaagatatctgcaatacagatacctaataactgaaccccatacagtgatggtgggaatttaagttcgtacaatcattttagaaaattgcttggcagtatctactagatctgaacatgtgatccagtaattacactcataattataagccagtaaaaaggcatgtttatgtcaccaaaagatatatacaagaatgttcattacactattatacataagagccaaaaactggaaacaaaccaaatatccattaacagtagaatgaataaataaaagctgtaatagtaatacagtggaatactacacagcaatgtaaatgaactactgctgtacaaaacaacatggtttaatctcacagacaaaatgttaaatgaaagacacagacgagtacatattgcgaacttctgtttataattcaagaactggcaagaactgtttactgtgttagaagtccaggtaatggtaacctataaaaaggaaaaagggtggaatgattgggagggggcatcttctggggtcttgataatgtgctatgtattggtcagtttagtgtttaaacaggctcatttactttgtgaaaacttacactaaaattgtgtgtattttttgaatatatgttatacattaataaatagggtttttaaacctgtagttcataatttagtgaaagtagaatatccaaacatttagttttaaaccaatcaattatagtgctaccatcatttttatgcattattgagaagtttattttacctttctttccactcttatttcaaggctccaaaatttctctccccaacgtatattgggggcaacatgaatgcccccaatgtatatttgacccatacatgagtcagtagttccatgtactttttagaaatgcatgttaaatgatgctgttactgtctattttgcttcttttagatgtaacatgtaacattaagaatggcagatgcgagcagttttgtaaaaatagtgctgataacaaggtggtttgctcctgtactgagggatatcgacttgcagaaaaccagaagtcctgtgaaccagcaggtcataatctgaataagattttttaaagaaaatctgtatctgaaacttcagcattttaacaaacctacataattttaattcctacttgaatctgcttccttttgaaatcatagaaaatatcagtagcttgaattagaccaattaattttctagattgcatcatattttaaatataactatgtaatcatctacaacctgaattctttctgtgtccaatttgtccaatttttttctctaacatttatatcacaaagcaattaatttgtgtgatttctgcatatgtatttgtaattcatcaagtcaaatcaatgtagtaatactatatcataaaatatacacaaataattgagtgataggcttctagtataaggacggtaagtttgaagcatgattctatctgggctggctagtttactctgagaaagttattttttattgttgggtcttaagctgagtttacacacttggtgtcagaatgattccggcaatgaactgttttatgttctgctaggctgatcagcacaatctatatggctgtgaacaaaacaatgtttcccagtcataccaaccatgccaccattttaacagctgattagtgtattcagaacatctccactccatgttcgtatggctgttatctaaagatgaaagcagtagacacttttattttttgaaaaatttaggctctgcagggtcaattatatttgataaatgaggggcttttttgaagcaaactagatataatttcttttgcatttctaaagcctgatatcttattaattggtacattaaattgtgcaccatttctctgtaactgtttcagtacctgtctcagcactataccaggcagaagaaataaagaaagaaccagtgccagatcagcttggtcaggagaccctaatcctgcggcactagaggaattaaagacacacacacagaaatatagagtatggagtgggaaatcaggggtctcacagccttcagagctgagagccccgaacagagatttacccacatatttattgacagcaagccagtcataagatttactgaaagtattccttatgggaaataaagggatgagtctggctagttatctgcagcaggaacatgtccttaaggcacaaatcacttatgcaattgtctgtggtttaagaacacctttaagcagttttccgccctgggtgggccaggtgttccttgccctcattctggtaaacccacaaccttccagtgtggatatcaaggccatcacgagcatatcacagtgctgcagagattttgtttatggccagttttggggccagtttatggccagatttggaggcctgttcccaacaaaccagaagctaggaatatatatcctgcaaataaaatgaagaatctctaaggcttcagggcctgcccacttgttcttctgcctggttcttcacatacactgtctcaaagctagtctaccttgagaggagcatgaatatgtgtgtgggtgtgtgtctgtgtattttaaccttaaaaacctaacttccagtatagacagatggcatactagctaaacccttacaagttcttctatgctataaaagagaaacagaattgagaaccacctccaactattaagtgttatatttgaatatagccttagctttagcagaataagtaggccaaacttaaaataagcttttctgccttttcaatgataaaggtcccttttctgtagccattgttgattgtgtacacttatacataagtattttgaactaatttcctgttttctcaaccacttgctgtcttcatgatactttgtcgcagctggttgctatagaaatgtctgttacaaggaatgtggcttgaaggaaagtgataaatgaaaatgaaatgtgaagtgactttgtttgactacaaattcccattctggtagtccccagtgtatcaatacattatttttctttagaaaataaaccaacccaaggaaaaatggtgggcaggtcctggtgaatatggctgtgataattatattagcaatctctttggctaatatttgaagcccaaataattgaatcacaatgatctctccccagaaaatatataaaatgcaccttggaatctagaaggccttttagtctgcaaaagaaaccttcttaatcataagcagcagaagtcccatttaccaaattggaaagttaaagttacaaagcatcaatcatcagacttccattcagggatggcaattgggagtaagactttttagtaaagaaactaaacacaaagtcattagactctgtaaaagtcttaccaaatttgattctggaacacctattctatttccgtaaagatgatgaattcggagccaaatgttcttttcatgaaggatttgaaaactgtccatgaaaataacgcaatcaaccttttagcttgagactctattcactgattagatttttttaaatactgatgggcctgcttctcagaagtgacaaggatgggcctcaatctcaatttttgtaatacatgttccatttgccaatgagaaatatcaggttactaatttttcttctatttttctagtgccatttccatgtggaagagtttctgtttcacaaacttctaagctcacccgtgctgaggctgtttttcctgatgtggactatgtaaattctactgaagctgaaaccattttggataacatcactcaaagcacccaatcatttaatgacttcactcgggttgttggtggagaagatgccaaaccaggtcaattcccttggcaggtactttatactgatggtgtgtcaaaactggagctcagctggcaagacacaggccaggtgggagactgaggctattttactagacagacctattgggatgtgagaagtatttaggcaagtttcagcactaaccaatgtgagaaggcctccagagatgagcagttggtgaaagagaggctcaaaaccagctaccatacaggtcaagaagaatttggcattaaggaaacagcatagcaggattccagacaggcaactggtcaacaacatgaaggtctggaagaaaggtcgcagtactcaggttcagggcactacttcagcttcagcccttgcaaaaactggtgagagttggaaagtctttaggctaagaaaaattggattatttaaaaggggtaaagaaagggactcaaggaggaaggattaaggcaagaactaggttccaagaaacagggcatgagagagagtcttgatctaccactatagttctcgtggtagcatcagaatcacctgggaacgtagaaatgcaaattctcctgctctacactagacctaccaaatcagaatatctagggggtggggcccagcagtctgtgcgcaaacaagcactgcaggtgattttgatgcacattatagtttgaaaactaggccaggtgcagtggctcatgccaataatcccagcactttgggagactgagacgggaggattgcttaaacccaggagtttgagaccagcctgggcaacacggcgaaaccccacctctaattaaaaaaaatacaaaaattagctaggtgtgatggctcccacctgtgctcccagctattcaggaggctgaggtgggagaatcacctgagcctggaaagtcgaggctgcagtgaattgtgatcacaccactgcacttcagcctgagtgacagagtaagaccctatctcaaaaaacagaaaaagaaaaacactggcccaaaggaaatgaacttgttacagaagccggggttcaaaacaccaaataatgcacttgtacctagtccttcccgggtgctctgcagacatttctccaagcgtagtctgcaaacaacctacatatgtagaattacctatgcacatttttcatttaacaaccaagagctacatttgtagcaaaatctgggttgtaacttagcctacagctgaagcctaagagattccgtctgtgagaagaaataacccacctctttggcccccctccccaggcaggaagccaggatggtccttatataaagttgtgctgtccaataggtaaccactagccacatatggctatttaaatttaaattaactacaattaagagaaattaaaaattcaattcctcaattgcacctgccaaattttaagcacataacaaccacatgtggctagtaactactgtattggagagtgcaagcggagatagaacactctattactgcagaaatttctattggatagcacttataatagtttagtgtaacttaaaactccctagttgccacagtcatgatttagtagtaatttcatggatttctctactgaggttagaatctctgccattagagactgataaatttaaagtttgcaattatcaaactggtgacaatttaagccagaatcaggtaatgtcctcagttttaacagcattggaattttctgggactagctgtgtatctatccaggattcttgagaatgcctgccatttttcaacataatggatgtaaggtattacacatatacctggggatggggaggtaggtataattgcacaagcattgtggagaatggtatcaaagagtggcagaacatcacaatcaaggttttccctttcttttacctttgctttttaaaaagacaatatttgctggacctgatcttataactcataaatgggacactgtatgttcctttttacctcctctgtttctacttaattgcaccctatgaggactgcttcccttacctaccataaccccttccttcactcatccatatctttactcttcttcacaactctgtaatattgaccttctttatgaacctttcctggaacaatccctcttaatgcaagcactgttattatgccttcaatgtatttaatatccatgtatctattctctctaattttgtcattttgtgttctcatgtattttcattcattatgtgtccaacttccatggataacatggttacaacaaaagatcctactttatgacaattatcttccttgggtttgtgggacatagaacagtgcacagagtaggggatccaagaacccaggagaatatattagctaagaagataacttccgtttttaaaagtccaagattcaggagatcaaaaccatcctggctaacatagtgaaaccccgtctcttccaaaaatacaaaaaattagcccggcgtggtggcaggcgcctatagtcccagctacacgggaggctgaggcaggagaatggcgtgaaccggggaggcggagctggcagtgagccgagatcccgccactgcactccagcctgggcgacagagcgagactccgtctcaaaaaaaaaaaaaaaaaaaaagtccaagattttaaaaaaaaaaaaaaaaaggatgtctgctttgtgagtttagcattgtctccttgtcattccagaaatgaaatggcaaatacatttaaatcagaactaaaaaggggaacagggtataaaggctcaatttagtcacatcatttccgtttctcacccaccccctttaaaccagatgtttgccaatgcattaacaatgcagatgtttcctgaaagaaagtttagtaactcaagcagacaccttattttcttttcaagcagaaaagactatgagatggtggttgtggttgttccgggagggagaagatataaatgatacacattatttcaaatcatttcatgacctcactgcacacttatagttattgtacctgttgtctttttgctgtcaagcctagctaagatcatttggaatgttcaagatcactcatacatgcatgtgcacacatacacatgcacatatgttcactccctatttcatccacatgaactaagattactgatgtgtacagattcaaagcacttttattcttttccaaaggcaagaagctgagctactttccagaatagttgtgaaagaccctgtcatacttctgcattgtttcctccacaccacctccatccagttccttatgaatggttactggttttcaaaaatatgagataaattgagtgtataaaagtcatttttagacaaaatgaaacaggaaatgaaagaaaccagaatctctcctcatttgtggatgggccagctccaccatgtcatggttaatctgcagggaggaaatactagatttgattgcagatcagactgcagcaaacctgctgtgactaaggcatcaagagaaagcaagcaacagactggggcttcagtggtgaaaacattatatatctagctttgaaatatgaaatactgtttagcagtgtcacctagaaaagagtgtttcaaaatgctgatgcttcataagaacctttctcttcagagttggtttcttttatctttcaaattagccagggtgggaaataaagtgatcacttggtgaagaaatctcacaaagaagaacatagagagttcactttcatctggagtaatgaacagattgaacaaactagaaatggttagtctgttaaagaaaaggtgtaggtgagctgtttgcaagagccacaagggaaaggggaagacaacttctttgtggacttaagggtgaaagttgcaagcaggcaagacgattctgacctccattaagaaagcccaaaccaaccaacaaccactgggttggttacgcaggttgggcagcattgggagcaaatgttgattgaacaaatgtttgtcggaattgttgacttaaagagctgttctgtcactggggacagcagcggctagatagccccattcagggagagggcatttgttcacctggccagagatcagagcaggctaagggactgctgggatcctgtccagctttgagaccctacagagccatgttcacctagcacgtatcccgtctgcggtcacggtcatttcttaccttattccagggctttcacctcagcttgccaggctggagccaagggccaacgcagccgcgccttgttcgcgatggtagcttcccaggagccccctatggttccggaacgcgctgccggccccatcctgtttgctacctcctaaagccaaaggcactggcgggccgggccagcttctaaagtcgcgcaaggttagaaggttccggacaggaacggcgtgaggccaatggaaggaggtacttcagtttccctccagatgcccagcgatgggctcagagctccttgagaactcgggaaaggaagcagggtctctgaagaaatacttcaggagtagaaagaggaagctagagggttaaatgcactacacaggaacagaaatgagtttttcttagagttagtatatgtctagaggtgtagtaaactaaaacaagtcttgaattgcataccgccacgtagggaagaaatgaaaacctttgaatattagtgaaaaaagggaaactgcaacgcctgtattactagatagctttcatcaacagctcaaaaccgacagatttaaagaagcaacaccgcattttggctttctaaagctttaatttggtttggatcccatgcccatgaccctgccagctgacaattctaagcatgcgcaaactggccccaaaaattcctcccacatttccgaagaactatttggccctttatgtgaagtacctggtttttccattttctgttttaccataggcctcagttcggtgtgtggcgtatttattctacatttaacaatttgaagatcattctattagattaaaaaaaaagaatacaatggaagccaagtgattaagctttccttatgcttatattaagttgtagcatatgcatttaccgatagttaaccgtattaacctacagaaaatgtccagggaaatggtctatttcttattctatttttgacctaaagaaaatctttaaaatgtcttagcattttccccagtctccatccacttccctcagctttggcctgaagctatctttaaaggtaccctgtacaagctcttgccctgtacagctagctacagagattcaatcctttctgttcgattaggacacatctcagtggcagataacatgcaaagttattatatgtatgaaccagaacttgtttttccttaggggccaggatgttacactaaggtcttaagactatagtaatatcttcacttgaaaaagccctctattattcctatctcagatgataaaaattcaattaagagaaataagaacgtgacatgtgtaatcgcacctggctctacaaagctagtctggacagacatttaaacaattatcctctaagattatttgatgaaatgcatttcaatgactagttaaccattaaaaaccaaagtgagcatcccatctgttcccagtcaaatgacctagagcaaaggactaggcaaaccacatctgtgggcatagcaagctgtacatcacaaacaaatgaatttgctttgtatatgagtgagagcaaacactctttattgtacaacttgggtgggtaagtagggagaataatggttttactgaaatcgcaggtaacggttacgttggagttaaaggttaggaagaaaaccaaagggtaagagctgttgttctgggctggcattgtcaatgaagagcataaattcagatgtgaatgtatattttgtagaagcatgtgtgttgttggtttttgtgtatgtgtgagtctgaaagagggaaaacaggctcccattagactatgactaacaaaaatgtttgacagattataactcagatgtcttactcagagcatatgccttcccattttccccattattccccaacatgatgtctttaagaacttgtccttgaccgagcagacatctcataccccaaatagctaatattttgatagctatgatcctgaacggccaaacattccaaaaccaagtagtttgtaatatctttaatgcaaatatattttaggccttttccttggcaaggatgtttggtcaggggttggcaaaaataatgctcttcagacttaaaagaacacaaccatatttcttagccatccaccagaaagtagtagaacgctccaggaagcaagtctttgtcaggagtcagactagctacatcataatctctctgcccaggggctgtggatgtcatccatcctggcctaactagcctactgagctgagagatgtccaatttccccccaatacactaaccagaggagaaggaccgtgatatcattgcatgtgaattcttaattccaattgcttaaacaaatatgttcagttgtaactatcaataccagtatataacagtgttggccaagttttattgatgctgacaatcaattggagttacagccagacacatggtcttatgaccggcgtacttacgcagggctttgcactgagacaggtcgtgcatctgaggtttactgctttgcatttttgttttgtaactgaagtctgatgagacagccagagcatgtgctacctagggacttgaatccctgcagccccatttcacttctcaccaccttccggggtggtttctcgacctcccactcccctaccacctggtgccttagccagccctggctctccctccaaacacctgcccaatgagcactgccaccccatggtgcccagacatgctctccctcctcatccctacctagctaccattgccactcccctcccccagcggggacatgggcataggagcagggagagttaaggttggtcaggtgcacgtgccctatgctatcttggaagggggcttggccatgtggcatctctggaccaagaatgcgccacagcacatttggagggtgaatggtgggggcacaccccttgtccacctctatttcaggcatggaacacatcctggcatgaaagttgcagtcccttgggaatcacctctccaccttgattgccacagtaggccagtgacaagggaagattgacacatcatccctgctggggcccagtgtcctgtggctggcaggcaggggatcctaaggacatgtgggtcttaaattgtagggtgcacttcctgggcacctttgagggtctgcactgccccagcaaatatccccatgctagaaggagcaaaatattaaatggcaaattttaaaaatgtaacaagatgggttgcaaaagagactacagaggaaagcaaaagttttgtattttagtatcttccatggcacttttcttcctagcttttgaacaaggggccccacatttttatttctcactgagccccacaaagtatgtagccattcctgcccggagtgaggacttttaaaacataaagattatcaagtcttggaaattctgattcagtagatatataacaggtctcaaacttaattatgtaaagaatattctggagagcttccttttacccagtcccacccaccaaatattctgataaattaagcttcgattagcccccagatctgcattttataaggatccccagatgattctactgcaattggtccacagaccatgcctggaccgaatttgggtgcttaggagcacaaattctggagccgggcagacttgagtttgcttcctagctttaccaactgatctcaggggagttaatgtttacctctaaactttagctcatgcatctataaataaatatattaatatcatgtcataaggatattatgttgtattaaatgtctttaaaacaccacaatgattagcccaaagtaaacactcaataaatgttcaaaaatttaggaaaattgttaagactgggttgtatgcacactggtgtttattatattatgtagttttttctgtatttttacaacatttcagaattaaaagcaacagctagaaaaagagggaaatggccgggtgcagtcgtcacgcctgtaatcccagcactttgggaggccaaggcgggcggatcacgaggtcgagagatcgagaccatcctggccaacatggtgaaaccccatctctactaaaaatacaaaaattaactgggcatggtggcatgcgcctgtagtcccaggagaattgcttgaacctgggaggcggaggttgcagtgagccaagatctcaccactgctctccagcctggtgacagggcaagactccgtcaaaaaaaaaaaagagagggagagccagagtatgaaaaaggaagtcagagccctttaatgagtcagctttgtaggtctccaggtaggaggctagtgcttcagtgtctaggacatagtaggtgttcagtaaattaaattcaggacaaaaagaacatgccccaaggaccatctgatatccacttaaagtgatggactacctcgtttcccttgtttatgaatgggttcatgcctaagactgtgtgcactttaatacaagggcagtcgttcagaactagtcaggtcctgaaaaggatttaccaaatgttgagtgtgccctctagtgttcacacttcccagctttcttcctataaaggtggatcaaggcacttgcttacaactggaactgaaatcctccaagtcgatctagacattgagatggagaaaatattcattgtcgactgtaattatgcaacgaatatccagttgagataatggacttgcctcttatctaataatacccaggctcaatgcgtcactgctttgtccactttgcccaaaattcaagcacagctaagttgatattttaggacaaaggcagcttactatccagccagaggggagtagaatatggttaagagagagtggaaagaatgaatgagccctgctattcctcactgcctggatggctataagcacagcccttatggaggccttaggtcttgcttcacaatattccagtttgaaaagggtttgaaaagacctcctagaaaaatcagtagtttttctcttttgagtaacatgtagcaaaaaaaatttcatcatgtaggtacagggaacaccctagtaactattaatctcaaggagtcaagccagtgtgtttcctaatgtatctgctgtatccccatgaagcaaattttgccatcagagaaactgactcatggggaaaaaatccaaggacctcaaatcaccaaaagaagccattcctcagatttgcctaagcttaagcttccctgtctctcattgtgtgttgctttcaatgcagttacataaatggcttttttgtttatgcaccaaaaacactaattcatctgcaaagctcacatttccagaaacattccatttctgccagcacctagaagccaatattttgcctattcctgtaaccagcacacatatttatttttttctagatcaaatgtattatgcagtaagagtcttaattttgttttcacaggttgttttgaatggtaaagttgatgcattctgtggaggctctatcgttaatgaaaaatggattgtaactgctgcccactgtgttgaaactggtgttaaaattacagttgtcgcaggtaaatacacagaaagaataataatctgcagcaccactagctctttaatatgattggtacaccatattttactaaggtctaataaaattgttgttgaataaattgggctaaaggcagaagggtcataatttcagaacccacgtcgcaccgtcctccaagcatccatagttcttttgatatacccctattatcactcatttcagtgaggtacaattagttcttgatgtagccatttccataccagaaggccttcccaaaaatcagtgtcatgtcaccgatccttttatctctggtgcttggcacaacctgtagcaggtcctcagaaaacaaacatttgaattaatggccaaatgagtttgtgctcaaaaaaggggtgaggatacttgaaatttggaaaatctaggataattcatgactagtggattcattatcaccaatgaaaggcttataacagcatgagtgaacagaaccatctctatgatagtcctgaatggctttttggtctgaaaaatatgcattggctctcattacatttaaccaaaattatcacaatataagaatgagatctttaacattgccaattaggtcagtggtcccaagtagtcacttagaaaatctgtgtatgtgaaatactgtttgtgacttaaaatgaaatttatttttaataggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagaggaatataatgtgttaggaaattacagtcatttctaagggcccagcccttgacaaaattgtgaagttaaattctccactctgtccatcagatactatggttctccactatggcaactaactcactcaattttccctccttagcagcattccatcttcccgatcttctttgcttctccaaccaaaacatcaatgtttattagttctgtatacagtacaggatctttggtctactctatcacaaggccagtaccacactcatgaagaaagaacacaggagtagctgagaggctaaaactcatcaaaaacactactccttttcctctaccctattcctcaatcttttaccttttccaaatcccaatccccaaatcagtttttctctttcttactccctctctcccttttaccctccatggtcgttaaaggagagatggggagcatcattctgttatacttctgtacacagttatacatgtctatcaaacccagacttgcttccatagtggagacttgcttttcagaacatagggatgaagtaaggtgcctgaaaagtttgggggaaaagtttctttcagagagttaagttattttatatatataatatatatataaaatatataatatacaatataaatatatagtgtgtgtgtgtatgcgtgtgtgtagacacacacgcatacacacatataatggaagcaataagccattctaagagcttgtatggttatggaggtctgactaggcatgatttcacgaaggcaagattggcatatcattgtaactaaaaaagctgacattgacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttccctttgtgaattaataaactggtgttctggttcataccttggctttttgtggattccattgatgtgaatcagtcaccctgtatttgatgatgcatgggactactgacaaaatcactctgaccctgccaagctgctgccttctcctgccccaacctcacccccagccaggcctcactcttgctagttcctttagttcttttagtcaatatatttttgtcttcgcatataagtataaataaacatatttttaaatttcttggctgggcccagtggctcacgcctataatcccagcacttctggaggccaaggtgggcggatcacctgaggttaggagtttcaggccagcctggccaacatggtgaaaccctgtctctactaaaaatagaacaattagctgggcttggtaatgtgcacctataatcccagctactggggaggctgaggcaggagaatcacttgagcctggggagcagggggtgcgggaggttgcagtgagacaagatcgcaccagtgcactccccatcctgggtgacagagtgagactctgtctcaaagaaaataaataaataaatacatttcttgaggcgtttcttgttaaatcattcatggagaggcatcccaaacaccacattcaacaaaacactctgaaaaatgttttcaaatgcaatataacacagcagagatttgatgctctgttatccagttttcatataaggctgtgtgagctgtgtcccagagaggacagtggtctgaatccacctgagacagaattgggtctaactaactgtgagtatggccttcaataagtcactctccatttgggaatttgatttctccacttgtataatgagagtatttgacaggatgctctcccaaatcccttgcaattttgttagtctgtgatttcatgtttttatttttattccttcatccaacaaatagtcaaggagtaattgctgtgtgccaaataccaacagtattcattaaattgtaattcagattttatatatatataaataatgtataatgtgtataaattgctttgtgagtgcctactacactgctagacagtagttgctcaataacttgttagctgaatcagaatccatgtttatcccagagtagcaattagtcttgcatcgagtatcgtgaaagaaggccacacttaaataagaataatgcctggggtttaggttttatgaaaaaatgaaaggaaattagttctgcttttgttgactaaaggaagggaagagagaagagacactataattgtctgcctcagatttaaggaggaggctaattcatgcattaaacacgttacttcaaatttgaatgaccaaaggtctgtagcctcagcacttcaaaattggtaaaagtaagacactctggccttgtttccatagagaccaccccttacaaaggcaccaatgggaaactggcctcaggactcctgttattggtcttctctgtggcagagaaaggagctcttggacccataaatctctgagccacagttctttttgccatgggctcaaaaatgattgaattcatcatgagccacctgtggcatattgccacactaaacatgtggggcctttaagctcactaagagccaatgtcttcagagccagccctggcttgattctacctagggcatttgcagttgccatataagaatcattagtgctttcaaaattactgtagatactttgcctaaatagactaaaacatgctgccgtcatattggaagtgacagattaaaatagaactcttgccaagtgaaggaaagtgtgctaatataatgcagtcattttaacttgctgtttaagtgtgattgtttttagttcttttgaatattatttgttttatactgacaggaacgaagtactgtccaattttctctgccaaggaaaaaagaaaaggtgttcttccttacttacctgaaccaaaacagaccagtttacaaaattgcctaattataattgctaaacaagttccgaatgcttacagtctaatccaagaatgtcagagctgcaagggcccttaaacaccatccaatccactccactcatttagcagatgaagagattgagggcaacataaggccaggcccaagataacacaatgacagccaggactagagctcaagtctcccaccctgcactttgaaagaataatgctttcaactggagtacattaactctactgtctatatttttagggcagctggggcattctgcattggtggcaatcctctcaacaaccctgggactgaaaactgcctggaattcttactaacaattctctaattgaccaaaaggtgacgaaatcaaggagaccaataaggtagccttggaaagcaagagtggc

SEQ ID NO: 103 - DNA, 1855 bp, Homo sapiens
  Genbank Accession No. J05428
tgcattgcaccaggatgtctgtgaaatggacttcagtaattttgctaatacaactgagcttttgctttagctctgggaattgtggaaaggtgctggtgtgggcagcagaatacagccattggatgaatataaagacaatcctggatgagcttattcagagaggtcatgaggtgactgtactggcatcttcagcttccattctttttgatcccaacaactcatccgctcttaaaattgaaatttatcccacatctttaactaaaactgagttggagaatttcatcatgcaacagattaagagatggtcagaccttccaaaagatacattttggttatatttttcacaagtacaggaaatcatgtcaatatttggtgacataactagaaagttctgtaaagatgtagtttcaaataagaaatttatgaaaaaagtacaagagtcaagatttgacgtcatttttgcagatgctatttttccctgtagtgagctgctggctgagctatttaacataccctttgtgtacagtctcagcttctctcctggctacacttttgaaaagcatagtggaggatttattttccctccttcctacgtacctgttgttatgtcagaattaactgatcaaatgactttcatggagagggtaaaaaatatgatctatgtgctttactttgacttttggttcgaaatatttgacatgaagaagtgggatcagttttatagtgaagttctaggaagacccactacgttatctgagacaatggggaaagctgacgtatggcttattcgaaactcctggaattttcagtttcctcatccactcttaccaaatgttgattttgttggaggactccactgcaaacctgccaaacccctgcctaaggaaatggaagactttgtacagagctctggagaaaatggtgttgtggtgttttctctggggtcaatggtcagtaacatgacagaagaaagggccaacgtaattgcatcagccctggcccagatcccacaaaaggttctgtggagatttgatgggaataaaccagataccttaggtctcaatactcggctgtataagtggataccccagaatgaccttctaggtcatccaaagaccagagcttttataactcatggtggagccaatggcatctacgaggcaatctaccatgggatccctatggtggggattccattgtttgccgatcaacctgataacattgctcacatgaaggccaggggagcagctgttagagtggacttcaacacaatgtcgagtacagacttgctgaatgcattgaagagagtaattaatgatccttcatataaagagaatgttatgaaattatcaagaattcaacatgatcaaccagtgaagcccctggatcgagcagtcttctggattgaatttgtcatgcgccacaaaggagctaaacaccttcgggttgcagcccacgacctcacctggttccagtaccactctttggatgtgattgggttcctgctggtctgtgtggcaactgtgatatttatcgtcacaaaatgttgtctgttttgtttctggaagtttgctagaaaagcaaagaagggaaaaaatgattagttatatctgagatttgaagctggaaaacctgataggtgagactacttcagtttattccagcaagaaagattgtgatgcaagatttctttcttcctgagacaaaaaaaaaaaaaagaaaaaaaaatcttttcaaaatttactttgtcaaataaaaatttgtttttcagagatttaccacccagttcatggttagaaatattttgtggcaatgaagaaaacactacggaaaataaaaaataagataaagcctt

SEQ ID NO: 104 - DNA, 547 bp, Homo sapiens
  Genbank Accession No. AI819354
ttttatgtttagtttaagaattttattttaaaggaatttctgtggcataacataaggtttatggtacttttactaaaagtcacttataatgaccaaattataacaatttttgcaataagctctcattaaattttcctaaaagtagaaaaagtacacattatataccattttgcacttaattacttctttaaaatctcaaaataattcagtgtaaaatgttagtttcaaagacaatttatgggaaattacaaagcaactacaaagttcttcataataaatccaagataaaagtaactttttacaggttgaaagtttttcaaaatgcctaagtggattataaatacagtttggccatctgatgaatttagaggcactttaaaaggtgtctagttgaactactacagataggacatgtttgaggaaatttaatctttaagtctaataccagtgttcatttcttcctgccaaaacaagaaatttattggaaaaatccatgagcagcatgattagtttgatttctttctctttttttagagacagggtcttg

SEQ ID NO: 105 - DNA, 2282 bp, Homo sapiens
  Genbank Accession No. L32140
ccccgagtctctgcgccttcacatagttgtcacaggactaaagcaaattgatccagggggaaacactgtagaccgtgtatataaaaacactctataaactgcaatgctcaattcttagtataactattgttgttgtattgatatttattagtattggtgctcacaaaaagagtctaaattccataagtctttatattcaggctactctttatttttgaaaactcattttctatcacctttttctattttactccatattgaggcctcataaatccaattttttatttctttcttttgtaaatgtggtttctacaaagatgaaactactaaaacttacaggttttatttttttcttgttttttttgactgaatccctaaccctgcccacacaacctcgggatatagagaacttcaatagtactcaaaaatttatagaagataatattgaatacatcaccatcattgcatttgctcagtatgttcaggaagcaacctttgaagaaatggaaaagctggtgaaagacatggtagaatacaaagacagatgtatggctgacaagacgctcccagagtgttcaaaattacctaataatgttttacaggaaaaaatatgtgctatggaggggctgccacaaaagcataatttctcacactgctgcagtaaggttgatgctcaaagaagactctgtttcttctataacaagaaatctgatgtgggatttctgcctcctttccctaccctggatcccgaagagaaatgccaggcttatgaaagtaacagagaatcccttttaaatcactttttatatgaagttgccagaaggaacccatttgtcttcgcccctacacttctaactgttgctgttcattttgaggaggtggccaaatcatgttgtgaagaacaaaacaaagtcaactgccttcaaacaagggcaatacctgtcacacaatatttaaaagcattttcttcttatcaaaaacatgtctgtggggcacttttgaaatttggaaccaaagttgtacactttatatatattgcgatactcagtcaaaaattccccaagattgaatttaaggagcttatttctcttgtagaagatgtttcttccaactatgatggatgctgtgaaggggatgttgtgcagtgcatccgtgacacgagcaaggttatgaaccatatttgttcaaaacaagattctatctccagcaaaatcaaagagtgctgtgaaaagaaaataccagagcgcggccagtgcataattaactcaaacaaagatgatagaccaaaggatttatctctaagagaaggaaaatttactgacagtgaaaatgtgtgtcaagaacgagatgctgacccagacaccttctttgcgaagtttacttttgaatactcaaggagacatccagacctgtctataccagagcttttaagaattgttcaaatatacaaagatctcctgagaaattgctgcaacacagaaaaccctccaggttgttaccgttacgcggaagacaaattcaatgagacaactgagaaaagcctcaagatggtacaacaagaatgtaaacatttccagaatttggggaaggatggtttgaaataccattacctcatcaggctcacgaagatagctccccaactctccactgaagaactggtgtctcttggcgagaaaatggtgacagctttcactacttgctgtacgctaagtgaagagtttgcctgtgttgataatttggcagatttagtttttggagagttatgtggagtaaatgaaaatcgaactatcaaccctgctgtggaccactgctgtaaaacaaactttgccttcagaaggccctgctttgagagtttgaaagctgataaaacatatgtgcctccacctttctctcaagatttatttacctttcacgcagacatgtgtcaatctcagaatgaggagcttcagaggaagacagacaggtttcttgtcaacttagtgaagctgaagcatgaactcacagatgaagagctgcagtctttgtttacaaatttcgcaaatgtagtggataagtgctgcaaagcagagagtcctgaagtctgctttaatgaagagagtccaaaaattggcaactgaagccagctgctggagatatgtaaagaaaaaagcaccaaagggaaggcttcctatctgtgtggtgatgaatcgcatttcctgagaacaaaataaaaggatttttctgtaactgtcacctgaaataatacattgcagcaagcaataaacacaacattttgtaaagtt

SEQ ID NO: 106 - DNA, 1935 bp, Homo sapiens
  Genbank Accession No. M10943
tctagacagtggcgcaagagactggggttgcactgggactccaggaaaggcttagctgttgacgaaggaccggggcggggccggggggcggggcgaaggccaggatctccaggtacccggaaccccaaggggcgggtgtagcaggcaatcttggcgaaactgggaagggcgggcaggagggcagggaagccgctcacccaggcacaaagcgcctcccgcttgagcggactccaaagggacggtccgcggtgtgcagcgagctgcgctcaggggaccttgcgcccggcccttctgctgcacacagcccacccaggacctcccgcagcgctgacaggcggggcgggtgcaaagacggggcggggtctctgcgcccggccccctcccctgactatcaaagcagcggccggctgtttgggtccaccacgccttccacctgccccactgcttcttcgcttctctcttggaaagtccagtctctcctcggcttgcaatggaccccaactgctcctgcgccgctggtaaggaacgccgggttccgtgcctggggatgctcgattcccagacaccatagagagtgttcctgggtttgagaaggtcgtattttgagatctcaactgtaggggactccttgacttagtccagtgctttcctcttggccaagatcctgagagcatttccttcctctctgtgcctctgtgtcagcgttgagggtactgaggctcaaggctgtcctgctccacgtcatgcggtttgtcccagggctgttggctgagccccagtgctctgaccaggctttgagcagcaggattagataggaggcaggggacattgcctcttcggggttcaggacagaaagtcgaagtcgccgtcttcccaggctgtgcctggagcctgggactttcctttggagtgcaaacaggaggctgcttggccttcccagcatgaagggagaggacatggggcttctcttcctctgctctgagtgggaaaggagctctgagggctggccccgcacagaggagggggcaatggagactcattaactcactgctgtacctcctgcaggtcactcgccgctcactggcttttttttctctttctcgcaggtgtctcctgcacctgcgctggttcctgcaagtgcaaagagtgcaaatgcacctcctgcaagaagagtgagtgtgaggccatctccatggtctggggctgtggctaaggttgggatggaacccaaggctggccctgagtgcatgcttctggggaactggccttcctttgtccccgtaggttgtcactgcctttctagtcttctgccctgtgcagggcgcctgggcagctttctcataggaagacccaccccagatatttcccagttgtctcctgacaaagccataccctcctgaactgagggtcctttgtggctggaggctctgttgggggcctctgttggggagggaggtccctgggcaagttggctgtgacctctcatgctcctcttcttccccaggctgctgctcctgctgccccgtgggctgtagcaagtgtgcccagggctgtgtttgcaaaggggcgtcagagaagtgcagctgctgcgactgatgccaggacaacctttctcccagatgtaaacagagagacatgtacaaacctggattttttttttataccaccttgacccatttgctacattccttttcctgtgaaatatgtgagtgataattaaacactttagacctgattctgacttcagtttcccttatgtgcttcagaaatcagagactggggtgggggatcgaactagggttgcagactcctgggctctaaatggaaatctgagtccctaacaatcagagtgcattaaggcaagccaggctgcctcactgtgcttcctcttctgtagaatggaataacacttcatcaggtcattggtggggatcc

SEQ ID NO: 107 - DNA, 2532 bp, Homo sapiens
  Genbank Accession No. X03350
agtgcactcaagcagagaagaaatccacaaagactcaccagtctgctggtgggcagagaagacagaaacgacatgagcacagcaggaaaagtaatcaaatgcaaagcagctgtgctatgggaggtaaagaaacccttttccattgaggatgtggaggttgcacctcctaaggcttatgaagttcgcattaagatggtggctgtaggaatctgtcgcacagatgaccacgtggttagtggcaacctggtgaccccccttcctgtgattttaggccatgaggcagccggcatcgtggagagtgttggagaaggggtgactacagtcaaaccaggtgataaagtcatcccgctctttactcctcagtgtggaaaatgcagagtttgtaaaaacccggagagcaactactgcttgaaaaatgatctaggcaatcctcgggggaccctgcaggatggcaccaggaggttcacctgcagggggaagcccattcaccacttccttggcaccagcaccttctcccagtacacggtggtggatgagaatgcagtggccaaaattgatgcagcctcgcccctggagaaagtctgcctcattggctgtggattctcgactggttatgggtctgcagttaacgttgccaaggtcaccccaggctctacctgtgctgtgtttggcctgggaggggtcggcctatctgctgttatgggctgtaaagcagctggagcagccagaatcattgcggtggacatcaacaaggacaaatttgcaaaggccaaagagttgggtgccactgaatgcatcaaccctcaagactacaagaaacccatccaggaagtgctaaaggaaatgactgatggaggtgtggatttttcgtttgaagtcatcggtcggcttgacaccatgatggcttccctgttatgttgtcatgaggcatgtggcacaagcgtcatcgtaggggtacctcctgcttcccagaacctctcaataaaccctatgctgctactgactggacgcacctggaagggggctgtttatggtggctttaagagtaaagaaggtatcccaaaacttgtggctgattttatggctaagaagttttcactggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacgagatctggagcaacagctaggaaatatcattaattcagctcttcagagatgttatcaataaattacacatgggggctttccaaagaaatggaaattgatgggaaattatttttcaggaaaatttaaaattcaagtcagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagttttcactgtcgacattttcctttttctaataaaaatgtaccaaatccctggggtaaaagctagggtaaggtaaaggatagactcacatttacaagtagtgaaggtccaagagttctaaatacaggaaatttcttaggaactcaaataaaatgcccacattttactacagtaaatggcagtgtttttatgacttttatactatttctttatggtcgatatacaattgattttttaaaataatagcagatttcttgcttcatatgacaaagcctcaattactaattgtaaaaactgaactattcccagaatcatgttcaaaaaatctgtaattttgctgatgaaagtgcttcattgactaaacagtattagtttgtggctataaatgattatttaggatgatgactgaaaatgtgtataagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaagacctgtgttaatgctttgttaatgcttttcccactctcatttgttaatgctttcccactctcaggggaaggatttgcattttgagctttatctctaaatgtgacatgcaaagattattcctggtaaaggaggtagctgtctccaaaaatgctattgttgcaatatctacattctatttcatattatgaaagaccttagacataaagtaaaatagtttatca

SEQ ID NO: 108 - DNA, 3890 bp, Homo sapiens
  Genbank Accession No. J03507
atgaaggtgataagcttattcattttggtgggatttataggagagttccaaagtttttcaagtgcctcctctccagtcaactgccagtgggacttctatgccccttggtcagaatgcaatggctgtaccaagactcagactcgcaggcggtcagttgctgtgtatgggcagtatggaggccagccttgtgttggaaatgcttttgaaacacagtcctgtgaacctacaagaggatgtccaacagaggagggatgtggagagcgtttcaggtgcttttcaggtcagtgcatcagcaaatcattggtttgcaatggggattctgactgtgatgaagacagtgctgatgaagacagatgtgaggactcagaaaggagaccttcctgtgatatcgataaacctcctcctaacatagaacttactggaaatggttacaatgaactcactggccagtttaggaacagagtcatcaataccaaaagttttggtggtcaatgtagaaaggtgtttagtggggatggaaaagatttctacaggctgagtggaaatgtcctgtcctatacattccaggtgaaaataaataatgattttaattatgaattttacaatagtacttggtcttatgtaaaacatacgtcgacagaacacacatcatctagtcggaagcgctccttttttagatcttcatcatcttcttcacgcagttatacttcacataccaatgaaatccataaaggaaagagttaccaactgctggttgttgagaacactgttgaagtggctcagttcattaataacaatccagaatttttacaacttgctgagccattctggaaggagctttcccacctcccctctctgtatgactacagtgcctaccgaagattaatcgaccagtacgggacacattatctgcaatctgggtcgttaggaggagaatacagagttctattttatgtggactcagaaaaattaaaacaaaatgattttaattcagtcgaagaaaagaaatgtaaatcctcaggttggcattttgtcgttaaattttcaagtcatggatgcaaggaactggaaaacgctttaaaagctgcttcaggaacccagaacaatgtattgcgaggagaaccgttcatcagagggggaggtgcaggcttcatatctggccttagttacctagagctggacaatcctgctggaaacaaaaggcgatattctgcctgggcagaatctgtgactaatcttcctcaagtcataaaacaaaagctgacacctttatatgagctggtaaaggaagtaccttgtgcctctgtgaaaaaactatacctgaaatgggctcttgaagagtatctggatgaatttgacccctgtcattgccggccttgtcaaaatggtggtttggctactgttgaggggacccattgtctgtgccattgcaaaccgtacacatttggtgcggcgtgtgagcaaggagtcctcgtagggaatcaagcaggaggggttgatggaggttggagttgctggtcctcttggagcccctgtgtccaagggaagaaaacaagaagccgtgaatgcaataacccacctcccagtgggggtgggagatcctgcgttggagaaacgacagaaagcacacaatgcgaagatgaggagctggagcacttgaggttgcttgaaccacattgctttcctttgtctttggttccaacagaattctgtccatcacctcctgccttgaaagatggatttgttcaagatgaaggtccaatgtttcctgtggggaaaaatgtagtgtacacttgcaatgaaggatactctcttattggaaacccagtggccagatgtggagaagatttacggtggcttgttggggaaatgcattgtcagaaaattgcctgtgttctacctgtactgatggatggcatacagagtcacccccaaaaacctttctacacagttggtgagaaggtgactgtttcctgttcaggtggcatgtccttagaaggtccttcagcatttctctgtggctccagccttaagtggagtcctgagatgaagaatgcccgctgtgtacaaaaagaaaatccgttaacacaggcagtgcctaaatgtcagcgctgggagaaactgcagaattcaagatgtgtttgtaaaatgccctacgaatgtggaccttccttggatgtatgtgctcaagatgagagaagcaaaaggatactgcctctgacagtttgcaagatgcatgttctccactgtcagggtagaaattacacccttactggtagggacagctgtactctgcctgcctcagctgagaaagcttgtggtgcctgcccactgtggggaaaatgtgatgctgagagcagcaaatgtgtctgccgagaagcatcggagtgcgaggaagaagggtttagcatttgtgtggaagtgaacggcaaggagcagacgatgtctgagtgtgaggcgggcgctctgagatgcagagggcagagcatctctgtcaccagcataaggccttgtgctgcggaaacccagtaggctcctggaggccatggtcagcttgcttggaatccagcaggcagctggggctgagtgaaaacatctgcacaactgggcactggacagcttttccttcttctccagtgtctaccttcctcctcaactcccagccatctgtataaacacaatcctttgttctcccaaatctgaatcgaattactcttttgcctcctttttaatgtcagtaaggatatgagcctttgcacaggctggctgcgtgttcttgaaataggtgttaccttctctgggccttggttttttaaaatctgtaaaattagaggattgcactagagaaacttgaatgctccattcaggcctatcattttattaagtatgattgacacagcccatgggccagaacacactctacaaaatgactaggataacagaaagaacgtgatctcctgattagagagggtggttttcctcaatggaaccaaatataaagaggacttgaacaaaaatgacagatacaaactatttctatcctgagtagtaatctcacacttcatcctatagagtcaaccaccacagataggaattccttattctttttttaatttttttaagacagagtctcactttgttgcccaggctggagcgcagtggggtgatctcatctccctgcaacctccgcctcctgggttgaagcgattcttgtgcctcagcttcccaagcagctgggattacaggtgcccgccaccacgcccagctaatttttgcatttttagtagagatgggtttcaccatgttggccatgctcgtctccaactcctgacctcaggtaatccgtctgccttggcctcccaaatgctgggattacagacatgaaccaccacgcctggctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaatatatttcaaaacttcagatgacaaggattagaacactcattaagatgctattcttc

SEQ ID NO: 109 - DNA, 2941 bp, Homo sapiens
  Genbank Accession No. K01383
gaattcatgcttggactacagcttcccgccacctcccccatcttgctttttagtttaaagcagggtcagcacatcacatgaagtcatctcctttttaggggatatcccacatgccaagactaccaagcggtaggggggtggccggctaggctgtggggagcacggagatttatttgcaaaggaggacctagacaaaagagcccccacatcctctcaggcgaggagaatggacgagagtgagaggccgacccgtgttcccgtgttactgtgtacggagtagtgggtccgagggacctaggtgtggacagggacaggcaaggcgacagcgaggagaaacgaaaatcacatcggtggcggttgctctgcacacaaactccgctcgctaccgcacgctccacgctctgcactacgccgaccggggacaggagcaggaggctgtggctgcactcagacttcgggacaggccgagctgaaaaccgtgagaggggtggggtggaggcgaccgaaacgccaaggctgggttcccggaacgcgcggggaccagggtggaaggcaacttcggggaaacgggaaaggcgaccgggacctcggggacgccccgtaccccgggcgtaaactcactcccgcgttagcgggcgccaaagcggggagggggtggtcccgtggtccgcacccaggggagctcagtggactgtgcgccttgcctttctgctgcgcaaagcccagtccaggtcatcacctcgggcggggcggactcggctgggcggactcagcggggcgggcgcaggcgcagggcgggtcctttgcgtccggccctctttcccctgaccataaaagcagccgctggctgctgggccctaccaagccttccacgtgcgccttatagcctctcaacttcttgcttgggatctccaacctcaccgcggctcgaaatggaccccaactgctcctgcgccactggtaagggatgctaggtttctggtccttaggatacctatttccccgccacaggatagatgtccctaggagtagaggtgttttttgagttctagctaagtggagtcatttatttcattgatctagtgcttttccactcagcgccttcatcatccctagaacattcctattctaatgcctcccatttcgagaggcgagaggactcaggctcataattctcctgctccatgtcacccaggtagtcggggactgctagctgagccccagtactctgcgcagttcctgggcggatgggcgacacggacttattggtttttcgggattaaggacataagcccatcctagcctctacagatagaagtaagattagcttcatgtgccctgagttggacaggagctatctatcaggcctggcaatgcactcagctggcagcatttgttgaccaactgctgttatcttctgtgtaaagttcactgcctttttctcttccttgcaggtggctcctgcacctgcactggctcctgcaaatgcaaagagtgcaaatgcaactcctgcaagaagagtgagtgcggggccatctctaggaactggggctgagccaagtcagaggcaggaaaccagagctgggctaggaggagtaggccaatgatccatttcccacatcccttcccagcaactgattcaggatcagagccagatctttagacgtgatggattcccaagtttcgttcttaaaatagacaaactgaggccaagagagtgcaccaacctaccaaacacaaacatgacacctaaggactttcctcccctaagtgtgtggttctggggagccagccttcctttgtccttctaaccccagtcactgcctttccagccttctgccaggtctggggctcagatggagataagcttttcacagaagaccctcactcgaaagatccaccacttatctcccatctccgacagtgcatgccatcctgaactaagtgtcctctggggctggggacagagcttgggccaggcttctctgggggcagggaagtcccggtcaagtctagtctgacctctcactctcccttcttccccaggctgctgctcctgctgccccatgagctgtgccaagtgtgcccagggctgcatatgcaaaggggcatcagagaagtgcagctgctgtgcctgatgtccggacagccctgctcgaagatatagaaagagtgacctgcacaaacttggaattttttttccatacaaccctgacccatttactgtattttttttaatgaaatatgtgaatgataataaaagttgctgacttaatgctggctctggttttctttgtgtgccttgaaataagggaccatacaacagagctgggatggtggattgcattgagagtcagagacctgggtgctgggaccttgtgctattatcttacacactggaggcctgttaaggcaaatcaggtactcgcctcaggtcagtttcttaaataaaaatgtaagagcgtgatgacagatgatataatgccacaggaatctgtcacagacttaagctttggtaagataaaatctattagtatgacaaaaaaaaacaccaaatccattttttatattgaacaggacaatttcagtaaatatatggaagacaagataggaaaaataaaattacactacacatttgttctggatgtatattcagacttggaaacagttcacagactggccccagtatgtgggtcacacaccttcaacaccagccaagacatccccacatggaaccatcagtcttggcctgtccaggtcactgatgtgatacttggtatctctgagagctccatgttgtgacttgtctctgaatcttcggaagtttctccctaggtcatgctgaaatccgttcctcctcaagtccagcccttggttaattcaggcaagcaaagtgactgggggagtgggggaaattacccaggatcctcccagcaagctg